(19) United States
(12) Patent Application Publication
(10) Pub. No.: US 2014/0329242 A1
(43) Pub. Date: Nov. 6, 2014

(54) CHARACTERIZING MULTIPLE SCLEROSIS

(71) Applicants: Thomas M. Aune, Nashville, TN (US); Philip S. Crooke, Nashville, TN (US); Nancy J. Olsen, Dallas, TX (US); John T. Tossberg, Nashville, TN (US)

(72) Inventors: Thomas M. Aune, Nashville, TN (US); Philip S. Crooke, Nashville, TN (US); Nancy J. Olsen, Dallas, TX (US); John T. Tossberg, Nashville, TN (US)

(21) Appl. No.: 14/344,430

(22) PCT Filed: Sep. 12, 2012
(86) PCT No.: PCT/US12/54903

§ 371 Date: Jul. 7, 2014

Related U.S. Application Data
(60) Provisional application No. 61/533,599, filed on Sep. 12, 2011.

(51) Int. Cl. C12Q 1/68

(52) U.S. Cl. CPC C12Q 1/6883; C12Q 2600/112
USPC 435/6.11; 435/6.12

(57) ABSTRACT

A method for characterizing multiple sclerosis in a subject involves comparing ratios of expression levels of genes in a biological sample from a subject to references, wherein the multiple sclerosis is characterized based on a difference in the ratios of the expression values of genes in the biological sample from the subject as compared to the references.

### RELATED APPLICATIONS

This application claims priority from U.S. Provisional Application Ser. No. 61/533,599 filed Sep. 12, 2011, the entire disclosure of which is incorporated herein by this reference.

### GOVERNMENT INTEREST

This invention was made with U.S. government support under contract numbers AI53984 and AI044924 awarded by the National Institutes of Health. The U.S. government has certain rights in the invention.

### TECHNICAL FIELD

The presently-disclosed subject matter relates to the characterization of multiple sclerosis (MS) in a subject, including diagnosis of MS and exclusion of a diagnosis of MS.

### INTRODUCTION

Detection of brain lesions disseminated in space and time by magnetic resonance imaging (MRI) with gadolinium contrast is a cornerstone in the diagnosis of multiple sclerosis (MS)¹⁻³. Laboratory and clinical findings include detection of immunologic abnormalities in cerebrospinal fluid and evoked potential testing^{4, 5, 31, 32}. Clinically isolated syndrome (CIS) is a first neurologic episode lasting at least 24 hours possibly caused by focal inflammation or demyelination^{33, 34}. Approximately 10,000-15,000 new diagnoses of MS are made in the United States each year³⁵. Approximately 2-3 times that number experience a CIS each year indicating that a far greater number of subjects experience a CIS than develop MS^{36, 37, 38, 39}. The cost to healthcare of determining if a subject with a CIS has MS is significant considering the cost of MRI and additional testing that is performed and the fact that many more subjects have a CIS than develop MS.

Therefore, improved tests that can effectively, efficiently, and noninvasively characterize MS are needed, including tests to diagnose MS and/or to exclude a diagnosis of MS.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes methods useful for characterizing an auto-immune disease, and more particularly, for characterizing multiple sclerosis. The presently-disclosed subject matter further includes kits and devices useful for characterizing an auto-immune disease.

In some embodiments, a method for characterizing multiple sclerosis (MS) in a subject involves providing a biological sample from the subject; determining expression levels of at least two genes in the biological sample; calculating one or more ratios of the expression levels of the at least two genes; and comparing each ratios to a reference, wherein the is multiple sclerosisis characterized based on a difference in the ratios of the expression values of the at least two genes in the biological sample from the subject as compared to the references.

The at least two genes can be selected from those represented by SEQ ID NOs: 1-47, those corresponding to the genes set forth in Table A, or those corresponding to the genes set forth in Table B. In embodiments of the method, the expression levels of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 genes can be determined. In some embodiments, expression levels of the genes corresponding to CD55, FOS, JUN, PMAIP1, SPIB, TAF11, and TBP are determined. In some embodiments, expression levels of the genes corresponding to ACTB, CDKN1B, CTSS, GAPDH-1, KRAS, PGK1, and TBP are determined.

In accordance with the presently-disclosed subject matter, ratios of expression levels of genes are used to characterize an auto-immune disease. In this regard, ratios of interest for use in characterizing MS in a subject include the one or more ratios of expression levels of genes corresponding to those set forth in Table A, wherein each ratio is calculated by dividing the expression level of a first gene in Table A by the expression level of a second gene in Table A. In some embodiments, the at least one ratio is selected from the ratios set forth in Table B. In some embodiments, the one or more ratios consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, or 83 ratios set forth in Table B. In some embodiments, the one or more ratios consist of the ratios set forth in Column 1 (MS vs. CTRL) of Table B. In some embodiments, the one or more ratios consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 ratios set forth in Column 1 (MS vs. CTRL) of Table B. In some embodiments, the one or more ratios consist of the ratios set forth in Column 2 (MS vs. OND) of Table B. In some embodiments, the one or more ratios consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 ratios set forth in Column 2 (MS vs. OND) of Table B.

Various references can be selected for use in accordance with the presently-disclosed subject matter. In some embodiments, the reference is a standard reference ratio or a threshold value. For another example, in some embodiments, the reference is a reference ratio of a comparator group. In some embodiments, a “comparator group” or “reference group” includes individuals having a common characterization, for example, healthy control individuals, individuals who have been diagnosed with a condition often confused with an auto-immune disease of interest in the context of clinical diagnosis, individuals who have been diagnosed with an auto-immune disease of interest, or individuals who have another common characterization of interest. Expression values of biomarkers obtained from biological samples of individuals in a comparator group can be used to calculate reference ratios.

Methods of the presently-disclosed subject matter and also include comparing each subject ratio to a second reference. For example, in some embodiments, the reference can be a healthy control, and the second reference is not a healthy control. In some embodiments, the second reference comprises other neurologic disorders (OND).

Characterizing MS in a subject is inclusive of providing a diagnosis, prognosis and/or theranosis of the condition. As such, in some embodiments, characterization comprises diagnosing or prognosticating MS. In some embodiments, MS is predicted. In some embodiments, MS is not predicted. In some embodiments, the characterization comprises an exclusion of a diagnosis of MS. In some embodiments, the method also includes providing a series of biological sample obtained from the subject over a period of time. A change in the ratios in each of the biological samples from the subject can be useful for characterizing MS in the subject.

The presently-disclosed subject matter further includes kits and devices useful for detecting and/or determining expression levels of at least two genes in a biological sample.

The kits of the presently-disclosed subject matter can include primer pairs for determining expression levels of at least two genes, which can be useful for calculating ratios as disclosed herein. In some embodiments, the kit includes primer pairs for determining expression levels of at least two genes represented by SEQ ID NOs: 1-47. In some embodiments, the kit includes primer pairs for determining expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 genes represented by SEQ ID NOs: 1-47. In some embodiments, the kit includes primer pairs for determining expression levels of at least two genes corresponding to those set forth in Table A. In some embodiments, the kit includes primer pairs for determining expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 genes corresponding to those set forth in Table A. In some embodiments, the kit includes primer pairs for determining expression levels of the genes corresponding to CD55, FOS, JUN, PMAIP1, SPIB, TAF11, and TBP. In some embodiments, the kit includes primer pairs for determining expression levels of the genes corresponding to ACTB, CDKN1B, CTSS, GAPDH-1, KRAS, PGK1, and TBP. In some embodiments, the kit includes primer pairs for determining expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 genes corresponding to those set forth in Table B.

The devices of the presently-disclosed subject matter can include a probe for selectively binding each of at least two gene expression products to detect at least two genes, which can be useful for determining expression levels of the genes and for calculating ratios as disclosed herein. Such probes can selectively bind the gene products, for example, by hybridization of the probe and a nucleotide gene product. In some embodiments, the device includes probes for detecting each of at least two genes represented by SEQ ID NOs: 1-47. In some embodiments, the device includes probes for detecting each of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 genes represented by SEQ ID NOs: 1-47. In some embodiments, the device includes probes for detecting each of at least two genes corresponding to those set forth in Table A. In some embodiments, the device includes probes for detecting each of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 genes corresponding to those set forth in Table A. In some embodiments, the device includes probes for detecting each of the genes corresponding to CD55, FOS, JUN, PMAIP1, SPIB, TAF11, and TBP. In some embodiments, the device includes probes for detecting each of the genes corresponding to ACTB, CDKN1B, CTSS, GAPDH-1, KRAS, PGK1, and TBP. In some embodiments, the device includes probes for detecting each of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 genes corresponding to those set forth in Table B.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 Gene expression profiles across multiple autoimmune diseases. Expression levels of 44 target genes were determined by quantitative RT-PCR and normalized to expression of GAPDH. Expression levels of 31 genes are shown; expression levels of the remainder were not statistically different between CTRL and any disease cohort. Genes are identified that showed statistically significant [P <0.05 after Bonferroni's correction] increased or decreased expression in individual disease cohorts relative to CTRL subjects. Numerical expression ratios [disease group average/CTRL average] are displayed within the colored boxes.

FIG. 2 Discrimination between MS and CTRL subjects with an 8 ratio scoring system. (a) Performance of the single ratio, ANAPC1/CHEK2 to discriminate MS and CTRL subjects. (b) Genes making up 8 unique discriminatory ratios. P values compare expression levels of ratios between MS and CTRL subjects. (c) Increased sensitivity with increasing numbers of ratios. (d) Score distributions among subjects using 8 ratios. (e) Validation of results by analyzing 40 new MS subjects and 40 new CTRL subjects. (f) Score distribution between OND-I and OND-NI subjects. (g) Mean scores±std. dev. among subjects with CIS, initial diagnosis of MS, and established MS. P value is not significant among groups. (h) Mean scores±std. dev. among MS subjects from different geographic locations. P value is not significant among groups.

FIG. 3 Discrimination of MS subjects from subjects with inflammatory neurologic diseases, TM or NMO. Most discriminatory gene expression ratios were identified that segregate MS subjects from TM and NMO subjects (CTRL is included for reference). The point system was applied to combine ratio performance into a single discriminator.

FIG. 4 Discrimination of subjects with Parkinson's disease from MS and CTRL. Most discriminatory gene expression ratios were identified that segregate Parkinson's disease subjects from MS subjects and CTRL subjects. Using the point system, the % of Parkinson's subjects with a score >0, Y-axis, relative to the number of ratios, X-axis, for the different comparator groups was determined.

FIG. 5 Discrimination of MS subjects from heterogeneous comparator groups. The top 15 gene expression ratios with the greatest ability to discriminate MS from OND-I, OND-NI, or ALL (OND-I, OND-NI, and CTRL) were identified. Using the point system, the % of MS subjects with a score >0, Y-axis, relative to the number of ratios, X-axis, for the different comparator groups [CTRL is included for reference] were determined.

FIG. 6 Discrimination between MS and OND-I subjects using 10 gene expression ratios. (a) Genes making up 10 unique discriminatory ratios. P values compare individual ratio values between MS and OND-I subjects. (b) Increasing number of ratios increases sensitivity or ability to discriminate between MS and OND-I subjects. (c) The score distribution in MS and OND-I subjects using 10 ratios. (d) Validation of results by analyzing 40 new MS subjects and 40 new OND-I subjects (20 TM+20 NMO). (e) Mean scores±std. dev. among subjects with CIS, initial diagnosis of MS and established MS. P is not significant for CIS versus MS naïve, 0.03 for CIS versus established MS, and <0.0001 for MS naïve versus established MS. (f) Mean scores±std. dev. among subjects based upon geographic sites. P is not significant for Nashville versus Europe, <0.0001 for Nashville versus U.S. non-Nashville, and <0.0001 for Europe versus U.S. non-Nashville. (g) Score distributions between [CIS and MS-naïve] and established MS.

FIG. 7 Discrimination between MS and OND-NI subjects using 10 gene expression ratios. (a) Identification of genes making up the 10 unique discriminatory ratios. P values compare individual ratio values between MS and OND-NI subjects. (b) Increasing the number of gene expression ratio increases the ability to discriminate between MS and OND-NI subjects. (c) Score distribution using 10 ratios in the training set. (d) Validation of results by analyzing 40 new MS subjects and 40 new OND-NI subjects. (e) Mean scores±std. dev. among subjects with CIS, initial diagnosis of MS and established MS. P values were not significant among any of the comparisons. (f) Mean scores±std. dev. among subjects based upon geographic sites. P values were not significant for any of the comparisons.

FIG. 8 Flow chart describing sample collection and processing, data generation, and methods of data analysis.

FIG. 9 Gene-expression profiles in subjects with CIS, MS-naïve or MS-established. (a) Expression levels of 23 target genes were determined by quantitative reverse-transcription PCR and normalized to expression of GAPDH. Results are expressed as the ratio of the expression level of the indicated genes in the disease cohort relative to the CTRL cohort, log₂. Genes are identified that showed statistically significant (P <0.05 after Bonferroni's correction for multiple testing) increased or decreased expression. Numerical expression ratios, log), of the test/CTRL cohorts are displayed within the boxes. (b) Cumulative percentage of over- and under-expressed genes in each disease cohort relative to CTRL. (c) Statistical significance of the expression level of each target gene between each disease cohort and CTRL was determined using Student's T test. P values are expressed as log₁₀.

FIG. 10 (a) Ratios that make up the ratioscore discriminating MS from CTRL. Columns represent individual ratios. Rows represent individual subjects within the MS cohort. Black/dark grey in the heatmap denotes individual subjects with the value of the individual ratio greater than the value of the ratio in all subjects within the CTRL cohort. Light grey/white denotes individual subjects with the value of the individual ratio less than or equal to the highest ratio value in all subjects within the CTRL cohort. (b) Results from inputting independent CIS→MS subjects into the ratioscore algorithm.

FIG. 11 (a) The ratioscore method discriminates between MS and OND subjects. Ratios that make up the ratioscore to discriminate MS from OND. Columns represent individual ratios. Rows represent individual subjects within the MS cohort. Black/dark grey in the heatmap denotes individual subjects with the value of the individual ratio greater than the value of the ratio in all subjects within the CTRL cohort. Light grey/white denotes individual subjects with the value of the individual ratio less than or equal to the highest ratio value in all subjects within the CTRL cohort. (b) Results from inputting independent CIS→MS subjects into the ratioscore algorithm.

FIG. 12 a. Ability of the radioscore method to discriminate between MS and combined CTRL plus OND subjects. Columns represent individual ratios. Rows represent individual subjects within the MS cohort. Black/dark grey in the heatmap denotes individual subject with the value of the individual ratio greater than the value of the ratio in all subjects within the CTRL cohort. Light grey/white denotes individual subjects with the value of the individual ratio less than or equal to the highest ratio value in all subjects within the CTRL cohort. b. Results from inputting independent CIS→MS subjects into the ratioscore alcorithm.

FIG. 13 Ratios making up the ratioscore that discriminate MS from OND-NI or OND-I. a. Optimum ratios to discriminate MS from OND-I. b. Results for individual CIS→MS subjects using the MS: OND-I ratioscore. c. Optimum ratios to discriminate MS from OND-NI. d. Results for individual CIS→MS subjects using the OND-NI ratioscore.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes methods, devices, and kits useful for characterizing an auto-immune disease in a subject and, more particularly, for characterizing multiple sclerosis (MS) in a subject. In some embodiments, the method involves providing a biological sample from the subject; determining expression values of at least two genes in the biological sample; calculating one or more ratios of the expression values of the at least two genes; and comparing each ratios to a reference, wherein the MS is characterized based on a difference in the ratios of the expression values of the at least two genes in the biological sample from the subject as compared to the references. In some embodiments, the biological sample is blood obtained from the subject or another biological sample containing a cell obtained from the subject, e.g., a subject suspected of having MS. The method can be used, in some embodiments, to diagnose the subject with MS. In some embodiments, the method can be used to exclude the subject from a diagnosis of MS.

Methods of the presently-disclosed methods include determining expression values of genes in biological samples. As such, nucleic acid molecules or nucleotides are relevant to the disclosed subject matter. Nucleotides or genes, the expression of which is desired to be determined for characterizing an auto-immune disease, include, but are not limited to those identified in Table A, the isolated nucleic acid molecules of any one of SEQ ID NOs: 1-47, fragments of the isolated nucleic acid molecules of any one of SEQ ID NOs: 1-47 where detection of such fragments are indicative of expression of an associated gene, e.g., as identified in Table A, complementary nucleic acid molecules, isolated nucleic acid molecules capable of hybridizing to any one of the SEQ ID NOs: 1-47 under conditions disclosed herein, and corresponding RNA and/or DNA molecules.

As used herein, “nucleic acid” and “nucleic acid molecule” refer to any of deoxyribonucleic acid (DNA), ribonucleic acid (RNA), oligonucleotides, fragments generated by the polymerase chain reaction (PCR), and fragments generated by any of ligation, scission, endonuclease action, and exonuclease action. The term “isolated”, when used in the context of an isolated DNA molecule or an isolated polypeptide, is a DNA molecule or polypeptide that, by the hand of man, exists apart from its native environment and is therefore not a product of nature.

Unless otherwise indicated, a particular nucleotide sequence also implicitly encompasses complementary sequences, subsequences, elongated sequences, as well as the sequence explicitly indicated. The terms “nucleic acid molecule” or “nucleotide sequence” can also be used in place of “gene”, “cDNA”, or “mRNA”. Nucleic acids can be derived from any source, including any organism. In one embodiment, a nucleic acid is derived from a biological sample isolated from a subject.

The terms “complementary” and “complementary sequences”, as used herein, refer to two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth herein, or is defined as being capable of hybridizing to the nucleic acid segment in question under conditions such as those described herein. In one embodiment, a complementary sequence is at least 80% complementary to the nucleotide sequence with which is it capable of pairing. In another embodiment, a complementary sequence is at least 85% complementary to the nucleotide sequence with which is it capable of pairing. In another embodiment, a complementary sequence is at least 90% complementary to the nucleotide sequence with which is it capable of pairing. In another embodiment, a complementary sequence is at least 95% complementary to the nucleotide sequence with which is it capable of pairing. In another embodiment, a complementary sequence is at least 98% complementary to the nucleotide sequence with which is it capable of pairing. In another embodiment, a complementary sequence is at least 99% complementary to the nucleotide sequence with which is it capable of pairing. In still another embodiment, a complementary sequence is at 100% complementary to the nucleotide sequence with which is it capable of pairing. A particular example of a complementary nucleic acid segment is an antisense oligonucleotide.

“Stringent hybridization conditions” in the context of nucleic acid hybridization experiments are both sequence- and environment-dependent. Longer sequences hybridize specifically at higher temperatures. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Typically, under “stringent conditions” a probe hybridizes specifically to its target sequence, but to no other sequences. An extensive guide to the hybridization of nucleic acids is found in Tijssen 1993, which is incorporated herein by this reference. In general, a signal to noise ratio of 2-fold (or higher) than that observed for a negative control probe in a same hybridization assay indicates detection of specific or substantial hybridization.

It is understood that in order to determine a gene expression level by hybridization, a full-length cDNA need not be employed. To determine the expression level of a gene represented by one of SEQ ID NOs: 1-47, any representative fragment or subsequence of the sequences set forth in SEQ ID NOs: 1-47 can be employed in conjunction with the hybridization conditions disclosed herein. As a result, a nucleic acid sequence used to assay a gene expression level can comprise sequences corresponding to the open reading frame (or a portion thereof), the 5′ untranslated region, and/or the 3′ untranslated region. It is understood that any nucleic acid sequence that allows the expression level of a reference gene to be specifically determined can be employed with the methods and compositions of the presently disclosed subject matter.

As used herein, the terms “corresponding to” and “representing”, “represented by” and grammatical derivatives thereof, when used in the context of a nucleic acid sequence corresponding to or representing a gene, refers to a nucleic acid sequence that results from transcription, reverse transcription, or replication from a particular genetic locus, gene, or gene product (for example, an mRNA). In other words, a partial cDNA, or full-length cDNA corresponding to a particular reference gene is a nucleic acid sequence that one of ordinary skill in the art would recognize as being a product of either transcription or replication of that reference gene (for example, a product produced by transcription of the reference gene). One of ordinary skill in the art would understand that the partial cDNA, or full-length cDNA itself is produced by in vitro manipulation to convert the mRNA into a cDNA, for example by reverse transcription of an isolated RNA molecule that was transcribed from the reference gene. One of ordinary skill in the art will also understand that the product of a reverse transcription is a double-stranded DNA molecule, and that a given strand of that double-stranded molecule can embody either the coding strand or the non-coding strand of the gene. The sequences presented in the Sequence Listing are single-stranded, however, and it is to be understood that the presently claimed subject matter is intended to encompass the genes represented by the sequences presented in SEQ ID NOs: 1-47, including the specific sequences set forth as well as the reverse/complement of each of these sequences.

The term “gene expression” generally refers to the cellular processes by which a biologically active polypeptide is produced from a DNA sequence. Generally, gene expression comprises the processes of transcription and translation, along with those modifications that normally occur in the cell to modify the newly translated protein to an active form and to direct it to its proper subcellular or extracellular location.

The terms “gene expression level” and “expression level” as used herein refer to an amount of gene-specific RNA or polypeptide that is present in a biological sample. When used in relation to an RNA molecule, the term “abundance” can be used interchangeably with the terms “gene expression level” and “expression level”.

Determination of expression levels of genes of interest can be achieved using any technique known the skilled artisan. For example, in some embodiments, RNA can be purified from the biological sample, converted to the more-stable complementary DNA (cDNA), before the gene expression products of genes of interest are detected. As will be recognized by the skilled artisan, where amplification of the sample is desired, polymerase chain reaction amplification can be employed. Determining the expression levels can be achieved, for example, using reverse transcription-polymerase chain reaction (RT-PCR), microarray analysis, or other techniques known to the skilled artisan.

In some embodiments, determining the expression levels of genes in the biological sample includes determining the expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, or 47 genes represented by SEQ ID NOs: 1-47. In some embodiments, determining the expression levels of genes in the biological sample includes determining the expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 genes corresponding to those set forth in Table A.

TABLE A
Genes

| Gene | | | ABI Assay | SEQ ID |
|---|---|---|---|---|
| Abbreviation | Gene | NCBI Ref. No. | Number: | NO: |
| ABR | active BCR-related gene, transcript | NM_001159746.1 | Hs00254300_m1 | 1 |
| | variant 3 | | | |
| ACTB | actin, beta | NM_001101.3 | Hs99999903_m1 | 2 |
| ACTR1A | ARP1 actin-related protein 1 | NM_005736.3 | Hs00194913_m1 | 3 |
| | homolog A, centractin alpha (yeast) | | | |
| ADAMTSL4 | ADAMTS-like 4 (ADAMTSL4), | NM_019032.4 | Hs00296775_m1 | 4 |
| | transcript variant 1 | | | |
| ANAPC1 | anaphase promoting complex | NM_022662.2 | Hs00224096_m1 | 5 |
| | subunit 1 | | | |
| APOBEC3F | apolipoprotein B mRNA editing | NM_145298.5 | Hs00272529_m1 | 6 |
| | enzyme, catalytic polypeptide-like | | | |
| | 3F | | | |
| ASL | argininosuccinate lyase | NM_001024943.1 | Hs00163695_m1 | 7 |
| B2M | beta-2-microglobulin | NM_004048.2 | Hs99999907_m1 | 8 |
| BRCA1 | breast cancer 1, early onset | NR_027676.1 | Hs00173237_m1 | 9 |
| | (BRCA1), transcript variant 6, non- | | | |
| | coding RNA | | | |
| CD55 | CD55 molecule, decay accelerating | NM_000574.3 | Hs00167090_m1 | 10 |
| | factor for complement (Cromer | | | |
| | blood group), transcript variant 1 | | | |
| CDH1 | cadherin 1, type 1, E-cadherin | NM_004360.3 | Hs00170423_m1 | 11 |
| | (epithelial) | | | |
| CDKN1B | cyclin-dependent kinase inhibitor | NM_004064.3 | Hs00153277_m1 | 12 |
| | 1B (p27, Kip1) | | | |
| CHEK2 | checkpoint kinase 2 (CHEK2), | NM_001005735.1 | Hs00200485_m1 | 13 |
| | transcript variant 3 | | | |
| CSF3R | colony stimulating factor 3 receptor | NM_156039.3 | Hs00167918_m1 | 14 |
| | (granulocyte), transcript variant 3 | | | |
| CTSS | cathepsin S, transcript variant 1 | NM_004079.4 | Hs00175403_m1 | 15 |
| EPHX2 | epoxide hydrolase 2, cytoplasmic | NM_001979.4 | Hs00157403_m1 | 16 |
| EXT2 | exostosin 2, transcript variant 2 | NM_207122.1 | Hs00181158_m1 | 17 |
| FOS | FBJ murine osteosarcoma viral | NM_005252.3 | Hs00170630_m1 | 18 |
| | oncogene homolog | | | |
| FOSL1 | FOS-like antigen 1 | NM_005438.3 | Hs00759776_s1 | 19 |
| FOXN3 | forkhead box N3, transcript variant 1 | NM_001085471.1 | Hs00231993_m1 | 20 |
| GAPDH-1 | glyceraldehyde-3-phosphate | NM_002046.3 | Hs99999905_m1 | 21 |
| | dehydrogenase | | | |
| GAPDH-2 | glyceraldehyde-3-phosphate | NM_002046.3 | Hs99999905_m1 | 22 |
| | dehydrogenase | | | |
| GATA3 | GATA binding protein 3 | NM_001002295.1 | Hs00231122_m1 | 23 |
| GNB5 | guanine nucleotide binding protein | NM_006578.3 | Hs00275095_m1 | 24 |
| | (G protein), beta 5, transcript | | and | |
| | variant 1 | | Hs01034253_m1 | |
| GSTM4 | glutathione S-transferase mu 4, | NM_147148.2 | Hs00426432_m1 | 25 |
| | transcript variant 2 | | | |
| HLA-DRA | major histocompatibility complex, | NM_019111.4 | Hs00219575_m1 | 26 |
| | class II, DR alpha | | | |
| HRAS | v-Ha-ras Harvey rat sarcoma viral | NM_001130442.1 | Hs00610483_m1 | 27 |
| | oncogene homolog (HRAS), | | | |
| | transcript variant 3 | | | |
| IFI27 | interferon, alpha-inducible protein | NM_001130080.1 | Hs00271467_m1 | 28 |
| | 27 (IFI27), transcript variant 1 | | | |
| IL11RA | interleukin 11 receptor, alpha, | NM_001142784.1 | Hs00234415_m1 | 29 |
| | transcript variant 3 | | | |
| JUN | jun proto-oncogene | NM_002228.3 | Hs00277190_s1 | 30 |
| KRAS | v-Ki-ras2 Kirsten rat sarcoma viral | NM_004985.3 | Hs00270666_m1 | 31 |
| | oncogene homolog, transcript | | | |
| | variant b | | | |
| LEPREL4 | leprecan-like 4 | NM_006455.2 | Hs00197668_m1 | 32 |
| LLGL2 | lethal giant larvae homolog 2 | NM_001015002.1 | Hs00189729_m1 | 33 |
| | (Drosophila), transcript variant 2 | | | |
| NRAS | neuroblastoma RAS viral (v-ras) | NM_002524.4 | Hs00180035_m1 | 34 |
| | oncogene homolog | | | |
| OAS1 | 2′-5′-oligoadenylate synthetase 1, | NM_001032409.1 | Hs00242943_m1 | 35 |
| | 40/46 kDa, transcript variant 3, | | | |
| ORC1 | origin recognition complex, subunit | NM_001190819.1 | Hs00172751_m1 | 36 |
| | 1 (ORC1), transcript variant 3 | | | |
| PGK1 | phosphoglycerate kinase 1 | NM_000291.3 | Hs99999906_m1 | 37 |
| PMAIP1 | phorbol-12-myristate-13-acetate- | NM_021127.2 | Hs00560402_m1 | 38 |
| | induced protein 1 | | | |
| POU6F1 | POU class 6 homeobox 1, | NR_026893.1 | Hs00231276_m1 | 39 |
| | transcript variant 2 | | | |
| RANGAP1 | Ran GTPase activating protein 1 | NM_002883.2 | Hs00610049_m1 | 40 |
| SPIB | Spi-B transcription factor (Spi-1/PU.1 | NM_003121.3 | Hs00162150_m1 | 41 |
| | related) | | | |
| TAF11 | TAF11 RNA polymerase II, TATA | NM_005643.2 | Hs00194573_m1 | 42 |
| | box binding protein (TBP)- | | | |
| | associated factor, 28 kDa | | | |
| TBP | TATA box binding protein, | NM_001172085.1 | Hs00427620_m1 | 43 |
| | transcript variant 2 | | | |
| TGFBR2 | transforming growth factor, beta | NM_001024847.2 | Hs00559661_m1 | 44 |
| | receptor II (70/80 kDa), transcript | | | |
| | variant 1 | | | |
| TP53 | tumor protein p53 (TP53), | NM_001126113.1 | Hs00153340_m1 | 45 |
| | transcript variant 4 | | | |
| TP53-2 | tumor protein p53 (TP53), | NM_001126112.1 | Hs01034253_m1 | 46 |
| | transcript variant 2 | | | |
| TXK | TXK tyrosine kinase | NM_003328.2 | Hs00177433_m1 | 47 |
| IL11R1 | | | | |

In some embodiments, determining the expression levels of genes in the biological sample includes determining the expression levels of the genes corresponding to CD55, FOS, JUN, PMAIP1, SPIB, TAF11, and TBP. In some embodiments, determining the expression levels of genes in the biological sample includes determining the expression levels of the genes corresponding to ACTB, CDKN1B, CTSS, GAPDH-1, KRAS, PGK1, and TBP. In some embodiments, determining the expression levels of genes in the biological sample includes determining the expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 genes corresponding to those set forth in Table B.

As used herein, a “ratio” or “expression ratio” is the expression value of a first biomarker (numerator) divided by the expression value of a second biomarker (denominator), e.g., Gene A/Gene B. As such, once the expression levels of at least two genes are determined, a ratio can be calculated. Ratios can be calculated using expression levels of genes in a biological sample obtained from a subject. In some embodiments, a reference can be a ratio calculated using expression levels of genes from another source. As such, the term “subject ratio” can used herein to refer to a ratio calculated using expression values of a gene pair in a biological sample obtained from a subject, while the term “reference ratio” can be used to refer to a ratio of the same biomarker pair in a reference sample, which serves as a reference to which the subject ratio is compared.

TABLE B
Ratios

| | |
|---|---|
| MS vs. CTRL | MS vs. OND |
| Expression Ratios | Expression Ratios |

| Numerator | Denominator | Numerator | Denominator |
|---|---|---|---|
| JUN | CD55 | PGK1 | CTSS |
| JUN | SPIB | CDKN1B | GAPDH-1 |
| TAF11 | FOS | KRAS | CTSS |
| PMAIP1 | TBP | ACTB | TBP |
| TAF11 | FOSL1 | APOBEC3F | GAPDH-2 |
| KRAS | ASL | KRAS | OAS1 |
| GATA3 | ANAPC1 | KRAS | ASL |
| B2M | FOSL1 | CSF3R | CD55 |
| OAS1 | GAPDH-2 | OAS1 | GSTM4 |
| TBP | GSTM4 | CSF3R | TBP |
| CTSS | CDKN1B | APOBEC3F | LLGL2 |
| PMAIP1 | ASL | APOBEC3F | TAF11 |
| GSTM4 | CDKN1B | TP53-1 | CDKN1B |
| TP53 | LLGL2 | FOS | CD55 |
| GATA3 | LLGL2 | APOBEC3F | CDKN1B |
| GAPDH-2 | EXT2 | OAS1 | B2M |
| GAPDH-1 | EXT2 | GNB5 | EXT2 |
| RANGAP1 | ASL | RANGAP1 | IL11RA |
| TP53 | POU6F1 | FOS | B2M |
| CSF3R | LLGL2 | TGFBR2 | B2M |
| IL11RA | EXT2 | APOBEC3F | ASL |
| IL11R1 | TAF11 | TGFBR2 | CDKN1B |
| ANAPC1 | LLGL2 | ANAPC1 | EXT2 |
| FOS | OAS1 | APOBEC3F | TBP |
| ANAPC1 | POU6F1 | GSTM4 | EXT2 |
| CSF3R | CDKN1B | GNB5 | CDKN1B |
| GSTM4 | EXT2 | TGFBR2 | EXT2 |
| ANAPC1 | ASL | JUN | PMAIP1 |
| HLA-DRA | GNB5 | RANGAP1 | TBP |
| TP53-2 | KRAS | ANAPC1 | ASL |
| GSTM4 | EPHX2 | TP53-2 | B2M |
| GAPDH-1 | TBP | TBP | CTSS |
| EPHX2 | OAS1 | TP53-2 | CTSS |
| JUN | TAF11 | EPHX2 | PMAIP1 |
| RANGAP1 | EPHX2 | ACTB | ASL |
| CSF3R | TBP | ASL | PMAIP1 |
| HLA-DRA | LLGL2 | OAS1 | FOSL1 |
| ANAPC1 | TBP | CSF3R | TP53-1 |
| ANAPC1 | EPHX2 | EPHX2 | CDKN1B |
| CTSS | CD55 | CTSS | EXT2 |
| TP53-2 | ACTB | B2M | CD55 |
| CTSS | PMAIP1 | | |

In embodiments of the presently-disclosed subject matter, the method involves calculating one or more ratios of expression levels of genes corresponding to those set forth in Table A, wherein each ratio is calculated by dividing the expression level of a first gene in Table A by the expression level of a second gene in Table A.

In embodiments of the presently-disclosed subject matter, the method involves calculating one or more ratios set forth in Table B. In some embodiments, the method includes calculating 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, or 83 ratios set forth in Table B.

In embodiments of the presently-disclosed subject matter, the method involves calculating one or more ratios set forth in Column 1 (MS vs. CTRL) of Table B. In some embodiments, the method includes calculating 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 ratios set forth in Column 1 (MS vs. CTRL) of Table B.

In embodiments of the presently-disclosed subject matter, the method involves calculating one or more ratios set forth in Column 2 (MS vs. OND) of Table B. In some embodiments, the method includes calculating 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 ratios set forth in Column 2 (MS vs. OND) of Table B.

Various references are appropriate for use in connection with the presently-disclosed subject matter, with non-limiting examples described herein. In some embodiments, the reference comprises a reference ratio calculated using of the expression level of two genes in a biological sample taken from one or more individuals, which two genes are the same two genes used to calculate the subject ratio. The expression levels of genes in biological samples from one or more individuals can be a expression levels from a reference group or comparator group.

In some embodiments, a “comparator group” or “reference group” includes individuals having a common characterization, for example, healthy control individuals, individuals who have been diagnosed with a condition often confused with an auto-immune disease of interest in the context of clinical diagnosis, individuals who have been diagnosed with an auto-immune disease of interest, or individuals who have another common characterization of interest. Expression values of biomarkers obtained from biological samples of individuals in a comparator group can be used to calculate reference ratios. Data associated with one or more comparator groups can be stored, for example, in a database that can be accessed when practicing a method in accordance with the presently-disclosed subject matter.

With reference to Table B, for example, ratios-of-interest are provided for use with a healthy control comparator group (CTRL, column 1) or a comparator group of individuals having other neurologic disorders (OND, column 2). Examples of comparator groups relevant to characterization of MS include, but are not limited to: healthy control (CTRL), clinically isolated syndrome (CIS), CIS later developing MS (CIS→MS), MS diagnosed, newly diagnosed with MS who have not yet begun treatment (MS Naïve), established MS (>1 year), other neurologic disorders (OND), e.g., Alzheimer's disease, ataxia, Bell's palsy, cerebellar ataxia, cerebral bleed, cervical radiculopathy, Charcot-Marie tooth disease, CNS Lupus, dizziness/pituitary, drug-induced movement disorder, drug-induced tremor, dystonia, epilepsy, essential tremor, Huntington's disease, hydrocephalus, median neuropathy, meningioma, meningitis, migraine, Parkinson's disease, peripheral neuropathy, pituitary adenoma, pseudotumor cerebri, RLS, seizures, spasmodic torticollis, stroke, tension headache, Tourette's syndrome, transient ischemic attack, tremor, and trigeminal tremor. For some comparator groups, ONDs can be grouped by those typically considered inflammatory (OND-I) and those typically considered non-inflammatory (OND-NI).

Because a comparator group can include data from multiple individuals, as will be recognized by one of ordinary skill in the art, it is expected that the expression values of biomarkers in biological samples obtained from different individuals in the same comparator group might differ. As such, identification of a reference ratio for a particular gene pair can be made with reference to a “threshold reference ratio” for the gene pair within the comparator group. In some embodiments, for example, the threshold expression ratio could be a median, an average, a value based on statistical analysis of the distribution of ratios of expression levels of the gene pair within the comparator group, or another threshold value, e.g., top value in the group, second highest value in the group, third highest value in the group, etc.

In some embodiments, the reference comprises a reference ratio calculated using a standard sample containing standard biomarker amounts, which can be analyzed in the same manner or even concurrently with the biological sample. In some embodiments, the reference comprises ratio values, such as standard threshold values. Such values can be published in a format useful for the practitioner, such as in a list, table, database, or incorporated into a software or system for use in connection with the presently-disclosed subject matter. Such values can in some cases be based, for example, on information obtained from a comparator group.

Ratios of interest, or ratios of gene pairs that are useful for characterizing MS, have the ability to distinguish to groups, e.g., MS group and health control group. Table B includes examples of ratios of interest for MS vs. healthy control (CTRL) and MS vs. other neurologic disorders (OND). In this regard, an auto-immune disease can be characterized based on a difference in the ratios of the expression values of at least two genes in a biological sample from the subject as compared to a reference ratio.

In some embodiments, it can be useful to compare one or more subject ratios to one or more first reference ratios, e.g., from a first comparator group, and also to compare the one or more subject ratios to one or more second reference ratios, e.g., from a second comparator group. Such a multi-tiered approach can improve the efficacy of the characterization of the MS, as will be explained further in the Examples section.

Characterizing can include providing a diagnosis, prognosis, and/or theragnosis of an auto-immune disease in a subject.

“Making a diagnosis” or “diagnosing,” as used herein, are further inclusive of making a prognosis, which can provide for predicting a clinical outcome (with or without medical treatment), selecting an appropriate treatment (or whether treatment would be effective), or monitoring a potential auto-immune disease, based on calculated ratios of expression levels of genes. Diagnostic testing that involves treatment, such as treatment monitoring or decision making can be referred to as “theranosis.” Further, in some embodiments of the presently disclosed subject matter, multiple determinations of ratios of expression levels of genes over time can be made to facilitate diagnosis (including prognosis), evaluating treatment efficacy, and/or progression of a potential auto-immune disease or auto-immune disease. A temporal change in one or more ratios can be used to predict a clinical outcome, monitor the progression of the condition, and/or efficacy of administered therapies. In such an embodiment for example, one could observe a change in a particular ratio in a biological sample over time during the progression of a condition and/or during the course of a therapy.

The presently disclosed subject matter further provides in some embodiments a method for theranostic testing, such as evaluating progression of a condition and/or treatment efficacy in a subject. In some embodiments, the method comprises providing a series of biological samples over a time period from the subject; determining expression values of at least two genes in each of the biological samples; calculating one or more ratios of the expression values of the at least two genes for each of the biological samples; and determining any measurable change in the ratios in each of the biological samples from the series to thereby evaluate progression of the condition and/or treatment efficacy.

Any changes in the ratios, and changes in the ratios relative to references, over the time period can be used to make a diagnosis, predict clinical outcome, determine whether to initiate or continue the therapy, and whether a current therapy is effectively.

The phrase “determining the prognosis” as used herein refers to methods by which the skilled artisan can predict the course or outcome of a condition in a subject. The term “prognosis” can refer to the ability to predict the course or outcome of a condition with up to 100% accuracy, or predict that a given course or outcome is more or less likely to occur based on the ratios of expression values of genes of interest. The term “prognosis” can also refer to an increased probability that a certain course or outcome will occur; that is, that a course or outcome is more likely to occur in a subject when compared to individuals in a comparator group. For example, in individuals exhibiting subject ratios-of-interest that are higher than reference ratio-of-interest, the chance of a given outcome (e.g., MS diagnosis) may be very high. In certain embodiments, a prognosis is about a 5% chance of a given expected outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, or about a 95% chance.

The skilled artisan will understand that associating a prognostic indicator with a predisposition to an adverse outcome can be performed using statistical analysis. For example, subject ratios that are higher than reference ratios in some embodiments can signal that a subject is more likely to suffer from an auto-immune disease than subjects with ratios that are substantially equal to reference ratios, as determined by a level of statistical significance. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001. When performing multiple statistical tests, p values can be corrected for multiple comparisons using techniques known in the art.

Further with respect to the methods of the presently disclosed subject matter, a preferred subject is a vertebrate subject. A preferred vertebrate is warm-blooded; a preferred warm-blooded vertebrate is a mammal. A mammal is most preferably a human. As used herein, the term “subject” includes both human and animal subjects. Thus, veterinary therapeutic uses are provided in accordance with the presently disclosed subject matter.

As such, the presently disclosed subject matter provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The presently-disclosed subject matter further includes kits and devices useful for detecting and/or determining expression levels of at least two genes in a biological sample.

The kits of the presently-disclosed subject matter can include primer pairs for determining expression levels of at least two genes, which can be useful for calculating ratios as disclosed herein. In some embodiments, the kit includes primer pairs for determining expression levels of at least two genes represented by SEQ ID NOs: 1-47. In some embodiments, the kit includes primer pairs for determining expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 genes represented by SEQ ID NOs: 1-47. In some embodiments, the kit includes primer pairs for determining expression levels of at least two genes corresponding to those set forth in Table A. In some embodiments, the kit includes primer pairs for determining expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 genes corresponding to those set forth in Table A. In some embodiments, the kit includes primer pairs for determining expression levels of the genes corresponding to CD55, FOS, JUN, PMAIP1, SPIB, TAF11, and TBP. In some embodiments, the kit includes primer pairs for determining expression levels of the genes corresponding to ACTB, CDKN1B, CTSS, GAPDH-1, KRAS, PGK1, and TBP. In some embodiments, the kit includes primer pairs for determining expression levels of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 genes corresponding to those set forth in Table B.

The devices of the presently-disclosed subject matter can include a probe for selectively binding each of at least two gene expression products to detect at least two genes, which can be useful for determining expression levels of the genes and for calculating ratios as disclosed herein. Such probes can selectively bind the gene products, for example, by hybridization of the probe and a nucleotide gene product. In some embodiments, the device includes probes for detecting each of at least two genes represented by SEQ ID NOs: 1-47. In some embodiments, the device includes probes for detecting each of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 genes represented by SEQ ID NOs: 1-47. In some embodiments, the device includes probes for detecting each of at least two genes corresponding to those set forth in Table A. In some embodiments, the device includes probes for detecting each of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 genes corresponding to those set forth in Table A. In some embodiments, the device includes probes for detecting each of the genes corresponding to CD55, FOS, JUN, PMAIP1, SPIB, TAF11, and TBP. In some embodiments, the device includes probes for detecting each of the genes corresponding to ACTB, CDKN1B, CTSS, GAPDH-1, KRAS, PGK1, and TBP. In some embodiments, the device includes probes for detecting each of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 genes corresponding to those set forth in Table B.

Some of the gene sequences disclosed herein are cross-referenced to GENBANK® accession numbers. The sequences cross-referenced in the GENBANK® database are expressly incorporated by reference as are equivalent and related sequences present in GENBANK® or other public databases. Also expressly incorporated herein by reference are all annotations present in the GENBANK® database associated with the sequences disclosed herein. Unless otherwise indicated or apparent, the references to the GENBANK® database are references to the most recent version of the database, as of the filing date of this application.

While the terms used herein are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples may include compilations of data that are representative of data gathered at various times during the course of development and experimentation related to the present invention.

### EXAMPLES

### Example 1

### Gene Expression Patterns in Distinct Neurologic Diseases

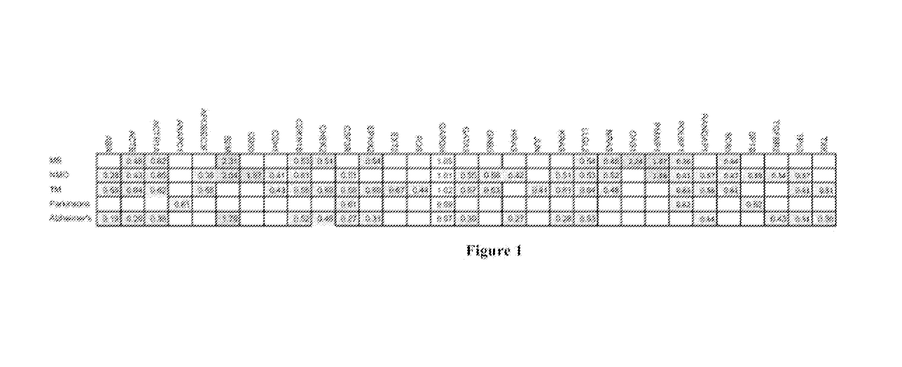

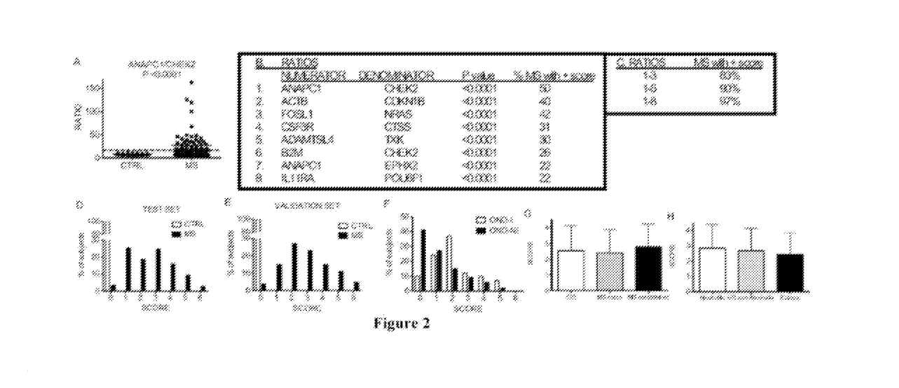

Expression patterns of a common set of genes assayed using a common platform in control subjects and subjects with different neurologic conditions, including autoimmune diseases, were measured. Expression levels of individual genes were determined by quantitative RT-PCR by normalization to GAPDH expression levels. A heatmap was employed to depict those genes differentially expressed in individual disease cohorts relative to the control cohort, P <0.05 (after Bonferroni correction for multiple testing) (FIG. 1). Ratios of expression levels of individual genes in the indicated disease cohort relative to the control cohort were calculated and depicted within each colored box. Each disease exhibited an underlying unique pattern of gene expression. However, these profiles were sufficiently overlapping to prohibit accurate discrimination of one disease from another disease using the expression profile alone. For example, LLGL2, RANGAP1, ACTB, and POU6F1 were under-expressed in 4, 3, 4, and 4 of 5 different conditions, respectively. In contrast, other genes, e.g., ANAPC1 in Parkinson's disease, EXT2 and FOS in TM, HRAS in NMO, were only differentially expressed in a single disease cohort. Overall, individual genes were either over-expressed, e.g. B2M, CD55, PMAIP1, or under-expressed, e.g. LLGL2, RANGAP1, ACTB, across multiple disease cohorts. Thus, each gene was differentially expressed in at least one disease cohort relative to the CTRL cohort. However, each individual disease cohort did not possess a unique expression profile distinguishing it from all other disease cohorts.

Discrimination of MS from Homogeneous Comparator Groups: Identification of an Optimum Panel of Gene Expression Ratios

Healthy control subjects, subjects with MS, and subjects with other inflammatory neurologic disorders (OND-I), and subjects with neurologic disorders typically considered non-inflammatory (OND-NI) were recruited from multiple U.S. and European sites (Table 1-A and Table 1-B). Demographic characteristics of the different disease groups, MS, OND-I, or OND-NI were matched to the CTRL cohort (compilation of patient characteristics data not shown). Subjects with MS included subjects with clinically isolated syndrome (CIS), newly diagnosed MS subjects who were treatment naïve and subjects with established disease (>1 yr duration) on different therapies. Expression levels of test and control genes in blood were determined by quantitative reverse transcription polymerase chain reaction (RT-PCR) (Table 1-C).

TABLE 1-A
Characteristics of Subjects

| | Sites | | |
|---|---|---|---|
| | Nashville | U.S.* | Europe** |
| MULTIPLE SCLEROSIS (total) | 84 | 81 | 80 |
| CIS | 14 | | 10 |
| Treatment naïve | 30 | 4 | 55 |
| Established disease (on meds) | 40 | 77 | 15 |
| OND-I (total) | 1 | 85 | |
| Acute disseminated | | 4 | |
| encephalomyelitis | | | |
| Bell's Palsy | | 3 | |
| CNS lupus | | 2 | |
| Guillaine Barre | | 4 | |
| Myasthenia Gravis | | 3 | |
| Neuromyelitis optica | | 27 | |
| Optic neuritis | 1 | 1 | |
| Transverse myelitis | | 41 | |
| OND-NI (total) | 1 | 128 | |
| Alzheimer's | | 6 | |
| Cerebral ataxia | | 2 | |
| Cerebral bleed | | 2 | |
| Cervical radiculopathy | | 6 | |
| Drug-induced movement | | 1 | |
| disorder | | | |
| Dystonia | | 1 | |
| Epilepsy | 1 | 4 | |
| Essential tremor | | 9 | |
| Huntington's disease | | 1 | |
| Hydrocephalus | | 1 | |
| Median Neuropathy | | 2 | |
| Meningioma | | 1 | |
| Migraine | | 30 | |
| Parkinson's | 3 | 0 | |
| Peripheral Neuropathy | | 1 | |
| Pseudotumor | | 3 | |
| Restless Leg Syndrome | | 1 | |
| Seizures | | 6 | |
| Spasmodic torticollis | | 1 | |
| Stroke | | 18 | |
| Tourette's Syndrome | | 1 | |
| Transient Ischemia | | 1 | |
| CONTROLS | 48 | 61 | |

*six additional sites in U.S.: MA, MD, NY, SC, AZ, TX, CA, samples from sites in MS, MD, NY, AZ, and CA were obtained through the Accelerated Cure Project.
**Denmark, Netherlands

TABLE 1-B
Demographic characteristics of the different subject populations.

| | | | | | ETHNIC- | |
|---|---|---|---|---|---|---|
| | | | GENDER | | ITY (%, | |
| | AGE | P* | (% F) | P | C/AA/As/H)** | P |
| MS | 43 ± 10 | NS | 76 | NS | 80/20/0/0 | NS |
| OND-NI | 46 ± 10 | NS | 67 | NS | 68/26/3/1 | NS |
| OND-I | 46 ± 10 | NS | 68 | NS | 67/33/0/0 | NS |
| CTRL | 41 ± 11 | | 77 | | 71/22/3/3 | |

*P calculated by Student's T-test (Age) or Fisher's exact test. NS: P > 0.05.
**C, Caucasian; AA, African American; As, Asian; H, Hispanic.

TABLE 1-C
Gene probes on TLDA plate

| | | |
|---|---|---|
| ABR | EPHX2 | OAS1 |
| ACTB | EXT2 | ORC1L |
| ACTR1A | FOS | PGK1 |
| ADAMTSL4 | FOSL1 | PMAIP1 |
| ANAPC1 | GAPDH | POU6F1 |
| APOBEC3F | GATA3 | RANGAP1 |
| ASL | GNB5 | SC65 |
| B2M | GSTM4 | SPIB |
| BRCA1 | HLA-DRA | TAF11 |
| CD55 | HRAS | TBP |
| CDH1 | IFI27 | TGFBR2 |
| CDKN1B | IL11RA | TP53 |
| GAPDH | JUN | TXK |
| CHEK2 | KRAS | GNB5 |
| CSF3R | LLGL2 | TP53 |
| CTSS | NRAS | |

A search algorithm was employed to identify those ratios of gene expression levels in which the greatest number of subjects in the test group possessed a ratio value greater than the highest ratio value in the comparator group. A second algorithm was employed to perform permutation testing of one subject group to identify the optimum set of discriminatory ratios.

It was reasoned that examination of expression levels of ratios of genes rather than individual genes would serve the following purposes. First, calculation of ratios normalized for differences in mRNA or cDNA template quantity and quality among different samples. Second, they obviated the need for inclusion of a ‘housekeeping’ gene in the analysis and the assumption that expression levels of ‘housekeeping’ genes did not vary among different subject populations. Third, comparisons of ratios or combinations of ratios may more accurately identify cellular phenotypes that may contribute to disease. For example, a ratio containing one gene in the numerator that is over-expressed in the test group relative to the comparator group and one gene in the denominator that is under-expressed in the test group relative to the comparator group should produce a greater ratio value difference between individuals in the two groups than a single expression value. A point system was employed to award one point to a subject if a ratio value of the test subject was greater than the ratio values of all subjects in the comparator group.

This approach was applied to determine how accurately it would distinguish subjects with MS from healthy control subjects. First, ratios capable of discriminating MS subjects from control subjects were identified. The single ratio with the greatest discriminatory power was ANAPC1/CHEK2 (FIG. 2a). Fifty % of MS subjects achieved a ratio value higher than all the CTRL subjects and were awarded one point. Second, those ratios that identified fewer than 20% of MS subjects were eliminated. Third, since many ratios identified the same MS subjects, another reduction was performed to preserve only one ratio with this characteristic. A total of 8 ratios remained after this minimization process (FIG. 2b). Using the point system, the combination of these 8 ratios positively identified 97% of MS subjects and eliminated 100% of CTRL subjects (FIG. 2c). The score distribution was 0-6 for MS subjects and 0 for CTRL subjects (FIG. 2d).

Discrimination of MS from Homogeneous Comparator Groups: Validation and Analysis

The analyses depended upon determination of multiple ratios, which may create Type 1 errors. Various methods are available to correct for false discovery rates. Rather than relying upon these methods, which all make underlying assumptions, a second evaluation was performed using an independent cohort of 40 new MS subjects and 40 new CTRL subjects to validate results obtained from the initial training set. These subjects were recruited separately and the PCR analyses were performed separately. The same ratio values were used, as defined from the original CTRL and MS test set to award points to subjects in the validation cohort. All 40 controls were awarded a score of 0 while 4% of MS subjects received a score of 0. The remaining 96% of MS subjects achieved a score of 1-6 and the distribution of scores was similar to that observed in the training set (FIG. 2e). Taken together, this demonstrates that results obtained in the training set can be replicated in an independent cohort of CTRL and MS subjects.

The point system was applied to OND-I and OND-NI subjects. In contrast to CTRL subjects, 90% of OND-I and 59% of OND-NI subjects scored ≧1 (FIG. 2f). Scores among subjects with CIS were compared, with newly diagnosed MS not yet on medications, and with established MS on different medications. Scores did not differ significantly among these three groups (FIG. 2g). Also compared were scores within the MS group as a function of geographic origin. Scores also did not vary significantly among MS subjects from different geographic sites (FIG. 2h). Thus, subjects with CIS or subjects after their initial diagnosis of MS had a similar mean score to subjects with established MS on therapies. However, a high percentage of subjects with other neurologic conditions, especially inflammatory neurologic conditions, also scored ≧0 in this analysis. Given its extremely high specificity and relatively low sensitivity, embodiments of this test have greater application to exclude an individual from the diagnosis of MS rather than to establish a diagnosis of MS.

Further, follow-up clinical information on 8 CIS subjects >2 yr. after the initial consent and blood draw were able to be obtained. Of these subjects, the 7 CIS subjects who achieved a score >0 in the analysis now have documented MS. The 1 CIS subject who achieved a score of 0 does not have a documented case of MS.

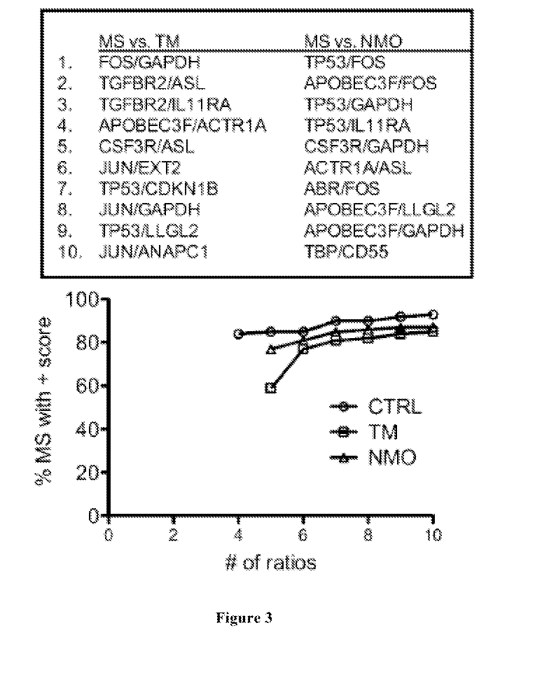

NMO and TM are inflammatory neurologic diseases that scored positive in the analysis. Therefore, it was determined whether a similar approach could be employed to discriminate MS from TM and MS from NMO. A series of ratios were identified that, when combined using the point system, were able to discriminate TM from MS and NMO from MS with similar overall accuracy to the MS and CTRL comparisons (FIG. 3). Thus, using the approach, it was possible to distinguish MS from TM and MS from NMO with a similar degree of accuracy as obtained for the comparison of MS to CTRL. However, since each disease possessed a unique signature, it was necessary to employ separate combinations of ratios to accurately distinguish MS from NMO and MS from TM.

Above results demonstrate it is possible to distinguish MS from either a control cohort or even a related inflammatory disease cohort if the disease cohort is a single disease.

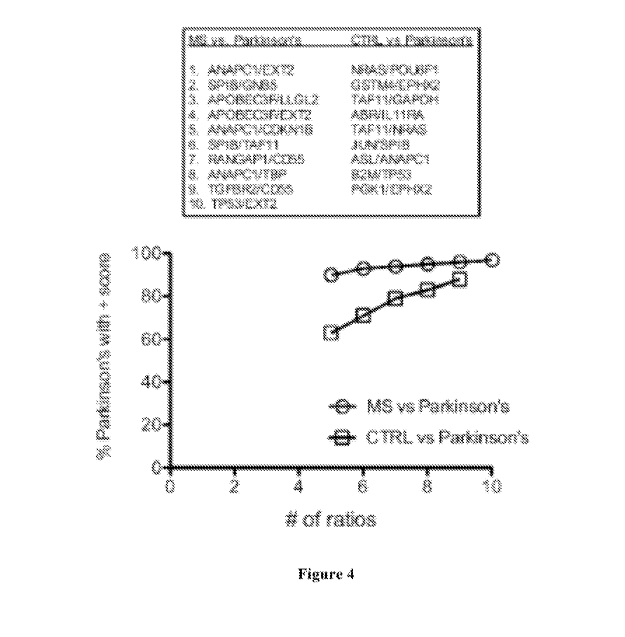

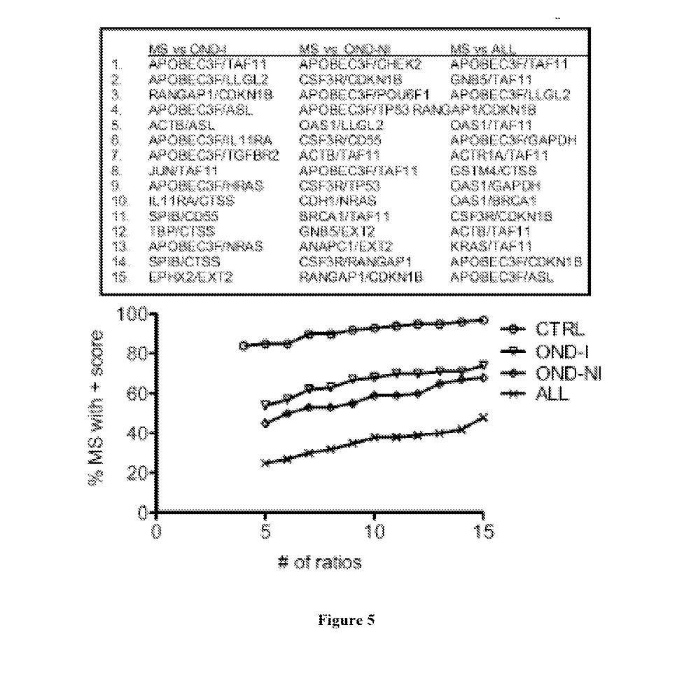

Next, it was determined whether MS could be discriminated from Parkinson's disease, a disorder typically considered non-inflammatory. To test this hypothesis in it was determined if subjects with Parkinson's disease (N=24) segregated from MS (N=182) and from CTRL (N=109) using the ratio and point system. Ten (10) ratios capable of discriminating 97% of MS subjects from 100% of Parkinson's subjects and 9 ratios capable of discriminating 88% of Parkinson's patients from 100% of CTRL subjects were identified (FIG. 4). These results demonstrate that subjects with Parkinson's disease express unique gene expression signatures in blood distinguishing them from CTRL and MS subjects.

Discrimination of MS from Heterogeneous Comparator Groups

Next, it was determined whether MS could be distinguished from more heterogeneous groups of subjects. To do so, subjects with neurologic conditions typically considered as inflammatory (other neurologic disorders-inflammatory, OND-I in Table 1-B) were combined into one group. Subjects with neurologic conditions typically considered non-inflammatory (other neurologic disorders-non-inflammatory, OND-NI, OND in Table 1-B) were combined into a second group. A third group consisting of CTRL+OND-I+OND-NI subjects (ALL) was prepared. The 15 best ratios were determined using permutation testing for each comparison. Overall, comparison of MS to these heterogeneous comparator groups resulted in a marked reduction in overall discrimination ability (FIG. 5). It was concluded that a binary comparison such as this exhibits much reduced accuracy as the heterogeneity of the comparator group is increased.

Discrimination of MS from OND-I: Identification of Optimum Panels of Gene Expression Ratios

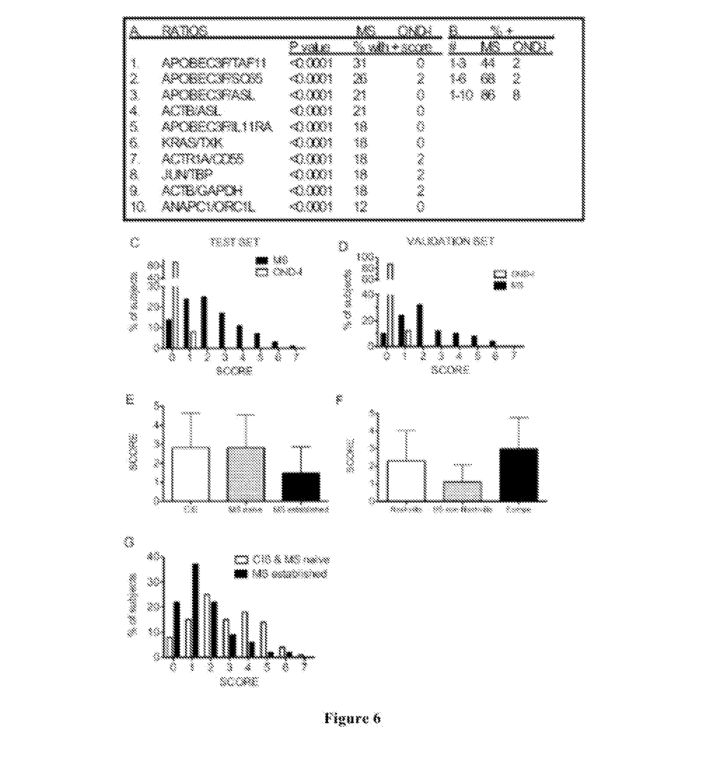

For additional analysis, OND-I was combined into one group of non-MS inflammatory neurologic disorders and investigated the ability of the approach to discriminate this combination of diseases from MS. The conditions were relaxed somewhat to identify ratios with the ability to detect 0 or 1 non-MS subjects. The best results were obtained with 10 ratios (FIG. 6a). The combination of which identified 86% of MS subjects with a score >0 and only 8% of OND-I subjects with a score >0 (FIG. 6b). Scores ranged from 0-7 for MS subjects and 0-1 for OND-I subjects (FIG. 6c).

Discrimination of MS from OND-I: Validation and Analysis

Additional analyses were performed with 40 new MS subjects and 40 new OND-I subjects (20 NMO and 20 TM) not included in the training set. In the validation set, 88% of MS subjects achieved a score ≧1 and 12% of OND-I subjects achieved a score of 1 (FIG. 6d), which was similar to the score distribution observed in the training set. The mean scores among subjects with CIS were determined, subjects with newly diagnosed MS prior to onset of therapies, and subjects with established MS on therapies using the 10 ratios identified above. Mean scores were significantly higher in the CIS and MS-naïve groups than in the MS group with established disease (FIG. 6e). Mean scores based upon geographic origins of MS subjects were also determined. Subjects from Nashville and Europe had mean scores significantly greater than U.S. subjects from locations other than Nashville (FIG. 6f). These results are consistent with results comparing CIS, MS-naïve, and MS-established. The majority of subjects from U.S. sites outside Nashville had established MS and were on therapies (76 of 80 subjects) while all European subjects were either CIS or newly diagnosed MS subjects not yet on therapies (N=101). The Nashville site also provided more samples with established disease (N=37) compared to CIS or treatment naïve MS (N=16) (P <0.0001, Chi-squared test for independence among three geographic locations). The distribution of scores in the CIS and newly diagnosed MS group was also higher than that found in the established MS group. Greater than 50% of subjects with established MS achieved scores of 0 or 1 while 48% of CIS and newly diagnosed MS subjects achieved scores ≧3 (FIG. 6g). Thus, subjects with CIS, newly diagnosed MS, and established MS from different geographic sites can be distinguished from subjects with OND-I with reasonable accuracy based upon gene expression profiles in whole blood.

Discrimination of MS from OND-NI: Identification of Optimum Panels of Gene Expression Ratios

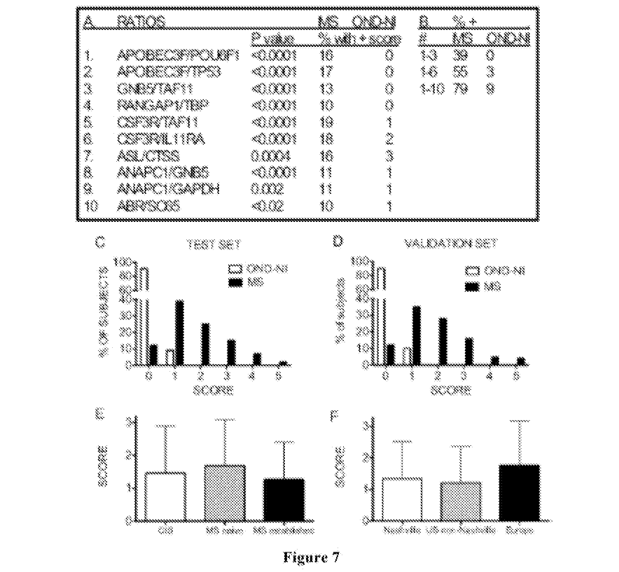

Next, gene expression differences between MS and OND-NI subjects were compared, which included Parkinson's disease, essential tremors, migraines, and strokes. The same search strategy used to compare MS and OND-I subjects was employed and identified 10 expression ratios to construct the point system. ABOBEC3F, CSF3R, and ANAPC1 were each in the numerators of two ratios and TAF11 was in the denominator of two ratios. Each ratio alone detected >10% of MS subjects relative to OND-NI subjects (FIG. 7a). Combining ratios using the point system improved overall ability to discriminate MS subjects from OND-NI subjects (FIG. 7b). Using the point system, 79% of MS subjects achieved a score ≧1 and 91% of OND-NI subjects achieved a score of 0, 9% achieved a score of 1 (FIG. 7c).

Discrimination of MS from OND-NI: Validation and Analysis

Additional analyses were performed with 40 new MS subjects and 40 new OND-NI subjects not included in the training set as outlined above. In the validation set, 88% of MS subjects achieved a score ≧1 (FIG. 7d), which was a similar frequency to that observed in the training set, and 90% of OND-I subjects achieved a score of 0, 10% achieved a score of 1. As above, mean scores of subjects with CIS, newly diagnosed MS and established MS were determined and these were not statistically different among the three MS groups (FIG. 7e). Similarly, mean scores of MS subjects from different geographic sites were not statistically different (FIG. 7f). Using the point system, ˜80% of MS subjects achieved a score ≧1 and 9% of OND-NI subjects achieved a score=1 in the test set. These results demonstrate that expression in whole blood of a different set of gene ratios discriminated subjects with MS from subjects with OND-NI with reasonable accuracy.

All comparisons in these analyses were binary. Therefore, exclusion of a specific disorder by the analysis may be more accurate than inclusion of a specific disorder (see flow chart).
Flow Chart: Tiered approach using expression ratios to determine probability of the presence or absence of MS

Analysis 1: MS versus control
score=0: >95% probability subject does not have MS
score ≧1: move to analysis 2

Analysis 2A: MS versus OND-I
score=0: ˜8-fold greater likelihood of OND-I than MS
score ≧1: ˜8-fold greater likelihood of MS than OND-I

Analysis 2B: MS versus OND-NI
score=0: ˜8-fold greater likelihood of OND-NI than MS
score ≧1: ˜8-fold greater likelihood of OND-NI than MS

Analysis 3A: MS versus NMO
score=0: ˜90% probability subject does not have MS
score ≧1: ˜90% probability subject does not have NMO

Analysis 3B: MS versus TM
score=0: ˜90% probability subject does not have MS
score ≧1: ˜90% probability subject does not have TM

Thus, a score of 0 in the MS versus CTRL test decreased the probability that a subject had MS. A second analysis comparing MS to OND-I and MS to OND-NI would be interpreted similarly. Scores of 0 decreased the probability of MS and favored the probability of OND-I or OND-NI, respectively. Finally, specific inflammatory neurologic disorders, NMO or TM, were distinguished from MS with high degrees of accuracy. Thus, results from this single platform can be analyzed in a tiered approach to provide meaningful disease classification.

Discussion

Although the focus was on MS and other inflammatory and non-inflammatory neurologic disorders, the results support the notion that this approach could be applicable to an array of diseases. First, discrimination between MS and healthy controls or subjects with individual diseases can be achieved with a relatively high degree of accuracy. However, subjects with OND-I and OND-NI also scored positive in MS-CTRL comparisons. As such, this single comparison has greater utility as an exclusionary test rather than a test of MS inclusion. Second, it is possible to discriminate MS from groups of diseases, such as inflammatory or non-inflammatory neurologic diseases, and validate results in independent cohorts, although overall accuracy is somewhat compromised. Third, discrimination of MS from a diverse comparator group including CTRL, OND-I, and OND-NI causes a further reduction in overall accuracy. Nevertheless, a score >0 in this analysis is highly predictive of the presence of MS. Fourth, it is possible to identify small numbers of ratios with high degrees of discriminatory power whose accuracy can be validated in independent cohorts analyzed separately.

One interpretation of the results is that many individual diseases express unique but overlapping gene expression signatures in whole blood. Given the attention paid to analyses of autoimmune diseases, it is not surprising that inflammatory neurologic diseases such as NMO and TM also express unique gene expression signatures. Perhaps somewhat surprising is that Parkinson's disease, a disorder typically considered non-inflammatory, also possesses a unique gene expression signature distinguishing it from both CTRL and MS. Implications may be that the immune system can sense specific neurologic damage caused by Parkinson's via responses to cytokine mediators, adhesion molecules, neurotransmitters, or other mediators read by immune cells. Alternatively, genetic risk factors associated with Parkinson's disease may contribute to altered gene expression signatures by either direct or indirect mechanisms.

Mechanisms underlying gene expression differences among study groups or relationships to MS disease mechanism are not altogether clear. However, defects in DNA damage repair, cellular responses to DNA damage, and regulation of cell cycle progression and arrest are common properties of lymphocytes in certain autoimmune diseases, including MS, and ANAPC1, CHEK2, CDKN1B, ACTB, FOSL1, LLGL2, and NRAS encode proteins playing key roles in these fundamental cellular processes²³⁻²⁷. These genes are highly represented in the ratios used to distinguish MS from comparator groups. Genes, such as ADAMTSL4, B2M, IL11RA, TXK and POU6F1, encode proteins playing key functions in regulating cells of both innate and adaptive arms of the immune system^{28, 29}. As such, alterations in expression of these genes may contribute to pathogenesis of MS or may represent an altered response by the immune system to MS pathogenesis.

The follow-up analysis of CIS patients supports the idea that initial scores >0 will correlate with progression to MS. Future longitudinal studies are planned to better evaluate utility of these tests in this setting. Further, the binary analysis is also predicated on the fact that MS is best represented by a single set of gene expression ratios and this may not be the case. Additional analyses, such as analyses of gene expression ratios in multi-dimensional space, will address this possibility. Several different combinations of gene expression ratios were identified, which performed equivalently in their ability to discriminate among subject groups. In conclusion, these minimally invasive and relatively inexpensive tests may have utility to either exclude the diagnosis of MS or to contribute to establishing a diagnosis of MS.

Materials and Methods

Patients.

Blood samples in PAXgene tubes were obtained from patients with a) clinically isolated syndrome (CIS), b) an initial diagnosis of MS before onset of therapy, and c) established relapsing-remitting MS on medication. Blood samples were also obtained from healthy control subjects (CTRL) and subjects with different inflammatory (OND-I) or non-inflammatory (OND-NI) neurologic conditions. MS samples were obtained from a total of 9 different sites in the U.S. and Europe. Samples from subjects with OND-I and OND-NI were obtained from 7 sites in the U.S. CTRL samples were obtained from 3 U.S. sites. Inclusion criteria for MS and other neurologic conditions were diagnosis by a neurologist using established methods and ability to provide informed consent, thus providing an un-biased study cohort. Age, race and gender were not statistically different among the different study groups. Time of the blood draw, e.g. morning/afternoon clinics, was also not statistically different among the different study groups. Relevant institutional review board approval from all participating sites was obtained.

Procedures.

Total RNA, purified using Qiagen's isolation kits by standard protocols, was reverse-transcribed using SuperScript III (Invitrogen). A TaqMan Low Density Array (TLDA) was designed to analyze expression levels of 44 genes previously identified from the microarray analysis and of 4 “housekeeping” genes in 300 ng cDNA per sample. Patient diagnosis was blinded for all experimental procedures. Relative expression levels were determined directly from the observed threshold cycle (C_{T}), the cycle number at which fluorescence generated within reactions crosses an assigned threshold reflecting the point where sufficient amplicons have accumulated to be statistically significant above baseline. Linear expression values were determined using the formula, 2^{(40-CT)}.

Identification of Discriminatory Gene Expression Ratios.

A computational algorithm was designed to identify the most discriminatory combinations of ratios²². All possible gene expression ratios were computed (e.g. ACTR1A/BRCA1, TAF11/ACTR1A, etc). To analyze individual results, R_{i,j}^{control} was used to denote the i^{th} ratio for the j^{th} control and let R_{i,k}^{MS} was used to denote the i^{th} ratio for the k^{th} MS patient. Here, j=1, . . . , N_{control} and k=1, . . . , N_{MS}, where N_{control} equals the total number of controls and N_{MS} equals the total number of MS patients in the data set. The second largest member of each data set of ratios was calculated first by {R_{i,1}^{control}, R_{i,2}^{control}, . . . , R_{i,N}_{_{control}}^{control}}, and designated Rᵢ⁽²⁾. This was then applied to the MS data set {R_{i,1}^{MS}, R_{i,2}^{MS}, . . . , R_{i,N}_{_{MS}}^{MS}}. Cᵢ was used to designate the number of MS set of ratios larger than Rᵢ⁽²⁾ such that 0≦Cᵢ≦N_{MS}. This process was repeated for each possible ratio. The ratio that produced the largest Cᵢ was selected as the discriminator of the two sets. This process was repeated using all possible ratios. Although more than one optimal ratio could be identified for each number of components queried, only one discriminator has been presented for each combination. Ratios were included only if >20% of subjects within the MS group had expression values greater than all subjects in the CTRL group. A scoring system was developed to combine multiple ratios. To do so, subjects were assigned one point for each ratio in which their expression value was higher than the highest expression value within the CTRL subject group. By this approach, it was also possible to relax search criteria by setting cutoffs to the second highest expression ratio, third highest expression ratio, etc., of the comparator subject group. Using these relaxed criteria, an individual was awarded one point if the value of their expression ratio was higher than the second or third, etc., highest expression value of individuals in the comparator group, respectively. These combined ratios established a score discriminating the MS group from comparator groups.

Search Algorithm for Best Ratios.

Let D denote the set of 44 gene-expression levels associated with the disease group and C denote the set of gene-expression levels associated with the control group. For example, when D is the set of MS patients, then D is a set of 182 44-tuples; if C is associated with the Controls, then C is a set of 51 44-tuples. The algorithm that searches for the “best” set of gene ratios is the following:
80% of the control group was randomly selected and compared to the disease group in the following manner. Gene-expression level ratios were formed for elements in D and C. For each ratio, the number of elements in the disease group that were larger than the largest ratio in the control group was computed. The top 500 ratios that separate elements in D and C were saved. This calculation was repeated 200 times resulting in a set of 200 subsets of ratios (each subset having 500 ratios).
The 500 subsets were processed to identify the smallest number of ratio, R={r₁, r₂, . . . , rₙ}, that produced the maximum of separation of D and C. Associated with each of the ratios in R, there were threshold values, T={t₁, t₂, . . . , tₙ}, which corresponded to the highest value in the control group for each of the ratios in R.
For each member of the disease group D, the ratios in R were computed, {α₁, α₂, . . . , αₙ}. If α₁≧t₁, then the ratio a was assigned 1; otherwise, it was assigned a 0. In this way, an n-tuple of 1's and 0's was generated for each member of D. For example, if n=6, then a typical 6-tuple would be {1,1,0,0,1,0}. This meant that this individual in the disease group would have 3 ratios that exceeded the corresponding ratios in the control group.
Lastly, the percentage of members in the disease group that had nonzero n-tuples was calculated. The larger the percentage, the better the separation of D and C.

### Statistical Analysis

The Welch's corrected T-test not assuming equal variances was used to calculate P values in two-way comparisons. The Chi-squared test for independence was used to calculate P values in three-way comparisons. The Bonferroni method was employed to correct for multiple testing³⁰.

### Example 2

### Using Biomarkers to Predict Progression from Clinically Isolated Syndrome to Multiple Sclerosis

Patients.

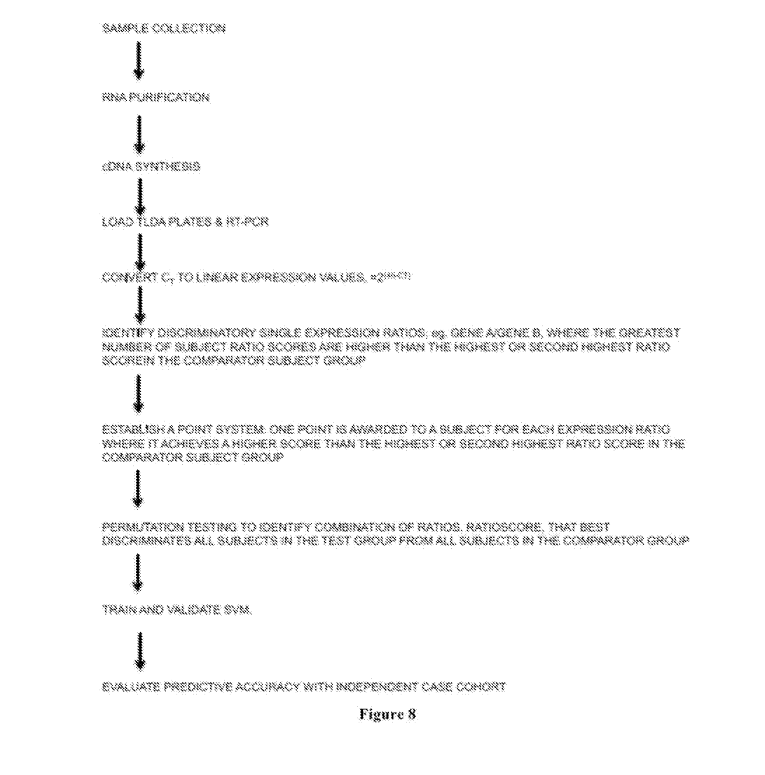

A total of 562 subjects were included in the study: 199 with clinically definite MS, 203 with OND segregated into 84 OND-I subjects and 119 OND-NI subjects, 114 healthy control subjects and 46 subjects whose blood sample was obtained at the time of their CIS but who now have progressed to clinically definite MS, CIS→MS (Table 2-A). MS patients were divided into two additional categories: those at their initial diagnosis of MS but before initiation of therapies; MS-naïve, and those ≧1 year after diagnosis of MS and on different therapies; MS-established. The overall laboratory and analytic processes are summarized in FIG. 8.

TABLE 2-A
Demographic characteristics of the different subject populations.

| | | | | | | ETHNIC- | |
|---|---|---|---|---|---|---|---|
| | | | | GENDER | | ITY (%, | |
| | # | AGE | P* | (% F) | P | C/AA/As/H)** | P |
| MS | 199 | 43 ± 10 | NS | 76 | NS | 80/20/0/0 | NS |
| OND-I | 84 | 46 ± 10 | NS | 68 | NS | 67/33/0/0 | NS |
| OND-NI | 119 | 46 ± 10 | NS | 67 | NS | 68/26/3/1 | NS |
| CTRL | 114 | 41 ± 11 | | 77 | | 71/22/3/3 | |
| CIS →MS | 46 | 35 ± 6 | NS | 72 | NS | 82/14/4/0 | NS |

MS = MS-treatment naïve (N = 85), MS with established disease on medications (N = 114), OND-I = other inflammatory neurologic disorders, acute disseminated encephalomyelitis (N = 4), Bell's Palsy (N = 3), CNS lupus (N = 2), Guillaine Barre (N = 4), Myasthenia Gravis (N = 3), Neuromyelitis optica (N = 26), Optic neuritis (N = 1), Transverse myelitis_(N = 41), OND-NI = other non-inflammatory neurologic disorders, Alzheimer's (N = 6), cerebral ataxia (N = 2), cerebral bleed (N = 2), cervical radiculopathy (N = 6), drug-induced movement disorder (N = 1), dystonia (N = 1), epilepsy (N = 4), essential tremor (N = 9), Huntington's disease (N = 1), hydrocephalus (N = 1), median neuropathy (N = 2), meningioma (N = 1), migraine (N = 30), Parkinsons (N = 23), peripheral neuropathy (N = 1), pseudotumor (N = 3), restless leg syndrome (N = 6), seizures (N = 9), stroke (N = 10),
CIS →MS: subjects who had clinically isolated syndrome at the time of the blood draw who have developed clinically definite MS.
U.S. sites: TN, MA, MD, NY, SC, AZ, TX, CA, samples from sites in MS, MD, NY, AZ, and CA were obtained through the Accelerated Cure Project, European sites: Denmark, Netherlands
*P calculated by Student's T-test²¹ or Fisher's exact test, NS: P > 0.05, calculated relative to CTRL.
**C, Caucasian; AA, African American; As, Asian; H, Hispanic.

Transcript Profiles.

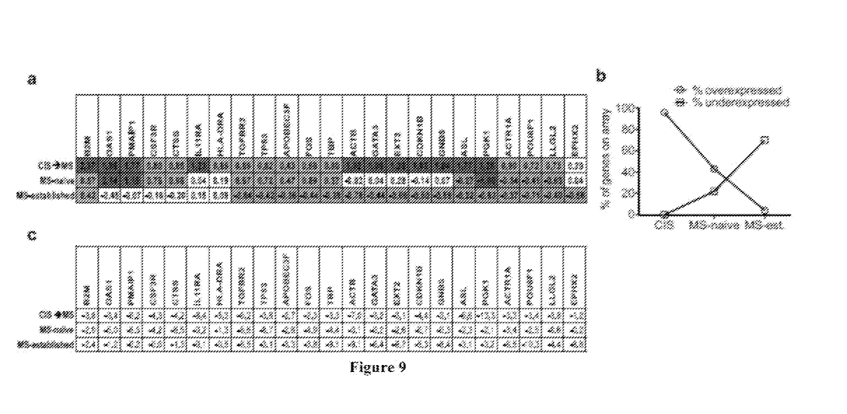

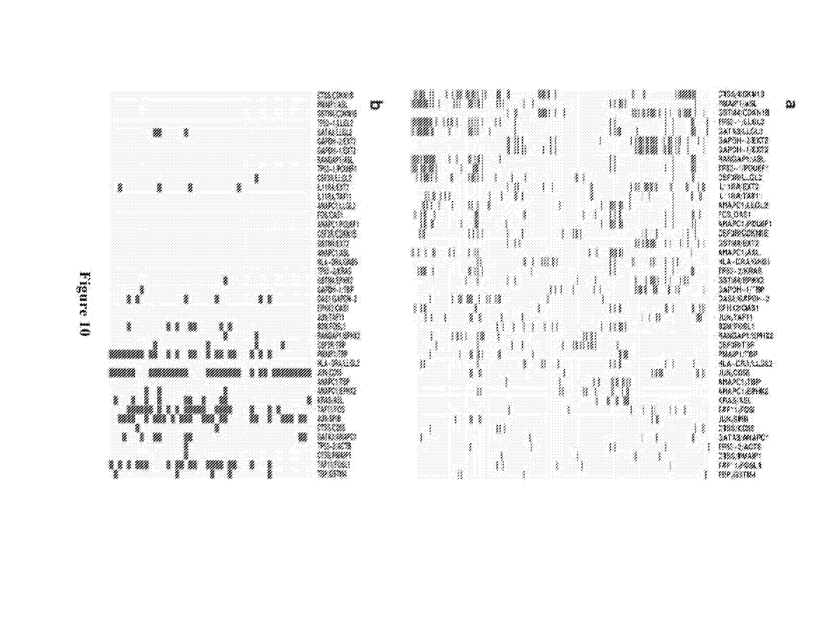

The transcript level in blood was determined for each target gene relative to GAPDH in the three study groups, CIS→MS, MS-naïve, MS-established and the CTRL group using TLDA plates. Target genes were selected from previous microarray studies.¹⁷⁻¹⁹ The ratio, log₂, of the expression level of each gene in each study group was calculated relative to CTRL and results are presented in a heatmap. Numerical ratios, log₂, are displayed within each box (FIG. 9a). Transcript profiles in the three study groups, CIS→MS, MS-naïve, and MS-established were highly dynamic. In the CIS→MS cohort, most genes were significantly over-expressed relative to CTRL. In contrast, the majority of target genes were significantly under-expressed in the MS-established cohort. The MS-naïve cohort was intermediate with an almost equal number of over- and under-expressed genes (FIG. 9b). Using the student's T test, P-values, log₁₀, were determined comparing each study group cohort to the CTRL cohort (FIG. 9c). Differences in transcript levels of many genes were highly significant among the different study groups. Of note, the P-value, log₁₀, for PGK1 expression between the CIS→MS cohort and CTRL cohort was −13.3. Similarly, expression differences of LLGL2 was most significant in the MS-naïve cohort, log₁₀=−9.6 and expression differences of POU6F1 was most significant in the MS-established cohort, log₁₀=10.3. One interpretation of these results is that each subject within each of these three disease cohorts, CIS→MS, MS-naïve, and MS-established, has a very similar target gene transcript profile suggesting that each is mediated by a common underlying molecular pathway(s) or event(s). Even though this is a cross-sectional rather than a longitudinal study, a second interpretation of these results is that target gene transcript profiles are highly dynamic as a subject progresses from CIS to clinically definite MS to MS disease of some duration.

Ratioscore Algorithm.

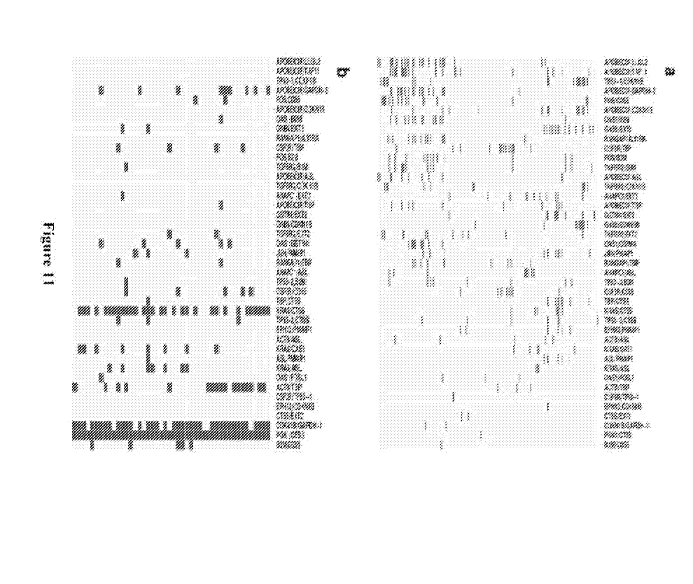

The previously described ratioscore method was used to compute all gene expression ratios and permutation testing to identify the set best able to discriminate the MS cohort, naïve and established combined, from the CTRL cohort⁴⁰. A heatmap was generated to depict which ratios (columns) were positive for each MS subject (rows) (FIG. 10a). One or more positive ratios produces a score ≧1 making a subject positive for the indicated disease, in this case, MS. A total of 173 of 199 MS subjects (87%) were assigned to the MS category using the ratioscore method and 100% of CTRL subjects were excluded from the MS category. Using these gene expression ratios, data was input from the CIS→MS cohort to determine if these subjects would fall into the MS or CTRL category. As above, a heatmap was constructed to depict which ratios (columns) were positive in each CIS→MS subject (rows). A total of 44 of 46 CIS→MS subjects (96%) were assigned to the MS category using the ratioscore defined for MS (FIG. 10b).

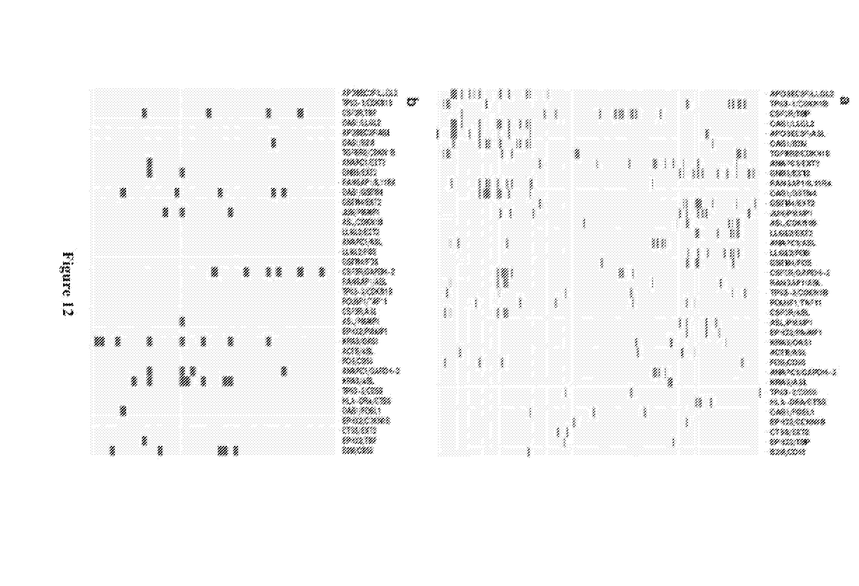

Using a similar approach, the ratioscore algorithm was used to compute ratios to discriminate MS, combined MS-naïve and MS-established from OND. As above, a heatmap was generated to depict which ratios (columns) were positive for each MS subject (rows) (FIG. 11a). A total of 140 of 199 MS subjects (70%) were assigned to the MS category using the ratioscore method and 203 of 203 (100%) of OND subjects were excluded from the MS category. As above, using these gene expression ratios, data was input from the CIS→MS cohort to determine if these subjects would fall into the MS or CTRL category. A similar heatmap was constructed to depict which ratios (columns) were positive in each CIS→MS subject (rows). A total of 46 of 46 CIS→MS subjects (100%) fell into the MS category using the ratioscore method (FIG. 11b).

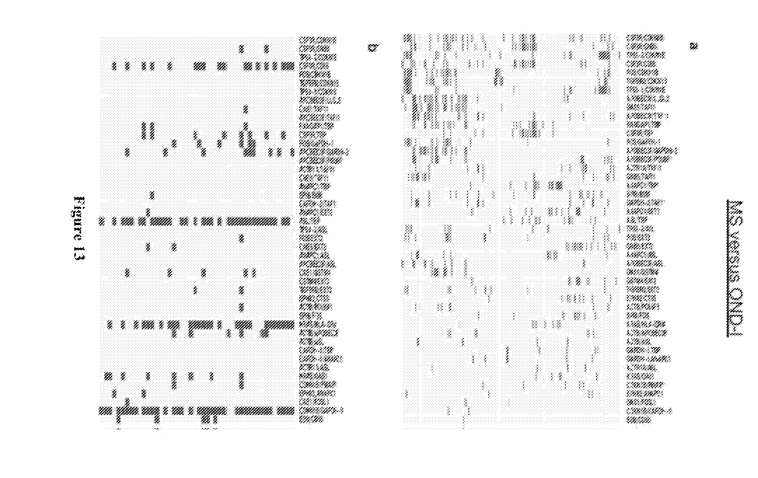

The rationale for performing this two-tier analysis rather than combining the CTRL and OND subjects into one cohort was that previous studies demonstrated that accuracy was severely compromised. To confirm that this was the case in this analysis the MS cohort was compared to the combined CTRL plus OND cohort and these data were inputted into the ratioscore algorithm. As expected, overall ability to discriminate MS from this combined cohort was compromised. Only 58% of MS subjects were assigned to the MS category while 100% of subjects in the combined CTRL plus OND cohort were excluded from the MS category (FIG. 12a). When data was input from the CIS→MS cohort, only 28 of 46 subjects (61%) were categorized as MS (FIG. 12b). Thus, overall accuracy of the ratioscore method was much improved by performing two tiers of analysis, first MS versus CTRL, then MS versus OND.

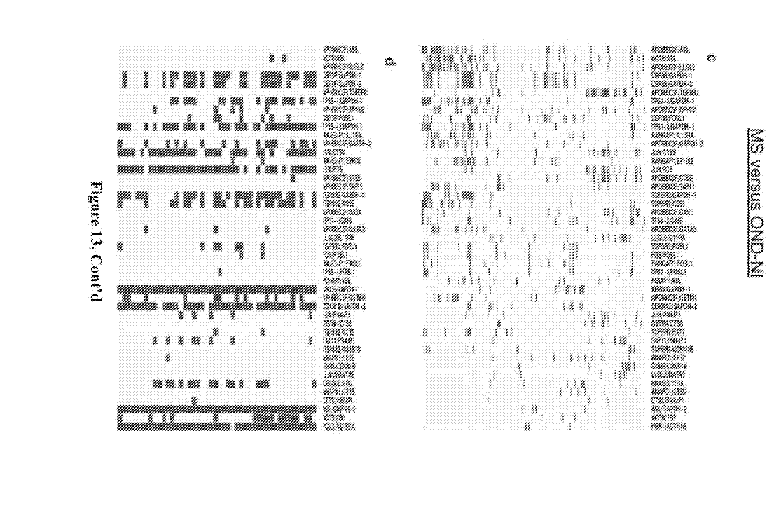

The OND cohort was also subdivided into OND-I and OND-NI (Table 2-A) and the ratioscore algorithm was repeated to assess how well these sub-groups could be distinguished from MS (FIG. 13a &13 b). In the OND-I versus MS comparison, 90% of MS subjects were assigned to the MS class and 100% of OND-I subjects were excluded from the MS class. When data was input from the CIS→MS cohort, 46 of 46 subjects (100%) were categorized as MS. In the OND-NI versus MS comparison, 86% of MS subjects were assigned to the MS class and 100% of OND-NI subjects were excluded from the MS class. When data was input from the CIS→MS cohort, 46 of 46 subjects (100%) were categorized as MS. It was conclude that this further subdivision of OND subjects produces only limited improvement in overall accuracy.

Accuracy of Ratioscore and SVM Methods.

A support vector machine (SVM) was also trained with ratios identified by the ratioscore method using 60% of CTRL subjects and 60% of cases (see Methods). SVM was validated with the remaining 40% of CTRLs and cases. Subjects within the CIS→MS cohort were input into the SVM to ascertain if the SVM would identify them as controls or cases. New SVMs were created using 60% of OND, OND-NI, and OND-I cohorts as controls, respectively and 60% of MS subjects as the case cohort. SVMs were validated with the remaining 40% of the respective control cohort and remaining 40% of the case cohort²⁰. As above, subjects within the CIS→MS cohort were input into each SVM to ascertain if the SVM would identify them as controls or cases. Results from the SVM method were compared to results from the ratioscore method by calculating sensitivity and specificity (Table 2-B). Overall, ratioscore and SVM produced comparable sensitivity and specificity in control: case comparisons. More relevant, subjects within the CIS→MS cohort were identified as MS by both methods with a high degree of specificity. Thus, this tiered approach, MS:CTRL then MS:OND, could be employed to predict if a subject with CIS will develop MS with a reasonable level of overall accuracy.

TABLE 2-B
Sensitivity and specificity of ratioscore and SVM methods

| | | RATIOSCORE | | SVM | |
|---|---|---|---|---|---|
| | | sensi- | speci- | sensi- | speci- |
| CONTROL | CASE | tivity | ficity | tivity | ficity |
| #1 CONTROL | MS | .87 | 1.00 | 0.86 | 0.93 |
| CONTROL | CIS → MS | .96 | | 0.95 | |
| #2 OND | MS | 0.70 | 1.00 | 0.82 | 0.78 |
| OND | CIS → MS | 1.00 | | 1.00 | |
| #3 OND-NI | MS | 0.86 | 1.00 | 0.84 | 0.94 |
| OND-NI | CIS → MS | 1.00 | | 1.00 | |
| #4 OND-I | MS | 0.90 | 1.00 | 0.77 | 0.93 |
| OND-I | CIS → MS | 1.00 | | 0.98 | |

Optimum ratios for the ratioscore method were from FIGS. 10, 11 and 13. CIS → MS subject data were inputted and scores computed. For the SVM, 60% of controls and cases were randomly selected for the training set and 40% were used for the validation set. Sensitivity and specificity were calculated for the combined sets. These results defined the SVM. CIS → MS subject data were applied to the SVM and subjects received a score of 0 if assigned to the CONTROL cohort or 1 if assigned to the CASE cohort. Sensitivity was calculated from this output.
Sensitivity = # true positives/(# true positives + # false negatives)
Specificity = # true negatives/(# true negatives + # false positives)

To summarize, overall transcript profiles in the CIS→MS, MS-naïve, and MS-established were markedly different and these dynamic transitions may reflect differences in underlying etiology. Studying the molecular origins of the robust transcript signature in CIS→MS subjects may produce insights into the origins of MS. In spite of the differences in overall transcript profiles in these three subject groups, ratioscore and SVM methods were able to assign CIS→MS subjects to the MS category with a high degree of accuracy. This is due, in part, to the fact that the ratioscore method does not require that all subjects within these three cohorts representing three distinct stages of disease progression possess identical gene expression signatures. In contrast, many other standard methods of analysis of gene expression signatures are dependent upon identification of overall differences between or among groups.

This study did not include subjects with an initial CIS that did not develop MS. The rationale for not including this parameter is three-fold. First, there is not a uniform clinical definition of CIS. Second, subjects with a CIS may or may not have MRI findings indicating inflammation or demyelination and the probability that a subject with CIS will develop MS is greater if MRI lesions are also detected. Third, with the current knowledge, it is uncertain if it is experimentally possible to absolutely conclude that a person with CIS will not develop MS. In fact, the period of time between an initial CIS and diagnosis of clinically definite MS is quite variable and can exceed 5 years.

Methods

Patients.

Blood samples in PAXgene tubes were obtained from CTRL, MS, OND-I and OND-NI subjects. Demographic characteristics of these cohorts have been previously described. Age, race and gender were not statistically different among the different study groups. Time of blood draw, for example, morning/afternoon clinics, was also not statistically significant among the different study groups. Relevant institutional review board approval was obtained from all participating sites.

Samples were also obtained from subjects with a clinically isolated syndrome (CIS) at the time of the blood draw. All of these subjects have gone on to develop MS according to the McDonald's criteria for the diagnosis of MS.

Transcript Determinations.

Total RNA purification, cDNA synthesis, and analysis using the 384-well Taqman Low Density Array (TLDA) were as previously described (FIG. 8).⁴⁰ Patient diagnosis was blinded for all experimental procedures. Relative expression levels were determined directly from the observed threshold cycle (C_{T}). Linear expression levels were determined using the formula, 2^{(40-CT)}.

Ratioscore and Support Vector Machine Algorithms.

The identification of the gene expression ratios and permutation testing strategy employed to identify discriminatory combinations of ratios to create the ratioscore have been previously described.^{40 and Example 1} Briefly, all possible gene-expression ratios of the 35 genes were computed. Ratios in which the greatest number of subjects in case groups possessed a ratio value greater than the highest ratio value in the control group were saved. Permutation testing was performed by randomly selecting 80% of the control group to compare with the case group and repeating this process 200 times producing 200 subsets of ratios. From these subsets of ratios, the smallest number of ratios to identify the ratioscore with maximum separation between case groups and control groups were identified. For example, MS versus CTRL, MS versus OND, etc. were compared. Each comparison produced a unique set of ratios that were used to define the ratioscore algorithm for that pairing of the case-control groups.

A support vector machine (SVM) was created from each set of ratioscores using LS-SVMLab software (http://www.esat.kuleuven.be/sista/Issvmab). For example, the gene-expression ratios from the MS versus CTRL were used to create a SVM for this type of comparison. The SVM was trained with L-fold cross-validation using 60% of the data. In this type of training a certain fraction of the training set was omitted from training and the remaining portion of the partial training set was used to estimate the parameters in the SVM. Once the SVM was trained, the SVM was applied to the total data set. Numbers of correct and incorrect classifications were tabulated for total sets (training and validation), training sets and validation sets. As expected, the overall accuracy in the training sets was greater than overall accuracy of the validation sets.

### SEQUENCES

The following are complementary DNA (cDNA) sequences of genes-of-interest identified in Table A. The portion of the sequences bolded and underlined are Applied BioSystems context sequences, the region of that can be amplified in some embodiments of the presently-disclosed subject matter. ABI assay numbers for the sequences are provided in Table A.

SEQ ID NO: 1
- Homo sapiens active BCR-related gene (ABR),
transcript variant 3, mRNA
GGACTGCAGAGGGAACTTGCCTTGAAGAGGCCTGGTCCTTAAAGAGACACAGCACACACGGCCCGACCGG
CAGCCCCAGAGCAGAGGCTCCACTGATGGCAGGCGCCCCTGGCTAGGCTCTGAGGTTCCTTTGCCCTCGC
CTTGCTGAATGGTGAGCCGCTGCCTCTCGGAGCCCGTCTCCTTGACAGCCTGCCCTCGGCTCCTGCAGCC
ACTCCTGGGCCTGATGGGGACAGGGCCAGCCTGGTGGGTGGTGTCAGAGGTCCTGGCAGAGCAGCGTAGG
CCTGGGATGCGTCTGCAGAATTCTGGCTGAACGAGCGAGGAGCACGGCCAGCTTCGGGGCCGTCGTGACC
ACAGGAGGGCAGAGGGCCAGCCCGTGAGCTCTGACCCCAGCTGGACGTGCTCTTGTTTCCCTTGGGGCTA
AGGAGATTGGAGCCACTGAACTGAATCTCTGGGTTTTGGAGACTTAGAGAATCCATTGGACTCTTCTGCT
GGCGTCTTTCTGAATGCTGATGGGGACTTGGTGACTTCAGCTACGGGACGGACGAGTACGACGGAGAGGG
GAATGAGGAGCAGAAGGGGCCCCCGGAGGGCTCAGAGACCATGCCGTACATCGATGAGTCGCCCACCATG
TCCCCGCAGCTCAGCGCCCGCAGCCAGGGCGGGGGGGATGGCGTCTCCCCGACTCCACCTGAGGGACTGG
CTCCTGGGGTGGAAGCAGGGAAAGGCCTGGAGATGAGGAAGCTGGTTCTCTCGGGGTTCTTGGCCAGCGA
AGAGATCTACATTAACCAGCTGGAAGCCCTGTTGCTGCCCATGAAACCCCTGAAGGCCACCGCCACCACC
TCCCAGCCCGTGCTCACCATCCAGCAGATCGAGACCATCTTCTACAAGATCCAGGACATCTATGAGATCC
ACAAGGAGTTCTATGACAACCTGTGCCCCAAGGTGCAACAGTGGGACAGCCAGGTCACCATGGGCCACCT
CTTCCAGAAGCTGGCCAGCCAGCTCGGTGTGTACAAAGCGTTTGTCGATAACTATAAAGTCGCTCTGGAG
ACAGCTGAGAAGTGCAGCCAGTCCAACAACCAGTTCCAGAAGATCTCAGAGGAACTCAAAGTGAAAGGTC
CCAAGGACTCCAAGGACAGCCACACGTCTGTCACCATGGAAGCTCTGCTCTACAAGCCCATTGACCGGGT
CACTCGGAGCACCCTAGTCCTACACGACCTGCTGAAGCACACACCTGTGGACCACCCCGACTACCCGCTG
CTGCAGGATGCCCTCCGCATCTCCCAGAACTTCCTGTCCAGCATCAACGAGGACATCGACCCCCGCCGGA
CTGCAGTGACAACGCCCAAGGGGGAGACGCGACAGCTGGTGAAGGACGGCTTCCTGGTGGAAGTGTCAGA
GAGCTCCCGGAAGCTGCGGCACGTCTTCCTCTTTACAGATGTCCTACTGTGTGCCAAGCTGAAGAAGACC
TCTGCAGGGAAGCACCAGCAGTATGACTGTAAGTGGTACATCCCCCTGGCCGACCTGGTGTTTCCATCCC
CCGAGGAGTCTGAGGCCAGCCCCCAGGTGCACCCCTTCCCAGACCATGAGCTGGAGGACATGAAGATGAA
GATCTCTGCCCTCAAGAGTGAAATCCAGAAGGAGAAAGCCAACAAAGGCCAGAGCCGGGCCATCGAGCGC
CTGAAGAAGAAGATGTTTGAGAATGAGTTCCTGCTGCTGCTCAACTCCCCCACAATCCCGTTCAGGATCC
ACAATCGGAATGGAAAGAGTTACCTGTTCCTACTGTCCTCGGACTACGAGAGGTCAGAGTGGAGAGAAGC
AATTCAGAAACTACAGAAGAAGGATCTCCAGGCCTTTGTCCTGAGCTCAGTGGAGCTCCAGGTGCTCACA
GGATCCTGTTTCAAGCTTAGGACTGTACACAACATTCCTGTCACCAGCAATAAAGACGACGATGAGTCTC
CAGGACTCTATGGCTTCCTTCATGTCATCGTCCACTCTGCCAAGGGATTTAAGCAATCAGCCAACCTGTA
CTGTACCCTGGAGGTGGATTCCTTCGGCTATTTTGTCAGCAAAGCCAAAACCAGGGTGTTCCGGGACACA
GCGGAGCCCAAGTGGGATGAGGAGTTTGAGATCGAGCTGGAGGGCTCCCAGTCCCTGAGGATCCTGTGCT
ATGAGAAGTGCTATGACAAGACCAAGGTCAACAAGGACAACAATGAGATCGTGGACAAGATCATGGGCAA
AGGACAGATCCAGCTGGACCCACAAACCGTGGAGACCAAGAACTGGCACACGGACGTGATTGAGATGAAC
GGGATCAAAGTGGAATTTTCCATGAAATTCACCAGCCGAGATATGAGCCTGAAGAGGACCCCGTCCAAAA
AGCAGACCGGCGTCTTCGGTGTGAAGATCAGCGTGGTGACGAAGCGGGAGCGCTCCAAGGTGCCCTACAT
CGTCCGGCAGTGTGTGGAGGAGGTGGAGAAGAGGGGTATCGAGGAGGTTGGCATCTACAGGATATCGGGC
GTGGCCACGGACATCCAGGCGCTCAAGGCCGTCTTCGATGCCAATAACAAGGACATCCTGCTGATGCTGA
GTGACATGGACATCAACGCCATCGCCGGGACGCTCAAGCTGTACTTCCGGGAACTGCCCGAGCCGCTCCT
CACGGACCGACTCTACCCAGCCTTCATGGAGGGCATCGCCCTGTCAGACCCTGCTGCCAAGGAAAACTGC
ATGATGCACCTGCTCCGCTCCCTGCCCGACCCCAACCTCATCACCTTCCTCTTCCTGCTGGAACACTTGA
AAAGGGTTGCCGAGAAGGAGCCCATCAACAAAATGTCACTTCACAACCTGGCTACCGTGTTTGGACCCAC
GTTACTGAGACCCTCAGAAGTGGAGAGCAAAGCACACCTCACCTCGGCTGCGGACATCTGGTCCCATGAC
GTCATGGCGCAGGTCCAGGTCCTCCTCTACTACCTGCAGCACCCCCCCATTTCCTTCGCAGAACTCAAGC
GGAACACACTGTACTTCTCCACCGACGTGTAGCCCGAGGCAGGGTGGCTGCGGGCGGGTGGTGGAACCAG
CCCCTCCAGCCTGGGGTCCAACTCAGACTTGAAAGACTGCAATAGAAAACTCCCAAACCCAGCACTCCAG
ACTCGAGGGAAGCCAGCTTCCAAGAACTGGAATGCGTACGTCTTTTGTGCCACCTTGTACAAAGCCGGCT
GCCCAGCCCCAGCCTCACCACCGCATCCCACCTCCTGCCCTCCATACCTCTAGTTGTGTCTGATGCTCCG
TGCTGTTCGGGAATTGTTTTATGTACACTTGTCAGGCAGAAAAGGTAGTGACCGGCCCGGCGTGGGCACA
CAGACAGCCCGCTTTGTTCTTTCATTTCCTCCAGCACTTTCTTTCCGCCTGAGTCCAGCCCAAGGCCTTT
TATTTTGCGCTGTGTAACTGCTGCCAGCTTCTCTCTTGGCCCTGCTCCCAGATGGCGGTCTCCTGGCAGC
CTCCCCTCAGTCTTCCTCCACCCGCTCTTCCTTCCCAGCCTGCCTGCATGCATGTGCACCCTTGGTCTTC
GCTCCATCGCCTTGAAAGCTCTGAAGAGGCCCTGGGTTGCCGCGGCAGCAGTGGTCTGTTTGATGCTGCC
GTTTGCCGCTGCCGGCCCCTCCTCAGACTCCGCCTTTGGGAGCACACCTGCTTTGCCTTGCTGCCTGTGC
AAATGTTGGACAAGCAGACACACTCACACTCGTCCCCAGCTTAGCACAGAGCTGGAGCGCCCATTTCTGG
AATTTTCCGTTTGGGAATCTCCACTTCTGGGGTTTACCTGTTCGGCCTCCTGTCTATCAGTGAGGCATCT
CTGACTGTTTCTTCTACTGCTTTTCAGTTCCCTTCCCTGCTGTTCTATTTCCTTTGAGTGTAAAGACTCA
CAGGTGACCTGCTATCGAGATAGCCAGAGGGTCAGGAGAGAATGGGGGAGGAGGCGGTCAGGCTGCTGAG
GAAACACCACAGGCTGAACGGGGGAGGAATGCACATGCCACGCTGGGTGTCCCGGGTCGCGGGGAGGCAG
CTCAGCTCTTAGGAGCAAGTTGTGGGGGCTTTTCAAGAGGGGCCAGGCTTCCTGGAGGGTGACTGATGTG
GCCGAAGCAGGTGTCCAGGCAGGTAGGCTGCAGCCAGGAGCTCCCTGGCACCGCAGGACCTCGTGGTACT
CTTGCCTTAGATTTTACACACACTCCACAGCCAAGCACTGCCACGGTCCTCCAGGACCTGGGAAGCAAAG
GCACAGGCCCACGGTGGCCAGCCATTGTGGTGCCGCCCCAGCTTCTGGATACAGCCTTTTGGGTAAACAC
TGGGAACTCCAGAAGTTGTGGGGAGAGTGGGGAATCAGACAGCCGCCTCTAGGGGCTGGGTTCTGCTGGG
GCCTCCTTGTTGGTGCTGTAGGCACCCGCCAGGGAGCAGGGACCCGACTTGCAGACGCATTGCCCGGTAC
TAGGAAGGAGTGAGGTGTGTTCCCACCGTACACTTCCCACACGAGCTGCGGCTGCCAGCCTCGGGCCATC
AGCCTAGGAGAGCAGATGCAGCTCCAGGGGCTCGACTTATAGCCAGTTACAGCTCCCCGGCTCTTCTGTG
TGGCAGAGCGTCGTTTCCGGGCCCTCAGGGCTGGGGAGCTCAGTTCCCATTGCTTGTGCTCAGGGCTGAG
TCTTAAAGAAGGGTTTGCCGGCCCTAACGCTGCAGCGCGTGCGCGGTGAGAGGCCCTTTTTGAGCCTGTT
TACTCCTGTGGCCTTGGGCAGAACAGTAAATACTCTGTGCACGGAGGAAAGACATGCCCAAGAGGAAGGA
AGTACTGACCATCGGCTGCCTGTGAGCAGCTTAGCAAGGAGCCCTTGCTCCCTGGGAAAGGCGGTGAACT
TGAGTCTAAAGATGCAGTGCCTGGCCCTTCCTAAGGTCCCTGCCTGGCATCCGAGTGTCGGTGTGTGGCA
CAGAAGGCTCCTGCTTGCTTCCAAAGTGATGGACAGGAAGGGGCAGAGTGAGTCACGGCCCAGACTGGGC
ACCTTCGCGTCTCAGCCTCAGGGAGCCCCACAGCCCCAAGCTCGCTGAGGCAACGTGAGAACAGGCTATG
GGAAGGCTGCAAAGGCTGAGAAATGCAAAGGCTCATATTTATAAATCCCACCCCCAGAGTGGGGAGGGTC
AGGTGCCAGACCTGGACTAAACTGCACCAAGGAAACACCCAGCAGGGTCTCCTGTGAGCCGGGGACCATG
CAGCCCGAAACCTCCAGTCACTGCGCCCGGCAGGAGTCAGGAGCCAGGGACTGTGCAGCCTGGAACCTCC
AGTCACTGTGCCCAGCAGGGTGGGCTGTGCCCAGCAGGAGTCAGGCTAAGAAACGCCAGGTCTGCCTGTT
CTTGCTGGGCAATGGCTGATGGCTGCCAGTTTCTGCTGATACACAGGTAGGATGGGACCCTTCATGAATA
TCTGACTTTAATAAGTTGGTAAGGATATATTTTTTTGTCTATGTTCTGTTTCAACTTATGTAGATTATTA
TAAATTGATGTAAACCACGTGAGAGGAAAATGTTAATAAAAAATGCAAAGCCCCATCATTTGCACAAAAC
TCA
SEQ ID NO: 2
- Homo sapiens actin, beta (ACTB), mRNA
ACCGCCGAGACCGCGTCCGCCCCGCGAGCACAGAGCCTCGCCTTTGCCGATCCGCCGCCCGTCCACACCC
GCCGCCAGCTCACCATGGATGATGATATCGCCGCGCTCGTCGTCGACAACGGCTCCGGCATGTGCAAGGC
CGGCTTCGCGGGCGACGATGCCCCCCGGGCCGTCTTCCCCTCCATCGTGGGGCGCCCCAGGCACCAGGGC
GTGATGGTGGGCATGGGTCAGAAGGATTCCTATGTGGGCGACGAGGCCCAGAGCAAGAGAGGCATCCTCA
CCCTGAAGTACCCCATCGAGCACGGCATCGTCACCAACTGGGACGACATGGAGAAAATCTGGCACCACAC
CTTCTACAATGAGCTGCGTGTGGCTCCCGAGGAGCACCCCGTGCTGCTGACCGAGGCCCCCCTGAACCCC
AAGGCCAACCGCGAGAAGATGACCCAGATCATGTTTGAGACCTTCAACACCCCAGCCATGTACGTTGCTA
TCCAGGCTGTGCTATCCCTGTACGCCTCTGGCCGTACCACTGGCATCGTGATGGACTCCGGTGACGGGGT
CACCCACACTGTGCCCATCTACGAGGGGTATGCCCTCCCCCATGCCATCCTGCGTCTGGACCTGGCTGGC
CGGGACCTGACTGACTACCTCATGAAGATCCTCACCGAGCGCGGCTACAGCTTCACCACCACGGCCGAGC
GGGAAATCGTGCGTGACATTAAGGAGAAGCTGTGCTACGTCGCCCTGGACTTCGAGCAAGAGATGGCCAC
GGCTGCTTCCAGCTCCTCCCTGGAGAAGAGCTACGAGCTGCCTGACGGCCAGGTCATCACCATTGGCAAT
GAGCGGTTCCGCTGCCCTGAGGCACTCTTCCAGCCTTCCTTCCTGGGCATGGAGTCCTGTGGCATCCACG
AAACTACCTTCAACTCCATCATGAAGTGTGACGTGGACATCCGCAAAGACCTGTACGCCAACACAGTGCT
GTCTGGCGGCACCACCATGTACCCTGGCATTGCCGACAGGATGCAGAAGGAGATCACTGCCCTGGCACCC
AGCACAATGAAGATCAAGATCATTGCTCCTCCTGAGCGCAAGTACTCCGTGTGGATCGGCGGCTCCATCC
TGGCCTCGCTGTCCACCTTCCAGCAGATGTGGATCAGCAAGCAGGAGTATGACGAGTCCGGCCCCTCCAT
CGTCCACCGCAAATGCTTCTAGGCGGACTATGACTTAGTTGCGTTACACCCTTTCTTGACAAAACCTAAC
TTGCGCAGAAAACAAGATGAGATTGGCATGGCTTTATTTGTTTTTTTTGTTTTGTTTTGGTTTTTTTTTT
TTTTTTGGCTTGACTCAGGATTTAAAAACTGGAACGGTGAAGGTGACAGCAGTCGGTTGGAGCGAGCATC
CCCCAAAGTTCACAATGTGGCCGAGGACTTTGATTGCACATTGTTGTTTTTTTAATAGTCATTCCAAATA
TGAGATGCGTTCTTACAGGAAGTCCCTTCCCATCCTAAAAGCCACCCCACTTCTCTCTAAGGAGAATGGC
CCAGTCCTCTCCCAAGTCCACACAGGGGAGGTGATAGCATTGCTTTCGTGTAAATTATGTAATGCAAAAT
TTTTTTAATCTTCGCCTTAATACTTTTTTATTTTGTTTTATTTTGAATGATGAGCCTTCGTGCCCCCCCT
TCCCCCTTTTTTGTCCCCCAACTTGAGATGTATGAAGGCTTTTGGTCTCCCTGGGAGTGGGTGGAGGCAG
CCAGGGCTTACCTGTACACTGACTTGAGACCAGTTGAATAAAAGTGCACACCTTAAAAATGAAAAAAAAA
AAAAAAAAAAAAAAAAAAAAAAAAAAAAAAAA
SEQ ID NO: 3
- Homo sapiens ARP1 actin-related protein 1 homolog A,
centractin alpha (yeast) (ACTR1A), mRNA
GCTCCCTCGCCGCCCTGAACCGGCGGCTAGACTGCGCATGCGTGTCAGTGGCGCTAGCGGCGGACCCGGC
TGGGCAGTTCCTTCCCCAGAAGGAGAGATTCCTCTGCCATGGAGTCCTACGATGTGATCGCCAACCAGCC
TGTCGTGATCGACAACGGATCCGGTGTGATTAAAGCTGGTTTTGCTGGTGATCAGATCCCCAAATACTGC
TTTCCAAACTATGTGGGCCGACCCAAGCACGTTCGTGTCATGGCAGGAGCCCTTGAAGGCGACATCTTCA
TTGGCCCCAAAGCTGAGGAGCACCGAGGGCTGCTTTCAATCCGCTATCCCATGGAGCATGGCATCGTCAA
GGATTGGAACGACATGGAACGCATTTGGCAATATGTCTATTCTAAGGACCAGCTGCAGACTTTCTCAGAG
GAGCATCCTGTGCTCCTGACTGAGGCGCCTTTAAACCCACGAAAAAACCGGGAACGAGCTGCCGAAGTTT
TCTTCGAGACCTTCAATGTGCCCGCTCTTTTCATCTCCATGCAAGCTGTACTCAGCCTTTACGCTACAGG
CAGGACCACAGGGGTGGTGCTGGATTCTGGGGATGGAGTCACCCATGCTGTGCCCATCTATGAGGGCTTT
GCCATGCCCCACTCCATCATGCGCATCGACATCGCGGGCCGGGACGTCTCTCGCTTCCTGCGCCTCTACC
TGCGTAAGGAGGGCTACGACTTCCACTCATCCTCTGAGTTTGAGATTGTCAAGGCCATAAAAGAAAGAGC
CTGTTACCTATCCATAAACCCCCAAAAGGATGAGACGCTAGAGACAGAGAAAGCTCAGTACTACCTGCCT
GATGGCAGCACCATTGAGATTGGTCCTTCCCGATTCCGGGCCCCTGAGTTGCTCTTCAGGCCAGATTTGA
TTGGAGAGGAGAGTGAAGGCATCCACGAGGTCCTGGTGTTCGCCATTCAGAAGTCAGACATGGACCTGCG
GCGCACGCTTTTCTCTAACATTGTCCTCTCAGGAGGCTCTACCCTGTTCAAAGGTTTTGGTGACAGGCTC
CTGAGTGAAGTGAAGAAACTAGCTCCAAAAGATGTGAAGATCAGGATATCTGCACCTCAGGAGAGACTGT
ATTCCACGTGGATTGGGGGCTCCATCCTTGCCTCCCTGGACACCTTTAAGAAGATGTGGGTCTCCAAAAA
GGAATATGAGGAAGACGGTGCCCGATCCATCCACAGAAAAACCTTCTAATGTCGGGACATCATCTTCACC
TCTCTCTGAAGTTAACTCCACTTTAAAACTCGCTTTCTTGAGTCGGAGTGTTTGCGAGGAACTGCCTGTG
TGTGAGTGCGTGTGTGGATATGAGTGTGTGTGCACATGCGAGTGCCGTGTGGCCCTGGGACCCTGGGCCC
AGAAAGGACGATGAACTACCTGCAGTGGTGATGGCCTGAGGCCTGGGGTTGACCACTAACTGGCTCCTGA
CAGGGAAGAGCGCTGGCAGAGGCTGTGCTCCCTCCTCAGGTGGCCTCTGGCTGGCTGTGGGGGACTCCGT
TTACTACCACAGGGAGACAGAGGGAGGTAAGCCATCCCCCGGGAGACCTTGCTGCTGACCATCCTAGGCT
GGGCTGGCCCCACCCTCACCCCCACCCCCAGGGTGCCCTGAGGCCCCAGGCAGCTGCTGCCTCCACTATC
GATGCCTCCTGACTGCACACTGAGGACTGGGACTGGGGTTGAGTTCTGTCTGGTTTTGTTGCCATTTTGG
TTTGGGAGGCTGGAAAAGCACCCCAAGAGCTATTACAGAGACTGGAGTCAGGAGAGAGCAGGAGGCCCTC
ATGTTCACCAGGGAACAGGACCACACCGGCCACTGGAGGAGGGCAGGAGCAGTCCTCACTCTGAATGGCT
GCAGAGTTAATGTTCCCAGCCCAGTCCCCTTTCGGGGGCCTTGGGAGAGTTTAAGGCACCTGCTGGTTCC
AGGACCTCGCTTTCCATCTGTTCTTGTTGCAATGCCATCTTCAAACCGTTTTATTTATTGAAGTGTTTGT
TCAGTTAGGGGCTGGAGAGAGGGAGCTTGCTGCCTCCTGCCTTGCTACACTAATGTTTACAGCACCTAAG
CTTAGCCTCCAGGGCCCCACCTCTCCCAGCTGATGGTGAGCTGACAGTGTCCACAGGTTCCAGGACCATT
TGAGATTGGAAGCTACACTCAAAGACACTCCCACCAGGCTCTTTCTCCCTTTTCCTCTTGCTCACTGCCC
TGGAATCAACAGGCTGGTTGCTGGTTAGATTTTCTGAAACAGGAGGTAAAATTTTTCTTTGGCAGAGGCC
CCTAAGCAAGGGAGGGGTGTTGGAGAGCCAGTGCCCTTAAGACTGGAGAAAGCTGCAATTTACCAAGTTG
CCTTTTGCCACTGTAGCTGACCAGGGGACTAGGTTGTAGAGGTGGGAAGGCCCCCTCTGGGCTGATCTTG
TGCCATTCTTGACCTTGGACCTGCTTGGTTAAGGAGGGAGTGGGCCAGACCAGAGTGCCAGGAGCTAATG
GAGCCAGGCCTGACTCCTAGGAGTGGTCCAAAGGCCTTCAGCCTAGATGGTGCAAAGCTGGGGCCAGCCT
GTCTTCACCGGCACCCTCACCTGTGACACCAAGACCCACCCCAATCCCAGACTTCACACAGTATTCTCCC
CCACGCCGTCCTATGACCAAAGGCCCCTGCCAGGTGTGGGCCACAGCAGCAGGTATGTGTGAAAGCAACG
TAGCGCCCCGCGGACTGCAGTGCGCTTAACCAACTCACCTCCCTTCTCTTAGCCCAAGCCTGTCCCTCGC
ACAGCCTCGCACAAACCACATTGCCTGGTGGGGCCCAGTGTACTGAAATAAAGTCGTTCCGATAGACACG
TCAAAAAAAAAAAAAAAAAAA
SEQ ID NO: 4
- Homo sapiens ADAMTS-like 4 (ADAMTSL4), transcript
variant 1, mRNA
CCGCCGCGGAGCGAGGTTGCCTGGAGAGAGCGCCTGGGCGCAGAAGGGTTAACGGGCCACCGGGGGCTCG
CAGAGCAGGAGGGTGCTCTCGGACGGTGTGTCCCCCACTGCACTCCTGAACTTGGAGGACAGGGTCGCCG
CGAGGGACGCAGAGAGCACCCTCCACGCCCAGATGCCTGCGTAGTTTTTGTGACCAGTCCGCTCCTGCCT
CCCCCTGGGGCAGTAGAGGGGGAGCGATGGAGAACTGGACTGGCAGGCCCTGGCTGTATCTGCTGCTGCT
TCTGTCCCTCCCTCAGCTCTGCTTGGATCAGGAGGTGTTGTCCGGACACTCTCTTCAGACACCTACAGAG
GAGGGCCAGGGCCCCGAAGGTGTCTGGGGACCTTGGGTCCAGTGGGCCTCTTGCTCCCAGCCCTGCGGGG
TGGGGGTGCAGCGCAGGAGCCGGACATGTCAGCTCCCTACAGTGCAGCTCCACCCGAGTCTGCCCCTCCC
TCCCCGGCCCCCAAGACATCCAGAAGCCCTCCTCCCCCGGGGCCAGGGTCCCAGACCCCAGACTTCTCCA
GAAACCCTCCCCTTGTACAGGACACAGTCTCGGGGAAGGGGTGGCCCACTTCGAGGTCCCGCTTCCCACC
TAGGGAGAGAGGAGACCCAGGAGATTCGAGCGGCCAGGAGGTCCCGGCTTCGAGACCCCATCAAGCCAGG
AATGTTCGGTTATGGGAGAGTGCCCTTTGCATTGCCACTGCACCGGAACCGCAGGCACCCTCGGAGCCCA
CCCAGATCTGAGCTGTCCCTGATCTCTTCTAGAGGGGAAGAGGCTATTCCGTCCCCTACTCCAAGAGCAG
AGCCATTCTCCGCAAACGGCAGCCCCCAAACTGAGCTCCCTCCCACAGAACTGTCTGTCCACACCCCATC
CCCCCAAGCAGAACCTCTAAGCCCTGAAACTGCTCAGACAGAGGTGGCCCCCAGAACCAGGCCTGCCCCC
CTACGGCATCACCCCAGAGCCCAGGCCTCTGGCACAGAGCCCCCCTCACCCACGCACTCCTTAGGAGAAG
GTGGCTTCTTCCGTGCATCCCCTCAGCCACGAAGGCCAAGTTCCCAGGGTTGGGCCAGTCCCCAGGTAGC
AGGGAGACGCCCTGATCCTTTTCCTTCGGTCCCTCGGGGCCGAGGCCAGCAGGGCCAAGGGCCTTGGGGA
ACGGGGGGGACTCCTCACGGGCCCCGCCTGGAGCCTGACCCTCAGCACCCGGGCGCCTGGCTGCCCCTGC
TGAGCAACGGCCCCCATGCCAGCTCCCTCTGGAGCCTCTTTGCTCCCAGTAGCCCTATTCCAAGATGTTC
TGGGGAGAGTGAACAGCTAAGAGCCTGCAGCCAAGCGCCCTGCCCCCCTGAGCAGCCAGACCCCCGGGCC
CTGCAGTGCGCAGCCTTTAACTCCCAGGAATTCATGGGCCAGCTGTATCAGTGGGAGCCCTTCACTGAAG
TCCAGGGCTCCCAGCGCTGTGAACTGAACTGCCGGCCCCGTGGCTTCCGCTTCTATGTCCGTCACACTGA
AAAGGTCCAGGATGGGACCCTGTGTCAGCCTGGAGCCCCTGACATCTGTGTGGCTGGACGCTGTCTGAGC
CCCGGCTGTGATGGGATCCTTGGCTCTGGCAGGCGTCCTGATGGCTGTGGAGTCTGTGGGGGTGATGATT
CTACCTGTCGCCTTGTTTCGGGGAACCTCACTGACCGAGGGGGCCCCCTGGGCTATCAGAAGATCTTGTG
GATTCCAGCGGGAGCCTTGCGGCTCCAGATTGCCCAGCTCCGGCCTAGCTCCAACTACCTGGCACTTCGT
GGCCCTGGGGGCCGGTCCATCATCAATGGGAACTGGGCTGTGGATCCCCCTGGGTCCTACAGGGCCGGCG
GGACCGTCTTTCGATATAACCGTCCTCCCAGGGAGGAGGGCAAAGGGGAGAGTCTGTCGGCTGAAGGCCC
CACCACCCAGCCTGTGGATGTCTATATGATCTTTCAGGAGGAAAACCCAGGCGTTTTTTATCAGTATGTC
ATCTCTTCACCTCCTCCAATCCTTGAGAACCCCACCCCAGAGCCCCCTGTCCCCCAGCTTCAGCCGGAGA
TTCTGAGGGTGGAGCCCCCACTTGCTCCGGCACCCCGCCCAGCCCGGACCCCAGGCACCCTCCAGCGTCA
GGTGCGGATCCCCCAGATGCCCGCCCCGCCCCATCCCAGGACACCCCTGGGGTCTCCAGCTGCGTACTGG
AAACGAGTGGGACACTCTGCATGCTCAGCGTCCTGCGGGAAAGGTGTCTGGCGCCCCATTTTCCTCTGCA
TCTCCCGTGAGTCGGGAGAGGAACTGGATGAACGCAGCTGTGCCGCGGGTGCCAGGCCCCCAGCCTCCCC
TGAACCCTGCCACGGCACCCCATGCCCCCCATACTGGGAGGCTGGCGAGTGGACATCCTGCAGCCGCTCC
TGTGGCCCCGGCACCCAGCACCGCCAGCTGCAGTGCCGGCAGGAATTTGGGGGGGGTGGCTCCTCGGTGC
CCCCGGAGCGCTGTGGACATCTCCCCCGGCCCAACATCACCCAGTCTTGCCAGCTGCGCCTCTGTGGCCA
TTGGGAAGTTGGCTCTCCTTGGAGCCAGTGCTCCGTGCGGTGCGGCCGGGGCCAGAGAAGCCGGCAGGTT
CGCTGTGTTGGGAACAATGGTGATGAAGTGAGCGAGCAGGAGTGTGCGTCAGGCCCCCCGCAGCCCCCCA
GCAGAGAGGCCTGTGACATGGGGCCCTGTACTACTGCCTGGTTCCACAGCGACTGGAGCTCCAAGTGCTC
AGCCGAGTGTGGGACGGGAATCCAGCGGCGCTCTGTGGTCTGCCTTGGGAGTGGGGCAGCCCTCGGGCCA
GGCCAGGGGGAAGCAGGAGCAGGAACTGGGCAGAGCTGTCCAACAGGAAGCCGGCCCCCTGACATGCGCG
CCTGCAGCCTGGGGCCCTGTGAGAGAACTTGGCGCTGGTACACAGGGCCCTGGGGTGAGTGCTCCTCCGA
ATGTGGCTCTGGCACACAGCGTAGAGACATCATCTGTGTATCCAAACTGGGGACGGAGTTCAACGTGACT
TCTCCGAGCAACTGTTCTCACCTCCCCAGGCCCCCTGCCCTGCAGCCCTGTCAAGGGCAGGCCTGCCAGG
ACCGATGGTTTTCCACGCCCTGGAGCCCATGTTCTCGCTCCTGCCAAGGGGGAACGCAGACACGGGAGGT
CCAGTGCCTGAGCACCAACCAGACCCTCAGCACCCGATGCCCTCCTCAACTGCGGCCCTCCAGGAAGCGC
CCCTGTAACAGCCAACCCTGCAGCCAGCGCCCTGATGATCAATGCAAGGACAGCTCTCCACATTGCCCCC
TGGTGGTACAGGCCCGGCTCTGCGTCTACCCCTACTACACAGCCACCTGTTGCCGCTCTTGCGCACATGT
CCTGGAGCGGTCTCCCCAGGATCCCTCCTGAAAGGGGTCCGGGGCACCTTCACGGTTTTCTGTGCCACCA
TCGGTCACCCATTGATCGGCCCACTCTGAACCCCCTGGCTCTCCAGCCTGTCCCAGTCTCAGCAGGGATG
TCCTCCAGGTGACAGAGGGTGGCAAGGTGACTGACACAAAGTGACTTTCAGGGCTGTGGTCAGGCCCATG
TGGTGGTGTGATGGGTGTGTGCACATATGCCTCAGGTGTGCTTTTGGGACTGCATGGATATGTGTGTGCT
CAAACGTGTATCACTTTTCAAAAAGAGGTTACACAGACTGAGAAGGACAAGACCTGTTTCCTTGAGACTT
TCCTAGGTGGAAAGGAAAGCAAGTCTGCAGTTCCTTGCTAATCTGAGCTACTTAGAGTGTGGTCTCCCCA
CCAACTCCAGTTTTGTGCCCTAAGCCTCATTTCTCATGTTCAGACCTCACATCTTCTAAGCCGCCCTGTG
TCTCTGACCCCTTCTCATTTGCCTAGTATCTCTGCCCCTGCCTCCCTAATTAGCTAGGGCTGGGGTCAGC
CACTGCCAATCCTGCCTTACTCAGGAAGGCAGGAGGAAAGAGACTGCCTCTCCAGAGCAAGGCCCAGCTG
GGCAGAGGGTGAAAAAGAGAAATGTGAGCATCCGCTCCCCCACCACCCCGCCCAGCCCCTAGCCCCACTC
CCTGCCTCCTGAAATGGTTCCCACCCAGAACTAATTTATTTTTTATTAAAGATGGTCATGACAAATGAGA
AAAAAAAAA
SEQ ID NO: 5
- Homo sapiens anaphase promoting complex subunit 1
(ANAPC1), mRNA
CGCGTCCATTTGAACGTCTCGCACGCCTTCCTGCCATTAGCACTCGAGCCCGCTGCTGTTGCCCGTTCTT
CCTCCAGAATAGGGGAGGGAGAGGGAATGAGAAGCTGCTGCGGCCCAAGAGTCACTGTGAAGGACCCCGC
CGCTGCCCTCGGGCCTCCTCGGCCCCTGCGCCTCCGGGGAGCAGCCGGGGCTCGCCGCGCCTGACGCGTC
CCGAGTTATACAGAAATAATGTTGATATTTGGAACCCATGTCGAACTTCTATGAAGAAAGGACAACGATG
ATTGCAGCAAGGGATTTGCAGGAATTTGTTCCTTTTGGTCGAGACCACTGCAAGCACCACCCTAATGCTT
TGAACCTTCAACTTCGCCAGCTGCAGCCAGCTTCTGAATTATGGTCTTCTGATGGTGCTGCTGGCTTGGT
GGGATCCCTTCAGGAGGTTACAATCCACGAGAAACAGAAGGAAAGCTGGCAGTTAAGGAAAGGAGTAAGT
GAAATTGGAGAAGATGTGGACTATGATGAGGAACTCTATGTTGCTGGAAATATGGTGATATGGAGCAAAG
GAAGTAAAAGCCAGGCATTGGCAGTTTATAAAGCATTTACAGTTGACAGTCCTGTTCAGCAGGCATTGTG
GTGTGACTTCATTATATCACAGGATAAGTCTGAAAAGGCCTACAGTAGCAATGAAGTAGAAAAATGCATA
TGTATATTGCAAAGCTCATGTATTAACATGCATAGCATAGAAGGAAAGGATTACATAGCTTCATTACCAT
TTCAGGTTGCAAATGTTTGGCCCACTAAATATGGATTGCTGTTTGAACGAAGCGCTTCTTCACATGAAGT
ACCTCCAGGTTCACCCAGAGAACCTTTACCTACTATGTTCAGCATGCTGCACCCACTAGATGAAATAACT
CCACTTGTTTGTAAATCTGGAAGTCTTTTTGGTTCATCACGGGTGCAATATGTTGTAGATCATGCAATGA
AAATTGTTTTCCTCAATACTGACCCCTCTATTGTAATGACTTATGATGCTGTTCAAAATGTGCATTCTGT
GTGGACTCTCCGGAGAGTCAAATCAGAGGAAGAGAATGTTGTTTTAAAGTTCTCTGAACAGGGGGGAACC
CCACAGAATGTGGCCACTAGCAGCTCCCTCACAGCACATCTCAGAAGCCTCTCCAAAGGAGATTCCCCTG
TGACTTCACCTTTCCAGAATTACTCCTCCATTCACAGCCAGAGTCGCTCAACCTCATCACCCAGTCTACA
TTCTCGCTCACCTTCTATTTCCAACATGGCAGCTCTAAGTCGTGCTCATTCTCCTGCGTTAGGAGTGCAC
TCTTTTTCAGGGGTGCAAAGGTTCAACATTTCAAGCCATAATCAGTCTCCAAAGAGACATAGTATTTCTC
ATTCTCCAAATAGTAATTCTAATGGCTCCTTTCTTGCACCAGAAACGGAGCCAATTGTTCCTGAACTGTG
TATTGACCATTTGTGGACAGAAACGATTACTAATATAAGAGAGAAAAATTCACAAGCCTCAAAAGTGTTT
ATTACATCTGACCTATGTGGGCAAAAGTTCCTGTGCTTTTTAGTAGAGTCCCAGCTCCAGTTACGCTGTG
TAAAGTTTCAAGAGAGTAATGATAAAACCCAGCTCATCTTTGGTTCAGTGACCAACATACCAGCAAAGGA
TGCAGCACCAGTGGAGAAAATAGACACCATGCTGGTCTTGGAAGGCAGTGGAAACCTGGTGCTATACACA
GGAGTGGTTCGGGTGGGAAAGGTTTTTATTCCTGGACTGCCAGCTCCCTCTCTGACGATGTCCAACACAA
TGCCTCGGCCCAGTACTCCACTAGATGGCGTTAGTACTCCAAAGCCTCTTAGTAAACTCCTTGGATCATT
GGACGAGGTTGTTCTGTTGTCCCCAGTTCCAGAACTGAGGGATTCTTCAAAACTTCATGATTCTCTCTAT
AATGAGGATTGTACTTTCCAACAGCTTGGAACTTACATTCATTCTATCAGAGATCCTGTCCATAACAGAG
TCACCCTGGAACTGAGTAATGGCTCCATGGTTAGGATCACTATTCCTGAAATTGCCACCTCTGAGTTAGT
ACAAACGTGTTTGCAAGCAATTAAGTTTATCCTGCCAAAAGAAATAGCAGTTCAGATGCTTGTCAAGTGG
TACAATGTCCACAGTGCTCCAGGAGGACCCAGTTATCACTCAGAGTGGAATTTATTTGTGACTTGTCTCA
TGAACATGATGGGTTATAACACAGACCGCTTAGCATGGACTAGAAATTTTGACTTTGAAGGATCACTTTC
TCCTGTCATTGCGCCCAAAAAAGCAAGGCCTTCCGAGACTGGATCTGATGATGACTGGGAATATTTACTA
AATTCAGACTACCACCAGAATGTTGAGTCTCATCTTTTGAACAGATCTTTATGTCTGAGTCCTTCAGAAG
CTTCACAGATGAAGGATGAGGATTTTTCACAGAATCTCAGTCTGGATTCTTCTACACTTCTCTTTACTCA
CATACCTGCAATTTTTTTCGTTCTTCACCTTGTGTATGAGGAGCTTAAGTTGAATACTCTAATGGGAGAA
GGAATTTGTTCACTTGTTGAACTTCTCGTTCAGTTGGCAAGGGACTTAAAATTGGGGCCTTATGTAGATC
ATTACTATAGAGACTACCCAACGCTTGTCAGAACTACTGGACAAGTGTGCACAATTGATCCAGGTCAAAC
AGGATTTATGCATCATCCATCATTTTTTACGTCTGAGCCACCAAGTATTTATCAGTGGGTGAGTTCTTGT
CTGAAGGGTGAAGGAATGCCACCTTATCCTTACCTCCCTGGAATCTGTGAAAGAAGCAGACTTGTAGTCT
TGAGTATTGCACTGTACATACTTGGTGATGAGAGCTTGGTTTCTGATGAATCCTCACAGTATTTAACCAG
AATAACTATAGCCCCCCAGAAGTTGCAAGTAGAACAAGAGGAAAACAGGTTTAGTTTCAGGCATTCTACA
TCTGTTTCTAGTCTAGCTGAAAGATTGGTTGTCTGGATGACTAATGTAGGATTCACTTTAAGAGATTTGG
AAACTCTTCCCTTTGGAATTGCTCTTCCCATCAGAGATGCAATTTATCACTGTCGTGAGCAGCCTGCCTC
AGACTGGCCAGAAGCTGTCTGTCTCTTGATTGGACGTCAGGATCTTTCCAAGCAGGCCTGCGAAGGAAAC
TTACCCAAAGGGAAGTCTGTGCTCTCATCAGATGTTCCTTCAGGAACAGAAACTGAGGAGGAAGATGACG
GCATGAATGACATGAATCACGAGGTCATGTCATTAATATGGAGTGAAGATTTAAGGGTGCAGGATGTGCG
AAGGCTTCTTCAGAGTGCGCATCCTGTCCGTGTCAACGTAGTGCAGTACCCAGAGCTCAGTGACCACGAG
TTCATCGAGGAAAAGGAAAACAGATTGCTCCAATTGTGTCAGCGAACTATGGCTCTTCCTGTAGGACGAG
GAATGTTTACCTTGTTTTCGTACCATCCTGTTCCAACAGAGCCATTGCCTATTCCTAAATTGAATCTGAC
TGGGCGTGCCCCTCCTCGGAACACAACAGTAGACCTTAATAGTGGAAACATCGATGTGCCTCCCAACATG
ACAAGCTGGGCCAGCTTTCATAATGGTGTGGCTGCTGGCCTGAAGATAGCTCCTGCCTCCCAGATCGACT
CAGCTTGGATTGTTTACAATAAGCCCAAGCATGCTGAGTTGGCCAATGAGTATGCTGGCTTTCTCATGGC
TCTGGGTTTGAATGGGCACCTTACCAAGCTGGCGACTCTCAATATCCATGACTACTTGACCAAGGGCCAT
GAAATGACAAGCATTGGACTGCTACTTGGTGTTTCTGCTGCAAAACTAGGCACCATGGATATGTCTATTA
CTCGGCTTCTTAGCATTCACATTCCTGCTCTCTTACCCCCAACGTCCACAGAGCTGGATGTTCCTCACAA
TGTCCAAGTGGCTGCAGTGGTTGGCATTGGCCTTGTATATCAAGGGACAGCTCACAGACATACTGCAGAA
GTCCTGTTGGCTGAGATAGGACGGCCTCCTGGTCCTGAAATGGAATACTGCACTGACAGAGAGTCATACT
CCTTAGCTGCTGGCTTGGCCCTGGGCATGGTCTGCTTGGGGCATGGCAGCAATTTGATAGGTATGTCTGA
TCTCAATGTGCCTGAGCAGCTCTATCAGTACATGGTTGGAGGACATAGGCGCTTTCAAACAGGAATGCAT
AGGGAGAAACATAAATCACCAAGTTATCAAATCAAAGAAGGAGATACCATAAATGTGGATGTGACTTGTC
CAGGTGCTACTCTAGCTTTGGCTATGATCTACTTAAAAACCAATAACAGATCTATTGCAGATTGGCTCCG
AGCCCCTGACACCATGTATTTGTTGGACTTTGTGAAGCCAGAATTTCTCTTGCTTAGGACACTTGCTCGA
TGCCTGATTTTGTGGGATGATATTTTACCAAATTCCAAGTGGGTTGACAGCAATGTTCCTCAAATTATAA
GAGAAAATAGTATCTCTCTCAGTGAAATCGAATTGCCGTGCTCAGAGGATTTGAATTTGGAAACTTTGTC
CCAAGCACATGTCTACATAATTGCAGGAGCCTGCTTGTCTCTGGGTTTTCGATTTGCTGGCTCAGAAAAC
TTATCAGCATTTAACTGTTTGCATAAATTTGCCAAAGATTTTATGACTTATTTGTCCGCACCTAATGCTT
CTGTTACAGGTCCTCATAACCTAGAAACTTGTCTGAGCGTGGTGCTGCTGTCTCTCGCCATGGTCATGGC
TGGCTCAGGAAACCTAAAGGTTTTGCAGCTTTGTCGCTTCTTACACATGAAAACGGGTGGTGAAATGAAC
TATGGTTTTCACTTAGCCCACCACATGGCCCTTGGACTTCTATTTTTGGGAGGAGGAAGGTACTCTTTGA
GCACATCAAATTCTTCCATTGCCGCTCTTCTCTGTGCCCTTTATCCGCACTTCCCAGCTCACAGCACTGA
CAACCGGTATCATCTCCAGGCTCTCCGGCACCTCTATGTGCTGGCCGCGGAGCCCAGGCTTCTAGTGCCT
GTGGATGTGGACACAAACACGCCCTGCTATGCCCTCTTAGAAGTTACCTACAAGGGCACTCAGTGGTATG
AACAAACCAAAGAAGAATTGATGGCTCCTACCCTTCTTCCAGAACTCCATCTTTTAAAGCAGATTAAAGT
AAAAGGCCCAAGATACTGGGAACTGCTCATAGATTTAAGCAAAGGAACACAACACTTGAAGTCCATCCTT
TCCAAGGATGGGGTTTTATATGTTAAACTCCGGGCGGGTCAGCTCTCCTACAAAGAAGATCCAATGGGAT
GGCAAAGTTTGTTGGCTCAGACTGTTGCTAACAGGAACTCTGAAGCCCGGGCTTTCAAGCCAGAAACAAT
CTCAGCATTCACTTCTGATCCAGCACTTCTGTCATTTGCTGAATATTTCTGCAAGCCAACTGTGAACATG
GGTCAGAAACAGGAAATTCTGGATCTCTTTTCTTCAGTACTCTATGAATGTGTTACCCAGGAGACCCCAG
AGATGTTGCCTGCATACATAGCAATGGATCAGGCTATAAGAAGACTTGGGAGAAGAGAAATGTCTGAGAC
TTCTGAACTTTGGCAGATAAAGTTGGTGTTAGAGTTTTTCAGCTCCCGAAGCCATCAGGAGCGGCTGCAG
AACCACCCTAAGCGGGGGCTCTTTATGAACTCGGAATTCCTCCCTGTTGTGAAGTGCACCATTGATAATA
CCCTGGACCAGTGGCTACAAGTCGGGGGTGATATGTGTGTGCACGCCTACCTCAGCGGGCAGCCCTTGGA
GGAATCACAGCTGAGCATGCTGGCCTGCTTCCTCGTCTACCACTCTGTGCCAGCTCCACAGCACCTGCCA
CCTATAGGACTAGAAGGGAGCACAAGCTTTGCTGAACTGCTCTTCAAATTTAAGCAGCTAAAAATGCCAG
TGCGAGCTTTGCTGAGATTGGCTCCTTTGCTTCTTGGAAATCCACAGCCAATGGTGATGTGACCGTGTCT
GGCGGTGAACCTACCCTGAAACGTGACTTCTGCACAACAAACGTGACCAAACATCAAAGCTAAAGCAATG
TTTATAAAGTTTTATGGTATAACTAGGGGGAAATGAGCTGCACAAACCTCAATGTATTTTAAATCTGTTG
CTGTCATCATTAACGGTATATGACATATAAAAGCAAGTTAAAATTTACTTTTGTAAATAAAGTTTTTGGT
TTGTTTCCAAAAAAAAAAAAAAAAAAAAA
SEQ ID NO: 6
- Homo sapiens apolipoprotein B mRNA editing enzyme,
catalytic polypeptide-like 3F (APOBEC3F), transcript
variant 1, mRNA
TTCCCTTTGCAATTGCCTTGGGTCCTGCCGCACAGAGCGGCCTGTCTTTATCAGAGGTCCCTCTGCCAGG
GGGAGGGCCCCAGAGAAAACCAGAAAGAGGGTGAGAGACTGAGGAAGATAAAGCGTCCCAGGGCCTCCTA
CACCAGCGCCTGAGCAGGAAGGGGGAGGGGCCATGACTACGAGGCCCTGGGAGGTCACTTTAGGGAGGGC
TGTCCTGAAACCTGGAGCCTGGAGCAGAAAGTGAAACCCTGGTGCTCCAGACAAAGATCTTAGTCGGGAC
TAGCCGGCCAAGGATGAAGCCTCACTTCAGAAACACAGTGGAGCGAATGTATCGAGACACATTCTCCTAC
AACTTTTATAATAGACCCATCCTTTCTCGTCGGAATACCGTCTGGCTGTGCTACGAAGTGAAAACAAAGG
GTCCCTCAAGGCCCCGTTTGGACGCAAAGATCTTTCGAGGCCAGGTGTATTCCCAGCCTGAGCACCACGC
AGAAATGTGCTTCCTCTCTTGGTTCTGTGGCAACCAGCTGCCTGCTTACAAGTGTTTCCAGATCACCTGG
TTTGTATCCTGGACCCCCTGCCCGGACTGTGTGGCGAAGCTGGCCGAATTCCTGGCTGAGCACCCCAATG
TCACCCTGACCATCTCCGCCGCCCGCCTCTACTACTACTGGGAAAGAGATTACCGAAGGGCGCTCTGCAG
GCTGAGTCAGGCAGGGGCCCGCGTGAAGATTATGGACGATGAAGAATTTGCATACTGCTGGGAAAACTTT
GTGTACAGTGAAGGTCAGCCATTCATGCCTTGGTACAAATTCGATGACAATTATGCATTCCTGCACCGCA
CGCTAAAGGAGATTCTCAGAAACCCGATGGAGGCAATGTATCCACACATATTCTACTTCCACTTTAAAAA
CCTACGCAAAGCCTATGGTCGGAACGAAAGCTGGCTGTGCTTCACCATGGAAGTTGTAAAGCACCACTCA
CCTGTCTCCTGGAAGAGGGGCGTCTTCCGAAACCAGGTGGATCCTGAGACCCATTGTCATGCAGAAAGGT
GCTTCCTCTCTTGGTTCTGTGACGACATACTGTCTCCTAACACAAACTACGAGGTCACCTGGTACACATC
TTGGAGCCCTTGCCCAGAGTGTGCAGGGGAGGTGGCCGAGTTCCTGGCCAGGCACAGCAACGTGAATCTC
ACCATCTTCACCGCCCGCCTCTACTACTTCTGGGATACAGATTACCAGGAGGGGCTCCGCAGCCTGAGTC
AGGAAGGGGCCTCCGTGGAGATCATGGGCTACAAAGATTTTAAATATTGTTGGGAAAACTTTGTGTACAA
TGATGATGAGCCATTCAAGCCTTGGAAAGGACTAAAATACAACTTTCTATTCCTGGACAGCAAGCTGCAG
GAGATTCTCGAGTGAGGGGTCTCCCCGGGCCTCATGGTCTGTCTCCTCTAGCCTCCTGCTCATGTTGTGC
AGGCCTCCCCTCCATCCTGGACCAGCTGTGCTTTTGCCTGGTCATCCTGAGCCCCTCCTGGCCTCAGGGC
CATTCCATAGTGCTCCCCTGCCTCACCACCTCCTCTCCGCTCTCCCAGGCTCTTCCTGCAGAGGCCTCTT
TCTGCCTCCATGGCTATCCATCCACCCACCAAGACCCTGTTCCCTGAGCCTGCATGCCCCTAACCTGCCT
TTTCCCATCTCCCCAGCATAACCTAATATTTTTTTTTTTTTTTTGAGACGGAATTTCGCTCTGTCACCCA
GACTGGAGTGCAATGGCTTGATCTTGGCTCACTGCAAACTCTGCCTACCAGGTTCAAGCGATTCTCCTGC
CTCCGCCTCCCGAGTAGCTGGAATTACAGACGCCTGCCACCACGCACAGCTAACTTTTTTTTTTTTTGTA
TTTTTAGTAGTGACTGGGTTTCACCATGTTGGCCAGGCTGGTCTTGAACTCCTGACCTCAGGTGATCCGC
CTATCTCAGCCTCCCAAAGTGCTGGGATTACAGGCGTGAGCCACTGGCCCGGCGGCACAACCAAATCTTA
TTAAACTCACCCTAGGCTGGCCGCGGTGACTCATGCCTATAATCCCCCAGCAATTTGGGAGGCAGAGGTG
AGAGAATCGCTTGAGCCCAGGAATTCGAGACCAGCCTGGGCCACATGACAAAGCCCCATCTCTACAAAAA
AATTACAAAAAAAAAAAAAACAGGTGTGGTGGCATGCACCTGTAGTTGAAGCTACTTGGAAGGATGAAGT
GGGAGGATTGCTTGAGCCGGGGAGGTGGAGGCTGCAGTGAACTGAGATCACGTCACTGAACTCCAGTCTG
AGCAACAGATCGAGACCCTGCCTGAAAATAAATCAATAAATAAACTCAACCGAAATGGGTATGAAAGTTG
AAATGGGTATGTAAGTTGAAAACCAGAAGTTTTGAGAAACATCCTTTGTTAACTTTCATCCTACAAATTG
GGTCATTCATGTCCTACGCAGCTAAAACAGAGCCCAGGAGCCAGGGAGGAAAAGCAGTCAGGCCACACAC
CATTGCTCCCAAAATGGACTTCTCTGCAAGCCTGACTCCTGAAACTGTGCATTGTACCCTGAAACCAGCT
TTATCCATAGCTTCTGCAATAAATGGCTGTAAGTCTTGGACTCCTTGCTATAATCGCAGCTATTCAGCAA
TGGAACCTCCCAGTTCCCAACCCTTCCTAGTGCCCATGGGCTTTCCCATAGGACAAGAGAACATTTCTCC
TTTTCTTTTTTTTTTTCTTTGAAATGGAGTCTCGCCCTGTCACCCAGGCTGGAGTGCAATGGTGCGGTCT
CGGCTCACTGCAACCTCTGCCTCCCTTGTTCAAGTGATTCTCCTGTCTCAGCCTCCCGAGTAGCTGGGAT
TACAGGCGTCCACCACCAAACCAGGCTAATTTTTGTATTTTTCATAAAAACGGGTTTCATCATGTTTCCC
AGGCTGGTCTTATTTTTATTTTATTTTTTGAGATGGAGTCTTGCTCTGTTGCCCAGGCTGGGGTGCAGTG
GTGCAATCTGGGTTCACTGCAGCCTCTGCCGCCTGAGTTCAAGCTATTTTCCTACCTAAGCCTCCCAAGT
AGCTGGGATTACATGCGCGTGCCACCACGCCTAGCTAATTTTTGTGTTTTTAGTAGAGACGGGGTTTCAA
CATCTTGACCAGGCTGGTCTTGAACTCCTGACCTCGTGATCCACCCGTCTCGGCCTCCCAAAGTGCTGGG
ATTACAGGCGTGAGCCACCTGGCCAGGCTTAGGCTGGTCTTAAACTCCTGACCTCAAGTGATCCAACCTC
CTTGGCCTCCCAAATTGCTGGGATTGCTGGTGTGAGCCACAGCGCCTAGCCCATTTCTCCTTTTAATAGG
ACCTGTTGCTGTCTCTGTTCTCCCAACATGGTGAACACCACCCGGACTGCGTGTATGTCCCAAATTACAA
TTCTTTCTTTGCAAATGAAATGTGAAATTTAGAGGCCCTTCTCCACACTTTAAATTTGACTTGACATTTT
CTAGGCAGATATAAGTTATTAGAGAATGAGATTCTCTATAAAAATGATCCCTTCATGCTGTGGCCTCCAC
AGAAGATGCCCTGGGCCAGGTGCCCACATGAATAATGCGGGCCACAGGCAGGCATTTATTTTCTCACAGA
TATGGAGGCTACAAGTCCAAGGTGGAGGGGTCGGCGGGGTTGTTTGCTCTGAGGCCGCTCCTCCTGGATG
GCAGGGATCCCTTCTGGCTGTGTCCTCTGTGGCCTTTCCTCTATGAACCTGTACTGTACCTCTGGGGTCT
CTCTGCTTCCAAATATCTTTTTTTTTTTTTTCAGACAGTTTTGCTCTTGTTTTCTAGGCTGGAGTGCAAT
GGCACAATCTCAGCTCACTGCAACCTCTGCCTTCCGAGTTCAAGCGATTCTCGTGCCTCAGCCTCCTGAG
TAGCTGGGACTACAGGCGTGTGCCACCACGCCTGGCTAATTTTGTAGTTTTAGTAGAGACGGGGTTTCTC
CATGTTGCTCAGGCTGGTCTTGAACTCATGAGCTCAGGCGATCCACTCTCCTCAGCCTCCCAAAGTGCTG
GGATTACAGATATAAGCCACCATACACAACTTTTTTTTTTTTTTGAGATGGAGTTTCACTCTGTTGCCCA
GGCTGGAGTGCTAAATAGCAGAATCACTGCTCACTGCAACCTCTGCCTGCTGGGTTCAAGCAATTCTCCC
ACCTCAGCCTCCTGAGTAGCTGGGATTACAGATGCCCAGAACCAATCTCTGCTAATTTTTCTATTTTTTA
GTAGAGATGGGGTTTCACTGAGGAAGGAGACCACCTCTCTCATTGTCTCCTATTTCAGAAGGAAGCAAAA
AGTTAGAAAGATGCAGAAGTAAGATCAATGGCCAGACTGTTTGGCGCTGCTACCTGGGCCTGGTAGTTAA
AGATCAACTCCTGACCTGACCGCTTGTTTTATCTAAAGATTCCAGACATTGTATGAGGAAGCATTGTGAA
ACTTTCTGGTCTGTTCTGCTAGCCCCCACCACTGATGCATGTAGCCCCCCAGTCACGTAGCCCACGCTTG
CACAATCTATCACGACCCTTTCACGTGGACCCCTTAGAATTGTAAGCCCTTAAAAGGGCCAGGGACTTCT
TCAGGGAGCTCCAATCTTCAGATGCAAGTCTGTCAACGCTCCCAGCTGATTAAAGCCTCTTCCTTCCTAA
AAAAAAAAAAAAAAAA
SEQ ID NO: 7
- Homo sapiens argininosuccinate lyase (ASL),
transcript variant 1, mRNA
CCAGGCGGAGGTGAGTGCGCGGCGGCCGGATGGGCGGGACGGGCGTGGAGGACGCCGAGCACCGTGGCGC
GCGCTCACGTCCGCGTCCCCAAGGGCTGCGCTCCCTCAAGCGCAGTGCCCAGAACTCGGAGCCAGCCCGG
CCCGGGGGACCCTGCTGGCCAAGGAGGTCGTCAGTCCGGTCTTGTCTTCCAGACCCGGAGGACCGAAGCT
TCCGGACGACGAGGAACCGCCCAACATGGCCTCGGAGAGTGGGAAGCTTTGGGGTGGCCGGTTTGTGGGT
GCAGTGGACCCCATCATGGAGAAGTTCAACGCGTCCATTGCCTACGACCGGCACCTTTGGGAGGTGGATG
TTCAAGGCAGCAAAGCCTACAGCAGGGGCCTGGAGAAGGCAGGGCTCCTCACCAAGGCCGAGATGGACCA
GATACTCCATGGCCTAGACAAGGTGGCTGAGGAGTGGGCCCAGGGCACCTTCAAACTGAACTCCAATGAT
GAGGACATCCACACAGCCAATGAGCGCCGCCTGAAGGAGCTCATTGGTGCAACGGCAGGGAAGCTGCACA
CGGGACGGAGCCGGAATGACCAGGTGGTCACAGACCTCAGGCTGTGGATGCGGCAGACCTGCTCCACGCT
CTCGGGCCTCCTCTGGGAGCTCATTAGGACCATGGTGGATCGGGCAGAGGCGGAACGTGATGTTCTCTTC
CCGGGGTACACCCATTTGCAGAGGGCCCAGCCCATCCGCTGGAGCCACTGGATTCTGAGCCACGCCGTGG
CACTGACCCGAGACTCTGAGCGGCTGCTGGAGGTGCGGAAGCGGATCAATGTCCTGCCCCTGGGGAGTGG
GGCCATTGCAGGCAATCCCCTGGGTGTGGACCGAGAGCTGCTCCGAGCAGAACTCAACTTTGGGGCCATC
ACTCTCAACAGCATGGATGCCACTAGTGAGCGGGACTTTGTGGCCGAGTTCCTGTTCTGGGCTTCGCTGT
GCATGACCCATCTCAGCAGGATGGCCGAGGACCTCATCCTCTACTGCACCAAGGAATTCAGCTTCGTGCA
GCTCTCAGATGCCTACAGCACGGGAAGCAGCCTGATGCCCCAGAAGAAAAACCCCGACAGTTTGGAGCTG
ATCCGGAGCAAGGCTGGGCGTGTGTTTGGGCGGTGTGCCGGGCTCCTGATGACCCTCAAGGGACTTCCCA
GCACCTACAACAAAGACTTACAGGAGGACAAGGAAGCTGTGTTTGAAGTGTCAGACACTATGAGTGCCGT
GCTCCAGGTGGCCACTGGCGTCATCTCTACGCTGCAGATTCACCAAGAGAACATGGGACAGGCTCTCAGC
CCCGACATGCTGGCCACTGACCTTGCCTATTACCTGGTCCGCAAAGGGATGCCATTCCGCCAGGCCCACG
AGGCCTCCGGGAAAGCTGTGTTCATGGCCGAGACCAAGGGGGTCGCCCTCAACCAGCTGTCACTGCAGGA
GCTGCAGACCATCAGCCCCCTGTTCTCGGGCGACGTGATCTGCGTGTGGGACTACGGGCACAGTGTGGAG
CAGTATGGTGCCCTGGGCGGCACTGCGCGCTCCAGCGTCGACTGGCAGATCCGCCAGGTGCGGGCGCTAC
TGCAGGCACAGCAGGCCTAGGTCCTCCCACACCTGCCCCCTAATAAAGTGGGCGCGAGAGGAGGCTGCTG
TGTGTTTCCTGCCCCAGCCTGGCTCCCTCGTTGCTGGGCTTTCGGGGCTGGCCAGTGGGGACAGTCAGGG
ACTGGAGAGGCAGGGCAGGGTGGCCTGTAATCCCAGCACTTTGGAAGGGCAAGGTGCGAGGATGCTTGAG
GCCAGGAGTTTGACACAGCCTGGGCAACACAGGGAGACCCCCATCTCTACTCAATAATAAAACAAATAGC
CTGGCGTGGTGGCCCATGCATATAGTCCCAGCTACTTGTAAGGCTGAGGTGAGAGGACACTTGTGCCCAG
GAGTGGAGGCTGCAGTGAGCTATGATCACGCCACTGCATTCCAGCCTGGATAACAGAGTGAGAACCTATC
TCTAAAAATAAATAAATAAACGAAAAATAAA
SEQ ID NO: 8
- Homo sapiens beta-2-microglobulin (B2M), mRNA
AATATAAGTGGAGGCGTCGCGCTGGCGGGCATTCCTGAAGCTGACAGCATTCGGGCCGAGATGTCTCGCT
CCGTGGCCTTAGCTGTGCTCGCGCTACTCTCTCTTTCTGGCCTGGAGGCTATCCAGCGTACTCCAAAGAT
TCAGGTTTACTCACGTCATCCAGCAGAGAATGGAAAGTCAAATTTCCTGAATTGCTATGTGTCTGGGTTT
CATCCATCCGACATTGAAGTTGACTTACTGAAGAATGGAGAGAGAATTGAAAAAGTGGAGCATTCAGACT
TGTCTTTCAGCAAGGACTGGTCTTTCTATCTCTTGTACTACACTGAATTCACCCCCACTGAAAAAGATGA
GTATGCCTGCCGTGTGAACCATGTGACTTTGTCACAGCCCAAGATAGTTAAGTGGGATCGAGACATGTAA
GCAGCATCATGGAGGTTTGAAGATGCCGCATTTGGATTGGATGAATTCCAAATTCTGCTTGCTTGCTTTT
TAATATTGATATGCTTATACACTTACACTTTATGCACAAAATGTAGGGTTATAATAATGTTAACATGGAC
ATGATCTTCTTTATAATTCTACTTTGAGTGCTGTCTCCATGTTTGATGTATCTGAGCAGGTTGCTCCACA
GGTAGCTCTAGGAGGGCTGGCAACTTAGAGGTGGGGAGCAGAGAATTCTCTTATCCAACATCAACATCTT
GGTCAGATTTGAACTCTTCAATCTCTTGCACTCAAAGCTTGTTAAGATAGTTAAGCGTGCATAAGTTAAC
TTCCAATTTACATACTCTGCTTAGAATTTGGGGGAAAATTTAGAAATATAATTGACAGGATTATTGGAAA
TTTGTTATAATGAATGAAACATTTTGTCATATAAGATTCATATTTACTTCTTATACATTTGATAAAGTAA
GGCATGGTTGTGGTTAATCTGGTTTATTTTTGTTCCACAAGTTAAATAAATCATAAAACTTGATGTGTTA
TCTCTTA
SEQ ID NO: 9
- Homo sapiens breast cancer 1, early onset (BRCA1),
transcript variant 6, non-coding RNA
AGATAACTGGGCCCCTGCGCTCAGGAGGCCTTCACCCTCTGCTCTGGGTAAAGGTAGTAGAGTCCCGGGA
AAGGGACAGGGGGCCCAAGTGATGCTCTGGGGTACTGGCGTGGGAGAGTGGATTTCCGAAGCTGACAGAT
GGTTCATTGGAACAGAAAGAAATGGATTTATCTGCTCTTCGCGTTGAAGAAGTACAAAATGTCATTAATG
CTATGCAGAAAATCTTAGAGTGTCCCATCTGTCTGGAGTTGATCAAGGAACCTGTCTCCACAAAGTGTGA
CCACATATTTTGCAAATTTTGCATGCTGAAACTTCTCAACCAGAAGAAAGGGCCTTCACAGTGTCCTTTA
TGAGCCTACAAGAAAGTACGAGATTTAGTCAACTTGTTGAAGAGCTATTGAAAATCATTTGTGCTTTTCA
GCTTGACACAGGTTTGGAGTATGCAAACAGCTATAATTTTGCAAAAAAGGAAAATAACTCTCCTGAACAT
CTAAAAGATGAAGTTTCTATCATCCAAAGTATGGGCTACAGAAACCGTGCCAAAAGACTTCTACAGAGTG
AACCCGAAAATCCTTCCTTGGAAACCAGTCTCAGTGTCCAACTCTCTAACCTTGGAACTGTGAGAACTCT
GAGGACAAAGCAGCGGATACAACCTCAAAAGACGTCTGTCTACATTGAATTGGGATCTGATTCTTCTGAA
GATACCGTTAATAAGGCAACTTATTGCAGTGTGGGAGATCAAGAATTGTTACAAATCACCCCTCAAGGAA
CCAGGGATGAAATCAGTTTGGATTCTGCAAAAAAGGCTGCTTGTGAATTTTCTGAGACGGATGTAACAAA
TACTGAACATCATCAACCCAGTAATAATGATTTGAACACCACTGAGAAGCGTGCAGCTGAGAGGCATCCA
GAAAAGTATCAGGGTAGTTCTGTTTCAAACTTGCATGTGGAGCCATGTGGCACAAATACTCATGCCAGCT
CATTACAGCATGAGAACAGCAGTTTATTACTCACTAAAGACAGAATGAATGTAGAAAAGGCTGAATTCTG
TAATAAAAGCAAACAGCCTGGCTTAGCAAGGAGCCAACATAACAGATGGGCTGGAAGTAAGGAAACATGT
AATGATAGGCGGACTCCCAGCACAGAAAAAAAGGTAGATCTGAATGCTGATCCCCTGTGTGAGAGAAAAG
AATGGAATAAGCAGAAACTGCCATGCTCAGAGAATCCTAGAGATACTGAAGATGTTCCTTGGATAACACT
AAATAGCAGCATTCAGAAAGTTAATGAGTGGTTTTCCAGAAGTGATGAACTGTTAGGTTCTGATGACTCA
CATGATGGGGAGTCTGAATCAAATGCCAAAGTAGCTGATGTATTGGACGTTCTAAATGAGGTAGATGAAT
ATTCTGGTTCTTCAGAGAAAATAGACTTACTGGCCAGTGATCCTCATGAGGCTTTAATATGTAAAAGTGA
AAGAGTTCACTCCAAATCAGTAGAGAGTAATATTGAAGACAAAATATTTGGGAAAACCTATCGGAAGAAG
GCAAGCCTCCCCAACTTAAGCCATGTAACTGAAAATCTAATTATAGGAGCATTTGTTACTGAGCCACAGA
TAATACAAGAGCGTCCCCTCACAAATAAATTAAAGCGTAAAAGGAGACCTACATCAGGCCTTCATCCTGA
GGATTTTATCAAGAAAGCAGATTTGGCAGTTCAAAAGACTCCTGAAATGATAAATCAGGGAACTAACCAA
ACGGAGCAGAATGGTCAAGTGATGAATATTACTAATAGTGGTCATGAGAATAAAACAAAAGGTGATTCTA
TTCAGAATGAGAAAAATCCTAACCCAATAGAATCACTCGAAAAAGAATCTGCTTTCAAAACGAAAGCTGA
ACCTATAAGCAGCAGTATAAGCAATATGGAACTCGAATTAAATATCCACAATTCAAAAGCACCTAAAAAG
AATAGGCTGAGGAGGAAGTCTTCTACCAGGCATATTCATGCGCTTGAACTAGTAGTCAGTAGAAATCTAA
GCCCACCTAATTGTACTGAATTGCAAATTGATAGTTGTTCTAGCAGTGAAGAGATAAAGAAAAAAAAGTA
CAACCAAATGCCAGTCAGGCACAGCAGAAACCTACAACTCATGGAAGGTAAAGAACCTGCAACTGGAGCC
AAGAAGAGTAACAAGCCAAATGAACAGACAAGTAAAAGACATGACAGCGATACTTTCCCAGAGCTGAAGT
TAACAAATGCACCTGGTTCTTTTACTAAGTGTTCAAATACCAGTGAACTTAAAGAATTTGTCAATCCTAG
CCTTCCAAGAGAAGAAAAAGAAGAGAAACTAGAAACAGTTAAAGTGTCTAATAATGCTGAAGACCCCAAA
GATCTCATGTTAAGTGGAGAAAGGGTTTTGCAAACTGAAAGATCTGTAGAGAGTAGCAGTATTTCATTGG
TACCTGGTACTGATTATGGCACTCAGGAAAGTATCTCGTTACTGGAAGTTAGCACTCTAGGGAAGGCAAA
AACAGAACCAAATAAATGTGTGAGTCAGTGTGCAGCATTTGAAAACCCCAAGGGACTAATTCATGGTTGT
TCCAAAGATAATAGAAATGACACAGAAGGCTTTAAGTATCCATTGGGACATGAAGTTAACCACAGTCGGG
AAACAAGCATAGAAATGGAAGAAAGTGAACTTGATGCTCAGTATTTGCAGAATACATTCAAGGTTTCAAA
GCGCCAGTCATTTGCTCCGTTTTCAAATCCAGGAAATGCAGAAGAGGAATGTGCAACATTCTCTGCCCAC
TCTGGGTCCTTAAAGAAACAAAGTCCAAAAGTCACTTTTGAATGTGAACAAAAGGAAGAAAATCAAGGAA
AGAATGAGTCTAATATCAAGCCTGTACAGACAGTTAATATCACTGCAGGCTTTCCTGTGGTTGGTCAGAA
AGATAAGCCAGTTGATAATGCCAAATGTAGTATCAAAGGAGGCTCTAGGTTTTGTCTATCATCTCAGTTC
AGAGGCAACGAAACTGGACTCATTACTCCAAATAAACATGGACTTTTACAAAACCCATATCGTATACCAC
CACTTTTTCCCATCAAGTCATTTGTTAAAACTAAATGTAAGAAAAATCTGCTAGAGGAAAACTTTGAGGA
ACATTCAATGTCACCTGAAAGAGAAATGGGAAATGAGAACATTCCAAGTACAGTGAGCACAATTAGCCGT
AATAACATTAGAGAAAATGTTTTTAAAGAAGCCAGCTCAAGCAATATTAATGAAGTAGGTTCCAGTACTA
ATGAAGTGGGCTCCAGTATTAATGAAATAGGTTCCAGTGATGAAAACATTCAAGCAGAACTAGGTAGAAA
CAGAGGGCCAAAATTGAATGCTATGCTTAGATTAGGGGTTTTGCAACCTGAGGTCTATAAACAAAGTCTT
CCTGGAAGTAATTGTAAGCATCCTGAAATAAAAAAGCAAGAATATGAAGAAGTAGTTCAGACTGTTAATA
CAGATTTCTCTCCATATCTGATTTCAGATAACTTAGAACAGCCTATGGGAAGTAGTCATGCATCTCAGGT
TTGTTCTGAGACACCTGATGACCTGTTAGATGATGGTGAAATAAAGGAAGATACTAGTTTTGCTGAAAAT
GACATTAAGGAAAGTTCTGCTGTTTTTAGCAAAAGCGTCCAGAAAGGAGAGCTTAGCAGGAGTCCTAGCC
CTTTCACCCATACACATTTGGCTCAGGGTTACCGAAGAGGGGCCAAGAAATTAGAGTCCTCAGAAGAGAA
CTTATCTAGTGAGGATGAAGAGCTTCCCTGCTTCCAACACTTGTTATTTGGTAAAGTAAACAATATACCT
TCTCAGTCTACTAGGCATAGCACCGTTGCTACCGAGTGTCTGTCTAAGAACACAGAGGAGAATTTATTAT
CATTGAAGAATAGCTTAAATGACTGCAGTAACCAGGTAATATTGGCAAAGGCATCTCAGGAACATCACCT
TAGTGAGGAAACAAAATGTTCTGCTAGCTTGTTTTCTTCACAGTGCAGTGAATTGGAAGACTTGACTGCA
AATACAAACACCCAGGATCCTTTCTTGATTGGTTCTTCCAAACAAATGAGGCATCAGTCTGAAAGCCAGG
GAGTTGGTCTGAGTGACAAGGAATTGGTTTCAGATGATGAAGAAAGAGGAACGGGCTTGGAAGAAAATAA
TCAAGAAGAGCAAAGCATGGATTCAAACTTAGGTGAAGCAGCATCTGGGTGTGAGAGTGAAACAAGCGTC
TCTGAAGACTGCTCAGGGCTATCCTCTCAGAGTGACATTTTAACCACTCAGCAGAGGGATACCATGCAAC
ATAACCTGATAAAGCTCCAGCAGGAAATGGCTGAACTAGAAGCTGTGTTAGAACAGCATGGGAGCCAGCC
TTCTAACAGCTACCCTTCCATCATAAGTGACTCTTCTGCCCTTGAGGACCTGCGAAATCCAGAACAAAGC
ACATCAGAAAAAGCAGTATTAACTTCACAGAAAAGTAGTGAATACCCTATAAGCCAGAATCCAGAAGGCC
TTTCTGCTGACAAGTTTGAGGTGTCTGCAGATAGTTCTACCAGTAAAAATAAAGAACCAGGAGTGGAAAG
GTCATCCCCTTCTAAATGCCCATCATTAGATGATAGGTGGTACATGCACAGTTGCTCTGGGAGTCTTCAG
AATAGAAACTACCCATCTCAAGAGGAGCTCATTAAGGTTGTTGATGTGGAGGAGCAACAGCTGGAAGAGT
CTGGGCCACACGATTTGACGGAAACATCTTACTTGCCAAGGCAAGATCTAGAGGGAACCCCTTACCTGGA
ATCTGGAATCAGCCTCTTCTCTGATGACCCTGAATCTGATCCTTCTGAAGACAGAGCCCCAGAGTCAGCT
CGTGTTGGCAACATACCATCTTCAACCTCTGCATTGAAAGTTCCCCAATTGAAAGTTGCAGAATCTGCCC
AGAGTCCAGCTGCTGCTCATACTACTGATACTGCTGGGTATAATGCAATGGAAGAAAGTGTGAGCAGGGA
GAAGCCAGAATTGACAGCTTCAACAGAAAGGGTCAACAAAAGAATGTCCATGGTGGTGTCTGGCCTGACC
CCAGAAGAATTTATGCTCGTGTACAAGTTTGCCAGAAAACACCACATCACTTTAACTAATCTAATTACTG
AAGAGACTACTCATGTTGTTATGAAAACAGATGCTGAGTTTGTGTGTGAACGGACACTGAAATATTTTCT
AGGAATTGCGGGAGGAAAATGGGTAGTTAGCTATTTCTGGGTGACCCAGTCTATTAAAGAAAGAAAAATG
CTGAATGAGCATGATTTTGAAGTCAGAGGAGATGTGGTCAATGGAAGAAACCACCAAGGTCCAAAGCGAG
CAAGAGAATCCCAGGACAGAAAGATCTTCAGGGGGCTAGAAATCTGTTGCTATGGGCCCTTCACCAACAT
GCCCACAGATCAACTGGAATGGATGGTACAGCTGTGTGGTGCTTCTGTGGTGAAGGAGCTTTCATCATTC
ACCCTTGGCACAGGTGTCCACCCAATTGTGGTTGTGCAGCCAGATGCCTGGACAGAGGACAATGGCTTCC
ATGCAATTGGGCAGATGTGTGAGGCACCTGTGGTGACCCGAGAGTGGGTGTTGGACAGTGTAGCACTCTA
CCAGTGCCAGGAGCTGGACACCTACCTGATACCCCAGATCCCCCACAGCCACTACTGACTGCAGCCAGCC
ACAGGTACAGAGCCACAGGACCCCAAGAATGAGCTTACAAAGTGGCCTTTCCAGGCCCTGGGAGCTCCTC
TCACTCTTCAGTCCTTCTACTGTCCTGGCTACTAAATATTTTATGTACATCAGCCTGAAAAGGACTTCTG
GCTATGCAAGGGTCCCTTAAAGATTTTCTGCTTGAAGTCTCCCTTGGAAATCTGCCATGAGCACAAAATT
ATGGTAATTTTTCACCTGAGAAGATTTTAAAACCATTTAAACGCCACCAATTGAGCAAGATGCTGATTCA
TTATTTATCAGCCCTATTCTTTCTATTCAGGCTGTTGTTGGCTTAGGGCTGGAAGCACAGAGTGGCTTGG
CCTCAAGAGAATAGCTGGTTTCCCTAAGTTTACTTCTCTAAAACCCTGTGTTCACAAAGGCAGAGAGTCA
GACCCTTCAATGGAAGGAGAGTGCTTGGGATCGATTATGTGACTTAAAGTCAGAATAGTCCTTGGGCAGT
TCTCAAATGTTGGAGTGGAACATTGGGGAGGAAATTCTGAGGCAGGTATTAGAAATGAAAAGGAAACTTG
AAACCTGGGCATGGTGGCTCACGCCTGTAATCCCAGCACTTTGGGAGGCCAAGGTGGGCAGATCACTGGA
GGTCAGGAGTTCGAAACCAGCCTGGCCAACATGGTGAAACCCCATCTCTACTAAAAATACAGAAATTAGC
CGGTCATGGTGGTGGACACCTGTAATCCCAGCTACTCAGGTGGCTAAGGCAGGAGAATCACTTCAGCCCG
GGAGGTGGAGGTTGCAGTGAGCCAAGATCATACCACGGCACTCCAGCCTGGGTGACAGTGAGACTGTGGC
TCAAAAAAAAAAAAAAAAAAAGGAAAATGAAACTAGAAGAGATTTCTAAAAGTCTGAGATATATTTGCTA
GATTTCTAAAGAATGTGTTCTAAAACAGCAGAAGATTTTCAAGAACCGGTTTCCAAAGACAGTCTTCTAA
TTCCTCATTAGTAATAAGTAAAATGTTTATTGTTGTAGCTCTGGTATATAATCCATTCCTCTTAAAATAT
AAGACCTCTGGCATGAATATTTCATATCTATAAAATGACAGATCCCACCAGGAAGGAAGCTGTTGCTTTC
TTTGAGGTGATTTTTTTCCTTTGCTCCCTGTTGCTGAAACCATACAGCTTCATAAATAATTTTGCTTGCT
GAAGGAAGAAAAAGTGTTTTTCATAAACCCATTATCCAGGACTGTTTATAGCTGTTGGAAGGACTAGGTC
TTCCCTAGCCCCCCCAGTGTGCAAGGGCAGTGAAGACTTGATTGTACAAAATACGTTTTGTAAATGTTGT
GCTGTTAACACTGCAAATAAACTTGGTAGCAAACACTTCCAAAAAAAAAAAAAAAAAA
SEQ ID NO: 10
- Homo sapiens CD55 molecule, decay accelerating factor
for complement (Cromer blood group) (CD55), transcript
variant 1, mRNA
AGCGAGCTCCTCCTCCTTCCCCTCCCCACTCTCCCCGAGTCTAGGGCCCCCGGGGCGTATGACGCCGGAG
CCCTCTGACCGCACCTCTGACCACAACAAACCCCTACTCCACCCGTCTTGTTTGTCCCACCCTTGGTGAC
GCAGAGCCCCAGCCCAGACCCCGCCCAAAGCACTCATTTAACTGGTATTGCGGAGCCACGAGGCTTCTGC
TTACTGCAACTCGCTCCGGCCGCTGGGCGTAGCTGCGACTCGGCGGAGTCCCGGCGGCGCGTCCTTGTTC
TAACCCGGCGCGCCATGACCGTCGCGCGGCCGAGCGTGCCCGCGGCGCTGCCCCTCCTCGGGGAGCTGCC
CCGGCTGCTGCTGCTGGTGCTGTTGTGCCTGCCGGCCGTGTGGGGTGACTGTGGCCTTCCCCCAGATGTA
CCTAATGCCCAGCCAGCTTTGGAAGGCCGTACAAGTTTTCCCGAGGATACTGTAATAACGTACAAATGTG
AAGAAAGCTTTGTGAAAATTCCTGGCGAGAAGGACTCAGTGATCTGCCTTAAGGGCAGTCAATGGTCAGA
TATTGAAGAGTTCTGCAATCGTAGCTGCGAGGTGCCAACAAGGCTAAATTCTGCATCCCTCAAACAGCCT
TATATCACTCAGAATTATTTTCCAGTCGGTACTGTTGTGGAATATGAGTGCCGTCCAGGTTACAGAAGAG
AACCTTCTCTATCACCAAAACTAACTTGCCTTCAGAATTTAAAATGGTCCACAGCAGTCGAATTTTGTAA
AAAGAAATCATGCCCTAATCCGGGAGAAATACGAAATGGTCAGATTGATGTACCAGGTGGCATATTATTT
GGTGCAACCATCTCCTTCTCATGTAACACAGGGTACAAATTATTTGGCTCGACTTCTAGTTTTTGTCTTA
TTTCAGGCAGCTCTGTCCAGTGGAGTGACCCGTTGCCAGAGTGCAGAGAAATTTATTGTCCAGCACCACC
ACAAATTGACAATGGAATAATTCAAGGGGAACGTGACCATTATGGATATAGACAGTCTGTAACGTATGCA
TGTAATAAAGGATTCACCATGATTGGAGAGCACTCTATTTATTGTACTGTGAATAATGATGAAGGAGAGT
GGAGTGGCCCACCACCTGAATGCAGAGGAAAATCTCTAACTTCCAAGGTCCCACCAACAGTTCAGAAACC
TACCACAGTAAATGTTCCAACTACAGAAGTCTCACCAACTTCTCAGAAAACCACCACAAAAACCACCACA
CCAAATGCTCAAGCAACACGGAGTACACCTGTTTCCAGGACAACCAAGCATTTTCATGAAACAACCCCAA
ATAAAGGAAGTGGAACCACTTCAGGTACTACCCGTCTTCTATCTGGGCACACGTGTTTCACGTTGACAGG
TTTGCTTGGGACGCTAGTAACCATGGGCTTGCTGACTTAGCCAAAGAAGAGTTAAGAAGAAAATACACAC
AAGTATACAGACTGTTCCTAGTTTCTTAGACTTATCTGCATATTGGATAAAATAAATGCAATTGTGCTCT
TCATTTAGGATGCTTTCATTGTCTTTAAGATGTGTTAGGAATGTCAACAGAGCAAGGAGAAAAAAGGCAG
TCCTGGAATCACATTCTTAGCACACCTACACCTCTTGAAAATAGAACAACTTGCAGAATTGAGAGTGATT
CCTTTCCTAAAAGTGTAAGAAAGCATAGAGATTTGTTCGTATTTAGAATGGGATCACGAGGAAAAGAGAA
GGAAAGTGATTTTTTTCCACAAGATCTGTAATGTTATTTCCACTTATAAAGGAAATAAAAAATGAAAAAC
ATTATTTGGATATCAAAAGCAAATAAAAACCCAATTCAGTCTCTTCTAAGCAAAATTGCTAAAGAGAGAT
GAACCACATTATAAAGTAATCTTTGGCTGTAAGGCATTTTCATCTTTCCTTCGGGTTGGCAAAATATTTT
AAAGGTAAAACATGCTGGTGAACCAGGGGTGTTGATGGTGATAAGGGAGGAATATAGAATGAAAGACTGA
ATCTTCCTTTGTTGCACAAATAGAGTTTGGAAAAAGCCTGTGAAAGGTGTCTTCTTTGACTTAATGTCTT
TAAAAGTATCCAGAGATACTACAATATTAACATAAGAAAAGATTATATATTATTTCTGAATCGAGATGTC
CATAGTCAAATTTGTAAATCTTATTCTTTTGTAATATTTATTTATATTTATTTATGACAGTGAACATTCT
GATTTTACATGTAAAACAAGAAAAGTTGAAGAAGATATGTGAAGAAAAATGTATTTTTCCTAAATAGAAA
TAAATGATCCCATTTTTTGGTATCATGTAGTATGTGAAATTTATTCTTAAACGTGACTACTTTATTTCTA
AATAAGAAATTCCCTACCTGCTTCCTACAAGCAGTTCAGAATGCCATGCCTTGGTTGTCCTAGTGTGAAT
AATTTTCAGCTACTTTAAAATTATATTGTACTTTCTCAAGCATGTCATATCCTTTCCTATTAGAGTATCT
ATATTACTTGTTACTGATTTACCTGAAGGCAATCTGATTAATTTCTAGGTTTTTACCATATTCTTGTCAT
CTTGCCAATTACATTTTAAGTGTTAGACTAGACTAAGATGTACTAGTTGTATAGAATATAACTAGATTTA
TTATGGCAATGTTTATTTTGTCATTTTGCTTCATCTGTTTTGTTGTTGAAGTACTTTAAATTTCATACGT
TCATGGCATTTCACTGTAAAGACTTTAATGTGTATTTCTTAAAATAAAACTTTTTTTCCTCCTTAAAAAA
AAAAAAAAAAAA
SEQ ID NO: 11
- Homo sapiens cadherin 1, type 1, E-cadherin
(epithelial) (CDH1), mRNA
AGTGGCGTCGGAACTGCAAAGCACCTGTGAGCTTGCGGAAGTCAGTTCAGACTCCAGCCCGCTCCAGCCC
GGCCCGACCCGACCGCACCCGGCGCCTGCCCTCGCTCGGCGTCCCCGGCCAGCCATGGGCCCTTGGAGCC
GCAGCCTCTCGGCGCTGCTGCTGCTGCTGCAGGTCTCCTCTTGGCTCTGCCAGGAGCCGGAGCCCTGCCA
CCCTGGCTTTGACGCCGAGAGCTACACGTTCACGGTGCCCCGGCGCCACCTGGAGAGAGGCCGCGTCCTG
GGCAGAGTGAATTTTGAAGATTGCACCGGTCGACAAAGGACAGCCTATTTTTCCCTCGACACCCGATTCA
AAGTGGGCACAGATGGTGTGATTACAGTCAAAAGGCCTCTACGGTTTCATAACCCACAGATCCATTTCTT
GGTCTACGCCTGGGACTCCACCTACAGAAAGTTTTCCACCAAAGTCACGCTGAATACAGTGGGGCACCAC
CACCGCCCCCCGCCCCATCAGGCCTCCGTTTCTGGAATCCAAGCAGAATTGCTCACATTTCCCAACTCCT
CTCCTGGCCTCAGAAGACAGAAGAGAGACTGGGTTATTCCTCCCATCAGCTGCCCAGAAAATGAAAAAGG
CCCATTTCCTAAAAACCTGGTTCAGATCAAATCCAACAAAGACAAAGAAGGCAAGGTTTTCTACAGCATC
ACTGGCCAAGGAGCTGACACACCCCCTGTTGGTGTCTTTATTATTGAAAGAGAAACAGGATGGCTGAAGG
TGACAGAGCCTCTGGATAGAGAACGCATTGCCACATACACTCTCTTCTCTCACGCTGTGTCATCCAACGG
GAATGCAGTTGAGGATCCAATGGAGATTTTGATCACGGTAACCGATCAGAATGACAACAAGCCCGAATTC
ACCCAGGAGGTCTTTAAGGGGTCTGTCATGGAAGGTGCTCTTCCAGGAACCTCTGTGATGGAGGTCACAG
CCACAGACGCGGACGATGATGTGAACACCTACAATGCCGCCATCGCTTACACCATCCTCAGCCAAGATCC
TGAGCTCCCTGACAAAAATATGTTCACCATTAACAGGAACACAGGAGTCATCAGTGTGGTCACCACTGGG
CTGGACCGAGAGAGTTTCCCTACGTATACCCTGGTGGTTCAAGCTGCTGACCTTCAAGGTGAGGGGTTAA
GCACAACAGCAACAGCTGTGATCACAGTCACTGACACCAACGATAATCCTCCGATCTTCAATCCCACCAC
GTACAAGGGTCAGGTGCCTGAGAACGAGGCTAACGTCGTAATCACCACACTGAAAGTGACTGATGCTGAT
GCCCCCAATACCCCAGCGTGGGAGGCTGTATACACCATATTGAATGATGATGGTGGACAATTTGTCGTCA
CCACAAATCCAGTGAACAACGATGGCATTTTGAAAACAGCAAAGGGCTTGGATTTTGAGGCCAAGCAGCA
GTACATTCTACACGTAGCAGTGACGAATGTGGTACCTTTTGAGGTCTCTCTCACCACCTCCACAGCCACC
GTCACCGTGGATGTGCTGGATGTGAATGAAGCCCCCATCTTTGTGCCTCCTGAAAAGAGAGTGGAAGTGT
CCGAGGACTTTGGCGTGGGCCAGGAAATCACATCCTACACTGCCCAGGAGCCAGACACATTTATGGAACA
GAAAATAACATATCGGATTTGGAGAGACACTGCCAACTGGCTGGAGATTAATCCGGACACTGGTGCCATT
TCCACTCGGGCTGAGCTGGACAGGGAGGATTTTGAGCACGTGAAGAACAGCACGTACACAGCCCTAATCA
TAGCTACAGACAATGGTTCTCCAGTTGCTACTGGAACAGGGACACTTCTGCTGATCCTGTCTGATGTGAA
TGACAACGCCCCCATACCAGAACCTCGAACTATATTCTTCTGTGAGAGGAATCCAAAGCCTCAGGTCATA
AACATCATTGATGCAGACCTTCCTCCCAATACATCTCCCTTCACAGCAGAACTAACACACGGGGCGAGTG
CCAACTGGACCATTCAGTACAACGACCCAACCCAAGAATCTATCATTTTGAAGCCAAAGATGGCCTTAGA
GGTGGGTGACTACAAAATCAATCTCAAGCTCATGGATAACCAGAATAAAGACCAAGTGACCACCTTAGAG
GTCAGCGTGTGTGACTGTGAAGGGGCCGCTGGCGTCTGTAGGAAGGCACAGCCTGTCGAAGCAGGATTGC
AAATTCCTGCCATTCTGGGGATTCTTGGAGGAATTCTTGCTTTGCTAATTCTGATTCTGCTGCTCTTGCT
GTTTCTTCGGAGGAGAGCGGTGGTCAAAGAGCCCTTACTGCCCCCAGAGGATGACACCCGGGACAACGTT
TATTACTATGATGAAGAAGGAGGCGGAGAAGAGGACCAGGACTTTGACTTGAGCCAGCTGCACAGGGGCC
TGGACGCTCGGCCTGAAGTGACTCGTAACGACGTTGCACCAACCCTCATGAGTGTCCCCCGGTATCTTCC
CCGCCCTGCCAATCCCGATGAAATTGGAAATTTTATTGATGAAAATCTGAAAGCGGCTGATACTGACCCC
ACAGCCCCGCCTTATGATTCTCTGCTCGTGTTTGACTATGAAGGAAGCGGTTCCGAAGCTGCTAGTCTGA
GCTCCCTGAACTCCTCAGAGTCAGACAAAGACCAGGACTATGACTACTTGAACGAATGGGGCAATCGCTT
CAAGAAGCTGGCTGACATGTACGGAGGCGGCGAGGACGACTAGGGGACTCGAGAGAGGCGGGCCCCAGAC
CCATGTGCTGGGAAATGCAGAAATCACGTTGCTGGTGGTTTTTCAGCTCCCTTCCCTTGAGATGAGTTTC
TGGGGAAAAAAAAGAGACTGGTTAGTGATGCAGTTAGTATAGCTTTATACTCTCTCCACTTTATAGCTCT
AATAAGTTTGTGTTAGAAAAGTTTCGACTTATTTCTTAAAGCTTTTTTTTTTTTCCCATCACTCTTTACA
TGGTGGTGATGTCCAAAAGATACCCAAATTTTAATATTCCAGAAGAACAACTTTAGCATCAGAAGGTTCA
CCCAGCACCTTGCAGATTTTCTTAAGGAATTTTGTCTCACTTTTAAAAAGAAGGGGAGAAGTCAGCTACT
CTAGTTCTGTTGTTTTGTGTATATAATTTTTTAAAAAAAATTTGTGTGCTTCTGCTCATTACTACACTGG
TGTGTCCCTCTGCCTTTTTTTTTTTTTTAAGACAGGGTCTCATTCTATCGGCCAGGCTGGAGTGCAGTGG
TGCAATCACAGCTCACTGCAGCCTTGTCCTCCCAGGCTCAAGCTATCCTTGCACCTCAGCCTCCCAAGTA
GCTGGGACCACAGGCATGCACCACTACGCATGACTAATTTTTTAAATATTTGAGACGGGGTCTCCCTGTG
TTACCCAGGCTGGTCTCAAACTCCTGGGCTCAAGTGATCCTCCCATCTTGGCCTCCCAGAGTATTGGGAT
TACAGACATGAGCCACTGCACCTGCCCAGCTCCCCAACTCCCTGCCATTTTTTAAGAGACAGTTTCGCTC
CATCGCCCAGGCCTGGGATGCAGTGATGTGATCATAGCTCACTGTAACCTCAAACTCTGGGGCTCAAGCA
GTTCTCCCACCAGCCTCCTTTTTATTTTTTTGTACAGATGGGGTCTTGCTATGTTGCCCAAGCTGGTCTT
AAACTCCTGGCCTCAAGCAATCCTTCTGCCTTGGCCCCCCAAAGTGCTGGGATTGTGGGCATGAGCTGCT
GTGCCCAGCCTCCATGTTTTAATATCAACTCTCACTCCTGAATTCAGTTGCTTTGCCCAAGATAGGAGTT
CTCTGATGCAGAAATTATTGGGCTCTTTTAGGGTAAGAAGTTTGTGTCTTTGTCTGGCCACATCTTGACT
AGGTATTGTCTACTCTGAAGACCTTTAATGGCTTCCCTCTTTCATCTCCTGAGTATGTAACTTGCAATGG
GCAGCTATCCAGTGACTTGTTCTGAGTAAGTGTGTTCATTAATGTTTATTTAGCTCTGAAGCAAGAGTGA
TATACTCCAGGACTTAGAATAGTGCCTAAAGTGCTGCAGCCAAAGACAGAGCGGAACTATGAAAAGTGGG
CTTGGAGATGGCAGGAGAGCTTGTCATTGAGCCTGGCAATTTAGCAAACTGATGCTGAGGATGATTGAGG
TGGGTCTACCTCATCTCTGAAAATTCTGGAAGGAATGGAGGAGTCTCAACATGTGTTTCTGACACAAGAT
CCGTGGTTTGTACTCAAAGCCCAGAATCCCCAAGTGCCTGCTTTTGATGATGTCTACAGAAAATGCTGGC
TGAGCTGAACACATTTGCCCAATTCCAGGTGTGCACAGAAAACCGAGAATATTCAAAATTCCAAATTTTT
TTCTTAGGAGCAAGAAGAAAATGTGGCCCTAAAGGGGGTTAGTTGAGGGGTAGGGGGTAGTGAGGATCTT
GATTTGGATCTCTTTTTATTTAAATGTGAATTTCAACTTTTGACAATCAAAGAAAAGACTTTTGTTGAAA
TAGCTTTACTGTTTCTCAAGTGTTTTGGAGAAAAAAATCAACCCTGCAATCACTTTTTGGAATTGTCTTG
ATTTTTCGGCAGTTCAAGCTATATCGAATATAGTTCTGTGTAGAGAATGTCACTGTAGTTTTGAGTGTAT
ACATGTGTGGGTGCTGATAATTGTGTATTTTCTTTGGGGGTGGAAAAGGAAAACAATTCAAGCTGAGAAA
AGTATTCTCAAAGATGCATTTTTATAAATTTTATTAAACAATTTTGTTAAACCAT
SEQ ID NO: 12
- Homo sapiens cyclin-dependent kinase inhibitor
1B (p27, Kip1) (CDKN1B), mRNA
CTTCTTCGTCAGCCTCCCTTCCACCGCCATATTGGGCCACTAAAAAAAGGGGGCTCGTCTTTTCGGGGTG
TTTTTCTCCCCCTCCCCTGTCCCCGCTTGCTCACGGCTCTGCGACTCCGACGCCGGCAAGGTTTGGAGAG
CGGCTGGGTTCGCGGGACCCGCGGGCTTGCACCCGCCCAGACTCGGACGGGCTTTGCCACCCTCTCCGCT
TGCCTGGTCCCCTCTCCTCTCCGCCCTCCCGCTCGCCAGTCCATTTGATCAGCGGAGACTCGGCGGCCGG
GCCGGGGCTTCCCCGCAGCCCCTGCGCGCTCCTAGAGCTCGGGCCGTGGCTCGTCGGGGTCTGTGTCTTT
TGGCTCCGAGGGCAGTCGCTGGGCTTCCGAGAGGGGTTCGGGCTGCGTAGGGGCGCTTTGTTTTGTTCGG
TTTTGTTTTTTTGAGAGTGCGAGAGAGGCGGTCGTGCAGACCCGGGAGAAAGATGTCAAACGTGCGAGTG
TCTAACGGGAGCCCTAGCCTGGAGCGGATGGACGCCAGGCAGGCGGAGCACCCCAAGCCCTCGGCCTGCA
GGAACCTCTTCGGCCCGGTGGACCACGAAGAGTTAACCCGGGACTTGGAGAAGCACTGCAGAGACATGGA
AGAGGCGAGCCAGCGCAAGTGGAATTTCGATTTTCAGAATCACAAACCCCTAGAGGGCAAGTACGAGTGG
CAAGAGGTGGAGAAGGGCAGCTTGCCCGAGTTCTACTACAGACCCCCGCGGCCCCCCAAAGGTGCCTGCA
AGGTGCCGGCGCAGGAGAGCCAGGATGTCAGCGGGAGCCGCCCGGCGGCGCCTTTAATTGGGGCTCCGGC
TAACTCTGAGGACACGCATTTGGTGGACCCAAAGACTGATCCGTCGGACAGCCAGACGGGGTTAGCGGAG
CAATGCGCAGGAATAAGGAAGCGACCTGCAACCGACGATTCTTCTACTCAAAACAAAAGAGCCAACAGAA
CAGAAGAAAATGTTTCAGACGGTTCCCCAAATGCCGGTTCTGTGGAGCAGACGCCCAAGAAGCCTGGCCT
CAGAAGACGTCAAACGTAAACAGCTCGAATTAAGAATATGTTTCCTTGTTTATCAGATACATCACTGCTT
GATGAAGCAAGGAAGATATACATGAAAATTTTAAAAATACATATCGCTGACTTCATGGAATGGACATCCT
GTATAAGCACTGAAAAACAACAACACAATAACACTAAAATTTTAGGCACTCTTAAATGATCTGCCTCTAA
AAGCGTTGGATGTAGCATTATGCAATTAGGTTTTTCCTTATTTGCTTCATTGTACTACCTGTGTATATAG
TTTTTACCTTTTATGTAGCACATAAACTTTGGGGAAGGGAGGGCAGGGTGGGGCTGAGGAACTGACGTGG
AGCGGGGTATGAAGAGCTTGCTTTGATTTACAGCAAGTAGATAAATATTTGACTTGCATGAAGAGAAGCA
ATTTTGGGGAAGGGTTTGAATTGTTTTCTTTAAAGATGTAATGTCCCTTTCAGAGACAGCTGATACTTCA
TTTAAAAAAATCACAAAAATTTGAACACTGGCTAAAGATAATTGCTATTTATTTTTACAAGAAGTTTATT
CTCATTTGGGAGATCTGGTGATCTCCCAAGCTATCTAAAGTTTGTTAGATAGCTGCATGTGGCTTTTTTA
AAAAAGCAACAGAAACCTATCCTCACTGCCCTCCCCAGTCTCTCTTAAAGTTGGAATTTACCAGTTAATT
ACTCAGCAGAATGGTGATCACTCCAGGTAGTTTGGGGCAAAAATCCGAGGTGCTTGGGAGTTTTGAATGT
TAAGAATTGACCATCTGCTTTTATTAAATTTGTTGACAAAATTTTCTCATTTTCTTTTCACTTCGGGCTG
TGTAAACACAGTCAAAATAATTCTAAATCCCTCGATATTTTTAAAGATCTGTAAGTAACTTCACATTAAA
AAATGAAATATTTTTTAATTTAAAGCTTACTCTGTCCATTTATCCACAGGAAAGTGTTATTTTTCAAGGA
AGGTTCATGTAGAGAAAAGCACACTTGTAGGATAAGTGAAATGGATACTACATCTTTAAACAGTATTTCA
TTGCCTGTGTATGGAAAAACCATTTGAAGTGTACCTGTGTACATAACTCTGTAAAAACACTGAAAAATTA
TACTAACTTATTTATGTTAAAAGATTTTTTTTAATCTAGACAATATACAAGCCAAAGTGGCATGTTTTGT
GCATTTGTAAATGCTGTGTTGGGTAGAATAGGTTTTCCCCTCTTTTGTTAAATAATATGGCTATGCTTAA
AAGGTTGCATACTGAGCCAAGTATAATTTTTTGTAATGTGTGAAAAAGATGCCAATTATTGTTACACATT
AAGTAATCAATAAAGAAAACTTCCATAGCTATT
SEQ ID No: 13
- Homo sapiens checkpoint kinase 2 (CHEK2),
transcript variant 3, mRNA
GCAGGTTTAGCGCCACTCTGCTGGCTGAGGCTGCGGAGAGTGTGCGGCTCCAGGTGGGCTCACGCGGTCG
TGATGTCTCGGGAGTCGGATGTTGAGGCTCAGCAGTCTCATGGCAGCAGTGCCTGTTCACAGCCCCATGG
CAGCGTTACCCAGTCCCAAGGCTCCTCCTCACAGTCCCAGGGCATATCCAGCTCCTCTACCAGCACGATG
CCAAACTCCAGCCAGTCCTCTCACTCCAGCTCTGGGACACTGAGCTCCTTAGAGACAGTGTCCACTCAGG
AACTCTATTCTATTCCTGAGGACCAAGAACCTGAGGACCAAGAACCTGAGGAGCCTACCCCTGCCCCCTG
GGCTCGATTATGGGCCCTTCAGGATGGATTTGCCAATCTTGAGACAGAGTCTGGCCATGTTACCCAATCT
GATCTTGAACTCCTGCTGTCATCTGATCCTCCTGCCTCAGCCTCCCAAAGTGCTGGGATAAGAGGTGTGA
GGCACCATCCCCGGCCAGTTTGCAGTCTAAAATGTGTGAATGACAACTACTGGTTTGGGAGGGACAAAAG
CTGTGAATATTGCTTTGATGAACCACTGCTGAAAAGAACAGATAAATACCGAACATACAGCAAGAAACAC
TTTCGGATTTTCAGGGAAGTGGGTCCTAAAAACTCTTACATTGCATACATAGAAGATCACAGTGGCAATG
GAACCTTTGTAAATACAGAGCTTGTAGGGAAAGGAAAACGCCGTCCTTTGAATAACAATTCTGAAATTGC
ACTGTCACTAAGCAGAAATAAAGTTTTTGTCTTTTTTGATCTGACTGTAGATGATCAGTCAGTTTATCCT
AAGGCATTAAGAGATGAATACATCATGTCAAAAACTCTTGGAAGTGGTGCCTGTGGAGAGGTAAAGCTGG
CTTTCGAGAGGAAAACATGTAAGAAAGTAGCCATAAAGATCATCAGCAAAAGGAAGTTTGCTATTGGTTC
AGCAAGAGAGGCAGACCCAGCTCTCAATGTTGAAACAGAAATAGAAATTTTGAAAAAGCTAAATCATCCT
TGCATCATCAAGATTAAAAACTTTTTTGATGCAGAAGATTATTATATTGTTTTGGAATTGATGGAAGGGG
GAGAGCTGTTTGACAAAGTGGTGGGGAATAAACGCCTGAAAGAAGCTACCTGCAAGCTCTATTTTTACCA
GATGCTCTTGGCTGTGCAGTACCTTCATGAAAACGGTATTATACACCGTGACTTAAAGCCAGAGAATGTT
TTACTGTCATCTCAAGAAGAGGACTGTCTTATAAAGATTACTGATTTTGGGCACTCCAAGATTTTGGGAG
AGACCTCTCTCATGAGAACCTTATGTGGAACCCCCACCTACTTGGCGCCTGAAGTTCTTGTTTCTGTTGG
GACTGCTGGGTATAACCGTGCTGTGGACTGCTGGAGTTTAGGAGTTATTCTTTTTATCTGCCTTAGTGGG
TATCCACCTTTCTCTGAGCATAGGACTCAAGTGTCACTGAAGGATCAGATCACCAGTGGAAAATACAACT
TCATTCCTGAAGTCTGGGCAGAAGTCTCAGAGAAAGCTCTGGACCTTGTCAAGAAGTTGTTGGTAGTGGA
TCCAAAGGCACGTTTTACGACAGAAGAAGCCTTAAGACACCCGTGGCTTCAGGATGAAGACATGAAGAGA
AAGTTTCAAGATCTTCTGTCTGAGGAAAATGAATCCACAGCTCTACCCCAGGTTCTAGCCCAGCCTTCTA
CTAGTCGAAAGCGGCCCCGTGAAGGGGAAGCCGAGGGTGCCGAGACCACAAAGCGCCCAGCTGTGTGTGC
TGCTGTGTTGTGAACTCCGTGGTTTGAACACGAAAGAAATGTACCTTCTTTCACTCTGTCATCTTTCTTT
TCTTTGAGTCTGTTTTTTTATAGTTTGTATTTTAATTATGGGAATAATTGCTTTTTCACAGTCACTGATG
TACAATTAAAAACCTGATGGAACCTGGAAAA
SEQ ID NO: 14
- Homo sapiens colony stimulating factor 3 receptor
(granulocyte) (CSF3R), transcript variant 3, mRNA
GAGTACTGTGAAGATGTGGTCCCCAAGGCTAGAGCTGAAAAGAGGCTTAGGGCCGGGTGAGCCTTCCAGC
CAGGGCCTGCCTCCAAGTGATGCTCCCCCAGGGCAGGGGGCATAAGGATGGCACCCAGCCAGGTGGGAGC
CTGGGCCCTGCCCAGCCTCAAAGCTTTGAGCTCAGGAAATCCGGAGGCAGGGGAGGGGGACATCGTTGCC
ACATTCCCCAGCCCTTTAAGACCCCCAAGGCAGGAAGGCTGCCCGGGCCTCACCAGCTTCCCTCACAGGC
TCCTTCCTGGGAGGAAGGGGCTGCCTGTGCCCTCGAAGGCGCAAGGGAGGGCAGGAGGGAGGCTCGGAAG
GTGTTGCAATCCCCAGCCCCCGGGCCTGTCAGAGGCTGAGCCATTAACGACAGAGCTCGGGGAGAGAAGC
TGGACTGCAGCTGGTTTCAGGAACTTCTCTTGACGAGAAGAGAGACCAAGGAGGCCAAGCAGGGGCTGGG
CCAGAGGTGCCAACATGGGGAAACTGAGGCTCGGCTCGGAAAGGTGAAGTAACTTGTCCAAGATCACAAA
GCTGGTGAACATCAAGTTGGTGCTATGGCAAGGCTGGGAAACTGCAGCCTGACTTGGGCTGCCCTGATCA
TCCTGCTGCTCCCCGGAAGTCTGGAGGAGTGCGGGCACATCAGTGTCTCAGCCCCCATCGTCCACCTGGG
GGATCCCATCACAGCCTCCTGCATCATCAAGCAGAACTGCAGCCATCTGGACCCGGAGCCACAGATTCTG
TGGAGACTGGGAGCAGAGCTTCAGCCCGGGGGCAGGCAGCAGCGTCTGTCTGATGGGACCCAGGAATCTA
TCATCACCCTGCCCCACCTCAACCACACTCAGGCCTTTCTCTCCTGCTGCCTGAACTGGGGCAACAGCCT
GCAGATCCTGGACCAGGTTGAGCTGCGCGCAGGCTACCCTCCAGCCATACCCCACAACCTCTCCTGCCTC
ATGAACCTCACAACCAGCAGCCTCATCTGCCAGTGGGAGCCAGGACCTGAGACCCACCTACCCACCAGCT
TCACTCTGAAGAGTTTCAAGAGCCGGGGCAACTGTCAGACCCAAGGGGACTCCATCCTGGACTGCGTGCC
CAAGGACGGGCAGAGCCACTGCTGCATCCCACGCAAACACCTGCTGTTGTACCAGAATATGGGCATCTGG
GTGCAGGCAGAGAATGCGCTGGGGACCAGCATGTCCCCACAACTGTGTCTTGATCCCATGGATGTTGTGA
AACTGGAGCCCCCCATGCTGCGGACCATGGACCCCAGCCCTGAAGCGGCCCCTCCCCAGGCAGGCTGCCT
ACAGCTGTGCTGGGAGCCATGGCAGCCAGGCCTGCACATAAATCAGAAGTGTGAGCTGCGCCACAAGCCG
CAGCGTGGAGAAGCCAGCTGGGCACTGGTGGGCCCCCTCCCCTTGGAGGCCCTTCAGTATGAGCTCTGCG
GGCTCCTCCCAGCCACGGCCTACACCCTGCAGATACGCTGCATCCGCTGGCCCCTGCCTGGCCACTGGAG
CGACTGGAGCCCCAGCCTGGAGCTGAGAACTACCGAACGGGCCCCCACTGTCAGACTGGACACATGGTGG
CGGCAGAGGCAGCTGGACCCCAGGACAGTGCAGCTGTTCTGGAAGCCAGTGCCCCTGGAGGAAGACAGCG
GACGGATCCAAGGTTATGTGGTTTCTTGGAGACCCTCAGGCCAGGCTGGGGCCATCCTGCCCCTCTGCAA
CACCACAGAGCTCAGCTGCACCTTCCACCTGCCTTCAGAAGCCCAGGAGGTGGCCCTTGTGGCCTATAAC
TCAGCCGGGACCTCTCGTCCCACTCCGGTGGTCTTCTCAGAAAGCAGAGGCCCAGCTCTGACCAGACTCC
ATGCCATGGCCCGAGACCCTCACAGCCTCTGGGTAGGCTGGGAGCCCCCCAATCCATGGCCTCAGGGCTA
TGTGATTGAGTGGGGCCTGGGCCCCCCCAGCGCGAGCAATAGCAACAAGACCTGGAGGATGGAACAGAAT
GGGAGAGCCACGGGGTTTCTGCTGAAGGAGAACATCAGGCCCTTTCAGCTCTATGAGATCATCGTGACTC
CCTTGTACCAGGACACCATGGGACCCTCCCAGCATGTCTATGCCTACTCTCAAGAAATGGCTCCCTCCCA
TGCCCCAGAGCTGCATCTAAAGCACATTGGCAAGACCTGGGCACAGCTGGAGTGGGTGCCTGAGCCCCCT
GAGCTGGGGAAGAGCCCCCTTACCCACTACACCATCTTCTGGACCAACGCTCAGAACCAGTCCTTCTCCG
CCATCCTGAATGCCTCCTCCCGTGGCTTTGTCCTCCATGGCCTGGAGCCCGCCAGTCTGTATCACATCCA
CCTCATGGCTGCCAGCCAGGCTGGGGCCACCAACAGTACAGTCCTCACCCTGATGACCTTGACCCCAGAG
GGGTCGGAGCTACACATCATCCTGGGCCTGTTCGGCCTCCTGCTGTTGCTCACCTGCCTCTGTGGAACTG
CCTGGCTCTGTTGCAGCCCCAACAGGAAGAATCCCCTCTGGCCAAGTGTCCCAGACCCAGCTCACAGCAG
CCTGGGCTCCTGGGTGCCCACAATCATGGAGGAGCTGCCCGGACCCAGACAGGGACAGTGGCTGGGGCAG
ACATCTGAAATGAGCCGTGCTCTCACCCCACATCCTTGTGTGCAGGATGCCTTCCAGCTGCCCGGCCTTG
GCACGCCACCCATCACCAAGCTCACAGTGCTGGAGGAGGATGAAAAGAAGCCGGTGCCCTGGGAGTCCCA
TAACAGCTCAGAGACCTGTGGCCTCCCCACTCTGGTCCAGACCTATGTGCTCCAGGGGGACCCAAGAGCA
GTTTCCACCCAGCCCCAATCCCAGTCTGGCACCAGCGATCAGGTCCTTTATGGGCAGCTGCTGGGCAGCC
CCACAAGCCCAGGGCCAGGGCACTATCTCCGCTGTGACTCCACTCAGCCCCTCTTGGCGGGCCTCACCCC
CAGCCCCAAGTCCTATGAGAACCTCTGGTTCCAGGCCAGCCCCTTGGGGACCCTGGTAACCCCAGCCCCA
AGCCAGGAGGACGACTGTGTCTTTGGGCCACTGCTCAACTTCCCCCTCCTGCAGGGGATCCGGGTCCATG
GGATGGAGGCGCTGGGGAGCTTCTAGGGCTTCCTGGGGTTCCCTTCTTGGGCCTGCCTCTTAAAGGCCTG
AGCTAGCTGGAGAAGAGGGGAGGGTCCATAAGCCCATGACTAAAAACTACCCCAGCCCAGGCTCTCACCA
TCTCCAGTCACCAGCATCTCCCTCTCCTCCCAATCTCCATAGGCTGGGCCTCCCAGGCGATCTGCATACT
TTAAGGACCAGATCATGCTCCATCCAGCCCCACCCAATGGCCTTTTGTGCTTGTTTCCTATAACTTCAGT
ATTGTAAACTAGTTTTTGGTTTGCAGTTTTTGTTGTTGTTTATAGACACTCTTGGGTGTAAAAAAAAAAA
SEQ ID NO: 15
- CYHomo sapiens cathepsin S (CTSS),
transcript variant 1, mRNA
GACAAGGGCTCTTCTTGATGGCTTACTGTATCCACTTTGTCCCCAAGACCATAGGGAAATGACTAGAGGT
GACTGTACTAGCTAGATTTTAAATGAAACTGAAATGAAAGTTCACTTCCTCATTTTGAGTACCTCATGTG
ACAAGTTCCAATTTCTTTTCAAGTCAATTGAACTGAAATCTCCTTGTTGCTTTGAAATCTTAGAAGAGAG
CCCACTAATTCAAGGACTCTTACTGTGGGAGCAACTGCTGGTTCTATCACAATGAAACGGCTGGTTTGTG
TGCTCTTGGTGTGCTCCTCTGCAGTGGCACAGTTGCATAAAGATCCTACCCTGGATCACCACTGGCATCT
CTGGAAGAAAACCTATGGCAAACAATACAAGGAAAAGAATGAAGAAGCAGTACGACGTCTCATCTGGGAA
AAGAATCTAAAGTTTGTGATGCTTCACAACCTGGAGCATTCAATGGGAATGCACTCATACGATCTGGGCA
TGAACCACCTGGGAGACATGACCAGTGAAGAAGTGATGTCTTTGATGAGTTCCCTGAGAGTTCCCAGCCA
GTGGCAGAGAAATATCACATATAAGTCAAACCCTAATCGGATATTGCCTGATTCTGTGGACTGGAGAGAG
AAAGGGTGTGTTACTGAAGTGAAATATCAAGGTTCTTGTGGTGCTTGCTGGGCTTTCAGTGCTGTGGGGG
CCCTGGAAGCACAGCTGAAGCTGAAAACAGGAAAGCTGGTGTCTCTCAGTGCCCAGAACCTGGTGGATTG
CTCAACTGAAAAATATGGAAACAAAGGCTGCAATGGTGGCTTCATGACAACGGCTTTCCAGTACATCATT
GATAACAAGGGCATCGACTCAGACGCTTCCTATCCCTACAAAGCCATGGATCAGAAATGTCAATATGACT
CAAAATATCGTGCTGCCACATGTTCAAAGTACACTGAACTTCCTTATGGCAGAGAAGATGTCCTGAAAGA
AGCTGTGGCCAATAAAGGCCCAGTGTCTGTTGGTGTAGATGCGCGTCATCCTTCTTTCTTCCTCTACAGA
AGTGGTGTCTACTATGAACCATCCTGTACTCAGAATGTGAATCATGGTGTACTTGTGGTTGGCTATGGTG
ATCTTAATGGGAAAGAATACTGGCTTGTGAAAAACAGCTGGGGCCACAACTTTGGTGAAGAAGGATATAT
TCGGATGGCAAGAAATAAAGGAAATCATTGTGGGATTGCTAGCTTTCCCTCTTACCCAGAAATCTAGAGG
ATCTCTCCTTTTTATAACAAATCAAGAAATATGAAGCACTTTCTCTTAACTTAATTTTTCCTGCTGTATC
CAGAAGAAATAATTGTGTCATGATTAATGTGTATTTACTGTACTAATTAGAAAATATAGTTTGAGGCCGG
GCACGGTGGCTCACGCCTGTAATCCCAGTACTTGGGAGGCCAAGGCAGGCATATCAACTTGAGGCCAGGA
GTTAAAGAGCAGCCTGGCTAACATGGTGAAACCCCATCTCTACTAAAAATACAAAAAATTAGCCGAGCAC
GGTGGTGCATGCCTGTAATCCCAGCTACTTGGGAGGCTGAGGCACGAGATTCCTTGAACCCAAGAGGTTG
AGGCTATGTTGAGCTGAGATCACACCACTGTACTCCAGCCTGGATGACAGAGTGGAGACTCTGTTTCAAA
AAAACAGAAAAGAAAATATAGTTTGATTCTTCATTTTTTTAAATTTGCAAATCTCAGGATAAAGTTTGCT
AAGTAAATTAGTAATGTACTATAGATATAACTGTACAAAAATTGTTCAACCTAAAACAATCTGTAATTGC
TTATTGTTTTATTGTATACTCTTTGTCTTTTTAAGACCCCTAATAGCCTTTTGTAACTTGATGGCTTAAA
AATACTTAATAAATCTGCCATTTCAAATTTCTATCATTGCCACATACCATTCTTATTCCTAGGCAACTAT
TAATAATCTATCCTGAGAATATTAATTGTGGTATTCTGGTGATGGGGTTTAGCAACTTTGATGGAAGAAA
ATATTAGGCTATAAATGTCCTAAGGACTCAGATTGTATCTTTGTACAGAAGAGGATTCAAAACGCCACGT
GTAGTGGCTCATGCCTGTAATCCCAACACTTTGGGAGGCTGAAGTAGGAGGATCGTCTTGAGCCCAGGAG
TTCAAGACCAGCCTGGACAACATAGTGAGACCTTGTCTCCACAAAAATAAAAAAGAAACTATCCAGGAGT
GGTGGTGTGTGCCTGTGGTCCCTGCTATGCAGATGTCTAAGACAGGAGGATCACAAGAGCCCAGGAGGTT
GAGAATGCAGTGAGCTTGTAATTGCACCACTGCACTCCAGCCTGGGTGACAGAGCAAGACCCTGTCTTAA
AAAAAGAGGATTCAACACATATTTTTATATTATGTTAAAGTAAAGAAATGCATAAAAGACAAGCACTTTG
GAAGAATTATTTTAATGATCAACAATTTAATGTATTAGTCCAAATTATTTTTACGTAGTCATCAACAATT
TGACCAGGGCCTTTATTTGGCAAATAACTGAGCCAACCAGAATAAAATAACCAATACTCCACTGCTCATA
TTTTTATCTAATTCAGATGGATCTTCCTTACAACTGCTCTAGATTAGTAGATGCATCTAAGCAGGCAGCA
GGAACTTTAAATTTTTTAAGTTCATGTCTATGACATGAACAATGTGTGGGATAATGTCATTAATATATCC
TAAATTAACCTAAACGTATTTCACTAACTCTGGCTCCTTCTCCATAAAGCACATTTTAAGGAACAAGAAT
TGCTAAATATAAAAACATAAATAATACCATAATACATGGCTATCATCAAAAGTGTATAGAATATTATAGT
TTAAAAGTATTTAGTTGATTACTTTTCAGTTTTGTTTTGTTTTTTGAGACGGAGTCTCACTCTGTTGCCC
AGGCTGGAGTGCAGTGGCACCATCTCAGTTCACTGCAACTTCTGCCTCCCGAGTTCAAGCGATTCTCCTG
CCTCAGCCTCCCGAGTAGCTGGAATTATAGGCGTGCACCACCACGCCCAGCTAATTTTTGTATTTTTAGT
AAAGACAGGGTTTTGCCACATTAGCCAGGCTGGTCTCAAACTCCTGACCTCAGGTGATCCACCCACCCCA
GCCTCCCAAAGTGCTAAGATTACAGGCGTGAGCCACTGAGCCCAGCCTACTTTTCAGTTTTTAACATAAT
TTTTGTTTTATCCACAACTTTTCAAGTATTGAAAGTAGAATAAAAACATGGGTTCTTAGTCTTTAGCTAT
CTGTTAAAGCCTATGAATGCCTTCTTAAAATCATGTTTTTAAATGCATAAAATATATAGGATTACAAAGG
AATCTAATTATATCGAAATACAGTTATTAAAATGTTAAAAGATAAGTTTGTTATATATTAATATGCATGC
TTCTTTATAAATGCATTAAATAAGAGTTAATAGCTATCCTAAATTTGAAATAGTGATAAGCATAATGAAA
ATAGATGCAAAAAACTAATGTGATATGAAAATATCTGGGTTTTTCTTTTGATGATGAAGTATTGCTAATA
TTACCGTGGTTTATGAACTATGTTCAGAATTGAAGAAAATCCTAACTTTCAGTTAGAGGTTAGTGACGGG
GTTCAGGACACCCTACACAAAATACAGCACTTTGACATATTGAATATTTTAAGCTGAAGGCATTTGAGGA
AATTGCAGAAGCAGGAAGGTGACTCTGACCTTCTGCCTGCTGTTCTCCCCAGAAGCAGCCATAAAACCTG
GGAAGGATTTTCTGACCTTCCCCTGAAGTAGATCATAAGACTGTCATGTAAGAGGTGCTCTCCTGGCACC
CAGAGAAAAGGAGCATCCTTACCTCCAAAAGCACAGGGACACAAAGAGGAATCTAAACAAACAGGCCTCT
CAGTTTCCCCCAGTTTATTACATTTAGCTTGTTCACACTTTGCCCTATGACATTTCTACATCACTGGCTG
CTCTTCATCAAACCTACTATAAAAAACATTCAAGTTCAACTGTTTCTTTGGGCCTTTATTTCCTTATGGA
GCCCCTCGTGTCGTGTAAAACTTATATTAAATAAATGTGCATGCTTT
SEQ ID NO: 16
- Homo sapiens epoxide hydrolase 2,
cytoplasmic (EPHX2), mRNA
CTGGGCGGGTCATGCGCCCTGGCCTTCGCGCATCTCCCAGGTTAGCTGCGTGTCCGGGTGCTAGGCTGCA
GACCCGCCGCCATGACGCTGCGCGCGGCCGTCTTCGACCTTGACGGGGTGCTGGCGCTGCCAGCGGTGTT
CGGCGTCCTCGGCCGCACGGAGGAGGCCCTGGCGCTGCCCAGAGGACTTCTGAATGATGCTTTCCAGAAA
GGGGGACCAGAGGGTGCCACTACCCGGCTTATGAAAGGAGAGATCACACTTTCCCAGTGGATACCACTCA
TGGAAGAAAACTGCAGGAAGTGCTCCGAGACCGCTAAAGTCTGCCTCCCCAAGAATTTCTCCATAAAAGA
AATCTTTGACAAGGCGATTTCAGCCAGAAAGATCAACCGCCCCATGCTCCAGGCAGCTCTCATGCTCAGG
AAGAAAGGATTCACTACTGCCATCCTCACCAACACCTGGCTGGACGACCGTGCTGAGAGAGATGGCCTGG
CCCAGCTGATGTGTGAGCTGAAGATGCACTTTGACTTCCTGATAGAGTCGTGTCAGGTGGGAATGGTCAA
ACCTGAACCTCAGATCTACAAGTTTCTGCTGGACACCCTGAAGGCCAGCCCCAGTGAGGTCGTTTTTTTG
GATGACATCGGGGCTAATCTGAAGCCAGCCCGTGACTTGGGAATGGTCACCATCCTGGTCCAGGACACTG
ACACGGCCCTGAAAGAACTGGAGAAAGTGACCGGAATCCAGCTTCTCAATACCCCGGCCCCTCTGCCGAC
CTCTTGCAATCCAAGTGACATGAGCCATGGGTACGTGACAGTAAAGCCCAGGGTCCGTCTGCATTTTGTG
GAGCTGGGCTCCGGCCCTGCTGTGTGCCTCTGCCATGGATTTCCCGAGAGTTGGTATTCTTGGAGGTACC
AGATCCCTGCTCTGGCCCAGGCAGGTTACCGGGTCCTAGCTATGGACATGAAAGGCTATGGAGAGTCATC
TGCTCCTCCCGAAATAGAAGAATATTGCATGGAAGTGTTATGTAAGGAGATGGTAACCTTCCTGGATAAA
CTGGGCCTCTCTCAAGCAGTGTTCATTGGCCATGACTGGGGTGGCATGCTGGTGTGGTACATGGCTCTCT
TCTACCCCGAGAGAGTGAGGGCGGTGGCCAGTTTGAATACTCCCTTCATACCAGCAAATCCCAACATGTC
CCCTTTGGAGAGTATCAAAGCCAACCCAGTATTTGATTACCAGCTCTACTTCCAAGAACCAGGAGTGGCT
GAGGCTGAACTGGAACAGAACCTGAGTCGGACTTTCAAAAGCCTCTTCAGAGCAAGCGATGAGAGTGTTT
TATCCATGCATAAAGTCTGTGAAGCGGGAGGACTTTTTGTAAATAGCCCAGAAGAGCCCAGCCTCAGCAG
GATGGTCACTGAGGAGGAAATCCAGTTCTATGTGCAGCAGTTCAAGAAGTCTGGTTTCAGAGGTCCTCTA
AACTGGTACCGAAACATGGAAAGGAACTGGAAGTGGGCTTGCAAAAGCTTGGGACGGAAGATCCTGATTC
CGGCCCTGATGGTCACGGCGGAGAAGGACTTCGTGCTCGTTCCTCAGATGTCCCAGCACATGGAGGACTG
GATTCCCCACCTGAAAAGGGGACACATTGAGGACTGTGGGCACTGGACACAGATGGACAAGCCAACCGAG
GTGAATCAGATCCTCATTAAGTGGCTGGATTCTGATGCCCGGAACCCACCGGTGGTCTCAAAGATGTAGA
ACGCAGCGTGTGCCCACGCTCAGCAGGTGTGCCATCCTTCCACCTGCTGGGGCACCATTCTTAGTATACA
GAGGTGGCCTTACACACATCTTGCATGGATGGCAGCATTGTTCTGAAGGGGTTTGCAGAAAAAAAAGATT
TTCTTTACATAAAGTGAATCAAATTTGACATTATTTTAGATCCCAGAGAAATCAGGTGTGATTAGTTCTC
CAGGCATGAATGCATCGTCCCTTTATCTGTAAGAACCCTTAGTGTCCTGTAGGGGGACAGAATGGGGTGG
CCAGGTGGTGATTTCTCTTTGACCAATGCATAGTTTGGCAGAAAAATCAGCCGTTCATTTAGAAGAATCT
TAGCAGAGATTGGGATGCCTTACTCAATAAAGCTAAGATGACTATGCTGCTGGCTGTCTTTGTTCTTGGA
GAGGTGGAGTGACTGTTCACGGAGAA
SEQ ID NO: 17
- Homo sapiens exostosin 2 (EXT2),
transcript variant 2, mRNA
CTGTCTGAGCATTTCACTGCGGAGCCTGAGCGCGCCTGCCTGGGAAAACACTGCAGCGGTGCTCGGACTC
CTCCTGTCCAGCAGGAGGCGCGGCCCGGCAGCTCCCGCATGCGCAGTGCGCTCGGTGTCAGACGGCCCGG
ATCCCGGTTACCGGCCCCTCGCTCGCTGCTCGCCAGCCCAGACTCGGCCCTGGCAGTGGCGGCTGGCGAT
TCGGACCGATCCGACCTGGGCGGAGGTGGCCCGCGCCCCGCGGCATGAGCCGGTGACCAAGCTCGGGGCC
GAGCGGGAGGCAGCCGTGGCCGAGGAGTGTGAGGAAGAGGCTGTCTGTGTCATTATGTGTGCGTCGGTCA
AGTATAATATCCGGGGTCCTGCCCTCATCCCAAGAATGAAGACCAAGCACCGAATCTACTATATCACCCT
CTTCTCCATTGTCCTCCTGGGCCTCATTGCCACTGGCATGTTTCAGTTTTGGCCCCATTCTATCGAGTCC
TCAAATGACTGGAATGTAGAGAAGCGCAGCATCCGTGATGTGCCGGTTGTTAGGCTGCCAGCCGACAGTC
CCATCCCAGAGCGGGGGGATCTCAGTTGCAGAATGCACACGTGTTTTGATGTCTATCGCTGTGGCTTCAA
CCCAAAGAACAAAATCAAGGTGTATATCTATGCTCTGAAAAAGTACGTGGATGACTTTGGCGTCTCTGTC
AGCAACACCATCTCCCGGGAGTATAATGAACTGCTCATGGCCATCTCAGACAGTGACTACTACACTGATG
ACATCAACCGGGCCTGTCTGTTTGTTCCCTCCATCGATGTGCTTAACCAGAACACACTGCGCATCAAGGA
GACAGCACAAGCGATGGCCCAGCTCTCTAGGTGGGATCGAGGTACGAATCACCTGTTGTTCAACATGTTG
CCTGGAGGTCCCCCAGATTATAACACAGCCCTGGATGTCCCCAGAGACAGGGCCCTGTTGGCTGGTGGCG
GCTTTTCTACGTGGACTTACCGGCAAGGCTACGATGTCAGCATTCCTGTCTATAGTCCACTGTCAGCTGA
GGTGGATCTTCCAGAGAAAGGACCAGGTCCACGGCAATACTTCCTCCTGTCATCTCAGGTGGGTCTCCAT
CCTGAGTACAGAGAGGACCTAGAAGCCCTCCAGGTCAAACATGGAGAGTCAGTGTTAGTACTCGATAAAT
GCACCAACCTCTCAGAGGGTGTCCTTTCTGTCCGTAAGCGCTGCCACAAGCACCAGGTCTTCGATTACCC
ACAGGTGCTACAGGAGGCTACTTTCTGTGTGGTTCTTCGTGGAGCTCGGCTGGGCCAGGCAGTATTGAGC
GATGTGTTACAAGCTGGCTGTGTCCCGGTTGTCATTGCAGACTCCTATATTTTGCCTTTCTCTGAAGTTC
TTGACTGGAAGAGAGCATCTGTGGTTGTACCAGAAGAAAAGATGTCAGATGTGTACAGTATTTTGCAGAG
CATCCCCCAAAGACAGATTGAAGAAATGCAGAGACAGGCCCGCTGGTTCTGGGAAGCGTACTTCCAGTCA
ATTAAAGCCATTGCCCTGGCCACCCTGCAGATTATCAATGACCGGATCTATCCATATGCTGCCATCTCCT
ATGAAGAATGGAATGACCCTCCTGCTGTGAAGTGGGGCAGCGTGAGCAATCCACTCTTCCTCCCGCTGAT
CCCACCACAGTCTCAAGGGTTCACCGCCATAGTCCTCACCTACGACCGAGTAGAGAGCCTCTTCCGGGTC
ATCACTGAAGTCTCCAAGGTGCCCAGTCTATCCAAACTACTTCTCGTCTGGAATAATCAGAATAAAAACC
CTCCAGAAGATTCTCTCTGGCCCAAAATCCGGGTTCCATTAAAAGTTGTGAGGACTGCTGAAAACAAGTT
AAGTAACCGTTTCTTCCCTTATGATGAAATCGAGACAGAAGCTGTTCTGGCCATTGATGATGATATCATT
ATGCTGACCTCTGACGAGCTGCAATTTGGTTATGAGGTCTGGCGGGAATTTCCTGACCGGTTGGTGGGTT
ACCCGGGTCGTCTGCATCTCTGGGACCATGAGATGAATAAGTGGAAGTATGAGTCTGAGTGGACGAATGA
AGTGTCCATGGTGCTCACTGGGGCAGCTTTTTATCACAAGTATTTTAATTACCTGTATACCTACAAAATG
CCTGGGGATATCAAGAACTGGGTAGATGCTCATATGAACTGTGAAGATATTGCCATGAACTTCCTGGTGG
CCAACGTCACGGGAAAAGCAGTTATCAAGGTAACCCCACGAAAGAAATTCAAGTGTCCTGAGTGCACAGC
CATAGATGGGCTTTCACTAGACCAAACACACATGGTGGAGAGGTCAGAGTGCATCAACAAGTTTGCTTCA
GTCTTCGGGACCATGCCTCTCAAGGTGGTGGAACACCGAGCTGACCCTGTCCTGTACAAAGATGACTTTC
CTGAGAAGCTGAAGAGCTTCCCCAACATTGGCAGCTTATGAAACGTGTCATTGGTGGAGGTCTGAATGTG
AGGCTGGGACAGAGGGAGAGAACAAGGCCTCCCAGCACTCTGATGTCAGAGTAGTAGGTTAAGGGTGGAA
GGTTGACCTACTTGGATCTTGGCATGCACCCACCTAACCCACTTTCTCAAGAACAAGAACCTAGAATGAA
TATCCAAGCACCTCGAGCTATGCAACCTCTGTTCTTGTATTTCTTATGATCTCTGATGGGTTCTTCTCGA
AAATGCCAAGTGGAAGACTTTGTGGCATGCTCCAGATTTAAATCCAGCTGAGGCTCCCTTTGTTTTCAGT
TCCATGTAACAATCTGGAAGGAAACTTCACGGACAGGAAGACTGCTGGAGAAGAGAAGCGTGTTAGCCCA
TTTGAGGTCTGGGGAATCATGTAAAGGGTACCCAGACCTCACTTTTAGTTATTTACATCAATGAGTTCTT
TCAGGGAACCAAACCCAGAATTCGGTGCAAAAGCCAAACATCTTGGTGGGATTTGATAAATGCCTTGGGA
CCTGGAGTGCTGGGCTTGTGCACAGGAAGAGCACCAGCCGCTGAGTCAGGATCCTGTCAGTTCCATGAGC
TATTCCTCTTTGGTTTGGCTTTTTGATATGATTAAAATTATTTTTTATTCCTTTTTCTACTGTGTCTTAA
ACACCAATTCCTGATAGTCCAAGGAACCACCTTTCTCCCTTGATATATTTAACTCCGTCTTTGGCCTGAC
AACAGTCTTCTGCCCATGTCTGGGAACACACGCCAGGAGGAATGTCTGATACCCTCTGCATCAAGCGTAA
GAAGGTCCCAAATCATAACCATTTTAAGAACAGATGACTCAGAAACCTCCAGAGGAATCTGTTTGCTTCC
TGATTAGATCCAGTCAATGTTTTAAAGGTATTGTCAGAGAAAAACAGAGGGTCTGTACTAGCCATGCAAG
GAGTCGCTCTAGCTGGTACCCGTAAAAGTTGTGGGAATTGTGACCCCCATCCCAAGGGGATGCCAAAATT
TCTCTCATTCTTTTGGTATAAACTTAACATTAGCCAGGGAGGTTCTGGCTAACGTTAAATGCTGCTATAC
AACTGCTTTGCAACAGTTGCTGGTATATTTAAATCATTAAATTTCAGCATTTACTAATACTGCAAAAAAA
AAAAAAAAAAA
SEQ ID NO: 18
- Homo sapiens FBJ murine osteosarcoma viral
oncogene homolog (FOS), mRNA
ATTCATAAAACGCTTGTTATAAAAGCAGTGGCTGCGGCGCCTCGTACTCCAACCGCATCTGCAGCGAGCA
TCTGAGAAGCCAAGACTGAGCCGGCGGCCGCGGCGCAGCGAACGAGCAGTGACCGTGCTCCTACCCAGCT
CTGCTCCACAGCGCCCACCTGTCTCCGCCCCTCGGCCCCTCGCCCGGCTTTGCCTAACCGCCACGATGAT
GTTCTCGGGCTTCAACGCAGACTACGAGGCGTCATCCTCCCGCTGCAGCAGCGCGTCCCCGGCCGGGGAT
AGCCTCTCTTACTACCACTCACCCGCAGACTCCTTCTCCAGCATGGGCTCGCCTGTCAACGCGCAGGACT
TCTGCACGGACCTGGCCGTCTCCAGTGCCAACTTCATTCCCACGGTCACTGCCATCTCGACCAGTCCGGA
CCTGCAGTGGCTGGTGCAGCCCGCCCTCGTCTCCTCCGTGGCCCCATCGCAGACCAGAGCCCCTCACCCT
TTCGGAGTCCCCGCCCCCTCCGCTGGGGCTTACTCCAGGGCTGGCGTTGTGAAGACCATGACAGGAGGCC
GAGCGCAGAGCATTGGCAGGAGGGGCAAGGTGGAACAGTTATCTCCAGAAGAAGAAGAGAAAAGGAGAAT
CCGAAGGGAAAGGAATAAGATGGCTGCAGCCAAATGCCGCAACCGGAGGAGGGAGCTGACTGATACACTC
CAAGCGGAGACAGACCAACTAGAAGATGAGAAGTCTGCTTTGCAGACCGAGATTGCCAACCTGCTGAAGG
AGAAGGAAAAACTAGAGTTCATCCTGGCAGCTCACCGACCTGCCTGCAAGATCCCTGATGACCTGGGCTT
CCCAGAAGAGATGTCTGTGGCTTCCCTTGATCTGACTGGGGGCCTGCCAGAGGTTGCCACCCCGGAGTCT
GAGGAGGCCTTCACCCTGCCTCTCCTCAATGACCCTGAGCCCAAGCCCTCAGTGGAACCTGTCAAGAGCA
TCAGCAGCATGGAGCTGAAGACCGAGCCCTTTGATGACTTCCTGTTCCCAGCATCATCCAGGCCCAGTGG
CTCTGAGACAGCCCGCTCCGTGCCAGACATGGACCTATCTGGGTCCTTCTATGCAGCAGACTGGGAGCCT
CTGCACAGTGGCTCCCTGGGGATGGGGCCCATGGCCACAGAGCTGGAGCCCCTGTGCACTCCGGTGGTCA
CCTGTACTCCCAGCTGCACTGCTTACACGTCTTCCTTCGTCTTCACCTACCCCGAGGCTGACTCCTTCCC
CAGCTGTGCAGCTGCCCACCGCAAGGGCAGCAGCAGCAATGAGCCTTCCTCTGACTCGCTCAGCTCACCC
ACGCTGCTGGCCCTGTGAGGGGGCAGGGAAGGGGAGGCAGCCGGCACCCACAAGTGCCACTGCCCGAGCT
GGTGCATTACAGAGAGGAGAAACACATCTTCCCTAGAGGGTTCCTGTAGACCTAGGGAGGACCTTATCTG
TGCGTGAAACACACCAGGCTGTGGGCCTCAAGGACTTGAAAGCATCCATGTGTGGACTCAAGTCCTTACC
TCTTCCGGAGATGTAGCAAAACGCATGGAGTGTGTATTGTTCCCAGTGACACTTCAGAGAGCTGGTAGTT
AGTAGCATGTTGAGCCAGGCCTGGGTCTGTGTCTCTTTTCTCTTTCTCCTTAGTCTTCTCATAGCATTAA
CTAATCTATTGGGTTCATTATTGGAATTAACCTGGTGCTGGATATTTTCAAATTGTATCTAGTGCAGCTG
ATTTTAACAATAACTACTGTGTTCCTGGCAATAGTGTGTTCTGATTAGAAATGACCAATATTATACTAAG
AAAAGATACGACTTTATTTTCTGGTAGATAGAAATAAATAGCTATATCCATGTACTGTAGTTTTTCTTCA
ACATCAATGTTCATTGTAATGTTACTGATCATGCATTGTTGAGGTGGTCTGAATGTTCTGACATTAACAG
TTTTCCATGAAAACGTTTTATTGTGTTTTTAATTTATTTATTAAGATGGATTCTCAGATATTTATATTTT
TATTTTATTTTTTTCTACCTTGAGGTCTTTTGACATGTGGAAAGTGAATTTGAATGAAAAATTTAAGCAT
TGTTTGCTTATTGTTCCAAGACATTGTCAATAAAAGCATTTAAGTTGAATGCGACCAA
SEQ ID NO: 19
- Homo sapiens FOS-like antigen 1 (FOSL1), mRNA
ACGGGCCAAGGCGGCGCGTCTCGGGGGTGGAGCCTGGAGGTGACCGCGCCGCTGCAACGCCCCCACCCCC
CGCGGTCGCAGTGGTTCAGCCCGAGAACTTTTCATTCATAAAAAGAAAAGACTCCGCACGGCGCGGGTGA
GTCAGAACCCAGCAGCCGTGTACCCCGCAGAGCCGCCAGCCCCGGGCATGTTCCGAGACTTCGGGGAACC
CGGCCCGAGCTCCGGGAACGGCGGCGGGTACGGCGGCCCCGCGCAGCCCCCGGCCGCAGCGCAGGCAGCC
CAGCAGAAGTTCCACCTGGTGCCAAGCATCAACACCATGAGTGGCAGTCAGGAGCTGCAGTGGATGGTAC
AGCCTCATTTCCTGGGGCCCAGCAGTTACCCCAGGCCTCTGACCTACCCTCAGTACAGCCCCCCACAACC
CCGGCCAGGAGTCATCCGGGCCCTGGGGCCGCCTCCAGGGGTACGTCGAAGGCCTTGTGAACAGATCAGC
CCGGAGGAAGAGGAGCGCCGCCGAGTAAGGCGCGAGCGGAACAAGCTGGCTGCGGCCAAGTGCAGGAACC
GGAGGAAGGAACTGACCGACTTCCTGCAGGCGGAGACTGACAAACTGGAAGATGAGAAATCTGGGCTGCA
GCGAGAGATTGAGGAGCTGCAGAAGCAGAAGGAGCGCCTAGAGCTGGTGCTGGAAGCCCACCGACCCATC
TGCAAAATCCCGGAAGGAGCCAAGGAGGGGGACACAGGCAGTACCAGTGGCACCAGCAGCCCACCAGCCC
CCTGCCGCCCTGTACCTTGTATCTCCCTTTCCCCAGGGCCTGTGCTTGAACCTGAGGCACTGCACACCCC
CACACTCATGACCACACCCTCCCTAACTCCTTTCACCCCCAGCCTGGTCTTCACCTACCCCAGCACTCCT
GAGCCTTGTGCCTCAGCTCATCGCAAGAGTAGCAGCAGCAGCGGAGACCCATCCTCTGACCCCCTTGGCT
CTCCAACCCTCCTCGCTTTGTGAGGCGCCTGAGCCCTACTCCCTGCAGATGCCACCCTAGCCAATGTCTC
CTCCCCTTCCCCCACCGGTCCAGCTGGCCTGGACAGTATCCCACATCCAACTCCAGCAACTTCTTCTCCA
TCCCTCTAATGAGACTGACCATATTGTGCTTCACAGTAGAGCCAGCTTGGGGCCACCAAAGCTGCCCACT
GTTTCTCTTGAGCTGGCCTCTCTAGCACAATTTGCACTAAATCAGAGACAAAATATTTCCCATTTGTGCC
AGAGGAATCCTGGCAGCCCAGAGACTTTGTAGATCCTTAGAGGTCCTCTGGAGCCCTAACCCCTTCCAGA
TCACTGCCACACTCTCCATCACCCTCTTCCTGTGATCCACCCAACCCTATCTCCTGACAGAAGGTGCCAC
TTTACCCACCTAGAACACTAACTCACCAGCCCCACTGCCAGCAGCAGCAGGTGATTGGACCAGGCCATTC
TGCCGCCCCCTCCTGAACCGCACAGCTCAGGAGGCGCCCTTGGCTTCTGTGATGAGCTGATCTGCGGATC
TCAGCTTTGAGAAGCCTTCAGCTCCAGGGAATCCAAGCCTCCACAGCGAGGGCAGCTGCTATTTATTTTC
CTAAAGAGAGTATTTTTATACAAACCTACCAAAATGGAATAAAAGGCTTGAAGCTGTG
SEQ ID NO: 20
- Homo sapiens forkhead box N3 (FOXN3),
transcript variant 1, mRNA
CGCGATCTGCTGCAGCTCGGCCGGGAGACGGCGCGACCCGGCGGCGGGGCCACCCGCGAGTCCAGCGTCG
CCGCAGCCCCCCAATGCGGCCGCGAGAAGCAGCGGGGGGGCAGGCGATCGAAGGAGCCTTCACGTAAATG
GGTCCAGTCATGCCTCCCAGTAAGAAGCCAGAAAGCTCAGGAATTAGTGTCTCCAGTGGACTGAGTCAGT
GTTACGGGGGCAGCGGTTTCTCCAAGGCCCTTCAGGAAGACGATGACCTCGACTTTTCTCTGCCTGACAT
CCGATTAGAAGAGGGGGCCATGGAAGATGAAGAGCTGACCAACCTGAACTGGCTGCACGAGAGCAAGAAC
TTGCTGAAGAGCTTTGGGGAGTCGGTCCTCAGGAGTGTCAGCCCCGTCCAGGACCTGGACGATGACACCC
CCCCATCCCCTGCCCACTCTGACATGCCCTACGATGCCAGGCAGAACCCCAACTGCAAACCCCCCTACTC
CTTCAGCTGCCTCATATTTATGGCCATCGAGGACTCTCCAACCAAGCGCCTGCCAGTGAAGGATATCTAC
AACTGGATCTTGGAACATTTTCCGTATTTTGCAAATGCACCTACTGGGTGGAAAAACTCAGTGAGACACA
ATTTATCATTGAATAAGTGTTTTAAGAAAGTGGACAAAGAGAGGAGTCAGAGTATTGGGAAAGGGTCGTT
GTGGTGCATAGACCCAGAGTATAGACAAAATCTAATTCAGGCTTTGAAAAAGACACCTTATCACCCACAC
CCACACGTGTTCAATACACCTCCCACCTGTCCTCAGGCATATCAAAGCACATCAGGTCCACCCATCTGGC
CGGGCAGTACCTTCTTCAAGAGAAATGGAGCCCTTCTCCAAGATCCTGACATTGATGCTGCCAGTGCCAT
GATGCTTTTGAATACTCCCCCTGAGATACAAGCAGGTTTTCCTCCAGGAGTGATCCAAAATGGAGCGCGG
GTCCTGAGCCGAGGGCTGTTTCCTGGCGTGCGGCCGCTGCCAATCACTCCCATTGGGGTGACAGCGGCCA
TGAGGAATGGCATCACCAGCTGCCGGATGCGGACTGAGAGTGAGCCATCTTGTGGCTCCCCAGTGGTCAG
CGGAGACCCCAAGGAGGATCACAACTACAGCAGTGCCAAGTCCTCCAACGCCCGGAGCACCTCGCCCACC
AGCGACTCCATCTCCTCCTCCTCCTCCTCAGCCGACGACCACTATGAGTTTGCCACCAAGGGGAGCCAGG
AGGGCAGCGAGGGCAGCGAGGGGAGCTTCCGGAGCCACGAGAGCCCCAGCGACACGGAAGAGGACGACAG
GAAGCACAGCCAGAAGGAGCCCAAGGATTCTCTGGGGGACAGCGGGTACGCATCCCAGCACAAGAAGCGC
CAGCACTTCGCCAAGGCCAGGAAGGTCCCCAGCGACACACTGCCCCTCAAAAAGAGACGCACCGAAAAGC
CCCCCGAGAGCGATGATGAGGAGATGAAAGAAGCGGCAGGGTCCCTCCTGCACTTAGCAGGGATCCGGTC
CTGTTTGAATAACATCACCAATCGGACGGCAAAGGGGCAGAAAGAGCAAAAGGAAACCACAAAAAATTAA
AAACAAGTCACTGATTTGTTTTGAACTTACGACCATTTGGTTTCAGCATGTCAGGAGATTTCTAATGATT
TGTGGCAATATCAGCAATTTTTTTTCTTTTTTCTTGTTTTTGGTTTGGTTTTCTTTCTTTTCTTTTCCTT
TTATTTTGTTTTAATTTGCCCCCTCTTCTTTGTTTTGGACCCTTAAGAATTTTATTTTTAAAGGAGATTG
AAGCCATAGAACTCATATTGACACTCAGCTGTTTTACAAAAGCTTTTCATTATCTGAAGACAAAACCGAA
AAAGCCAAAATTACCATTGCTTCCTCCAGCTTGTCAGAAACCTGTGGCTGAATCCGCAGGGATGTCAACG
TCAATATCACAGGAACACACATTCGGCACCTAGAAGGCACGTGGGCAAAGTAATCATCGTTCAGGCCCAA
CCCTTAGGTTTAAAAAGTCAGGTTGTCCATCCCATTGGGTTCACTGAGTGAAGGCACATAAAGCAATTGA
GGAGGAGGAGGAACCCCTCGTCCCCCTAGGAGCAGACCCAAGCTTGTGGCACCAGGCATCTGATGGTGCC
AGGAAAGCCACTGGAATTGTCACACGGCGAGCACAGAGGGCCGGCCACCAGTCCTCGATGCTTCTGAACC
CTGAAGCCCGATGACATCTTACGAGGTGGACGTTGGACTGTTCATGCGCATCGGGTGTCAGTGACTCATG
GAGAAGAAATGGGGTAAATTTTTAGTGATGTTGCTAATCATTGAATTCTGTTCTCTATTAAATTAAGAAA
ATGTTCCAAAAGCCATAAGCCTGAAGATTGGCCCTGTGCACGCACGCACACACACACACACACACACACA
CACACACACACACACACGAAGGAGAGAGAGAGAAAACTGATGGGGAAAACAAGCTGTGTCTTCTTAACTG
CCCAAGTGAAAAGCAACCAAGTCCAGGAAATTACAATAGCTGTTAAGGAAAGGAAATAATGGTACAGATC
TTTTTCTGTCTATCAAAACTATTTGATCCAAGTGAAAAAAAAAAAAAAACTAGAAAGCTACGGAACCTGC
CATTAGTATTGTGGTGTATTTTTAAGATTAAAGGTACACTGATGGACAAAAAAAAAAAGTAAAACATGGC
AAAAAATAAAATAACTCCTATACTGCCCTCAAAATGGAGTTTGCAATTAATATCAGGATTTATCTTTGCA
AAAATCAGTGATTTCCACATTCAGCCAGTATAGCCAGCAGAAATTTCTGATCCACAATGCATGGATTCCT
TTGAAGAAAAAAAAGAAAAAGAGAAAAAAATCACAAAAACAAACTTTTTTTATTCAAAAGTAACAAAGTT
CTTGTAAGGTAAATAATGTATTTAGCATGAAGCATGAATTATTTTCATATAAATATAGAAAATAGAGAAA
AGGCTATGCCTGTAATTTTTAAGCCCTTAGGCTTAGAGTTTCTTTTGGTTTTCTTCTTTTTTCTTTCCTT
TTCTTTGCTTTCTTTTTTTCCTTTTTGTTTTTGTTTTTGTTTTTTGTTTTTGTTTTTTTTTCGGGTTATT
TTGTTTTGGTTTTTTGAAGCAGGTGTTTAAGGTTTAACCTTCTTCAGGGACAAATTCTGACTGTTGGGGA
ACTTACTCTGCAATATAAAAATATCTTCATGCTCTGGTAGGGCTTGGATGGTTGAACTCTGTACTGCCTT
GTGTGCACTTCAGCCCCGACCCCCTCTGATTCTCTGTTGAAAAGTGTGTCCTTTCTCTCTGTCTGTACAT
GTTTAACATGACGCAATAATTTGAGGGCAAACTTAGTAGTGAGTGTGTATGATAGAATCAAGAGAATTAT
GGGACGCTTACTTGAGAAAATCATTACCATGATTTGGTTCTAGGAAAAAGGCAGTGAATAATTATGCAAA
TTAGCCAGAAGAAGGGGAACCGTGCTAATGGGCCTTATTGGGTGAGGGGACGAGATGGGGTTCATGTGAA
GGAGGAAGCGATGCCGAGGTAGGAAAGGCCAGCCCCAGACATCCTATCGCCACAATGCCATGTCGCAATA
GGAAGCAGGGGCCGGCCATCGCTACCTTCAGCACACTGACCAACCTGGAATTAAGACCACCTAGATTGCG
AGAGCTGAATTTAGAAACCAGACAACGTCATGCAGCCCAGAAACTCCTGTTGTTACCTTTGCCTAAGAAA
TTTTCTTTAATGGCGGGGGCGGGGGGCGGGGGTACAAAGAGAAATCTCTAAAAGAATATGATCTTCCATC
CAAGTGGAGGGAAACTTTAAAACAAAAACACCCAGTACTGTGGCTCAGGATATGATGCGTGAGGAGAGGG
AGGGAACAGAGATGACCTTAACTTTTAAAAAAGGGACTGCTGTGGGCCAAAGCCAAGCCCATCTGCCAGG
ACGAGGTAATGTCAGAGCTCCATCAGCCCGGACAGTGGGAACTAACTGGTGCATTCCCCACACTTACCTT
CCGGTGGGTTGCTGATGAGAGAACCTGAAAAAACCTACACCTCTACAGCAGGTCGAATTCATGACCTGAA
GCTGAATACTTCCAGCATATTTATTCAGGGTGTAGGTGGGAATAAAGTATCTTCGCAGTGCTCTGTTCCC
TCCGTCTCCCCAGACATCTGACACCCTAAAAGCCATCCACAGCTATGGAACCTGAGCGACACCTTGATTT
GTGTTGTCACCTGACCAAGCCTAAAGACCTCCAGCTCAGTCCCCCACCTTCATCCCACCCCACAGATGAT
AAAATTCAGACCTCTCTCCTGAAAGGCAGAGGTTCAACATTCAGGACTGTTTCTGGCCGAGGACTTCTTC
CAATTAAAACCCCCACCGTGGGCTGTCTCCCCTCATTTCATTTTTCTAAAGGGGCAGAGGCCTCTTTTAG
AAAATAATAAAATGCAATGTGTGTGATTTACTTTTCTGATCTCTTTGAGAAATAGAGAAATATAAAAGTG
TGTTCTTAACTCCAGAACCACTCTTTTTGCATAAATACCTCATCGGGCAGCTTTCTAAGTGTGATTTTCC
TGAGTCTCCCTTCGTTGGATCTGCCGGAAGACTTGTCGGGGAACCTTTAGTGAGGGTACTTCTTCCTATT
TTTCTTCTGTTTTTGGAGGCATACACATTATGCATAACCAAAACAATGGCTCAATTGTGTTTAACTTTGT
ATTTTGATTGTTGAGAACAAAAACAAAAAGTATCAATGTGTATGTGGCTGTTTGTAGTGAATTTATTGGA
GAATGAGGTTGTCCGTGTCCTTAACAAGCCAAGGGGCAGGAGGCACCCTCTCTTATCCCCTCCTCCAAGA
GCAGTAGAGAATTTAAGCACAAGCCTATTTGTGAAAGAATATTTTGCTTAAGTGTCATTCACTTTAGTCT
TGGAATTCCTTCCCAAACGTCAGGTGTTCTTTTAGCTTCCAAACTAGCATATGTATCCATTAGTCTGACA
GATCGCCTGAACACCATTAAGAGGTGTGGCGTTTTTGCTTTCATTTCTCCTGCTGGGAGAAGTGGCGGTT
CATGTGTCATTCCAGTATCTCACATACTCACACGGGGCAGGGGGGAGGGGGAAACGGGGAACTATAGCAA
TATTTAAAGATGCTTTGGAAACCAACCGTGAACACATCAACACCACGACGTCTACGATTACTTGCTATTG
GCCCTCGGATACATTTAAGAGAAAGAGACAGTCACTCTTTTTTTTCTTAAATGATATACATATAAACAGT
TATTTTTATCCTATTATAATTGTCTTTTGTCTTTATCTAGTACTATGTGGAAAGGGTTTGCATCATAGAT
TTTTCCCAGCCTTATAATATACCATAAGCTCCTACTTCCCTGCCCCTCCCTAATCAGTATTCTTTCAAGA
GTTCTTTGGTGAAGCCATCTATCTGAAACTAAAATGAACCAAACCCATATTTCACTGGTGGTTGGAGAAA
ACCATGGCCAAAACGATTGTGGCAGGTCTCAATCTTGGGAGTTTTTAAGAAGGAATGTGCCAGAGGCCGA
TTCCCAAGAACAGAGTTTTCTTTTGTTTTGCAGAGGCATTCAATGTGTCTAGTGCTTGCTGGCCACAGCA
GTTACTACCACAGAGCCTTCTGGGAGGGGCCGTTGTGTTGAAGGAGGCTCCTGCCTGAGGGACAGCATCA
GGCAGTGGGCTCTGTAGAGTGAGAACCAGGTGGAGGCCTTCTCTGCCCAGCTCAGAGTTCTGCACCACGC
CAGGACTGCCCAGGCCAAGGGCTACTGACGCAAGTTCCACTCATTCCACTCTGTGGGGGGCGCCTTGGGC
CTCTCCTGGAAGGGCTCTTGGAGAAGGAATTGGAGTTACGTACAAGTGACCTAAATGGGAAGCTTTTCTA
GATGAGATTGGATTAAATTCCATGTGATTTCTCTTTCCCTTTAATCCAGGTTGGGACTCGTTTCTTTCTG
GTGGATCACAGCTGCCCAGATGTTGCAATTGATTTTTATGTTTCTGTAGAGAAGTATTTTTCTTTCATCT
TCAGGATTTTTTTTGCCACCAAAAGAAAACATTGGAACTCTGTGTTTCCTCTTGATTGTGACTTCCCAGT
GTTGACAGTTAAGTCCTTAGTGTCGTAGGTCCCAGCCCACCAATACTATATCAAACACTGTTATGCACAT
AATGCAGCACTCTGATCTAATTTAAATAATACTTTTTTATTATTTATACTACTATATATAATATACATCA
ACACTTTTGCTATATAACCTAAGTGATAACCCTCTTTTAGTTACCTGCCAAACTCTGGACTTGGTTTATA
TTGCAGTTAACACAGTTACAAAGCTGTAATGGTGTCTTTTTTTCCTTTGTAACGGAATGTGTAAATCAAA
GTATATACATTGTGTGGTGTTCCTGTTTCTGGAGTTTCATGAGGATTTACACATGGCATTCAGTGTTCTG
TATAGATCTGCCTACCTTTGTGAATTCATCTGTTAACCCCTCTTCCTTTGAGAGAGCACCGGCGATGGTG
GTTAACTCCTTGTGTTTTCTCTCTCTCCTACTGGTTATTCTTGAATTAAGCACAGACTCGTCAGCTCGGT
TGCTTTATCATGAATAATGTGTGTGACCTTGCAGTTCTTCCACAGTTCAGCAAACAAGTGCTAGCTTCAC
TGACCAAAAATTAAGGAAGGAAAACACAGTTTTTAAAACGATCCATCTTTTAACAGCCGAAACCGATGTG
TCTATGGTGCTGCACCTTGCTGTTGTACTTCTGAAATCAGACGTGTGTGAACGATCATTTCTGACTTAAC
CGTGAGATGCTCACGAGTACCCTTCCTGTTGTTTTGTTAGCATTGAAATCGAGACTATTTATTTGGAATA
TATACAACAGTGTTTTTCCACTGTATTTCATTTGCAAAAGTTGAGAACTGCTTTCTCTACCTTTTGCAAA
ATAATTGATATTCCATATTGGATTCTCAAAGACTTCGATATGCTGAACCTATTAAACCTAGAAATTGTAT
TCATCCTTTCATGACTGTGGCCTGAGTTCCCCAGCCCCTCTCCTCCTTTTTTTTAGATGAGATTTAGCAC
ACTCTCAGTTATTTAAACATGCAACATTTCTTGAGTATGTATGTTGAGGCCATCTGAGCTCATAGCTGAT
TCAGTAACCAGTTTCATGCTGTGTCATTCACACTCACTACTTAATACTGCCATGGTGAAAATGTGGAGGA
AAAATGTATCCATGTGTGTCTGGGAAGCATATACACTTGTACATTTTTTAATACTCTGATTCTGTAACAT
TTCTGAGTTTTGTTTTGTTTTACAGAAAAAAAAAAAAAGTGATAAAGCAATCAGAAGACCAAGAGGTTTA
CTATTGATGCTTAGGGTCGTCTGACCTTGGCTGGCCAATAGACCTACACGGCCAAATTAATTTACGAGAG
TAATAATTTTTCAAAAGCCAATTTTTTTTCTGTATTTTCTGTATGAAACTGCCAATATCATGAATAGAAA
GGGAGAACCATAAAGGAGAAAGAACGTGATGTTCTGTTATGTTCATGTAAACCTAAAGAAACAGTGTGGA
GGCAGGCGCGATCAGCCGAACTCTAGGGACTTGGTGTTGCTTGGAAGGCATCCATACCTGCATTTTGCAT
TCTTCGTATGTAATCATATTGCCAAAGACAAACTATTTCATCATTTATTGTAAATAACACTTTTCCCCAG
ACCTACCATAAAGTTTCTGTGATGTATTGTCTTCCAGTTGCAATAAAAATTACTGAGTTGCATCAATTGA
AGAAAAACACCAAAAA
SEQ ID NO: 21
- Homo sapiens glyceraldehyde-3-phosphate
dehydrogenase (GAPDH), mRNA
AAATTGAGCCCGCAGCCTCCCGCTTCGCTCTCTGCTCCTCCTGTTCGACAGTCAGCCGCATCTTCTTTTG
CGTCGCCAGCCGAGCCACATCGCTCAGACACCATGGGGAAGGTGAAGGTCGGAGTCAACGGATTTGGTCG
TATTGGGCGCCTGGTCACCAGGGCTGCTTTTAACTCTGGTAAAGTGGATATTGTTGCCATCAATGACCCC
TTCATTGACCTCAACTACATGGTTTACATGTTCCAATATGATTCCACCCATGGCAAATTCCATGGCACCG
TCAAGGCTGAGAACGGGAAGCTTGTCATCAATGGAAATCCCATCACCATCTTCCAGGAGCGAGATCCCTC
CAAAATCAAGTGGGGCGATGCTGGCGCTGAGTACGTCGTGGAGTCCACTGGCGTCTTCACCACCATGGAG
AAGGCTGGGGCTCATTTGCAGGGGGGAGCCAAAAGGGTCATCATCTCTGCCCCCTCTGCTGATGCCCCCA
TGTTCGTCATGGGTGTGAACCATGAGAAGTATGACAACAGCCTCAAGATCATCAGCAATGCCTCCTGCAC
CACCAACTGCTTAGCACCCCTGGCCAAGGTCATCCATGACAACTTTGGTATCGTGGAAGGACTCATGACC
ACAGTCCATGCCATCACTGCCACCCAGAAGACTGTGGATGGCCCCTCCGGGAAACTGTGGCGTGATGGCC
GCGGGGCTCTCCAGAACATCATCCCTGCCTCTACTGGCGCTGCCAAGGCTGTGGGCAAGGTCATCCCTGA
GCTGAACGGGAAGCTCACTGGCATGGCCTTCCGTGTCCCCACTGCCAACGTGTCAGTGGTGGACCTGACC
TGCCGTCTAGAAAAACCTGCCAAATATGATGACATCAAGAAGGTGGTGAAGCAGGCGTCGGAGGGCCCCC
TCAAGGGCATCCTGGGCTACACTGAGCACCAGGTGGTCTCCTCTGACTTCAACAGCGACACCCACTCCTC
CACCTTTGACGCTGGGGCTGGCATTGCCCTCAACGACCACTTTGTCAAGCTCATTTCCTGGTATGACAAC
GAATTTGGCTACAGCAACAGGGTGGTGGACCTCATGGCCCACATGGCCTCCAAGGAGTAAGACCCCTGGA
CCACCAGCCCCAGCAAGAGCACAAGAGGAAGAGAGAGACCCTCACTGCTGGGGAGTCCCTGCCACACTCA
GTCCCCCACCACACTGAATCTCCCCTCCTCACAGTTGCCATGTAGACCCCTTGAAGAGGGGAGGGGCCTA
GGGAGCCGCACCTTGTCATGTACCATCAATAAAGTACCCTGTGCTCAACC
SEQ ID NO: 22
- Homo sapiens glyceraldehyde-3-phosphate
dehydrogenase (GAPDH), mRNA
AAATTGAGCCCGCAGCCTCCCGCTTCGCTCTCTGCTCCTCCTGTTCGACAGTCAGCCGCATCTTCTTTTG
CGTCGCCAGCCGAGCCACATCGCTCAGACACCATGGGGAAGGTGAAGGTCGGAGTCAACGGATTTGGTCG
TATTGGGCGCCTGGTCACCAGGGCTGCTTTTAACTCTGGTAAAGTGGATATTGTTGCCATCAATGACCCC
TTCATTGACCTCAACTACATGGTTTACATGTTCCAATATGATTCCACCCATGGCAAATTCCATGGCACCG
TCAAGGCTGAGAACGGGAAGCTTGTCATCAATGGAAATCCCATCACCATCTTCCAGGAGCGAGATCCCTC
CAAAATCAAGTGGGGCGATGCTGGCGCTGAGTACGTCGTGGAGTCCACTGGCGTCTTCACCACCATGGAG
AAGGCTGGGGCTCATTTGCAGGGGGGAGCCAAAAGGGTCATCATCTCTGCCCCCTCTGCTGATGCCCCCA
TGTTCGTCATGGGTGTGAACCATGAGAAGTATGACAACAGCCTCAAGATCATCAGCAATGCCTCCTGCAC
CACCAACTGCTTAGCACCCCTGGCCAAGGTCATCCATGACAACTTTGGTATCGTGGAAGGACTCATGACC
ACAGTCCATGCCATCACTGCCACCCAGAAGACTGTGGATGGCCCCTCCGGGAAACTGTGGCGTGATGGCC
GCGGGGCTCTCCAGAACATCATCCCTGCCTCTACTGGCGCTGCCAAGGCTGTGGGCAAGGTCATCCCTGA
GCTGAACGGGAAGCTCACTGGCATGGCCTTCCGTGTCCCCACTGCCAACGTGTCAGTGGTGGACCTGACC
TGCCGTCTAGAAAAACCTGCCAAATATGATGACATCAAGAAGGTGGTGAAGCAGGCGTCGGAGGGCCCCC
TCAAGGGCATCCTGGGCTACACTGAGCACCAGGTGGTCTCCTCTGACTTCAACAGCGACACCCACTCCTC
CACCTTTGACGCTGGGGCTGGCATTGCCCTCAACGACCACTTTGTCAAGCTCATTTCCTGGTATGACAAC
GAATTTGGCTACAGCAACAGGGTGGTGGACCTCATGGCCCACATGGCCTCCAAGGAGTAAGACCCCTGGA
CCACCAGCCCCAGCAAGAGCACAAGAGGAAGAGAGAGACCCTCACTGCTGGGGAGTCCCTGCCACACTCA
GTCCCCCACCACACTGAATCTCCCCTCCTCACAGTTGCCATGTAGACCCCTTGAAGAGGGGAGGGGCCTA
GGGAGCCGCACCTTGTCATGTACCATCAATAAAGTACCCTGTGCTCAACC
SEQ ID NO: 23
- Homo sapiens GATA binding protein 3 (GATA3),
transcript variant 1, mRNA
GGCGCCGTCTTGATACTTTCAGAAAGAATGCATTCCCTGTAAAAAAAAAAAAAAAATACTGAGAGAGGGA
GAGAGAGAGAGAAGAAGAGAGAGAGACGGAGGGAGAGCGAGACAGAGCGAGCAACGCAATCTGACCGAGC
AGGTCGTACGCCGCCGCCTCCTCCTCCTCTCTGCTCTTCGCTACCCAGGTGACCCGAGGAGGGACTCCGC
CTCCGAGCGGCTGAGGACCCCGGTGCAGAGGAGCCTGGCTCGCAGAATTGCAGAGTCGTCGCCCCTTTTT
ACAACCTGGTCCCGTTTTATTCTGCCGTACCCAGTTTTTGGATTTTTGTCTTCCCCTTCTTCTCTTTGCT
AAACGACCCCTCCAAGATAATTTTTAAAAAACCTTCTCCTTTGCTCACCTTTGCTTCCCAGCCTTCCCAT
CCCCCCACCGAAAGCAAATCATTCAACGACCCCCGACCCTCCGACGGCAGGAGCCCCCCGACCTCCCAGG
CGGACCGCCCTCCCTCCCCGCGCGCGGGTTCCGGGCCCGGCGAGAGGGCGCGAGCACAGCCGAGGCCATG
GAGGTGACGGCGGACCAGCCGCGCTGGGTGAGCCACCACCACCCCGCCGTGCTCAACGGGCAGCACCCGG
ACACGCACCACCCGGGCCTCAGCCACTCCTACATGGACGCGGCGCAGTACCCGCTGCCGGAGGAGGTGGA
TGTGCTTTTTAACATCGACGGTCAAGGCAACCACGTCCCGCCCTACTACGGAAACTCGGTCAGGGCCACG
GTGCAGAGGTACCCTCCGACCCACCACGGGAGCCAGGTGTGCCGCCCGCCTCTGCTTCATGGATCCCTAC
CCTGGCTGGACGGCGGCAAAGCCCTGGGCAGCCACCACACCGCCTCCCCCTGGAATCTCAGCCCCTTCTC
CAAGACGTCCATCCACCACGGCTCCCCGGGGCCCCTCTCCGTCTACCCCCCGGCCTCGTCCTCCTCCTTG
TCGGGGGGCCACGCCAGCCCGCACCTCTTCACCTTCCCGCCCACCCCGCCGAAGGACGTCTCCCCGGACC
CATCGCTGTCCACCCCAGGCTCGGCCGGCTCGGCCCGGCAGGACGAGAAAGAGTGCCTCAAGTACCAGGT
GCCCCTGCCCGACAGCATGAAGCTGGAGTCGTCCCACTCCCGTGGCAGCATGACCGCCCTGGGTGGAGCC
TCCTCGTCGACCCACCACCCCATCACCACCTACCCGCCCTACGTGCCCGAGTACAGCTCCGGACTCTTCC
CCCCCAGCAGCCTGCTGGGCGGCTCCCCCACCGGCTTCGGATGCAAGTCCAGGCCCAAGGCCCGGTCCAG
CACAGAAGGCAGGGAGTGTGTGAACTGTGGGGCAACCTCGACCCCACTGTGGCGGCGAGATGGCACGGGA
CACTACCTGTGCAACGCCTGCGGGCTCTATCACAAAATGAACGGACAGAACCGGCCCCTCATTAAGCCCA
AGCGAAGGCTGTCTGCAGCCAGGAGAGCAGGGACGTCCTGTGCGAACTGTCAGACCACCACAACCACACT
CTGGAGGAGGAATGCCAATGGGGACCCTGTCTGCAATGCCTGTGGGCTCTACTACAAGCTTCACAATATT
AACAGACCCCTGACTATGAAGAAGGAAGGCATCCAGACCAGAAACCGAAAAATGTCTAGCAAATCCAAAA
AGTGCAAAAAAGTGCATGACTCACTGGAGGACTTCCCCAAGAACAGCTCGTTTAACCCGGCCGCCCTCTC
CAGACACATGTCCTCCCTGAGCCACATCTCGCCCTTCAGCCACTCCAGCCACATGCTGACCACGCCCACG
CCGATGCACCCGCCATCCAGCCTGTCCTTTGGACCACACCACCCCTCCAGCATGGTCACCGCCATGGGTT
AGAGCCCTGCTCGATGCTCACAGGGCCCCCAGCGAGAGTCCCTGCAGTCCCTTTCGACTTGCATTTTTGC
AGGAGCAGTATCATGAAGCCTAAACGCGATGGATATATGTTTTTGAAGGCAGAAAGCAAAATTATGTTTG
CCACTTTGCAAAGGAGCTCACTGTGGTGTCTGTGTTCCAACCACTGAATCTGGACCCCATCTGTGAATAA
GCCATTCTGACTCATATCCCCTATTTAACAGGGTCTCTAGTGCTGTGAAAAAAAAAATGCTGAACATTGC
ATATAACTTATATTGTAAGAAATACTGTACAATGACTTTATTGCATCTGGGTAGCTGTAAGGCATGAAGG
ATGCCAAGAAGTTTAAGGAATATGGGAGAAATAGTGTGGAAATTAAGAAGAAACTAGGTCTGATATTCAA
ATGGACAAACTGCCAGTTTTGTTTCCTTTCACTGGCCACAGTTGTTTGATGCATTAAAAGAAAATAAAAA
AAAGAAAAAAGAGAAAAGAAAAAAAAAGAAAAAAGTTGTAGGCGAATCATTTGTTCAAAGCTGTTGGCCT
CTGCAAAGGAAATACCAGTTCTGGGCAATCAGTGTTACCGTTCACCAGTTGCCGTTGAGGGTTTCAGAGA
GCCTTTTTCTAGGCCTACATGCTTTGTGAACAAGTCCCTGTAATTGTTGTTTGTATGTATAATTCAAAGC
ACCAAAATAAGAAAAGATGTAGATTTATTTCATCATATTATACAGACCGAACTGTTGTATAAATTTATTT
ACTGCTAGTCTTAAGAACTGCTTTCTTTCGTTTGTTTGTTTCAATATTTTCCTTCTCTCTCAATTTTTGG
TTGAATAAACTAGATTACATTCAGTTGGCCTAAGGTGGTTGTGCTCGGAGGGTTTCTTGTTTCTTTTCCA
TTTTGTTTTTGGATGATATTTATTAAATAGCTTCTAAGAGTCCGGCGGCATCTGTCTTGTCCCTATTCCT
GCAGCCTGTGCTGAGGGTAGCAGTGTATGAGCTACCAGCGTGCATGTCAGCGACCCTGGCCCGACAGGCC
ACGTCCTGCAATCGGCCCGGCTGCCTCTTCGCCCTGTCGTGTTCTGTGTTAGTGATCACTGCCTTTAATA
CAGTCTGTTGGAATAATATTATAAGCATAATAATAAAGTGAAAATATTTTAAAACTACAA
SEQ ID NO: 24
- Homo sapiens guanine nucleotide binding protein
(G protein), beta 5 (GNB5), transcript variant 1,
mRNA
CCGGGGACGGCTGCTGGAGCGGCGCCCGCCGCGGCTCAGCGCATTCCCGCTCTCCGCTTCCCTCTCCGCT
GCGTCCCCGCGCGAAGATGGCAACCGAGGGGCTGCACGAGAACGAGACGCTGGCGTCGCTGAAGAGCGAG
GCCGAGAGCCTCAAGGGCAAGCTGGAGGAGGAGCGAGCCAAGCTGCACGATGTGGAGCTGCACCAGGTGG
CGGAGCGGGTGGAGGCCCTGGGGCAGTTTGTCATGAAGACCAGAAGGACCCTCAAAGGCCACGGGAACAA
AGTCCTGTGCATGGACTGGTGCAAAGATAAGAGGAGGATCGTGAGCTCGTCACAGGATGGGAAGGTGATC
GTGTGGGATTCCTTCACCACAAACAAGGAGCACGCGGTCACCATGCCCTGCACGTGGGTGATGGCATGTG
CTTATGCCCCATCGGGATGTGCCATTGCTTGTGGTGGTTTGGATAATAAGTGTTCTGTGTACCCCTTGAC
GTTTGACAAAAATGAAAACATGGCTGCCAAAAAGAAGTCTGTTGCTATGCACACCAACTACCTGTCGGCC
TGCAGCTTCACCAACTCTGACATGCAGATCCTGACAGCGAGCGGCGATGGCACATGTGCCCTGTGGGACG
TGGAGAGCGGGCAGCTGCTGCAGAGCTTCCACGGACATGGGGCTGACGTCCTCTGCTTGGACCTGGCCCC
CTCAGAAACTGGAAACACCTTCGTGTCTGGGGGATGTGACAAGAAAGCCATGGTGTGGGACATGCGCTCC
GGCCAGTGCGTGCAGGCCTTTGAAACACATGAATCTGACATCAACAGTGTCCGGTACTACCCCAGTGGAG
ATGCCTTTGCTTCAGGGTCAGATGACGCTACGTGTCGCCTCTATGACCTGCGGGCAGATAGGGAGGTTGC
CATCTATTCCAAAGAAAGCATCATATTTGGAGCATCCAGCGTGGACTTCTCCCTCAGTGGTCGCCTGCTG
TTTGCTGGATACAATGATTACACTATCAACGTCTGGGATGTTCTCAAAGGGTCCCGGGTCTCCATCCTGT
TTGGACATGAAAACCGCGTTAGCACTCTACGAGTTTCCCCCGATGGGACTGCTTTCTGCTCTGGATCATG
GGATCATACCCTCAGAGTCTGGGCCTAATCATCTTCTGACAGTGCACTCATGTATACCTGAGAATTTGAA
ATCTTCACATGTAAATAGATATTACTTCTAGAGGAGCTTAGAGTTTATTGCAGTGTAGCTTAGGGGAGCA
ACCCATGGCTCACAGGTCACTAAGCGTCTCCAATATGACTATTAAAACTGTCACCTCTGGAAATACACTA
GTGTGAGCCTTCAGCACTGCGAGAATACCTTCAAGTACAGTATTTTTCTTTTGGAACACTTTTTAAAATG
TATCTGTTTTTAAGGTTATTCTAAATTATAGTAGCCTCAACTCATTCTGTCACCAGTAGAATTCAGCAGT
TAATATATTCCATATTATTTCTTTGAATCAATTCATTTTCAGAGCACTTTAAAGTCTGATATTTCTCGAT
GTGCACTGTGATGCCTGGAACCTTCCTCTGGAAGTGCTGATTTTATGGACTGAGGACTGGTGACTGGTCT
GTGATAGAAGCAAATTCCAATTCCAAATGTAATTAGACAAAAATCATTTTTTTAGAATGTGTTTTTATTG
TAAAAGTATCTTTTTCAGCTTCCTGTTCTATTGTCTTTTTTCAGATACAACATTTTTGTCTATGGTGAAC
TGCTGTAAATGACGCAGAGAAATGCCTAAAAAGGACAGGTGGTTTGACTCATGGATGATGATGATGTCAC
TGTGCCACTTGGACAGGGCGTTTTCTCTGAATTGAAGGGAAAGCCAATGGTGTTTGTAAACAAATGCTTC
TGAGAGCAAAGAAAAGTCTTCTGTGTGGGAACACAAGATAGTAAACTTATTTAAAAACCTATTAGTAGAA
TTAGTGGAAACACTTAGGTTAAAGTGAATCTTGTCCATATAAATTATATTCATGGCCGGGCGCGGTGGCT
CACGCTTGTAATCCCAGCACTTTGGGAGGCCGAGGCGGGCGGATCACGAGGTCAGGAGTTCGAGACCACG
GTGAAACCCTGTCTCTACTAAAAAATACAAAAAATTAGCCGGGCGTGGTGGCGGGCGCCTGTAGTCCCAG
CTACTCGGAGAGGCTGAGGCAGGAGAATGGCGTGAACCCGGGAGGTGGAGCTTGCAGTGAGCCGAGGTCG
AGCCACTGCAGCCTGGGTGACAAAGCGAGACTCCGTCTCAAAAAAAAAAAAAAATTATATTCATATGTAT
TGCATTGCAATTATAATTACATATGCAGATTGATTGATAGTCATGAATAATAACGTCTGCTCCTCTTACA
TAGAAAAACGATATTAAAAGAAGATCTTCTCTTTATTTGAGACTCAGAATTCCTTCTAGAAGAAGGAAGT
GCTTTTTGTTATAGGATCCCTTCTTTTCCTTTTTTTGTTTTTTTGTAAGATGTAGATGCTTATTCTTTGC
TTTAGAAAACTTCTCACTTAAAAAGATGGCATGCACCTAGGGGAATAAAAGGTCACCTCAGACACCAGGT
GTCATTCCTGGTGAGGCCTGCCTCGTCGGTGGCCTGGGGTCTGCCGGCAGGTTCTGGCTGCACCTGAAGG
CTGCGTGCACCTTGTCCCCTGGACAGGTCTCCTTTCCTGGCCCTGCTCCAGCCCAGCCCTTCTTCTAGTG
GTAGCTCTGGCTTTGCAGGCCCAGCTCCAGGCCCTGCTCCTCAGAGAGACTCTTCCAGAGCTGGAGCTGG
GCACAGCCATAAGACAGGACTGGACCAGATGCTCCTGTAAACATCCAGGGGTGTGCCAGGCCCACCCTCA
CAACTGCTTGTTCAGGTATCGTGATGGGCCACTCGGTCCAAAATCAGCCAGGCCATCTTTTCCATCATCT
CACTTCAAATAAACATAATAATTATATTTGATCATTTGC
SEQ ID NO: 25
- Homo sapiens glutathione S-transferase mu 4
(GSTM4), transcript variant 2, mRNA
AAGCTGGCGAGGCCGAGCCCCTCCTAGTGCTTCCGGACCTTGCTCCCTGAACACTCGGAGGTGGCGGTGG
ATCTTACTCCTTCCAGCCAGTGAGGATCCAGCAACCTGCTCCGTGCCTCCCGCGCCTGTTGGTTGGAAGT
GACGACCTTGAAGATCGGCCGGTTGGAAGTGACGACCTTGAAGATCGGCGGGCGCAGCGGGGCCGAGGGG
GCGGGTCTGGCGCTAGGTCCAGCCCCTGCGTGCCGGGAACCCCAGAGGAGGTCGCAGTTCAGCCCAGCTG
AGGCCTGTCTGCAGAATCGACACCAACCAGCATCATGTCCATGACACTGGGGTACTGGGACATCCGCGGG
CTGGCCCACGCCATCCGCCTGCTCCTGGAATACACAGACTCAAGCTACGAGGAAAAGAAGTATACGATGG
GGGACGCTCCTGACTATGACAGAAGCCAGTGGCTGAATGAAAAATTCAAGCTGGGCCTGGACTTTCCCAA
TCTGCCCTACTTGATTGATGGGGCTCACAAGATCACCCAGAGCAACGCCATCCTGTGCTACATTGCCCGC
AAGCACAACCTCTGTGGGGAGACAGAAGAGGAGAAGATTCGTCTGGACATTTTGGAGAACCAGGCTATGG
ACGTCTCCAATCAGCTGGCCAGAGTCTGCTACAGCCCTGACTTTGAGAAACTGAAGCCAGAATACTTGGA
GGAACTTCCTACAATGATGCAGCACTTCTCACAGTTCCTGGGGAAGAGGCCATGGTTTGTTGGAGACAAG
ATCACCTTTGTAGATTTCCTCGCCTATGATGTCCTTGACCTCCACCGTATATTTGAGCCCAACTGCTTGG
ACGCCTTTCCAAATCTGAAGGACTTCATCTCCCGCTTTGAGGTTTCCTGTGGCATAATGTGATGGTCAAT
TTTCTGCATCAACTTGACTGGGCTAAGGGATGCTCAGATGGCAGGTAAAATCATTGTGCTTGTGAGGGTG
TTTCCAGAAGAGATTTGCCTTTGAATCAGAAGACAGCAAAGATTTCCTTCAGCAATGAAGGAGGCATCCA
CCAAACTGTCAGGGCCCAGAGAGAAGAAAAAGACAGGAAGGGTGAATTTGACCTCTCTGACTGGGACATC
CATCTCTGCCTATCCTGGGACCTCCACACTCCTGGTTCTCTGGCCTTCAGACTTGATCAGGGACTAACAC
CATCGCCTCCCACCCCCACCTTTGTTCTGAGGCCTTTAGCCTCTGAATGATACCACTGGCTTTCCTGCTT
CTCTATCCTGCAGTCGGCAGATCATGGGACTTCTTCACTCCAAAATTGTGTGAGCCAATTCCCATAACAG
ATAGATAAATTTATAAATAAACACACAAATTTCCTACAGCCT
SEQ ID NO: 26
- Homo sapiens major histocompatibility complex,
class II, DR alpha (HLA-DRA), mRNA
TTTTAATGGTCAGACTCTATTACACCCCACATTCTCTTTTCTTTTATTCTTGTCTGTTCTGCCTCACTCC
CGAGCTCTACTGACTCCCAACAGAGCGCCCAAGAAGAAAATGGCCATAAGTGGAGTCCCTGTGCTAGGAT
TTTTCATCATAGCTGTGCTGATGAGCGCTCAGGAATCATGGGCTATCAAAGAAGAACATGTGATCATCCA
GGCCGAGTTCTATCTGAATCCTGACCAATCAGGCGAGTTTATGTTTGACTTTGATGGTGATGAGATTTTC
CATGTGGATATGGCAAAGAAGGAGACGGTCTGGCGGCTTGAAGAATTTGGACGATTTGCCAGCTTTGAGG
CTCAAGGTGCATTGGCCAACATAGCTGTGGACAAAGCCAACCTGGAAATCATGACAAAGCGCTCCAACTA
TACTCCGATCACCAATGTACCTCCAGAGGTAACTGTGCTCACAAACAGCCCTGTGGAACTGAGAGAGCCC
AACGTCCTCATCTGTTTCATAGACAAGTTCACCCCACCAGTGGTCAATGTCACGTGGCTTCGAAATGGAA
AACCTGTCACCACAGGAGTGTCAGAGACAGTCTTCCTGCCCAGGGAAGACCACCTTTTCCGCAAGTTCCA
CTATCTCCCCTTCCTGCCCTCAACTGAGGACGTTTACGACTGCAGGGTGGAGCACTGGGGCTTGGATGAG
CCTCTTCTCAAGCACTGGGAGTTTGATGCTCCAAGCCCTCTCCCAGAGACTACAGAGAACGTGGTGTGTG
CCCTGGGCCTGACTGTGGGTCTGGTGGGCATCATTATTGGGACCATCTTCATCATCAAGGGATTGCGCAA
AAGCAATGCAGCAGAACGCAGGGGGCCTCTGTAAGGCACATGGAGGTGATGGTGTTTCTTAGAGAGAAGA
TCACTGAAGAAACTTCTGCTTTAATGGCTTTACAAAGCTGGCAATATTACAATCCTTGACCTCAGTGAAA
GCAGTCATCTTCAGCATTTTCCAGCCCTATAGCCACCCCAAGTGTGGATATGCCTCTTCGATTGCTCCGT
ACTCTAACATCTAGCTGGCTTCCCTGTCTATTGCCTTTTCCTGTATCTATTTTCCTCTATTTCCTATCAT
TTTATTATCACCATGCAATGCCTCTGGAATAAAACATACAGGAGTCTGTCTCTGCTATGGAATGCCCCAT
GGGGCATCTCTTGTGTACTTATTGTTTAAGGTTTCCTCAAACTGTGATTTTTCTGAACACAATAAACTAT
TTTGATGATCTTGGGTGGAAAAAAAAAAAAAAAAAAAAAAAAAAAAAAAAAA
SEQ ID NO: 27
- Homo sapiens v-Ha-ras Harvey rat sarcoma viral
oncogene homolog (HRAS), transcript variant 3, mRNA
TGCCCTGCGCCCGCAACCCGAGCCGCACCCGCCGCGGACGGAGCCCATGCGCGGGGCGAACCGCGCGCCC
CCGCCCCCGCCCCGCCCCGGCCTCGGCCCCGGCCCTGGCCCCGGGGGCAGTCGCGCCTGTGAACGGTGGG
GCAGGAGACCCTGTAGGAGGACCCCGGGCCGCAGGCCCCTGAGGAGCGATGACGGAATATAAGCTGGTGG
TGGTGGGCGCCGGCGGTGTGGGCAAGAGTGCGCTGACCATCCAGCTGATCCAGAACCATTTTGTGGACGA
ATACGACCCCACTATAGAGGATTCCTACCGGAAGCAGGTGGTCATTGATGGGGAGACGTGCCTGTTGGAC
ATCCTGGATACCGCCGGCCAGGAGGAGTACAGCGCCATGCGGGACCAGTACATGCGCACCGGGGAGGGCT
TCCTGTGTGTGTTTGCCATCAACAACACCAAGTCTTTTGAGGACATCCACCAGTACAGGGAGCAGATCAA
ACGGGTGAAGGACTCGGATGACGTGCCCATGGTGCTGGTGGGGAACAAGTGTGACCTGGCTGCACGCACT
GTGGAATCTCGGCAGGCTCAGGACCTCGCCCGAAGCTACGGCATCCCCTACATCGAGACCTCGGCCAAGA
CCCGGCAGGGAGTGGAGGATGCCTTCTACACGTTGGTGCGTGAGATCCGGCAGCACAAGCTGCGGAAGCT
GAACCCTCCTGATGAGAGTGGCCCCGGCTGCATGAGCTGCAAGTGTGTGCTCTCCTGACGCAGGTGAGGG
GGACTCCCAGGGCGGCCGCCACGCCCACCGGATGACCCCGGCTCCCCGCCCCTGCCGGTCTCCTGGCCTG
CGGTCAGCAGCCTCCCTTGTGCCCCGCCCAGCACAAGCTCAGGACATGGAGGTGCCGGATGCAGGAAGGA
GGTGCAGACGGAAGGAGGAGGAAGGAAGGACGGAAGCAAGGAAGGAAGGAAGGGCTGCTGGAGCCCAGTC
ACCCCGGGACCGTGGGCCGAGGTGACTGCAGACCCTCCCAGGGAGGCTGTGCACAGACTGTCTTGAACAT
CCCAAATGCCACCGGAACCCCAGCCCTTAGCTCCCCTCCCAGGCCTCTGTGGGCCCTTGTCGGGCACAGA
TGGGATCACAGTAAATTATTGGATGGTCTTGAAAAAAAAAAAAAAAAAA
SEQ ID NO: 28
- Homo sapiens interferon, alpha-inducible protein
27 (IFI27), transcript variant 1, mRNA
GGGAACACATCCAAGCTTAAGACGGTGAGGTCAGCTTCACATTCTCAGGAACTCTCCTTCTTTGGGTCTG
GCTGAAGTTGAGGATCTCTTACTCTCTAGGCCACGGAATTAACCCGAGCAGGCATGGAGGCCTCTGCTCT
CACCTCATCAGCAGTGACCAGTGTGGCCAAAGTGGTCAGGGTGGCCTCTGGCTCTGCCGTAGTTTTGCCC
CTGGCCAGGATTGCTACAGTTGTGATTGGAGGAGTTGTGGCCATGGCGGCTGTGCCCATGGTGCTCAGTG
CCATGGGCTTCACTGCGGCGGGAATCGCCTCGTCCTCCATAGCAGCCAAGATGATGTCCGCGGCGGCCAT
TGCCAATGGGGGTGGAGTTGCCTCGGGCAGCCTTGTGGCTACTCTGCAGTCACTGGGAGCAACTGGACTC
TCCGGATTGACCAAGTTCATCCTGGGCTCCATTGGGTCTGCCATTGCGGCTGTCATTGCGAGGTTCTACT
AGCTCCCTGCCCCTCGCCCTGCAGAGAAGAGAACCATGCCAGGGGAGAAGGCACCCAGCCATCCTGACCC
AGCGAGGAGCCAACTATCCCAAATATACCTGGGGTGAAATATACCAAATTCTGCATCTCCAGAGGAAAAT
AAGAAATAAAGATGAATTGTTGCAACTCTTCAAAA
SEQ ID NO: 29
- Homo sapiens interleukin 11 receptor, alpha
(IL11RA), transcript variant 3, mRNA
AGAGGGCGAGGGCGAGGGCAGAGGGCGCTGGCGGCAGCGGCCGCGGAAGATGAGCAGCAGCTGCTCAGGG
CTGAGCAGGGTCCTGGTGGCCGTGGCTACAGCCCTGGTGTCTGCCTCCTCCCCCTGCCCCCAGGCCTGGG
GCCCCCCAGGGGTCCAGTATGGGCAGCCAGGCAGGTCCGTGAAGCTGTGTTGTCCTGGAGTGACTGCCGG
GGACCCAGTGTCCTGGTTTCGGGATGGGGAGCCAAAGCTGCTCCAGGGACCTGACTCTGGGCTAGGGCAT
GAACTGGTCCTGGCCCAGGCAGACAGCACTGATGAGGGCACCTACATCTGCCAGACCCTGGATGGTGCAC
TTGGGGGCACAGTGACCCTGCAGCTGGGCTACCCTCCAGCCCGCCCTGTTGTCTCCTGCCAAGCAGCCGA
CTATGAGAACTTCTCTTGCACTTGGAGTCCCAGCCAGATCAGCGGTTTACCCACCCGCTACCTCACCTCC
TACAGGAAGAAGACAGTCCTAGGAGCTGATAGCCAGAGGAGGAGTCCATCCACAGGGCCCTGGCCATGCC
CACAGGATCCCCTAGGGGCTGCCCGCTGTGTTGTCCACGGGGCTGAGTTCTGGAGCCAGTACCGGATTAA
TGTGACTGAGGTGAACCCACTGGGTGCCAGCACACGCCTGCTGGATGTGAGCTTGCAGAGCATCTTGCGC
CCTGACCCACCCCAGGGCCTGCGGGTAGAGTCAGTACCAGGTTACCCCCGACGCCTGCGAGCCAGCTGGA
CATACCCTGCCTCCTGGCCGTGCCAGCCCCACTTCCTGCTCAAGTTCCGTTTGCAGTACCGTCCGGCGCA
GCATCCAGCCTGGTCCACGGTGGAGCCAGCTGGACTGGAGGAGGTGATCACAGATGCTGTGGCTGGGCTG
CCCCATGCTGTACGAGTCAGTGCCCGGGACTTTCTAGATGCTGGCACCTGGAGCACCTGGAGCCCGGAGG
CCTGGGGAACTCCGAGCACTGGGACCATACCAAAGGAGATACCAGCATGGGGCCAGCTACACACGCAGCC
AGAGGTGGAGCCTCAGGTGGACAGCCCTCCTCCTCCAAGGCCCTCCCTCCAACCACACCCTCCCCTACTT
GATCACAGGGACTCTGTGGAGCAGGTAGCTGTGCTGGCGTCTTTGGGAATCCTTTCTTTCCTGGGACTGG
TGGCTGGGGCCCTGGCACTGGGGCTCTGGCTGAGGCTGAGACGGGGTGGGAAGGATGGATCCCCAAAGCC
TGGGTTCTTGGCCTCAGTGATTCCAGTGGACAGGCGTCCAGGAGCTCCAAACCTGTAGAGGACCCAGGAG
GGCTTCGGCAGATTCCACCTATAATTCTGTCTTGCTGGTGTGGATAGAAACCAGGCAGGACAGTAGATCC
CTATGGTTGGATCTCAGCTGGAAGTTCTGTTTGGAGCCCATTTCTGTGAGACCCTGTATTTCAAATTTGC
AGCTGAAAGGTGCTTGTACCTCTGATTTCACCCCAGAGTTGGAGTTCTGCTCAAGGAACGTGTGTAATGT
GTACATCTGTGTCCATGTGTGACCATGTGTCTGTGAGGCAGGGAACATGTATTCTCTGCATGCATGTATG
TAGGTGCCTGGGGAGTGTGTGTGGGTCCTTGGCTCTTGGCCTTTCCCCTTGCAGGGGTTGTGCAGGTGTG
AATAAAGAGAATAAGGAAGTTCTTGGAAAAAAAAAAAAAAAAAAAAAAAAAAAAAAAAAAAAAAAAAAAA
AAAAAAAAAA
SEQ ID NO: 30
- Homo sapiens jun proto-oncogene (JUN), mRNA
GACATCATGGGCTATTTTTAGGGGTTGACTGGTAGCAGATAAGTGTTGAGCTCGGGCTGGATAAGGGCTC
AGAGTTGCACTGAGTGTGGCTGAAGCAGCGAGGCGGGAGTGGAGGTGCGCGGAGTCAGGCAGACAGACAG
ACACAGCCAGCCAGCCAGGTCGGCAGTATAGTCCGAACTGCAAATCTTATTTTCTTTTCACCTTCTCTCT
AACTGCCCAGAGCTAGCGCCTGTGGCTCCCGGGCTGGTGTTTCGGGAGTGTCCAGAGAGCCTGGTCTCCA
GCCGCCCCCGGGAGGAGAGCCCTGCTGCCCAGGCGCTGTTGACAGCGGCGGAAAGCAGCGGTACCCACGC
GCCCGCCGGGGGAAGTCGGCGAGCGGCTGCAGCAGCAAAGAACTTTCCCGGCTGGGAGGACCGGAGACAA
GTGGCAGAGTCCCGGAGCGAACTTTTGCAAGCCTTTCCGTCGTCTTAGGCTTCTCCACGGCGGTAAAGAC
CAGAAGGCGGCGGAGAGCCACGCAAGAGAAGAAGGACGTGCGCTCAGCTTCGCTCGCACCGGTTGTTGAA
CTTGGGCGAGCGCGAGCCGCGGCTGCCGGGCGCCCCCTCCCCCTAGCAGCGGAGGAGGGGACAAGTCGTC
GGAGTCCGGGCGGCCAAGACCCGCCGCCGGCCGGCCACTGCAGGGTCCGCACTGATCCGCTCCGCGGGGA
GAGCCGCTGCTCTGGGAAGTGAGTTCGCCTGCGGACTCCGAGGAACCGCTGCGCCCGAAGAGCGCTCAGT
GAGTGACCGCGACTTTTCAAAGCCGGGTAGCGCGCGCGAGTCGACAAGTAAGAGTGCGGGAGGCATCTTA
ATTAACCCTGCGCTCCCTGGAGCGAGCTGGTGAGGAGGGCGCAGCGGGGACGACAGCCAGCGGGTGCGTG
CGCTCTTAGAGAAACTTTCCCTGTCAAAGGCTCCGGGGGGCGCGGGTGTCCCCCGCTTGCCAGAGCCCTG
TTGCGGCCCCGAAACTTGTGCGCGCAGCCCAAACTAACCTCACGTGAAGTGACGGACTGTTCTATGACTG
CAAAGATGGAAACGACCTTCTATGACGATGCCCTCAACGCCTCGTTCCTCCCGTCCGAGAGCGGACCTTA
TGGCTACAGTAACCCCAAGATCCTGAAACAGAGCATGACCCTGAACCTGGCCGACCCAGTGGGGAGCCTG
AAGCCGCACCTCCGCGCCAAGAACTCGGACCTCCTCACCTCGCCCGACGTGGGGCTGCTCAAGCTGGCGT
CGCCCGAGCTGGAGCGCCTGATAATCCAGTCCAGCAACGGGCACATCACCACCACGCCGACCCCCACCCA
GTTCCTGTGCCCCAAGAACGTGACAGATGAGCAGGAGGGCTTCGCCGAGGGCTTCGTGCGCGCCCTGGCC
GAACTGCACAGCCAGAACACGCTGCCCAGCGTCACGTCGGCGGCGCAGCCGGTCAACGGGGCAGGCATGG
TGGCTCCCGCGGTAGCCTCGGTGGCAGGGGGCAGCGGCAGCGGCGGCTTCAGCGCCAGCCTGCACAGCGA
GCCGCCGGTCTACGCAAACCTCAGCAACTTCAACCCAGGCGCGCTGAGCAGCGGCGGCGGGGCGCCCTCC
TACGGCGCGGCCGGCCTGGCCTTTCCCGCGCAACCCCAGCAGCAGCAGCAGCCGCCGCACCACCTGCCCC
AGCAGATGCCCGTGCAGCACCCGCGGCTGCAGGCCCTGAAGGAGGAGCCTCAGACAGTGCCCGAGATGCC
CGGCGAGACACCGCCCCTGTCCCCCATCGACATGGAGTCCCAGGAGCGGATCAAGGCGGAGAGGAAGCGC
ATGAGGAACCGCATCGCTGCCTCCAAGTGCCGAAAAAGGAAGCTGGAGAGAATCGCCCGGCTGGAGGAAA
AAGTGAAAACCTTGAAAGCTCAGAACTCGGAGCTGGCGTCCACGGCCAACATGCTCAGGGAACAGGTGGC
ACAGCTTAAACAGAAAGTCATGAACCACGTTAACAGTGGGTGCCAACTCATGCTAACGCAGCAGTTGCAA
ACATTTTGAAGAGAGACCGTCGGGGGCTGAGGGGCAACGAAGAAAAAAAATAACACAGAGAGACAGACTT
GAGAACTTGACAAGTTGCGACGGAGAGAAAAAAGAAGTGTCCGAGAACTAAAGCCAAGGGTATCCAAGTT
GGACTGGGTTGCGTCCTGACGGCGCCCCCAGTGTGCACGAGTGGGAAGGACTTGGCGCGCCCTCCCTTGG
CGTGGAGCCAGGGAGCGGCCGCCTGCGGGCTGCCCCGCTTTGCGGACGGGCTGTCCCCGCGCGAACGGAA
CGTTGGACTTTTCGTTAACATTGACCAAGAACTGCATGGACCTAACATTCGATCTCATTCAGTATTAAAG
GGGGGAGGGGGAGGGGGTTACAAACTGCAATAGAGACTGTAGATTGCTTCTGTAGTACTCCTTAAGAACA
CAAAGCGGGGGGAGGGTTGGGGAGGGGCGGCAGGAGGGAGGTTTGTGAGAGCGAGGCTGAGCCTACAGAT
GAACTCTTTCTGGCCTGCCTTCGTTAACTGTGTATGTACATATATATATTTTTTAATTTGATGAAAGCTG
ATTACTGTCAATAAACAGCTTCATGCCTTTGTAAGTTATTTCTTGTTTGTTTGTTTGGGTATCCTGCCCA
GTGTTGTTTGTAAATAAGAGATTTGGAGCACTCTGAGTTTACCATTTGTAATAAAGTATATAATTTTTTT
ATGTTTTGTTTCTGAAAATTCCAGAAAGGATATTTAAGAAAATACAATAAACTATTGGAAAGTACTCCCC
TAACCTCTTTTCTGCATCATCTGTAGATACTAGCTATCTAGGTGGAGTTGAAAGAGTTAAGAATGTCGAT
TAAAATCACTCTCAGTGCTTCTTACTATTAAGCAGTAAAAACTGTTCTCTATTAGACTTTAGAAATAAAT
GTACCTGATGTACCTGATGCTATGGTCAGGTTATACTCCTCCTCCCCCAGCTATCTATATGGAATTGCTT
ACCAAAGGATAGTGCGATGTTTCAGGAGGCTGGAGGAAGGGGGGTTGCAGTGGAGAGGGACAGCCCACTG
AGAAGTCAAACATTTCAAAGTTTGGATTGTATCAAGTGGCATGTGCTGTGACCATTTATAATGTTAGTAG
AAATTTTACAATAGGTGCTTATTCTCAAAGCAGGAATTGGTGGCAGATTTTACAAAAGATGTATCCTTCC
AATTTGGAATCTTCTCTTTGACAATTCCTAGATAAAAAGATGGCCTTTGCTTATGAATATTTATAACAGC
ATTCTTGTCACAATAAATGTATTCAAATACCAAAAAAAAAAAAAAAAA
SEQ ID NO: 31
- Homo sapiens v-Ki-ras2 Kirsten rat sarcoma viral
oncogene homolog (KRAS), transcript variant b, mRNA
GGCCGCGGCGGCGGAGGCAGCAGCGGCGGCGGCAGTGGCGGCGGCGAAGGTGGCGGCGGCTCGGCCAGTA
CTCCCGGCCCCCGCCATTTCGGACTGGGAGCGAGCGCGGCGCAGGCACTGAAGGCGGCGGCGGGGCCAGA
GGCTCAGCGGCTCCCAGGTGCGGGAGAGAGGCCTGCTGAAAATGACTGAATATAAACTTGTGGTAGTTGG
AGCTGGTGGCGTAGGCAAGAGTGCCTTGACGATACAGCTAATTCAGAATCATTTTGTGGACGAATATGAT
CCAACAATAGAGGATTCCTACAGGAAGCAAGTAGTAATTGATGGAGAAACCTGTCTCTTGGATATTCTCG
ACACAGCAGGTCAAGAGGAGTACAGTGCAATGAGGGACCAGTACATGAGGACTGGGGAGGGCTTTCTTTG
TGTATTTGCCATAAATAATACTAAATCATTTGAAGATATTCACCATTATAGAGAACAAATTAAAAGAGTT
AAGGACTCTGAAGATGTACCTATGGTCCTAGTAGGAAATAAATGTGATTTGCCTTCTAGAACAGTAGACA
CAAAACAGGCTCAGGACTTAGCAAGAAGTTATGGAATTCCTTTTATTGAAACATCAGCAAAGACAAGACA
GGGTGTTGATGATGCCTTCTATACATTAGTTCGAGAAATTCGAAAACATAAAGAAAAGATGAGCAAAGAT
GGTAAAAAGAAGAAAAAGAAGTCAAAGACAAAGTGTGTAATTATGTAAATACAATTTGTACTTTTTTCTT
AAGGCATACTAGTACAAGTGGTAATTTTTGTACATTACACTAAATTATTAGCATTTGTTTTAGCATTACC
TAATTTTTTTCCTGCTCCATGCAGACTGTTAGCTTTTACCTTAAATGCTTATTTTAAAATGACAGTGGAA
GTTTTTTTTTCCTCTAAGTGCCAGTATTCCCAGAGTTTTGGTTTTTGAACTAGCAATGCCTGTGAAAAAG
AAACTGAATACCTAAGATTTCTGTCTTGGGGTTTTTGGTGCATGCAGTTGATTACTTCTTATTTTTCTTA
CCAATTGTGAATGTTGGTGTGAAACAAATTAATGAAGCTTTTGAATCATCCCTATTCTGTGTTTTATCTA
GTCACATAAATGGATTAATTACTAATTTCAGTTGAGACCTTCTAATTGGTTTTTACTGAAACATTGAGGG
AACACAAATTTATGGGCTTCCTGATGATGATTCTTCTAGGCATCATGTCCTATAGTTTGTCATCCCTGAT
GAATGTAAAGTTACACTGTTCACAAAGGTTTTGTCTCCTTTCCACTGCTATTAGTCATGGTCACTCTCCC
CAAAATATTATATTTTTTCTATAAAAAGAAAAAAATGGAAAAAAATTACAAGGCAATGGAAACTATTATA
AGGCCATTTCCTTTTCACATTAGATAAATTACTATAAAGACTCCTAATAGCTTTTCCTGTTAAGGCAGAC
CCAGTATGAAATGGGGATTATTATAGCAACCATTTTGGGGCTATATTTACATGCTACTAAATTTTTATAA
TAATTGAAAAGATTTTAACAAGTATAAAAAATTCTCATAGGAATTAAATGTAGTCTCCCTGTGTCAGACT
GCTCTTTCATAGTATAACTTTAAATCTTTTCTTCAACTTGAGTCTTTGAAGATAGTTTTAATTCTGCTTG
TGACATTAAAAGATTATTTGGGCCAGTTATAGCTTATTAGGTGTTGAAGAGACCAAGGTTGCAAGGCCAG
GCCCTGTGTGAACCTTTGAGCTTTCATAGAGAGTTTCACAGCATGGACTGTGTCCCCACGGTCATCCAGT
GTTGTCATGCATTGGTTAGTCAAAATGGGGAGGGACTAGGGCAGTTTGGATAGCTCAACAAGATACAATC
TCACTCTGTGGTGGTCCTGCTGACAAATCAAGAGCATTGCTTTTGTTTCTTAAGAAAACAAACTCTTTTT
TAAAAATTACTTTTAAATATTAACTCAAAAGTTGAGATTTTGGGGTGGTGGTGTGCCAAGACATTAATTT
TTTTTTTAAACAATGAAGTGAAAAAGTTTTACAATCTCTAGGTTTGGCTAGTTCTCTTAACACTGGTTAA
ATTAACATTGCATAAACACTTTTCAAGTCTGATCCATATTTAATAATGCTTTAAAATAAAAATAAAAACA
ATCCTTTTGATAAATTTAAAATGTTACTTATTTTAAAATAAATGAAGTGAGATGGCATGGTGAGGTGAAA
GTATCACTGGACTAGGAAGAAGGTGACTTAGGTTCTAGATAGGTGTCTTTTAGGACTCTGATTTTGAGGA
CATCACTTACTATCCATTTCTTCATGTTAAAAGAAGTCATCTCAAACTCTTAGTTTTTTTTTTTTACAAC
TATGTAATTTATATTCCATTTACATAAGGATACACTTATTTGTCAAGCTCAGCACAATCTGTAAATTTTT
AACCTATGTTACACCATCTTCAGTGCCAGTCTTGGGCAAAATTGTGCAAGAGGTGAAGTTTATATTTGAA
TATCCATTCTCGTTTTAGGACTCTTCTTCCATATTAGTGTCATCTTGCCTCCCTACCTTCCACATGCCCC
ATGACTTGATGCAGTTTTAATACTTGTAATTCCCCTAACCATAAGATTTACTGCTGCTGTGGATATCTCC
ATGAAGTTTTCCCACTGAGTCACATCAGAAATGCCCTACATCTTATTTCCTCAGGGCTCAAGAGAATCTG
ACAGATACCATAAAGGGATTTGACCTAATCACTAATTTTCAGGTGGTGGCTGATGCTTTGAACATCTCTT
TGCTGCCCAATCCATTAGCGACAGTAGGATTTTTCAAACCTGGTATGAATAGACAGAACCCTATCCAGTG
GAAGGAGAATTTAATAAAGATAGTGCTGAAAGAATTCCTTAGGTAATCTATAACTAGGACTACTCCTGGT
AACAGTAATACATTCCATTGTTTTAGTAACCAGAAATCTTCATGCAATGAAAAATACTTTAATTCATGAA
GCTTACTTTTTTTTTTTGGTGTCAGAGTCTCGCTCTTGTCACCCAGGCTGGAATGCAGTGGCGCCATCTC
AGCTCACTGCAACCTCCATCTCCCAGGTTCAAGCGATTCTCGTGCCTCGGCCTCCTGAGTAGCTGGGATT
ACAGGCGTGTGCCACTACACTCAACTAATTTTTGTATTTTTAGGAGAGACGGGGTTTCACCCTGTTGGCC
AGGCTGGTCTCGAACTCCTGACCTCAAGTGATTCACCCACCTTGGCCTCATAAACCTGTTTTGCAGAACT
CATTTATTCAGCAAATATTTATTGAGTGCCTACCAGATGCCAGTCACCGCACAAGGCACTGGGTATATGG
TATCCCCAAACAAGAGACATAATCCCGGTCCTTAGGTAGTGCTAGTGTGGTCTGTAATATCTTACTAAGG
CCTTTGGTATACGACCCAGAGATAACACGATGCGTATTTTAGTTTTGCAAAGAAGGGGTTTGGTCTCTGT
GCCAGCTCTATAATTGTTTTGCTACGATTCCACTGAAACTCTTCGATCAAGCTACTTTATGTAAATCACT
TCATTGTTTTAAAGGAATAAACTTGATTATATTGTTTTTTTATTTGGCATAACTGTGATTCTTTTAGGAC
AATTACTGTACACATTAAGGTGTATGTCAGATATTCATATTGACCCAAATGTGTAATATTCCAGTTTTCT
CTGCATAAGTAATTAAAATATACTTAAAAATTAATAGTTTTATCTGGGTACAAATAAACAGGTGCCTGAA
CTAGTTCACAGACAAGGAAACTTCTATGTAAAAATCACTATGATTTCTGAATTGCTATGTGAAACTACAG
ATCTTTGGAACACTGTTTAGGTAGGGTGTTAAGACTTACACAGTACCTCGTTTCTACACAGAGAAAGAAA
TGGCCATACTTCAGGAACTGCAGTGCTTATGAGGGGATATTTAGGCCTCTTGAATTTTTGATGTAGATGG
GCATTTTTTTAAGGTAGTGGTTAATTACCTTTATGTGAACTTTGAATGGTTTAACAAAAGATTTGTTTTT
GTAGAGATTTTAAAGGGGGAGAATTCTAGAAATAAATGTTACCTAATTATTACAGCCTTAAAGACAAAAA
TCCTTGTTGAAGTTTTTTTAAAAAAAGCTAAATTACATAGACTTAGGCATTAACATGTTTGTGGAAGAAT
ATAGCAGACGTATATTGTATCATTTGAGTGAATGTTCCCAAGTAGGCATTCTAGGCTCTATTTAACTGAG
TCACACTGCATAGGAATTTAGAACCTAACTTTTATAGGTTATCAAAACTGTTGTCACCATTGCACAATTT
TGTCCTAATATATACATAGAAACTTTGTGGGGCATGTTAAGTTACAGTTTGCACAAGTTCATCTCATTTG
TATTCCATTGATTTTTTTTTTCTTCTAAACATTTTTTCTTCAAACAGTATATAACTTTTTTTAGGGGATT
TTTTTTTAGACAGCAAAAACTATCTGAAGATTTCCATTTGTCAAAAAGTAATGATTTCTTGATAATTGTG
TAGTAATGTTTTTTAGAACCCAGCAGTTACCTTAAAGCTGAATTTATATTTAGTAACTTCTGTGTTAATA
CTGGATAGCATGAATTCTGCATTGAGAAACTGAATAGCTGTCATAAAATGAAACTTTCTTTCTAAAGAAA
GATACTCACATGAGTTCTTGAAGAATAGTCATAACTAGATTAAGATCTGTGTTTTAGTTTAATAGTTTGA
AGTGCCTGTTTGGGATAATGATAGGTAATTTAGATGAATTTAGGGGAAAAAAAAGTTATCTGCAGATATG
TTGAGGGCCCATCTCTCCCCCCACACCCCCACAGAGCTAACTGGGTTACAGTGTTTTATCCGAAAGTTTC
CAATTCCACTGTCTTGTGTTTTCATGTTGAAAATACTTTTGCATTTTTCCTTTGAGTGCCAATTTCTTAC
TAGTACTATTTCTTAATGTAACATGTTTACCTGGAATGTATTTTAACTATTTTTGTATAGTGTAAACTGA
AACATGCACATTTTGTACATTGTGCTTTCTTTTGTGGGACATATGCAGTGTGATCCAGTTGTTTTCCATC
ATTTGGTTGCGCTGACCTAGGAATGTTGGTCATATCAAACATTAAAAATGACCACTCTTTTAATTGAAAT
TAACTTTTAAATGTTTATAGGAGTATGTGCTGTGAAGTGATCTAAAATTTGTAATATTTTTGTCATGAAC
TGTACTACTCCTAATTATTGTAATGTAATAAAAATAGTTACAGTGACAAAAAAAAAAAAAAA
SEQ ID NO: 32
- Homo sapiens leprecan-like 4 (LEPREL4), mRNA
GCTTCCTGGGCTTCCCATCTCTGGCGGGAAGCGCTCCCCGACGCATTCTCTACCTAGGGGACACCCCCAA
GGCAGGAGCCCGGGCCGACGGAGAGGACTTAACGACACTATCGGACCCTCTGGGAAAAGAGGGGAGACGT
CGTGACCCAGGCCCCGCCCCACCTTGCCGCCTCGTGCCCGGCGCTAAGACCCAGCGGGCGCGCCGCCCGC
CCGGGGCCCGGCCCTGTCCCCTTCCGTCCGCGGGGCAGCCAGCTCAGCTCCGGAGAGCCGGCGGCGCGGC
GGGCATGGCTCGGGTGGCGTGGGGGCTGCTGTGGTTGCTGCTGGGCAGCGCCGGGGCGCAGTACGAGAAG
TACAGCTTCCGGGGCTTCCCGCCCGAGGACCTGATGCCGCTGGCCGCGGCGTACGGGCACGCTCTGGAGC
AGTACGAGGGAGAGAGCTGGCGCGAGAGCGCGCGCTACCTGGAGGCGGCGCTGCGGCTGCACCGGCTCCT
GCGCGACAGCGAGGCCTTCTGCCACGCCAACTGCAGCGGCCCCGCGCCCGCGGCCAAGCCCGATCCCGAC
GGCGGCCGCGCAGACGAGTGGGCCTGCGAGCTGCGGCTCTTCGGCCGCGTCCTGGAGCGAGCCGCCTGCC
TGCGGCGCTGCAAGCGGACGCTGCCCGCCTTCCAGGTGCCCTACCCGCCGCGGCAGCTGCTGCGTGACTT
CCAGAGCCGCCTGCCCTACCAGTACCTGCACTACGCGCTGTTCAAGGCTAACCGGCTGGAGAAGGCGGTG
GCGGCGGCCTACACCTTCCTCCAGAGGAACCCGAAGCACGAGCTGACCGCCAAGTATCTCAACTACTATC
AGGGGATGCTGGACGTCGCCGACGAGTCCCTCACGGACCTAGAGGCCCAGCCCTACGAGGCCGTGTTCCT
CCGGGCTGTGAAGCTCTACAACAGCGGGGATTTCCGCAGCAGCACGGAGGACATGGAGCGGGCCTTGTCA
GAGTACCTGGCAGTCTTTGCCCGGTGCCTGGCCGGCTGTGAAGGGGCCCATGAGCAGGTGGACTTCAAGG
ACTTCTACCCGGCCATAGCAGATCTCTTTGCAGAGTCCCTGCAGTGCAAGGTGGACTGTGAGGCCAATTT
GACCCCCAATGTGGGTGGCTACTTCGTGGACAAGTTCGTGGCCACCATGTACCACTACCTGCAGTTTGCC
TACTATAAGTTGAATGATGTGCGCCAGGCTGCCCGCAGCGCCGCCAGCTACATGCTCTTCGACCCCAAGG
ACAGCGTCATGCAGCAGAACCTGGTGTATTACCGGTTCCACCGGGCTCGCTGGGGCCTGGAAGAGGAGGA
CTTCCAGCCCCGGGAGGAGGCCATGCTCTACCACAACCAGACCGCCGAGCTGCGGGAGCTGCTGGAGTTC
ACCCACATGTACCTGCAGTCAGATGATGAGATGGAGCTGGAGGAGACAGAACCGCCCCTGGAGCCTGAGG
ATGCCCTATCTGACGCCGAGTTTGAGGGGGAGGGTGACTACGAGGAGGGCATGTATGCTGACTGGTGGCA
GGAGCCGGATGCCAAGGGTGACGAGGCCGAGGCTGAGCCAGAGCCTGAACTCGCATGAGAAGGGGACACC
CCACACCGCTCAAGCTTGGGAAGCCTGGTGCCGATGGCCCCACCCTCACCAGCCTGGGCAGCAGCAAGAA
CTATTTATTAAAAACTTAAGATGGGCCAGGTGCGGTGGCTCACACCTGTAATCCCAGCATTTTGGGAGGC
CAAGGTGGGTGGATCACTTGAGGCCAGGAGTTCAAGACCAGCCTGGCCAACATGATGAGACCTCCGTCTC
TACTAAAATACATAAATTAGCCGGGTGTGGTGGCAGGCGCCTGAAATCCCAGCTACTCAAGAGGCTGAGG
CAGGAGAATCGCTTGAACCTGGGAGGCAAAGGTTGCAGTGAACTGAGATTGCGCCACCGCACTCCAGCCT
GGGCGACAGAGCGAGACTCCATCTTTAAAAAAAAACAAGACGGGCCGGCACGGTGGCTCACGCCTGTAAT
CCCAGCACTGAGAGGCCGATCACTTGAGGTCAGGAGTTCAAGACCAGCCTGGCCAACATGGTGAAACCCC
ATCTCTACTAAAAAATACAAAAATTAGCCAGGCATGGTGGCACACACCTGTAATCGTAGCTGAGGCAGGA
GAATCGCCTGAACCCAGGAGGCGGAGCTTGCAGTGAGCCGAGATCGTGCCACTGCACTCCAGCCTGGGCG
ACAGAGTGAGACTCCATCTCAAAAAAAAAAAAAAAAACTTAAGATGGACACAGCTGACTGGACCCCCATC
CTGCCTCACCCATGGGTGCTGCACCCCAGACCCATCCTGCCACTTCTATGTCTCTGGACCACAGGATGGT
GGTGGCATTGCAGGTTGGCAAGTGGGCTGATGGGGTCCGCCCTCCTCACTGCTGAGCTCCTCACCTGGAC
AGTCTCCTGGACAAGGAGTTTCCAGCTGCTGGCTGGAGTCTCAGGCCAAATTGCAGAGGGTCCTCCAGGG
TCCTGAAGAGCACTGGACTAAGAGTCTAGTGGTTCCAGGGCCCTGACCAGTAGGTGCTCAATAAATGTTT
GTTGTTGAATGAAAAAAAAAAAAAAAAAA
SEQ ID NO: 33
- Homo sapiens lethal giant larvae homolog 2
(Drosophila) (LLGL2), transcript variant 2, mRNA
GGAGGTGAGCAGGAAGGAGACGGCCGCCCAGCAGCCCGTGGGCAGGCGCGGCGGAGCGAGCGGGGCCGGC
GGCGGGCGCCGAGGGACGCCGAGGCCTCGGGCGGGGGCTGGCCCGGGGTTCCAGGTCTCCAGTGGGGGCT
GCAGACTAAGCAAAATGAGGCGGTTCCTGAGGCCAGGGCATGACCCTGTGCGGGAGAGGCTCAAGCGGGA
CCTGTTCCAGTTTAACAAGACGGTGGAGCATGGCTTCCCGCACCAGCCCAGCGCCCTCGGCTACAGCCCG
TCCCTGCGCATCCTGGCCATCGGCACCCGTTCTGGAGCCATCAAGCTCTACGGAGCCCCAGGCGTGGAGT
TCATGGGGCTGCACCAGGAGAACAACGCTGTGACGCAGATCCACCTCCTGCCCGGCCAGTGCCAGCTGGT
CACCCTGCTGGATGACAACAGCCTGCACCTTTGGAGCCTGAAGGTCAAGGGCGGGGCATCGGAGCTGCAG
GAGGATGAGAGCTTCACACTGCGTGGACCCCCAGGGGCTGCCCCCAGTGCCACACAGATCACCGTGGTCC
TGCCACATTCCTCCTGCGAGCTGCTCTACCTGGGCACCGAGAGTGGCAACGTGTTTGTGGTGCAGCTGCC
AGCTTTTCGTGCGCTGGAGGACCGGACCATCAGCTCGGACGCGGTGCTGCAGCGGTTGCCAGAGGAGGCC
CGCCACCGGCGTGTGTTCGAGATGGTGGAGGCACTGCAGGAGCACCCTCGAGACCCCAACCAGATCCTGA
TCGGCTACAGCCGAGGCCTCGTTGTCATCTGGGACCTACAGGGCAGCCGCGTGCTCTACCACTTCCTCAG
CAGCCAGCAACTGGAGAACATCTGGTGGCAGCGGGACGGCCGCCTGCTCGTCAGCTGTCACTCTGACGGC
AGCTACTGCCAGTGGCCCGTGTCCAGCGAAGCCCAGCAACCAGAGCCCCTCCGCAGCCTCGTGCCTTACG
GTCCCTTTCCTTGCAAAGCGATTACCAGAATCCTCTGGCTGACCACTAGGCAGGGGTTGCCCTTCACCAT
CTTCCAGGGTGGCATGCCACGGGCCAGCTACGGGGACCGCCACTGCATCTCAGTGATCCACGATGGCCAG
CAGACGGCCTTCGACTTCACCTCCCGTGTCATCGGCTTCACTGTCCTCACAGAGGCAGACCCTGCAGCCA
GTAGGAGAGCTTCGGGAGTGGGTGCCCAGGGTTAGGTGTGGGAGGCATGGGGCAGGACCATCAGTAAAGA
CAGGGCCAGGTGCAGTGGCTCCTGCCTGTAACCCCAGTGCTGTGGGAGGCCAAGGTGGTAGGATCGCTTG
AACCCAGGAGTTCAAGTCCAGCCTGGACAACGTAGGGAGACCCTTGTCTCTACAAAAAATAAAAAAATTA
GCCAGGAAAAAAAAAAAAAAAAAAAAAAAAAAAAAAAAAA
SEQ ID NO: 34
- Homo sapiens neuroblastoma RAS viral (v-ras)
oncogene homolog (NRAS), mRNA
GAAACGTCCCGTGTGGGAGGGGCGGGTCTGGGTGCGGCCTGCCGCATGACTCGTGGTTCGGAGGCCCACG
TGGCCGGGGCGGGGACTCAGGCGCCTGGGGCGCCGACTGATTACGTAGCGGGCGGGGCCGGAAGTGCCGC
TCCTTGGTGGGGGCTGTTCATGGCGGTTCCGGGGTCTCCAACATTTTTCCCGGCTGTGGTCCTAAATCTG
TCCAAAGCAGAGGCAGTGGAGCTTGAGGTTCTTGCTGGTGTGAAATGACTGAGTACAAACTGGTGGTGGT
TGGAGCAGGTGGTGTTGGGAAAAGCGCACTGACAATCCAGCTAATCCAGAACCACTTTGTAGATGAATAT
GATCCCACCATAGAGGATTCTTACAGAAAACAAGTGGTTATAGATGGTGAAACCTGTTTGTTGGACATAC
TGGATACAGCTGGACAAGAAGAGTACAGTGCCATGAGAGACCAATACATGAGGACAGGCGAAGGCTTCCT
CTGTGTATTTGCCATCAATAATAGCAAGTCATTTGCGGATATTAACCTCTACAGGGAGCAGATTAAGCGA
GTAAAAGACTCGGATGATGTACCTATGGTGCTAGTGGGAAACAAGTGTGATTTGCCAACAAGGACAGTTG
ATACAAAACAAGCCCACGAACTGGCCAAGAGTTACGGGATTCCATTCATTGAAACCTCAGCCAAGACCAG
ACAGGGTGTTGAAGATGCTTTTTACACACTGGTAAGAGAAATACGCCAGTACCGAATGAAAAAACTCAAC
AGCAGTGATGATGGGACTCAGGGTTGTATGGGATTGCCATGTGTGGTGATGTAACAAGATACTTTTAAAG
TTTTGTCAGAAAAGAGCCACTTTCAAGCTGCACTGACACCCTGGTCCTGACTTCCCTGGAGGAGAAGTAT
TCCTGTTGCTGTCTTCAGTCTCACAGAGAAGCTCCTGCTACTTCCCCAGCTCTCAGTAGTTTAGTACAAT
AATCTCTATTTGAGAAGTTCTCAGAATAACTACCTCCTCACTTGGCTGTCTGACCAGAGAATGCACCTCT
TGTTACTCCCTGTTATTTTTCTGCCCTGGGTTCTTCCACAGCACAAACACACCTCTGCCACCCCAGGTTT
TTCATCTGAAAAGCAGTTCATGTCTGAAACAGAGAACCAAACCGCAAACGTGAAATTCTATTGAAAACAG
TGTCTTGAGCTCTAAAGTAGCAACTGCTGGTGATTTTTTTTTTCTTTTTACTGTTGAACTTAGAACTATG
CTAATTTTTGGAGAAATGTCATAAATTACTGTTTTGCCAAGAATATAGTTATTATTGCTGTTTGGTTTGT
TTATAATGTTATCGGCTCTATTCTCTAAACTGGCATCTGCTCTAGATTCATAAATACAAAAATGAATACT
GAATTTTGAGTCTATCCTAGTCTTCACAACTTTGACGTAATTAAATCCAACTTTCACAGTGAAGTGCCTT
TTTCCTAGAAGTGGTTTGTAGACTTCCTTTATAATATTTCAGTGGAATAGATGTCTCAAAAATCCTTATG
CATGAAATGAATGTCTGAGATACGTCTGTGACTTATCTACCATTGAAGGAAAGCTATATCTATTTGAGAG
CAGATGCCATTTTGTACATGTATGAAATTGGTTTTCCAGAGGCCTGTTTTGGGGCTTTCCCAGGAGAAAG
ATGAAACTGAAAGCACATGAATAATTTCACTTAATAATTTTTACCTAATCTCCACTTTTTTCATAGGTTA
CTACCTATACAATGTATGTAATTTGTTTCCCCTAGCTTACTGATAAACCTAATATTCAATGAACTTCCAT
TTGTATTCAAATTTGTGTCATACCAGAAAGCTCTACATTTGCAGATGTTCAAATATTGTAAAACTTTGGT
GCATTGTTATTTAATAGCTGTGATCAGTGATTTTCAAACCTCAAATATAGTATATTAACAAATTACATTT
TCACTGTATATCATGGTATCTTAATGATGTATATAATTGCCTTCAATCCCCTTCTCACCCCACCCTCTAC
AGCTTCCCCCACAGCAATAGGGGCTTGATTATTTCAGTTGAGTAAAGCATGGTGCTAATGGACCAGGGTC
ACAGTTTCAAAACTTGAACAATCCAGTTAGCATCACAGAGAAAGAAATTCTTCTGCATTTGCTCATTGCA
CCAGTAACTCCAGCTAGTAATTTTGCTAGGTAGCTGCAGTTAGCCCTGCAAGGAAAGAAGAGGTCAGTTA
GCACAAACCCTTTACCATGACTGGAAAACTCAGTATCACGTATTTAAACATTTTTTTTTCTTTTAGCCAT
GTAGAAACTCTAAATTAAGCCAATATTCTCATTTGAGAATGAGGATGTCTCAGCTGAGAAACGTTTTAAA
TTCTCTTTATTCATAATGTTCTTTGAAGGGTTTAAAACAAGATGTTGATAAATCTAAGCTGATGAGTTTG
CTCAAAACAGGAAGTTGAAATTGTTGAGACAGGAATGGAAAATATAATTAATTGATACCTATGAGGATTT
GGAGGCTTGGCATTTTAATTTGCAGATAATACCCTGGTAATTCTCATGAAAAATAGACTTGGATAACTTT
TGATAAAAGACTAATTCCAAAATGGCCACTTTGTTCCTGTCTTTAATATCTAAATACTTACTGAGGTCCT
CCATCTTCTATATTATGAATTTTCATTTATTAAGCAAATGTCATATTACCTTGAAATTCAGAAGAGAAGA
AACATATACTGTGTCCAGAGTATAATGAACCTGCAGAGTTGTGCTTCTTACTGCTAATTCTGGGAGCTTT
CACAGTACTGTCATCATTTGTAAATGGAAATTCTGCTTTTCTGTTTCTGCTCCTTCTGGAGCAGTGCTAC
TCTGTAATTTTCCTGAGGCTTATCACCTCAGTCATTTCTTTTTTAAATGTCTGTGACTGGCAGTGATTCT
TTTTCTTAAAAATCTATTAAATTTGATGTCAAATTAGGGAGAAAGATAGTTACTCATCTTGGGCTCTTGT
GCCAATAGCCCTTGTATGTATGTACTTAGAGTTTTCCAAGTATGTTCTAAGCACAGAAGTTTCTAAATGG
GGCCAAAATTCAGACTTGAGTATGTTCTTTGAATACCTTAAGAAGTTACAATTAGCCGGGCATGGTGGCC
CGTGCCTGTAGTCCCAGCTACTTGAGAGGCTGAGGCAGGAGAATCACTTCAACCCAGGAGGTGGAGGTTA
CAGTGAGCAGAGATCGTGCCACTGCACTCCAGCCTGGGTGACAAGAGAGACTTGTCTCCAAAAAAAAAGT
TACACCTAGGTGTGAATTTTGGCACAAAGGAGTGACAAACTTATAGTTAAAAGCTGAATAACTTCAGTGT
GGTATAAAACGTGGTTTTTAGGCTATGTTTGTGATTGCTGAAAAGAATTCTAGTTTACCTCAAAATCCTT
CTCTTTCCCCAAATTAAGTGCCTGGCCAGCTGTCATAAATTACATATTCCTTTTGGTTTTTTTAAAGGTT
ACATGTTCAAGAGTGAAAATAAGATGTTCTGTCTGAAGGCTACCATGCCGGATCTGTAAATGAACCTGTT
AAATGCTGTATTTGCTCCAACGGCTTACTATAGAATGTTACTTAATACAATATCATACTTATTACAATTT
TTACTATAGGAGTGTAATAGGTAAAATTAATCTCTATTTTAGTGGGCCCATGTTTAGTCTTTCACCATCC
TTTAAACTGCTGTGAATTTTTTTGTCATGACTTGAAAGCAAGGATAGAGAAACACTTTAGAGATATGTGG
GGTTTTTTTACCATTCCAGAGCTTGTGAGCATAATCATATTTGCTTTATATTTATAGTCATGAACTCCTA
AGTTGGCAGCTACAACCAAGAACCAAAAAATGGTGCGTTCTGCTTCTTGTAATTCATCTCTGCTAATAAA
TTATAAGAAGCAAGGAAAATTAGGGAAAATATTTTATTTGGATGGTTTCTATAAACAAGGGACTATAATT
CTTGTACATTATTTTTCATCTTTGCTGTTTCTTTGAGCAGTCTAATGTGCCACACAATTATCTAAGGTAT
TTGTTTTCTATAAGAATTGTTTTAAAAGTATTCTTGTTACCAGAGTAGTTGTATTATATTTCAAAACGTA
AGATGATTTTTAAAAGCCTGAGTACTGACCTAAGATGGAATTGTATGAACTCTGCTCTGGAGGGAGGGGA
GGATGTCCGTGGAAGTTGTAAGACTTTTATTTTTTTGTGCCATCAAATATAGGTAAAAATAATTGTGCAA
TTCTGCTGTTTAAACAGGAACTATTGGCCTCCTTGGCCCTAAATGGAAGGGCCGATATTTTAAGTTGATT
ATTTTATTGTAAATTAATCCAACCTAGTTCTTTTTAATTTGGTTGAATGTTTTTTCTTGTTAAATGATGT
TTAAAAAATAAAAACTGGAAGTTCTTGGCTTAGTCATAATTCTT
SEQ ID NO: 35
- Homo sapiens 2′-5′-oligoadenylate synthetase 1,
40/46 kDa (OAS1), transcript variant 3, mRNA
TCCCTTCTGAGGAAACGAAACCAACAGCAGTCCAAGCTCAGTCAGCAGAAGAGATAAAAGCAAACAGGTC
TGGGAGGCAGTTCTGTTGCCACTCTCTCTCCTGTCAATGATGGATCTCAGAAATACCCCAGCCAAATCTC
TGGACAAGTTCATTGAAGACTATCTCTTGCCAGACACGTGTTTCCGCATGCAAATCAACCATGCCATTGA
CATCATCTGTGGGTTCCTGAAGGAAAGGTGCTTCCGAGGTAGCTCCTACCCTGTGTGTGTGTCCAAGGTG
GTAAAGGGTGGCTCCTCAGGCAAGGGCACCACCCTCAGAGGCCGATCTGACGCTGACCTGGTTGTCTTCC
TCAGTCCTCTCACCACTTTTCAGGATCAGTTAAATCGCCGGGGAGAGTTCATCCAGGAAATTAGGAGACA
GCTGGAAGCCTGTCAAAGAGAGAGAGCATTTTCCGTGAAGTTTGAGGTCCAGGCTCCACGCTGGGGCAAC
CCCCGTGCGCTCAGCTTCGTACTGAGTTCGCTCCAGCTCGGGGAGGGGGTGGAGTTCGATGTGCTGCCTG
CCTTTGATGCCCTGGGTCAGTTGACTGGCGGCTATAAACCTAACCCCCAAATCTATGTCAAGCTCATCGA
GGAGTGCACCGACCTGCAGAAAGAGGGCGAGTTCTCCACCTGCTTCACAGAACTACAGAGAGACTTCCTG
AAGCAGCGCCCCACCAAGCTCAAGAGCCTCATCCGCCTAGTCAAGCACTGGTACCAAAATTGTAAGAAGA
AGCTTGGGAAGCTGCCACCTCAGTATGCCCTGGAGCTCCTGACGGTCTATGCTTGGGAGCGAGGGAGCAT
GAAAACACATTTCAACACAGCCCAGGGATTTCGGACGGTCTTGGAATTAGTCATAAACTACCAGCAACTC
TGCATCTACTGGACAAAGTATTATGACTTTAAAAACCCCATTATTGAAAAGTACCTGAGAAGGCAGCTCA
CGAAACCCAGGCCTGTGATCCTGGACCCGGCGGACCCTACAGGAAACTTGGGTGGTGGAGACCCAAAGGG
TTGGAGGCAGCTGGCACAAGAGGCTGAGGCCTGGCTGAATTACCCATGCTTTAAGAATTGGGATGGGTCC
CCAGTGAGCTCCTGGATTCTGCTGACCCAGCACACTCCAGGCAGCATCCACCCCACAGGCAGAAGAGGAC
TGGACCTGCACCATCCTCTGAATGCCAGTGCATCTTGGGGGAAAGGGCTCCAGTGTTATCTGGACCAGTT
CCTTCATTTTCAGGTGGGACTCTTGATCCAGAGAGGACAAAGCTCCTCAGTGAGCTGGTGTATAATCCAG
GACAGAACCCAGGTCTCCTGACTCCTGGCCTTCTATGCCCTCTATCCTATCATAGATAACATTCTCCACA
GCCTCACTTCATTCCACCTATTCTCTGAAAATATTCCCTGAGAGAGAACAGAGAGATTTAGATAAGAGAA
TGAAATTCCAGCCTTGACTTTCTTCTGTGCACCTGATGGGAGGGTAATGTCTAATGTATTATCAATAACA
ATAAAAATAAAGCAAATACCATTTAAAAAAAAAAA
SEQ ID NO: 36
- Homo sapiens origin recognition complex, subunit
1 (ORC1), transcript variant 3, mRNA
ACGGTCTGGGGGCGGGGCCACGCCGATTGGCGCGAAGTTTTCTTTTCTCCTTCCACCTTCTTTTCATTTC
TAGTGAGACACACGCTTTGGTCCTGGCTTTCGGCCCGTAGTTGTAGAAGGAGCCCTGCTGGTGCAGGTTA
GAGGTGCCGCATCCCCCGGAGCTCTCGAAGTGGAGGCGGTAGGAAACGGAGGGCTTGCGGCTAGCCGGAG
GAAGCTTTGGAGCCGGAAGCCATGGCACACTACCCCACAAGGCTGAAGACCAGAAAAACTTATTCATGGG
TTGGCAGGCCCTTGTTGGATCGAAAACTGCACTACCAAACCTATAGAGAAATGTGTGTGAAAACAGAAGG
TTGTTCCACCGAGATTCACATCCAGATTGGACAGTTTGTGTTGATTGAAGGGGATGATGATGAAAACCCG
TATGTTGCTAAATTGCTTGAGTTGTTCGAAGATGACTCTGATCCTCCTCCTAAGAAACGTGCTCGAGTAC
AGTGGTTTGTCCGATTCTGTGAAGTCCCTGCCTGTAAACGGCATTTGTTGGGCCGGAAGCCTGGTGCACA
GGAAATATTCTGGTATGATTACCCGGCCTGTGACAGCAACATTAATGCGGAGACCATCATTGGCCTTGTT
CGGGTGATACCTTTAGCCCCAAAGGATGTGGTACCGACGAATCTGAAAAATGAGAAGACACTCTTTGTGA
AACTATCCTGGAATGAGAAGAAATTCAGGCCACTTTCCTCAGAACTATTTGCGGAGTTGAATAAACCACA
AGAGAGTGCAGCCAAGTGCCAGAAACCCGTGAGAGCCAAGAGTAAGAGTGCAGAGAGCCCTTCTTGGACC
CCAGCAGAACATGTGGCCAAAAGGATTGAATCAAGGCACTCCCCCTCCAAATCTCGCCAAACTCCTACCC
ATCCTCTTACCCCAAGAGCCAGAAAGAGGCTGGAGCTTGGCAACTTAGGTAACCCTCAGATGTCCCAGCA
GACTTCATGTGCCTCCTTGGATTCTCCAGGAAGAATAAAACGGAAAGTGGCCTTCTCGGAGATCACCTCA
CCTTCTAAGAGATCTCAGCCTGATAAACTTCAAACCTTGTCTCCAGCTCTGAAAGCCCCAGAGAAAACCA
GAGAGACTGGACTCTCTTATACTGAGGATGACAAGAAGGCTTCACCTGAACATCGCATAATCCTGAGAAC
CCGAATTGCAGCTTCGAAAACCATAGACATTAGAGAGGAGAGAACACTTACCCCTATCAGTGGGGGACAG
AGATCTTCAGTGGTGCCATCCGTGATTCTGAAACCAGAAAACATCAAAAAGAGGGATGCAAAAGAAGCAA
AAGCCCAGAATGAAGCGACCTCTACTCCCCATCGTATCCGCAGAAAGAGTTCTGTCTTGACTATGAATCG
GATTAGGCAGCAGCTTCGGTTTCTAGGTAATAGTAAAAGTGACCAAGAAGAGAAAGAGATTCTGCCAGCA
GCAGAGATTTCAGACTCTAGCAGTGACGAAGAAGAGGCTTCCACACCGCCCCTTCCAAGGAGAGCACCCA
GAACTGTGTCCAGGAACCTGCGATCTTCCTTGAAGTCATCCTTACATACCCTCACGAAGCTCAAGCCTAG
AACGCCACGTTGTGCCGCTCCTCAGATCCGTAGTCGAAGCCTGGCTGCCCAGGAGCCAGCCAGTGTGCTG
GAGGAAGCCCGACTGAGGCTGCATGTTTCTGCTGTACCTGAGTCTCTTCCCTGTCGGGAACAGGAATTCC
AAGACATCTACAATTTTGTGGAAAGCAAACTCCTTGACCATACCGGAGGGTGCATGTACATCTCCGGTGT
CCCTGGGACAGGGAAGACTGCCACTGTTCATGAAGTGATACGCTGCCTGCAGCAGGCAGCCCAAGCCAAT
GATGTTCCTCCCTTTCAATACATTGAGGTCAATGGCATGAAGCTGACGGAGCCCCACCAAGTCTATGTGC
AAATCTTGCAGAAGCTAACAGGCCAAAAAGCAACAGCCAACCATGCGGCAGAACTGCTGGCAAAGCAATT
CTGCACCCGAGGGTCACCTCAGGAAACCACCGTCCTGCTTGTGGATGAGCTCGACCTTCTGTGGACTCAC
AAACAAGACATAATGTACAATCTCTTTGACTGGCCCACTCATAAGGAGGCCCGGCTTGTGGTCCTGGCAA
TTGCCAACACAATGGACCTGCCAGAGCGAATCATGATGAACCGGGTGTCCAGCCGACTGGGTCTTACCAG
GATGTGCTTCCAGCCCTATACATATAGCCAGCTGCAGCAGATCCTAAGGTCCCGGCTCAAGCATCTAAAG
GCCTTTGAAGATGATGCCATCCAGCTGGTAGCCAGGAAGGTAGCAGCACTGTCTGGAGATGCACGACGGT
GCCTGGACATCTGCAGGCGTGCCACAGAGATCTGTGAGTTCTCCCAGCAGAAGCCTGACTCCCCTGGCCT
GGTCACCATAGCCCACTCAATGGAAGCTGTGGATGAGATGTTTTCATCATCATACATCACGGCCATCAAA
AATTCCTCTGTTCTGGAACAGAGCTTCCTGAGAGCCATCCTCGCAGAGTTCCGTCGATCAGGACTGGAGG
AAGCCACGTTTCAACAGATATATAGTCAACATGTGGCACTGTGCAGAATGGAGGGACTGCCGTACCCCAC
CATGTCAGAGACCATGGCCGTGTGTTCTCACCTGGGCTCCTGTCGCCTCCTGCTTGTGGAGCCCAGCAGG
AACGATCTGCTCCTTCGGGTGCGGCTCAACGTCAGCCAGGATGATGTGCTGTATGCGCTGAAAGACGAGT
AAAGGGGCTTCACAAGTTAAAAGACTGGGGTCTTGCTGGGTTTTGTTTTTTGAGACAGGGTCTTGCTCTG
TCGCCCAGGCTGGAGTGCAGTGGCACGATCATGGCTCACTGCAGCCTTGACTTCTCAGGCTTAGGTGACC
CCCCAACCTCATCCTCCCAGGTGGCTGAAACTACAGGCACATGCCACCATGCCCAGCTGATTTTTTGTAG
AGACAGGGCTTCACCATGTTGCCAAGCTAGTCTACAAAGCATCTGATTTTGGAAGTACATGGAATTGTTG
TAACAAAGTATATTGAATGGAAATGGCTCTCATGTATTTTGGAATTTTCCATTAAATAATTTGCTTTTTC
CTGAAAAAAAAAAAAAAAAAAAAAAAA
SEQ ID NO: 37
- Homo sapiens phosphoglycerate kinase 1 (PGK1), mRNA
GAGAGCAGCGGCCGGGAAGGGGCGGTGCGGGAGGCGGGGTGTGGGGCGGTAGTGTGGGCCCTGTTCCTGC
CCGCGCGGTGTTCCGCATTCTGCAAGCCTCCGGAGCGCACGTCGGCAGTCGGCTCCCTCGTTGACCGAAT
CACCGACCTCTCTCCCCAGCTGTATTTCCAAAATGTCGCTTTCTAACAAGCTGACGCTGGACAAGCTGGA
CGTTAAAGGGAAGCGGGTCGTTATGAGAGTCGACTTCAATGTTCCTATGAAGAACAACCAGATAACAAAC
AACCAGAGGATTAAGGCTGCTGTCCCAAGCATCAAATTCTGCTTGGACAATGGAGCCAAGTCGGTAGTCC
TTATGAGCCACCTAGGCCGGCCTGATGGTGTGCCCATGCCTGACAAGTACTCCTTAGAGCCAGTTGCTGT
AGAACTCAAATCTCTGCTGGGCAAGGATGTTCTGTTCTTGAAGGACTGTGTAGGCCCAGAAGTGGAGAAA
GCCTGTGCCAACCCAGCTGCTGGGTCTGTCATCCTGCTGGAGAACCTCCGCTTTCATGTGGAGGAAGAAG
GGAAGGGAAAAGATGCTTCTGGGAACAAGGTTAAAGCCGAGCCAGCCAAAATAGAAGCTTTCCGAGCTTC
ACTTTCCAAGCTAGGGGATGTCTATGTCAATGATGCTTTTGGCACTGCTCACAGAGCCCACAGCTCCATG
GTAGGAGTCAATCTGCCACAGAAGGCTGGTGGGTTTTTGATGAAGAAGGAGCTGAACTACTTTGCAAAGG
CCTTGGAGAGCCCAGAGCGACCCTTCCTGGCCATCCTGGGCGGAGCTAAAGTTGCAGACAAGATCCAGCT
CATCAATAATATGCTGGACAAAGTCAATGAGATGATTATTGGTGGTGGAATGGCTTTTACCTTCCTTAAG
GTGCTCAACAACATGGAGATTGGCACTTCTCTGTTTGATGAAGAGGGAGCCAAGATTGTCAAAGACCTAA
TGTCCAAAGCTGAGAAGAATGGTGTGAAGATTACCTTGCCTGTTGACTTTGTCACTGCTGACAAGTTTGA
TGAGAATGCCAAGACTGGCCAAGCCACTGTGGCTTCTGGCATACCTGCTGGCTGGATGGGCTTGGACTGT
GGTCCTGAAAGCAGCAAGAAGTATGCTGAGGCTGTCACTCGGGCTAAGCAGATTGTGTGGAATGGTCCTG
TGGGGGTATTTGAATGGGAAGCTTTTGCCCGGGGAACCAAAGCTCTCATGGATGAGGTGGTGAAAGCCAC
TTCTAGGGGCTGCATCACCATCATAGGTGGTGGAGACACTGCCACTTGCTGTGCCAAATGGAACACGGAG
GATAAAGTCAGCCATGTGAGCACTGGGGGTGGTGCCAGTTTGGAGCTCCTGGAAGGTAAAGTCCTTCCTG
GGGTGGATGCTCTCAGCAATATTTAGTACTTTCCTGCCTTTTAGTTCCTGTGCACAGCCCCTAAGTCAAC
TTAGCATTTTCTGCATCTCCACTTGGCATTAGCTAAAACCTTCCATGTCAAGATTCAGCTAGTGGCCAAG
AGATGCAGTGCCAGGAACCCTTAAACAGTTGCACAGCATCTCAGCTCATCTTCACTGCACCCTGGATTTG
CATACATTCTTCAAGATCCCATTTGAATTTTTTAGTGACTAAACCATTGTGCATTCTAGAGTGCATATAT
TTATATTTTGCCTGTTAAAAAGAAAGTGAGCAGTGTTAGCTTAGTTCTCTTTTGATGTAGGTTATTATGA
TTAGCTTTGTCACTGTTTCACTACTCAGCATGGAAACAAGATGAAATTCCATTTGTAGGTAGTGAGACAA
AATTGATGATCCATTAAGTAAACAATAAAAGTGTCCATTGAAACCGTGATTTTTTTTTTTTTCCTGTCAT
ACTTTGTTAGGAAGGGTGAGAATAGAATCTTGAGGAACGGATCAGATGTCTATATTGCTGAATGCAAGAA
GTGGGGCAGCAGCAGTGGAGAGATGGGACAATTAGATAAATGTCCATTCTTTATCAAGGGCCTACTTTAT
GGCAGACATTGTGCTAGTGCTTTTATTCTAACTTTTATTTTTATCAGTTACACATGATCATAATTTAAAA
AGTCAAGGCTTATAACAAAAAAGCCCCAGCCCATTCCTCCCATTCAAGATTCCCACTCCCCAGAGGTGAC
CACTTTCAACTCTTGAGTTTTTCAGGTATATACCTCCATGTTTCTAAGTAATATGCTTATATTGTTCACT
TCTTTTTTTTTTATTTTTTAAAGAAATCTATTTCATACCATGGAGGAAGGCTCTGTTCCACATATATTTC
CACTTCTTCATTCTCTCGGTATAGTTTTGTCACAATTATAGATTAGATCAAAAGTCTACATAACTAATAC
AGCTGAGCTATGTAGTATGCTATGATTAAATTTACTTATGTAAAAAAAAAAAAAAAAAA
SEQ ID NO: 38
- Homo sapiens phorbol-12-myristate-13-acetate-
induced protein 1 (PMAIP1), mRNA
ACTGGACAAAAGCGTGGTCTCTGGCGCGGGGATCTCAGAGTTTCCCGGGCACTCACCGTGTGTAGTTGGC
ATCTCCGCGCGTCCGGACACCCGATCCCAGCATCCCTGCCTGCAGGACTGTTCGTGTTCAGCTCGCGTCC
TGCAGCTGTCCGAGGTGCTCCAGTTGGAGGCTGAGGTTCCCGGGCTCTGTAGCTGAGTGGGCGGCGGCAC
CGGCGGAGATGCCTGGGAAGAAGGCGCGCAAGAACGCTCAACCGAGCCCCGCGCGGGCTCCAGCAGAGCT
GGAAGTCGAGTGTGCTACTCAACTCAGGAGATTTGGAGACAAACTGAACTTCCGGCAGAAACTTCTGAAT
CTGATATCCAAACTCTTCTGCTCAGGAACCTGACTGCATCAAAAACTTGCATGAGGGGACTCCTTCAAAA
GAGTTTTCTCAGGAGGTGCACGTTTCATCAATTTGAAGAAAGACTGCATTGTAATTGAGAGGAATGTGAA
GGTGCATTCATGGGTGCCCTTGGAAACGGAAGATGGAATACATCAAAGTGAATTTCTGTTCAAGTTTTCC
CAGATTATCATTCTTTGGGATGAGAGAACATTATAAAACCACTTTGTTTATTTTAAAGCAAGAATGGAAG
ACCCTTGAAAATAAAGAAGTAATTATTGACACATTTCTTTTTTACTTAGAGAATCGTTCTAGTGTTTTTG
CCGAAGATTACCGCTGGCCTACTGTGAAGGGAGATGACCTGTGATTAGACTGGGCGGCTGGGGAGAAACA
GTTCAGTGCATTGTTGTTGTTGCTGTTTTTGGTGTTTTGCTTTTCAGTGCCAACTCAGCACATTGTATAT
GATTCGGTTTATACATATTACCTTGTTATAATGAAAAAACTCATTCTGAGAACACTGAAATGTTATACTC
AGTGTTGATTTCTTCGGTCACTACACAACGTAAAATCATTTGTTTCTTTTGACTCAAATTGTATTGCTTC
TGTTCAGATGATCTTTCATTCAATGTGTTCCTGTTGGGCGTTACTAGAAACTATGGAAAACTGGAAAATA
ACTTTGAAAAAATTGGATAAAGTATAGGAGGGTTACTTGGGGCCAGTAAATCAGTAGACTGAACATTCAA
TATAATAAAAGAACATGGGGATTTTGTATAACCAGGGATAATAAAAAGAAAAAAGAAGTTAATTTTTAAT
TGATGTTTTTGAAACTTAGTAGAACAAATATTCAGAAGTAACTTGATAAGATATGAATGTTTCTAAAGAA
GTTTCTAAAGGTTCGGAAAATGCTCCTTGTCACATTAGTGTGCATCCTACAAAAAGTGATCTCTTAATGT
AAATTAAGAATATTTTCATAATTGGAATATACTTTTCTTAAAAAAAAGGAACAGTTAGTTCTCATCTAGA
ATGAAAGTTCCATATATGCATTGGTGAATATATATGTATACACATACTTACATACTTATATGGGTATCTG
TATAGATAATTTGTATTAGAGTATTATATAGCTTCTTAGTAGGGTCTCAAGTAAGTTTCATTTTTTTTAT
CTGGGCTATATACAGTCCTCAAATAAATAATGTCTTGATTTTATTTCAGCAGGAATAATTTTATTTATTT
TGCCTATTTATAATTAAAGTATTTTTCTTTAGTTTGAAAATGTGTATTAAAGTTACATTTTTGAGTTACA
AGAGTCTTATAACTACTTGAATTTTTAGTTAAAATGTCTTAATGTAGGTTGTAGTCACTTTAGATGGAAA
ATTACCTCACATCTGTTTTCTTCAGTATTACTTAAGATTGTTTATTTAGTGGTAGAGAGTTTTTTTTTTC
AGCCTAGAGGCAGCTATTTTACCATCTGGTATTTATGGTCTAATTTGTATTTAAACATATGCACACATAT
AAAAGTTGATACTGTGGCAGTAAACTATTAAAAGTTTTCACTGTTCAAAAAAAAAAAAAAAAAA
SEQ ID NO: 39
- Homo sapiens POU class 6 homeobox 1 (POU6F1),
transcript variant 2, non-coding RNA
AATCGGTGGCCGCCAGACACCCGCGGCGAAGGCGGCTCGGGCTCGGGCTCCGGATGTGCTAGGTGTGGGC
CGGCCCCCACCCGACCCTGACAAGTGACCATGGATCCTGGAGCCGGGTCAGAGACATCTCTGACTGTCAA
TGAGCAGGTCATCGTGATGTCAGGTCATGAGACCATCCGAGTGCTGGAAGTCGGAGTGGATGCCCAACTC
CCTGCTGAGGAAGAGAGCAAAGGACTGGAGGGTGTGGCCGCCGAGGGCTCCCAGAGCGGAGACCCTGCTG
AAGCCAGTCAAGCTGCTGGTGAAGCTGGGCCAGACAACCTGGGCTCCTCTGCAGAGGCAACTGTGAAGTC
ACCCCCGGGGATCCCTCCGAGCCCTGCCCCTGCCATTGCCACCTTCAGCCAAGCCCCAAGCCAGCCTCAG
GCATCGCAGACCCTGACGCCACTGGCTGTACAAGCTGCCCCCCAGTATTGCAGGTCAAGTGGCTGGTCAG
CAGGGGCTGGCCGTGTGGACAATTCCTACAGCAACTGTGGCTGCCCTCCCAGGACTGACCGCTGCTTCTC
CTACGGGGGGAGTGTTCAAGCCACCTTTAGCCGGTCTCCAAGCAGCTGCTGTGCTGAACACCGCTCTTCC
GGCACCGGTACAAGCTGCCGCACCAGTACAGGCCTCCTCGACGGCCCAACCCCGGCCACCAGCCCAGCCC
CAGACGCTGTTCCAGACCCAGCCGCTGCTGCAGACCACACCTGCCATCCTCCCGCAGCCCACTGCTGCCA
CCGCTGCTGCCCCTACCCCCAAGCCAGTGGACACCCCCCCACAGATCACCGTCCAGCCTGCAGGCTTCGC
ATTTAGCCCAGGAATCATCAGTGCTGCTTCCCTCGGGGGACAGACCCAGATCCTGGGGTCCCTCACTACA
GCTCCAGTCATTACCAGCGCCATTCCCAGCATGCCAGGGATCAGCAGTCAGATCCTCACCAATGCTCAGG
GACAGGTTATTGGAACCCTTCCATGGGTAGTGAACTCAGCTAGTGTGGCGGCCCCAGCACCAGCCCAAAG
CCTGCAGGTCCAGGCCGTGACCCCCCAGCTGTTGTTGAACGCCCAGGGCCAGGTGATTGCGACCCTGGCT
AGCAGCCCCCTGCCTCCACCTGTGGCTGTCCGGAAGCCAAGCACACCTGAGTCCCCTGCTAAGAGTGAGG
TGCAGCCCATCCAGCCCACACCAACCGTGCCCCAGCCTGCTGTGGTCATTGCCAGCCCAGCTCCAGCCGC
CAAGCCATCTGCCTCTGCTCCTATCCCAATTACCTGCTCAGAGACCCCCACCGTCAGCCAGTTGGTGTCC
AAGCCACATACTCCAAGTCTGGATGAGGATGGGATCAACTTAGAAGAGATCCGGGAGTTTGCCAAGAACT
TTAAGATCCGGCGGCTCTCGCTGGGCCTTACACAGACCCAGGTGGGTCAGGCTCTGACTGCAACGGAAGG
TCCAGCCTACAGCCAGTCAGCCATCTGCCGGTTCGAGAAGCTAGACATCACACCCAAGAGTGCCCAGAAG
CTAAAGCCGGTGCTGGAAAAGTGGCTAAACGAAGCTGAACTGCGGAACCAGGAAGGCCAGCAGAACCTGA
TGGAGTTTGTGGGAGGCGAGCCCTCCAAGAAACGCAAACGCCGCACCTCCTTCACCCCCCAGGCCATAGA
GGCTCTCAATGCCTATTTTGAGAAGAACCCACTGCCCACAGGCCAGGAGATCACTGAAATTGCTAAGGAG
CTCAACTACGACCGTGAGGTAGTGCGGGTCTGGTTCTGCAATCGGCGCCAGACGCTCAAGAACACCAGCA
AGCTGAACGTCTTTCAGATCCCTTAGGGCTCAGCCCCTGGCCCTGTGTTCTAGCACTTTGTCCATTTCCC
GTGGCATCCGGCTGCAGCCACTGCCATGACAGCACCTGTCATTTTGCCACGTGCAGCTGTGCTCACCCCA
GGTCATCAGACTCCACCGTGTGCATGTGCATCAATGTCCCTCTTTTCTCCCACACATCTCACATCATGGG
GAGGCCAGAGGGGGCCACACGAGAGCTCCAGGCTCTGGGCTGGTCACTCCGAAGAAGAGGATTTGTGACG
TCACTTAGAGAAGCACCTTGCTAGCATGGTTTCTGAAGGGTGAATTCTGGTGGGGAACCAGAAACTCCCT
GTCTTTGGGGCAGGGCTAAAGCAGCTCCTAAGGACCACTGGCCATTAGCTCTTGCTTTTGATGGCATTCT
CTTTCCACCTTGTCTTCTCCTTTGCTCCTCTGTGTTAGTGTGGCAGGTATGACAACTCATCCAGTGGAAA
CACAGCCTCACACTGCCCTTCCGCCCCCCACACTTTGCCTGCAGGTGCACCGAAAGGACCTGGGAGATAA
AATTCAAAAAAGTGTGATGTGCTGCTCAGAAGGTCAGACTCCATGTCTGCCTTGACCTCAAGGTCAGAAG
GTTCCCAAACCCCTGGGGCTGGAACATGGGATCTCCTCTTCCACCTCTTCCTGGTTCCTTTGCGGGGAAA
ATTGCACTAAAACAGAACCTTTTCTTAATCCATGTTGGAAGGAAGCAACAGTGAACTCTACCTGTTCTGG
AGTTCTCCTGGGTCTGCAGAAGGTTGGGAATTTAGAAAATAAGGCTGTTCTTTCATATTTTAATTTAATC
TCTGTCAATGGCCATCCCTCCCACAAAAAAACGTGGGTTAAGAGAACTTGCAGACTGGATATGCAAGCAA
ACGGGCAACTCTGGAGAAAAATAAGGAAAGGAATGCTGACTTTCTCTTTCTTTCTCTTGTCCCCACACCC
ATTCCCAACCCAATACTGGGGCCTTCTCAAAAGGAGCAAATTAAACAATAAACCAGACAGCAAGGCCCTG
GGGGAAAGGACAACATCCTGAAATAAATGATGGAGCCCAGGAAGGTCTCTTGTGGAAGTTGACTTAACTC
TAATTTTCTTTGTAACTTTAAGCCTTGGATACGGGAGGAGAAATCTCATTTTGTCGAGTCTCAGACCATG
TCTGTGTGTAAGCAATCCCCACAGTCTCCTCTGAGCCAAGGACACCCCCAGATCAGATTGAGTTTTGCTT
CTAGACGGGGTAGCTATGGTACCTTGGGGGTTAGCTCTCATCCAAGCTGTTAAGTGAGTTTCCAGCCTCA
CTGTGGCTGGAAAGCCCCTAAAATTCAGTATGTAACTCCAGGAAGTCAGGAGAGAACTGAGATTTGCCTA
GATGACCACAGGCTTGCGGTGTAGATTATCCCTAAAGGGCCCCAAGTCACGGGGGTCAACCACCCCTGTC
TTCAGTACTCTTATCCTTACAGAGGCTGGTCTCTAACAGCTGCCTCCAGTGGACCTCCCATGATCCACCC
TGAGGGAAGGACCGTCAGCTGGGGACACATCACCACCTCTGTCAGTCACTGGTGCAGAGCCACCTCCTAG
CCTAGCTTCCTCTGGTGTCCTGTTTCCTTTCCCACTTACTGTTGGTGCCTCCCAGGCCCTGCAGTGCCAG
CGTGGCCACCCTCTTGGTAGCCTGGCCAGTAAGAGGAGGACAGTTGTGTGCTGAATTAGCACACGCACGT
GCAGCGCGCACAGACGCGCGCACACACACACACATACACGCTCTGCTGCATTTGGACAAACCATGCCTGC
CAGAGTGTAGCAGAGGTGAGGAAGCAGGTGGGCAGCTTGCCTGACCCAGCTTTTCAGGAGAGCGTGTCTC
CAACAGAGAGTCTCCACACTCTAGTTCAGGGTTATCGACCTGCCTCAATGAGATGACAGACTCATTTGGG
AGGGGTGTTGCAAACAAGTTTTCAGTGAGAATAGTTAAGTTCCAGAGCTTGTAAAGGATTCAGTGACTGA
CACTTCAGTAAATTAGGCCAGGCACATTGGCTTATGCCTGTAATTCCAACACTTTGGAAGGCCGAGGTGG
GCGGATCATTTGAGGTCTGGAGTTCGAGACCAGCCTGACCAACATGGTGAAACCCCGTCTCTACTAAAAA
TACAAAAATTAGCCAGGTGTGGTAGTGCACATCTGTAATCCCAGCTACTTGGGAGGTGGAGGCAGGAGAA
TTGCTTGAACCCTGGAGGTTGCAATGAGCTGAGATCACACTACTTCACTCCAGCCTGGGTGACAGAGCAA
GACTCGGTCTCAAACAAACAAAAACTTATGGCGATGCAGGTTTTCATGCTCAGACGCTTGCATTCAGGTA
TGCTTTCTTTTTTGAGAGAGACAAATGGGTCACAGCTGGCACCCTGGGAATAGCACATAATCCAGGGTGT
GTCTGTGGTGGTGGACGTGCAGGGGAACACCATCTGTCCTGTCTCATGATGGGAAAACAATCATGAACCA
CTGGTCTAAATTAGGCCTGGCCATGCTTTCTCAGCCCCTCCCTCATTTAAATTTGTCTTCCCAAAGCTGA
GCTAAAACTAAACCATTTCTCCTCTGCTGGAATGATGGATTGGTCATTCAGAGGAACAATACCAGGGGTG
GGAGGTTTGCAGGCTGAGTTCCCCAGGCATGGGGGTGCAGGGTGTCCCTGAGGTTTACCCAAAGCACAGC
TCGCTGGCCTGTGACCTCTGCCCTTCCTCCCACAGTGTAAGACCCCCCAGGAAGCAGCTGGGGCCTGAAC
CTCTCACCTAGGAGGTAGGTTTATTTTATTTTTTGTTAGCATCAGGCTCTGAAGGAGTTGGTATACATTT
TGTTTTGAAAACATCTTCTGGACTTACACCAGAGCTTAGTGTCGTCTTTACTATGGAAAGAGAGGAGAAT
GGACAGAAATGGTTTAACTGTGTGGAGTTTTGTTTGTTTTGTTTTAAATGGAAGAAAGACCAAAACTTTC
CTGGTGGATCAGCTAGGGCCTTTGACCCTGCATTACCACGGCATTTTATCCAGGTGAAGTCCAGGGAAAG
AACTCAGCCAAATGGACTAAGGAACACACGAGTTTGGAATGCGAGACTCTGACATTTTTGTGTTCTTGGA
AATCCAATTACCTTCCCATGCCCAGATTTCCTTCCTGCCTCTTGGACCAGGCTCTGGCACTGAGGTTCTC
ACTGTTCCCAACACAGACAAAGCTTCCTGAGGGCTGGAGGGGCAGCAAGGGGAGAGGAGAATGGGGAAGA
AGCGCTTGATGTAGTTGTGTGGAATAAACAGTATTTTTTCTTTTGTAAAAAAAAAAAAAAAAA
SEQ ID NO: 40
- Homo sapiens Ran GTPase activating protein
1 (RANGAP1), mRNA
AAATCCTCCTCCTCCGCCATCATCCGCCGCGGTGCGGAGAGCAGGTGGTGCTGGAAGCGCGTGAGGCCGG
GAGCTCGAGAGAGCTAACAGACTAGCCGGCTGGACATCTGGACCGCTGGATCCGGAGGTGGCGACCCCGG
CCTGACCCGGACCCTAAATCCGTCCCCGCCCCAGAGGGCGGAGGCGCGCGCTCGATTCCCCCCACGCGGC
GGCGCCGCCTGTTTACGTCTGCAGATCTCCAGGGGAGCCCACCAGCCTAGTCAACATGGCCTCGGAAGAC
ATTGCCAAGCTGGCAGAGACACTTGCCAAGACTCAGGTGGCCGGGGGACAGCTGAGTTTCAAAGGCAAGA
GCCTCAAACTCAACACTGCAGAAGATGCTAAAGATGTGATTAAAGAGATTGAAGACTTTGACAGCTTGGA
GGCTCTGCGTCTGGAAGGCAACACAGTGGGCGTGGAAGCAGCCAGGGTCATCGCCAAGGCCTTAGAGAAG
AAGTCGGAGTTGAAGCGCTGCCACTGGAGTGACATGTTCACGGGAAGGCTGCGGACCGAGATCCCACCAG
CCCTGATCTCACTAGGGGAAGGACTCATCACAGCTGGGGCTCAGCTGGTGGAGCTGGACTTAAGCGACAA
CGCATTCGGGCCCGACGGTGTGCAAGGCTTCGAGGCCCTGCTCAAGAGCTCAGCCTGCTTCACCCTGCAG
GAACTCAAGCTCAACAACTGTGGCATGGGCATTGGCGGCGGCAAGATCCTGGCTGCAGCTCTGACCGAAT
GTCACCGGAAATCCAGTGCCCAAGGCAAGCCTCTGGCCCTGAAGGTCTTTGTGGCTGGCAGAAACCGTCT
GGAGAATGATGGCGCCACTGCCTTGGCAGAAGCTTTTAGGGTCATCGGGACCCTGGAGGAGGTCCACATG
CCACAGAATGGGATCAACCACCCTGGCATCACTGCCCTGGCCCAGGCTTTCGCTGTCAACCCCCTGCTGC
GGGTCATCAACCTGAATGACAACACCTTCACTGAGAAGGGCGCCGTGGCCATGGCCGAGACCTTGAAGAC
CTTGCGGCAGGTGGAGGTGATTAATTTTGGGGACTGCCTGGTGCGCTCCAAGGGTGCAGTTGCCATTGCA
GATGCCATCCGCGGCGGCCTGCCCAAGCTAAAGGAGCTGAACTTGTCATTCTGTGAAATCAAGAGGGATG
CTGCCCTGGCTGTTGCTGAGGCCATGGCAGACAAAGCTGAGCTGGAGAAGCTGGACCTGAATGGCAACAC
CCTGGGAGAAGAAGGCTGTGAACAGCTTCAGGAGGTGCTGGAGGGCTTCAACATGGCCAAGGTGCTGGCG
TCCCTCAGTGATGACGAGGACGAGGAGGAGGAGGAGGAAGGAGAAGAGGAAGAAGAGGAAGCAGAAGAAG
AGGAGGAGGAAGATGAGGAAGAGGAGGAAGAAGAGGAGGAGGAGGAGGAAGAAGAGCCTCAGCAGCGAGG
GCAGGGAGAGAAGTCAGCCACGCCCTCACGGAAGATTCTGGACCCTAACACTGGGGAGCCAGCTCCCGTG
CTGTCCTCCCCACCTCCTGCAGACGTCTCCACCTTCCTGGCTTTTCCCTCTCCAGAGAAGCTGCTGCGCC
TAGGGCCCAAGAGCTCCGTGCTGATAGCCCAGCAGACTGACACGTCTGACCCCGAGAAGGTGGTCTCTGC
CTTCCTAAAGGTGTCATCTGTGTTCAAGGACGAAGCTACTGTGAGGATGGCAGTGCAGGATGCAGTAGAT
GCCCTGATGCAGAAGGCTTTCAACTCCTCGTCCTTCAACTCCAACACCTTCCTCACCAGGCTGCTCGTGC
ACATGGGTCTGCTCAAGAGTGAAGACAAGGTCAAGGCCATTGCCAACCTGTACGGCCCCCTGATGGCGCT
GAACCACATGGTGCAGCAGGACTATTTCCCCAAGGCCCTTGCACCCCTGCTGCTGGCGTTCGTGACCAAG
CCCAACAGCGCCCTGGAATCCTGCTCCTTCGCCCGCCACAGTCTGCTGCAGACGCTGTACAAGGTCTAGA
CTCAAAGCCTCTCCCATCCCTTGGCCTGGACCAGTGAGCTGGGGAGGGACTCGGATGAACTGAGGCGCAG
CCTACGCCATTGCCTTGGACAGGACTCTGGCCACAGGCAGGGCGGGTCTGTGTCCCATGTGTCCTGTCAG
TCCCCTGAGTATGTGTGTGGGTGTGGCGCATGTGCAGGTCTGTGCCTCCTGTCGGGATTTGGGTTTTAAC
GTCTTCTGCTGGCCCAGCCCTGCTCTGTTGTGGGGAGTTGGCCCCCAGGGGAAAGGGCTGTGAGCTGCTC
CGCCATTAAACTCACCTCCACCTGAGGGCGCTCTGCTGATCTCCGCCTGGGCCCTGATGGCCGTCCCCAC
CCACCTGCCTTCCGGCCCGGCTCCCTGGCGGAGCCAGAACCCAGGGAGTTGCCCGCGTGCTGTCCTTCCC
CTCTGTGTTGTGATTGGGTTGTTTCCTGCCCTGCCTGGGGCTGCTTCTCGTCACCAAGCCCTGGTCCTGC
GGCAGCTGTCACCCCTACCATCCATACCACTGTGCTGACCGCTCAGCCTGAAGAGCAGAGAATGCCATGG
GTGGGACTGTGGGGGTCGGATCGTGGGGTTGTTGGCAGAGGGCAACCCTGGGCCCCACACCGTGTGGACA
GGCAGACACCAGATTGTCCAGGAGCAGGAGCTGCTGGGACTGCGCTGGCCCCGGACCTAGTGGGCCTTCT
CCTGGCTGCTGAGATGTCGTCTGTGACTGGCCTGGCTGGAGGGGGAGTGTTGACAACCCAAAGCTGTTCT
CCAGTCTGGGGAGGGAGAGGCAGGGTCCCCAATGTCCGAGCTGCATCTGGACGCTGCTCTTAAAGGACCT
CCTGGGGCAGGGGAGCGGTAGGGTCTGGACTGGGCAGATGCTGTATGACCTCCCTGAGCACCCGTGACTG
CCCCATGCTTTCCCCTTTGTGCTCTGTGTGTGTCTGGGCTGTGCCCGGGGGCTTCACAAATAAAGTCGTG
TGGCAGCTTCAAAAAAAAAAAAAAAAAAAA
SEQ ID NO: 41
- Homo sapiens Spi-B transcription factor
(Spi-1/PU.1 related) (SPIB), mRNA
GGCAAACAGCCCGCCCGGCACCACCATGCTCGCCCTGGAGGCTGCACAGCTCGACGGGCCACACTTCAGC
TGTCTGTACCCAGATGGCGTCTTCTATGACCTGGACAGCTGCAAGCATTCCAGCTACCCTGATTCAGAGG
GGGCTCCTGACTCCCTGTGGGACTGGACTGTGGCCCCACCTGTCCCAGCCACCCCCTATGAAGCCTTCGA
CCCGGCAGCAGCCGCTTTTAGCCACCCCCAGGCTGCCCAGCTCTGCTACGAACCCCCCACCTACAGCCCT
GCAGGGAACCTCGAACTGGCCCCCAGCCTGGAGGCCCCGGGGCCTGGCCTCCCCGCATACCCCACGGAGA
ACTTCGCTAGCCAGACCCTGGTTCCCCCGGCATATGCCCCGTACCCCAGCCCTGTGCTATCAGAGGAGGA
AGACTTACCGTTGGACAGCCCTGCCCTGGAGGTCTCGGACAGCGAGTCGGATGAGGCCCTCGTGGCTGGC
CCCGAGGGGAAGGGATCCGAGGCAGGGACTCGCAAGAAGCTGCGCCTGTACCAGTTCCTGCTGGGGCTAC
TGACGCGCGGGGACATGCGTGAGTGCGTGTGGTGGGTGGAGCCAGGCGCCGGCGTCTTCCAGTTCTCCTC
CAAGCACAAGGAACTCCTGGCGCGCCGCTGGGGCCAGCAGAAGGGGAACCGCAAGCGCATGACCTACCAG
AAGCTGGCGCGCGCCCTCCGAAACTACGCCAAGACCGGCGAGATCCGCAAGGTCAAGCGCAAGCTCACCT
ACCAGTTCGACAGCGCGCTGCTGCCTGCAGTCCGCCGGGCCTGAGCACACCCGAGGCTCCCACCTGCGGA
GCCGCTGGGGGACCTCACGTCCCAGCCAGGATCCCCCTGGAAGAAAAAGGGCGTCCCCACACTCTAGGTG
ATAGGACTTACGCATCCCCACCTTTTGGGGTAAGGGGAGTGCTGCCCTGCCATAATCCCCAAGCCCAGCC
CGGGCCTGTCTGGGATTCCCCACTTGTGCCTGGGGTCCCTCTGGGATTTCTTTGTCATGTACAGACTCCC
TGGGATCCTCATGTTTTGGGTGACAGGACCTATGGACCACTATACTCGGGGAGGCAGGGTAGCAGTTCTT
CCAGAATCCCAAGAGCTTCTCTGGGATTTTCTTGTGATATCTGATTCCCCAGTGAGGCCTGGGACGTTTT
TAAGATCGCTGTGTGTCTGTAAACCCTGAATCTCATCTGGGGTGGGGGCCCTGCTGGCAACCCTGAGCCC
TGTCCAAGGTTCCCTCTTGTCAGATCTGAGATTTCCTAGTTATGTCTGGGGCCCTCTGGGAGCTGTTATC
ATCTCAGATCTCTTCGCCCATCTATGGCTGTGTTGTCACATCTGTCCCCTCATTTTTGAGATCCCCCAAT
TCTCTGGAACTATTCTGCTGCCCCTTTTTATGTGTCTGGAGTTCCCCAATCACATCTAGGGCTCCTCCAA
GAAAAAAAAAAAAAAAAAAAAAA
SEQ ID NO: 42
- Homo sapiens TAF11 RNA polymerase II, TATA box
binding protein (TBP)-associated factor, 28 kDa
(TAF11), mRNA
AAGATCCTGGCCTGTGCAGCTCGGGTTTCCGAGCTTCTGCCTCAGGCATCTCCGCGATCTCCTCTCCCCT
CCAATCCTATCCGTGATGGACGATGCCCACGAGTCGCCCTCCGACAAAGGTGGAGAGACAGGGGAGTCGG
ATGAGACGGCCCCTGTGCCCGGGGACCCGGGGGCTACCGACACCGATGGAATCCCAGAGGAAACTGACGG
AGACGCAGATGTGGACTTGAAAGAAGCTGCAGCGGAGGAAGGCGAGCTCGAGAGTCAGGATGTCTCAGAT
TTAACAACAGTTGAAAGGGAAGACTCATCATTACTTAATCCTGCAGCCAAAAAACTGAAAATAGATACCA
AAGAAAAGAAAGAGAAAAAGCAGAAAGTAGATGAAGATGAGATTCAGAAGATGCAAATCCTGGTTTCTTC
TTTTTCTGAGGAGCAGCTGAACCGTTATGAAATGTATCGCCGCTCAGCTTTCCCTAAGGCAGCCATCAAA
AGGCTGATCCAGTCCATCACTGGCACCTCTGTGTCTCAGAATGTTGTTATTGCTATGTCTGGTATTTCCA
AGGTTTTCGTCGGGGAGGTGGTAGAAGAAGCACTGGATGTGTGTGAGAAGTGGGGAGAAATGCCACCACT
ACAACCCAAACATATGAGGGAAGCCGTTAGAAGGTTAAAGTCAAAAGGACAGATCCCTAACTCGAAGCAC
AAAAAAATCATCTTCTTCTAGACCAAAGTCTAGAAAGGCCTATGTTACTGACGGAAGAAGTATTGGTTCC
AGACTTCCTATAAGACTGTCTGCATTGGTGCTTTAGTATCTCAGGCCTCCAAGGATTCCATGATGATTTT
AATGTCTTTCTCAAAACTCTGATATTTGTCACACCTAGAAAGTATGTAGCCTGATTGATACTTGCCTTGA
CTAAATTTTGGGACCTCTTGGGGCATTTTGAAGTATTTAACTGTCTTGACCAGTTGGAAGAAGATACGTG
GGCCATAAGCATCTTCTGGACAGGGGAACTGCTTTCAGAGAGAAAACCTTTCCAAGAGAGTTTTGTTTTG
TTTTGGTTTCGTTTTGTTTGAGATAGGGTCTTGCTCTATCACCTAGGCTGGAGTGCAGCGGCATGACTGC
AGCCTTGAACTCCTGGGCTTAAGTGACCCTCCCACCTCAGTCTCCTGAGTAGCTAGGACTACAGGCACAC
ACTACTGTGCCCAGCTAACTTATTTTTATTTTTTATGGAGATGGGGTCTTGCTTTGTTGCCCAGGCTGGT
CGTGAACTCCTGGCTTCAAGCAGTCCTCCTGCCTCAGCCTCCTAAAGTGCCGAGGGCTTTAATGGTTTCA
CATTGAAGCCTGAAGTTGCTAAGACTTAGGTTGTTTCTTATATCTGGTTTTAAGTAGATGAAACAACCAG
AAACTTTTACTTGTGATACTCTACCATGAAGGATGCGGTAATGGCAGGAATAGCAGAATAATTGGTGCTT
GTAAACATTTAAGATTCTCCTGTGGATTTTGGTGAGTGATCATTAAACTGTTTTCCAACTTGCAAAAAAA
AAAAAAAAAAAAAAAAAAAAAAAAAAAAAAAAAAAAAAAAAAAAAAAAAAAAAAAAAAA
SEQ ID NO: 43
- Homo sapiens TATA box binding protein (TBP),
transcript variant 2, mRNA
GGCGGAAGTGACATTATCAACGCGCGCCAGGGGTTCAGTGAGGTCGGGCAGGTTCGCTGTGGCGGGCGCC
TGGGCCGCCGGCTGTTTAACTTCGCTTCCGCTGGCCCATAGTGATCTTTGCAGTGACCCAGGGTGCCATG
ACTCCCGGAATCCCTATCTTTAGTCCAATGATGCCTTATGGCACTGGACTGACCCCACAGCCTATTCAGA
ACACCAATAGTCTGTCTATTTTGGAAGAGCAACAAAGGCAGCAGCAGCAACAACAACAGCAGCAGCAGCA
GCAGCAGCAGCAACAGCAACAGCAGCAGCAGCAGCAGCAGCAGCAGCAGCAGCAGCAGCAGCAGCAGCAG
CAGCAGCAACAGGCAGTGGCAGCTGCAGCCGTTCAGCAGTCAACGTCCCAGCAGGCAACACAGGGAACCT
CAGGCCAGGCACCACAGCTCTTCCACTCACAGACTCTCACAACTGCACCCTTGCCGGGCACCACTCCACT
GTATCCCTCCCCCATGACTCCCATGACCCCCATCACTCCTGCCACGCCAGCTTCGGAGAGTTCTGGGATT
GTACCGCAGCTGCAAAATATTGTATCCACAGTGAATCTTGGTTGTAAACTTGACCTAAAGACCATTGCAC
TTCGTGCCCGAAACGCCGAATATAATCCCAAGCGGTTTGCTGCGGTAATCATGAGGATAAGAGAGCCACG
AACCACGGCACTGATTTTCAGTTCTGGGAAAATGGTGTGCACAGGAGCCAAGAGTGAAGAACAGTCCAGA
CTGGCAGCAAGAAAATATGCTAGAGTTGTACAGAAGTTGGGTTTTCCAGCTAAGTTCTTGGACTTCAAGA
TTCAGAATATGGTGGGGAGCTGTGATGTGAAGTTTCCTATAAGGTTAGAAGGCCTTGTGCTCACCCACCA
ACAATTTAGTAGTTATGAGCCAGAGTTATTTCCTGGTTTAATCTACAGAATGATCAAACCCAGAATTGTT
CTCCTTATTTTTGTTTCTGGAAAAGTTGTATTAACAGGTGCTAAAGTCAGAGCAGAAATTTATGAAGCAT
TTGAAAACATCTACCCTATTCTAAAGGGATTCAGGAAGACGACGTAATGGCTCTCATGTACCCTTGCCTC
CCCCACCCCCTTCTTTTTTTTTTTTTAAACAAATCAGTTTGTTTTGGTACCTTTAAATGGTGGTGTTGTG
AGAAGATGGATGTTGAGTTGCAGGGTGTGGCACCAGGTGATGCCCTTCTGTAAGTGCCCACCGCGGGATG
CCGGGAAGGGGCATTATTTGTGCACTGAGAACACCGCGCAGCGTGACTGTGAGTTGCTCATACCGTGCTG
CTATCTGGGCAGCGCTGCCCATTTATTTATATGTAGATTTTAAACACTGCTGTTGACAAGTTGGTTTGAG
GGAGAAAACTTTAAGTGTTAAAGCCACCTCTATAATTGATTGGACTTTTTAATTTTAATGTTTTTCCCCA
TGAACCACAGTTTTTATATTTCTACCAGAAAAGTAAAAATCTTTTTTAAAAGTGTTGTTTTTCTAATTTA
TAACTCCTAGGGGTTATTTCTGTGCCAGACACATTCCACCTCTCCAGTATTGCAGGACAGAATATATGTG
TTAATGAAAATGAATGGCTGTACATATTTTTTTCTTTCTTCAGAGTACTCTGTACAATAAATGCAGTTTA
TAAAAGTGTTAGATTGTTGTTAAAAAAAAAAAAAAAAAA
SEQ ID NO: 44
- Homo sapiens transforming growth factor,
beta receptor II (70/80 kDa) (TGFBR2),
transcript variant 1, mRNA
GGAGAGGGAGAAGGCTCTCGGGCGGAGAGAGGTCCTGCCCAGCTGTTGGCGAGGAGTTTCCTGTTTCCCC
CGCAGCGCTGAGTTGAAGTTGAGTGAGTCACTCGCGCGCACGGAGCGACGACACCCCCGCGCGTGCACCC
GCTCGGGACAGGAGCCGGACTCCTGTGCAGCTTCCCTCGGCCGCCGGGGGCCTCCCCGCGCCTCGCCGGC
CTCCAGGCCCCCTCCTGGCTGGCGAGCGGGCGCCACATCTGGCCCGCACATCTGCGCTGCCGGCCCGGCG
CGGGGTCCGGAGAGGGCGCGGCGCGGAGGCGCAGCCAGGGGTCCGGGAAGGCGCCGTCCGCTGCGCTGGG
GGCTCGGTCTATGACGAGCAGCGGGGTCTGCCATGGGTCGGGGGCTGCTCAGGGGCCTGTGGCCGCTGCA
CATCGTCCTGTGGACGCGTATCGCCAGCACGATCCCACCGCACGTTCAGAAGTCGGATGTGGAAATGGAG
GCCCAGAAAGATGAAATCATCTGCCCCAGCTGTAATAGGACTGCCCATCCACTGAGACATATTAATAACG
ACATGATAGTCACTGACAACAACGGTGCAGTCAAGTTTCCACAACTGTGTAAATTTTGTGATGTGAGATT
TTCCACCTGTGACAACCAGAAATCCTGCATGAGCAACTGCAGCATCACCTCCATCTCTGAGAAGCCACAG
GAAGTCTGTGTGGCTGTATGGAGAAAGAATGACGAGAACATAACACTAGAGACAGTTTGCCATGACCCCA
AGCTCCCCTACCATGACTTTATTCTGGAAGATGCTGCTTCTCCAAAGTGCATTATGAAGGAAAAAAAAAA
GCCTGGTGAGACTTTCTTCATGTGTTCCTGTAGCTCTGATGAGTGCAATGACAACATCATCTTCTCAGAA
GAATATAACACCAGCAATCCTGACTTGTTGCTAGTCATATTTCAAGTGACAGGCATCAGCCTCCTGCCAC
CACTGGGAGTTGCCATATCTGTCATCATCATCTTCTACTGCTACCGCGTTAACCGGCAGCAGAAGCTGAG
TTCAACCTGGGAAACCGGCAAGACGCGGAAGCTCATGGAGTTCAGCGAGCACTGTGCCATCATCCTGGAA
GATGACCGCTCTGACATCAGCTCCACGTGTGCCAACAACATCAACCACAACACAGAGCTGCTGCCCATTG
AGCTGGACACCCTGGTGGGGAAAGGTCGCTTTGCTGAGGTCTATAAGGCCAAGCTGAAGCAGAACACTTC
AGAGCAGTTTGAGACAGTGGCAGTCAAGATCTTTCCCTATGAGGAGTATGCCTCTTGGAAGACAGAGAAG
GACATCTTCTCAGACATCAATCTGAAGCATGAGAACATACTCCAGTTCCTGACGGCTGAGGAGCGGAAGA
CGGAGTTGGGGAAACAATACTGGCTGATCACCGCCTTCCACGCCAAGGGCAACCTACAGGAGTACCTGAC
GCGGCATGTCATCAGCTGGGAGGACCTGCGCAAGCTGGGCAGCTCCCTCGCCCGGGGGATTGCTCACCTC
CACAGTGATCACACTCCATGTGGGAGGCCCAAGATGCCCATCGTGCACAGGGACCTCAAGAGCTCCAATA
TCCTCGTGAAGAACGACCTAACCTGCTGCCTGTGTGACTTTGGGCTTTCCCTGCGTCTGGACCCTACTCT
GTCTGTGGATGACCTGGCTAACAGTGGGCAGGTGGGAACTGCAAGATACATGGCTCCAGAAGTCCTAGAA
TCCAGGATGAATTTGGAGAATGTTGAGTCCTTCAAGCAGACCGATGTCTACTCCATGGCTCTGGTGCTCT
GGGAAATGACATCTCGCTGTAATGCAGTGGGAGAAGTAAAAGATTATGAGCCTCCATTTGGTTCCAAGGT
GCGGGAGCACCCCTGTGTCGAAAGCATGAAGGACAACGTGTTGAGAGATCGAGGGCGACCAGAAATTCCC
AGCTTCTGGCTCAACCACCAGGGCATCCAGATGGTGTGTGAGACGTTGACTGAGTGCTGGGACCACGACC
CAGAGGCCCGTCTCACAGCCCAGTGTGTGGCAGAACGCTTCAGTGAGCTGGAGCATCTGGACAGGCTCTC
GGGGAGGAGCTGCTCGGAGGAGAAGATTCCTGAAGACGGCTCCCTAAACACTACCAAATAGCTCTTCTGG
GGCAGGCTGGGCCATGTCCAAAGAGGCTGCCCCTCTCACCAAAGAACAGAGGCAGCAGGAAGCTGCCCCT
GAACTGATGCTTCCTGGAAAACCAAGGGGGTCACTCCCCTCCCTGTAAGCTGTGGGGATAAGCAGAAACA
ACAGCAGCAGGGAGTGGGTGACATAGAGCATTCTATGCCTTTGACATTGTCATAGGATAAGCTGTGTTAG
CACTTCCTCAGGAAATGAGATTGATTTTTACAATAGCCAATAACATTTGCACTTTATTAATGCCTGTATA
TAAATATGAATAGCTATGTTTTATATATATATATATATATCTATATATGTCTATAGCTCTATATATATAG
CCATACCTTGAAAAGAGACAAGGAAAAACATCAAATATTCCCAGGAAATTGGTTTTATTGGAGAACTCCA
GAACCAAGCAGAGAAGGAAGGGACCCATGACAGCATTAGCATTTGACAATCACACATGCAGTGGTTCTCT
GACTGTAAAACAGTGAACTTTGCATGAGGAAAGAGGCTCCATGTCTCACAGCCAGCTATGACCACATTGC
ACTTGCTTTTGCAAAATAATCATTCCCTGCCTAGCACTTCTCTTCTGGCCATGGAACTAAGTACAGTGGC
ACTGTTTGAGGACCAGTGTTCCCGGGGTTCCTGTGTGCCCTTATTTCTCCTGGACTTTTCATTTAAGCTC
CAAGCCCCAAATCTGGGGGGCTAGTTTAGAAACTCTCCCTCAACCTAGTTTAGAAACTCTACCCCATCTT
TAATACCTTGAATGTTTTGAACCCCACTTTTTACCTTCATGGGTTGCAGAAAAATCAGAACAGATGTCCC
CATCCATGCGATTGCCCCACCATCTACTAATGAAAAATTGTTCTTTTTTTCATCTTTCCCCTGCACTTAT
GTTACTATTCTCTGCTCCCAGCCTTCATCCTTTTCTAAAAAGGAGCAAATTCTCACTCTAGGCTTTATCG
TGTTTACTTTTTCATTACACTTGACTTGATTTTCTAGTTTTCTATACAAACACCAATGGGTTCCATCTTT
CTGGGCTCCTGATTGCTCAAGCACAGTTTGGCCTGATGAAGAGGATTTCAACTACACAATACTATCATTG
TCAGGACTATGACCTCAGGCACTCTAAACATATGTTTTGTTTGGTCAGCACAGCGTTTCAAAAAGTGAAG
CCACTTTATAAATATTTGGAGATTTTGCAGGAAAATCTGGATCCCCAGGTAAGGATAGCAGATGGTTTTC
AGTTATCTCCAGTCCACGTTCACAAAATGTGAAGGTGTGGAGACACTTACAAAGCTGCCTCACTTCTCAC
TGTAAACATTAGCTCTTTCCACTGCCTACCTGGACCCCAGTCTAGGAATTAAATCTGCACCTAACCAAGG
TCCCTTGTAAGAAATGTCCATTCAAGCAGTCATTCTCTGGGTATATAATATGATTTTGACTACCTTATCT
GGTGTTAAGATTTGAAGTTGGCCTTTTATTGGACTAAAGGGGAACTCCTTTAAGGGTCTCAGTTAGCCCA
AGTTTCTTTTGCTTATATGTTAATAGTTTTACCCTCTGCATTGGAGAGAGGAGTGCTTTACTCCAAGAAG
CTTTCCTCATGGTTACCGTTCTCTCCATCATGCCAGCCTTCTCAACCTTTGCAGAAATTACTAGAGAGGA
TTTGAATGTGGGACACAAAGGTCCCATTTGCAGTTAGAAAATTTGTGTCCACAAGGACAAGAACAAAGTA
TGAGCTTTAAAACTCCATAGGAAACTTGTTAATCAACAAAGAAGTGTTAATGCTGCAAGTAATCTCTTTT
TTAAAACTTTTTGAAGCTACTTATTTTCAGCCAAATAGGAATATTAGAGAGGGACTGGTAGTGAGAATAT
CAGCTCTGTTTGGATGGTGGAAGGTCTCATTTTATTGAGATTTTTAAGATACATGCAAAGGTTTGGAAAT
AGAACCTCTAGGCACCCTCCTCAGTGTGGGTGGGCTGAGAGTTAAAGACAGTGTGGCTGCAGTAGCATAG
AGGCGCCTAGAAATTCCACTTGCACCGTAGGGCATGCTGATACCATCCCAATAGCTGTTGCCCATTGACC
TCTAGTGGTGAGTTTCTAGAATACTGGTCCATTCATGAGATATTCAAGATTCAAGAGTATTCTCACTTCT
GGGTTATCAGCATAAACTGGAATGTAGTGTCAGAGGATACTGTGGCTTGTTTTGTTTATGTTTTTTTTTC
TTATTCAAGAAAAAAGACCAAGGAATAACATTCTGTAGTTCCTAAAAATACTGACTTTTTTCACTACTAT
ACATAAAGGGAAAGTTTTATTCTTTTATGGAACACTTCAGCTGTACTCATGTATTAAAATAGGAATGTGA
ATGCTATATACTCTTTTTATATCAAAAGTCTCAAGCACTTATTTTTATTCTATGCATTGTTTGTCTTTTA
CATAAATAAAATGTTTATTAGATTGAATAAAGCAAAATACTCAGGTGAGCATCCTGCCTCCTGTTCCCAT
TCCTAGTAGCTAAA
SEQ ID NO: 45
- Homo sapiens tumor protein p53 (TP53),
transcript variant 4, mRNA
GATTGGGGTTTTCCCCTCCCATGTGCTCAAGACTGGCGCTAAAAGTTTTGAGCTTCTCAAAAGTCTAGAG
CCACCGTCCAGGGAGCAGGTAGCTGCTGGGCTCCGGGGACACTTTGCGTTCGGGCTGGGAGCGTGCTTTC
CACGACGGTGACACGCTTCCCTGGATTGGCAGCCAGACTGCCTTCCGGGTCACTGCCATGGAGGAGCCGC
AGTCAGATCCTAGCGTCGAGCCCCCTCTGAGTCAGGAAACATTTTCAGACCTATGGAAACTACTTCCTGA
AAACAACGTTCTGTCCCCCTTGCCGTCCCAAGCAATGGATGATTTGATGCTGTCCCCGGACGATATTGAA
CAATGGTTCACTGAAGACCCAGGTCCAGATGAAGCTCCCAGAATGCCAGAGGCTGCTCCCCCCGTGGCCC
CTGCACCAGCAGCTCCTACACCGGCGGCCCCTGCACCAGCCCCCTCCTGGCCCCTGTCATCTTCTGTCCC
TTCCCAGAAAACCTACCAGGGCAGCTACGGTTTCCGTCTGGGCTTCTTGCATTCTGGGACAGCCAAGTCT
GTGACTTGCACGTACTCCCCTGCCCTCAACAAGATGTTTTGCCAACTGGCCAAGACCTGCCCTGTGCAGC
TGTGGGTTGATTCCACACCCCCGCCCGGCACCCGCGTCCGCGCCATGGCCATCTACAAGCAGTCACAGCA
CATGACGGAGGTTGTGAGGCGCTGCCCCCACCATGAGCGCTGCTCAGATAGCGATGGTCTGGCCCCTCCT
CAGCATCTTATCCGAGTGGAAGGAAATTTGCGTGTGGAGTATTTGGATGACAGAAACACTTTTCGACATA
GTGTGGTGGTGCCCTATGAGCCGCCTGAGGTTGGCTCTGACTGTACCACCATCCACTACAACTACATGTG
TAACAGTTCCTGCATGGGCGGCATGAACCGGAGGCCCATCCTCACCATCATCACACTGGAAGACTCCAGT
GGTAATCTACTGGGACGGAACAGCTTTGAGGTGCGTGTTTGTGCCTGTCCTGGGAGAGACCGGCGCACAG
AGGAAGAGAATCTCCGCAAGAAAGGGGAGCCTCACCACGAGCTGCCCCCAGGGAGCACTAAGCGAGCACT
GCCCAACAACACCAGCTCCTCTCCCCAGCCAAAGAAGAAACCACTGGATGGAGAATATTTCACCCTTCAG
ATGCTACTTGACTTACGATGGTGTTACTTCCTGATAAACTCGTCGTAAGTTGAAAATATTATCCGTGGGC
GTGAGCGCTTCGAGATGTTCCGAGAGCTGAATGAGGCCTTGGAACTCAAGGATGCCCAGGCTGGGAAGGA
GCCAGGGGGGAGCAGGGCTCACTCCAGCCACCTGAAGTCCAAAAAGGGTCAGTCTACCTCCCGCCATAAA
AAACTCATGTTCAAGACAGAAGGGCCTGACTCAGACTGACATTCTCCACTTCTTGTTCCCCACTGACAGC
CTCCCACCCCCATCTCTCCCTCCCCTGCCATTTTGGGTTTTGGGTCTTTGAACCCTTGCTTGCAATAGGT
GTGCGTCAGAAGCACCCAGGACTTCCATTTGCTTTGTCCCGGGGCTCCACTGAACAAGTTGGCCTGCACT
GGTGTTTTGTTGTGGGGAGGAGGATGGGGAGTAGGACATACCAGCTTAGATTTTAAGGTTTTTACTGTGA
GGGATGTTTGGGAGATGTAAGAAATGTTCTTGCAGTTAAGGGTTAGTTTACAATCAGCCACATTCTAGGT
AGGGGCCCACTTCACCGTACTAACCAGGGAAGCTGTCCCTCACTGTTGAATTTTCTCTAACTTCAAGGCC
CATATCTGTGAAATGCTGGCATTTGCACCTACCTCACAGAGTGCATTGTGAGGGTTAATGAAATAATGTA
CATCTGGCCTTGAAACCACCTTTTATTACATGGGGTCTAGAACTTGACCCCCTTGAGGGTGCTTGTTCCC
TCTCCCTGTTGGTCGGTGGGTTGGTAGTTTCTACAGTTGGGCAGCTGGTTAGGTAGAGGGAGTTGTCAAG
TCTCTGCTGGCCCAGCCAAACCCTGTCTGACAACCTCTTGGTGAACCTTAGTACCTAAAAGGAAATCTCA
CCCCATCCCACACCCTGGAGGATTTCATCTCTTGTATATGATGATCTGGATCCACCAAGACTTGTTTTAT
GCTCAGGGTCAATTTCTTTTTTCTTTTTTTTTTTTTTTTTTCTTTTTCTTTGAGACTGGGTCTCGCTTTG
TTGCCCAGGCTGGAGTGGAGTGGCGTGATCTTGGCTTACTGCAGCCTTTGCCTCCCCGGCTCGAGCAGTC
CTGCCTCAGCCTCCGGAGTAGCTGGGACCACAGGTTCATGCCACCATGGCCAGCCAACTTTTGCATGTTT
TGTAGAGATGGGGTCTCACAGTGTTGCCCAGGCTGGTCTCAAACTCCTGGGCTCAGGCGATCCACCTGTC
TCAGCCTCCCAGAGTGCTGGGATTACAATTGTGAGCCACCACGTCCAGCTGGAAGGGTCAACATCTTTTA
CATTCTGCAAGCACATCTGCATTTTCACCCCACCCTTCCCCTCCTTCTCCCTTTTTATATCCCATTTTTA
TATCGATCTCTTATTTTACAATAAAACTTTGCTGCCACCTGTGTGTCTGAGGGGTG
SEQ ID NO: 46
- Homo sapiens tumor protein p53 (TP53),
transcript variant 2, mRNA
GATTGGGGTTTTCCCCTCCCATGTGCTCAAGACTGGCGCTAAAAGTTTTGAGCTTCTCAAAAGTCTAGAG
CCACCGTCCAGGGAGCAGGTAGCTGCTGGGCTCCGGGGACACTTTGCGTTCGGGCTGGGAGCGTGCTTTC
CACGACGGTGACACGCTTCCCTGGATTGGCCAGACTGCCTTCCGGGTCACTGCCATGGAGGAGCCGCAGT
CAGATCCTAGCGTCGAGCCCCCTCTGAGTCAGGAAACATTTTCAGACCTATGGAAACTACTTCCTGAAAA
CAACGTTCTGTCCCCCTTGCCGTCCCAAGCAATGGATGATTTGATGCTGTCCCCGGACGATATTGAACAA
TGGTTCACTGAAGACCCAGGTCCAGATGAAGCTCCCAGAATGCCAGAGGCTGCTCCCCCCGTGGCCCCTG
CACCAGCAGCTCCTACACCGGCGGCCCCTGCACCAGCCCCCTCCTGGCCCCTGTCATCTTCTGTCCCTTC
CCAGAAAACCTACCAGGGCAGCTACGGTTTCCGTCTGGGCTTCTTGCATTCTGGGACAGCCAAGTCTGTG
ACTTGCACGTACTCCCCTGCCCTCAACAAGATGTTTTGCCAACTGGCCAAGACCTGCCCTGTGCAGCTGT
GGGTTGATTCCACACCCCCGCCCGGCACCCGCGTCCGCGCCATGGCCATCTACAAGCAGTCACAGCACAT
GACGGAGGTTGTGAGGCGCTGCCCCCACCATGAGCGCTGCTCAGATAGCGATGGTCTGGCCCCTCCTCAG
CATCTTATCCGAGTGGAAGGAAATTTGCGTGTGGAGTATTTGGATGACAGAAACACTTTTCGACATAGTG
TGGTGGTGCCCTATGAGCCGCCTGAGGTTGGCTCTGACTGTACCACCATCCACTACAACTACATGTGTAA
CAGTTCCTGCATGGGCGGCATGAACCGGAGGCCCATCCTCACCATCATCACACTGGAAGACTCCAGTGGT
AATCTACTGGGACGGAACAGCTTTGAGGTGCGTGTTTGTGCCTGTCCTGGGAGAGACCGGCGCACAGAGG
AAGAGAATCTCCGCAAGAAAGGGGAGCCTCACCACGAGCTGCCCCCAGGGAGCACTAAGCGAGCACTGCC
CAACAACACCAGCTCCTCTCCCCAGCCAAAGAAGAAACCACTGGATGGAGAATATTTCACCCTTCAGATC
CGTGGGCGTGAGCGCTTCGAGATGTTCCGAGAGCTGAATGAGGCCTTGGAACTCAAGGATGCCCAGGCTG
GGAAGGAGCCAGGGGGGAGCAGGGCTCACTCCAGCCACCTGAAGTCCAAAAAGGGTCAGTCTACCTCCCG
CCATAAAAAACTCATGTTCAAGACAGAAGGGCCTGACTCAGACTGACATTCTCCACTTCTTGTTCCCCAC
TGACAGCCTCCCACCCCCATCTCTCCCTCCCCTGCCATTTTGGGTTTTGGGTCTTTGAACCCTTGCTTGC
AATAGGTGTGCGTCAGAAGCACCCAGGACTTCCATTTGCTTTGTCCCGGGGCTCCACTGAACAAGTTGGC
CTGCACTGGTGTTTTGTTGTGGGGAGGAGGATGGGGAGTAGGACATACCAGCTTAGATTTTAAGGTTTTT
ACTGTGAGGGATGTTTGGGAGATGTAAGAAATGTTCTTGCAGTTAAGGGTTAGTTTACAATCAGCCACAT
TCTAGGTAGGGGCCCACTTCACCGTACTAACCAGGGAAGCTGTCCCTCACTGTTGAATTTTCTCTAACTT
CAAGGCCCATATCTGTGAAATGCTGGCATTTGCACCTACCTCACAGAGTGCATTGTGAGGGTTAATGAAA
TAATGTACATCTGGCCTTGAAACCACCTTTTATTACATGGGGTCTAGAACTTGACCCCCTTGAGGGTGCT
TGTTCCCTCTCCCTGTTGGTCGGTGGGTTGGTAGTTTCTACAGTTGGGCAGCTGGTTAGGTAGAGGGAGT
TGTCAAGTCTCTGCTGGCCCAGCCAAACCCTGTCTGACAACCTCTTGGTGAACCTTAGTACCTAAAAGGA
AATCTCACCCCATCCCACACCCTGGAGGATTTCATCTCTTGTATATGATGATCTGGATCCACCAAGACTT
GTTTTATGCTCAGGGTCAATTTCTTTTTTCTTTTTTTTTTTTTTTTTTCTTTTTCTTTGAGACTGGGTCT
CGCTTTGTTGCCCAGGCTGGAGTGGAGTGGCGTGATCTTGGCTTACTGCAGCCTTTGCCTCCCCGGCTCG
AGCAGTCCTGCCTCAGCCTCCGGAGTAGCTGGGACCACAGGTTCATGCCACCATGGCCAGCCAACTTTTG
CATGTTTTGTAGAGATGGGGTCTCACAGTGTTGCCCAGGCTGGTCTCAAACTCCTGGGCTCAGGCGATCC
ACCTGTCTCAGCCTCCCAGAGTGCTGGGATTACAATTGTGAGCCACCACGTCCAGCTGGAAGGGTCAACA
TCTTTTACATTCTGCAAGCACATCTGCATTTTCACCCCACCCTTCCCCTCCTTCTCCCTTTTTATATCCC
ATTTTTATATCGATCTCTTATTTTACAATAAAACTTTGCTGCCACCTGTGTGTCTGAGGGGTG
SEQ ID NO: 47
- Homo sapiens TXK tyrosine kinase (TXK), mRNA
GATTTCAGTTGAAAGATGTGTTTTTGTGAGTAGAGCACCGCAGAAGAACTGAAGACTGTTGTGTGCTCCC
CGCAGAAGGGGCTACCATGATCCTTTCCTCCTATAACACCATCCAGTCGGTTTTCTGTTGCTGCTGTTGC
TGTTCAGTGCAGAAGCGACAAATGAGAACACAGATAAGCCTGAGCACAGATGAAGAGCTTCCAGAAAAAT
ACACCCAGCGTCGCAGGCCGTGGCTCAGCCAATTGTCAAATAAGAAGCAATCCAACACGGGCCGTGTGCA
GCCGTCAAAACGAAAGCCACTGCCTCCCCTCCCACCCTCTGAGGTTGCTGAAGAGAAGATCCAAGTCAAG
GCACTTTATGATTTTCTGCCCAGAGAACCCTGTAATTTAGCCTTAAGGAGAGCAGAAGAATACCTGATAC
TGGAGAAATACAATCCTCACTGGTGGAAGGCAAGAGACCGTTTGGGGAATGAAGGCTTAATCCCAAGCAA
CTATGTGACTGAAAACAAAATAACTAATTTAGAAATATATGAGTGGTACCATAGAAACATTACCAGAAAT
CAGGCAGAACATCTATTGAGACAAGAGTCTAAAGAAGGTGCATTTATTGTCAGAGATTCAAGACATTTAG
GATCCTACACAATTTCCGTATTTATGGGAGCTAGAAGAAGTACGGAGGCTGCCATAAAACATTATCAGAT
AAAAAAGAATGACTCAGGACAGTGGTATGTGGCTGAAAGACACGCCTTTCAATCAATCCCTGAGTTAATC
TGGTATCACCAGCACAATGCAGCCGGTCTCATGACTCGTCTCCGATATCCAGTTGGGCTGATGGGCAGTT
GTTTACCAGCCACAGCTGGGTTTAGCTACGAAAAGTGGGAGATAGATCCATCTGAGTTGGCTTTTATAAA
GGAGATTGGAAGCGGTCAGTTTGGAGTGGTCCATTTAGGTGAATGGCGGTCACATATCCAGGTAGCTATC
AAGGCCATCAATGAAGGCTCCATGTCTGAAGAGGATTTCATTGAAGAGGCCAAAGTGATGATGAAATTAT
CTCATTCAAAGCTAGTGCAACTTTATGGAGTCTGTATACAGCGGAAGCCCCTTTACATTGTGACAGAGTT
CATGGAAAATGGCTGCCTGCTTAACTATCTCAGGGAGAATAAAGGAAAGCTTAGGAAGGAAATGCTACTG
AGTGTATGCCAGGATATATGTGAAGGAATGGAATATCTGGAGAGGAATGGCTATATTCATAGGGATTTGG
CGGCAAGGAATTGTTTGGTCAGTTCAACATGCATAGTAAAAATTTCAGACTTTGGAATGACAAGGTACGT
TTTGGATGATGAGTATGTCAGTTCTTTTGGAGCCAAGTTCCCAATCAAGTGGTCCCCTCCTGAAGTTTTT
CTTTTCAATAAGTACAGCAGTAAATCTGATGTCTGGTCATTTGGAGTTTTAATGTGGGAAGTTTTTACAG
AAGGAAAAATGCCTTTTGAAAATAAGTCAAATTTGCAAGTCGTGGAAGCTATTTCTGAAGGCTTCAGGCT
ATATCGCCCTCACCTGGCACCAATGTCCATATATGAAGTCATGTACAGCTGCTGGCATGAGAAACCTGAA
GGCCGCCCTACATTTGCCGAGCTGCTGCGGGCTGTCACAGAGATTGCGGAAACCTGGTGACCGGAAACAG
AATGCCAACCCAAAGAGTCATCTTGCAAAACTGTCATTTATTGTGAATATCTTCACCATATGGGGTCACT
TATGGTGAATATCTTTCTTCAGAGTTGCTGACTCTTGAAAACAGTGCAAAGATCACAGTTTTTAAAAGTT
TTAAAAATTTAAGAATATTCACACAATCGTTTTTCTATGTGTGAGAGGGATTTGCACACTCTTATTTTTC
TGTAAAATATTTCACATCCCAAATGTGAAGAAGTGAAAAAGACTTCGCAGCAGTCTTCATTGTGGTGCTC
TTCATGATCATAGCCCCAGGAACCCTTGAGGTTCTTCTTCACAAGGCTGAGAGTGCTTCCTTCTTGAAGA
CGAGTGACATTCATCACTTCAGTGATCCATGCATAGAATATGAAAATAAATTCTTCCAACTCATGGGATA
AAGGGGACTCCCTTGAAGAATTTCATGTTTTTGGGCTGTATAGCTCTTTACAGAAAATGCACCTTTATAA
ATCACATGAATGTTAGTATTCTGGAAATGTCTTTTGTTAATATAATCTTCCCATGTTATTTAACAAATTG
TTTTTGCACATATCTGATTATATTGAAAGCAGTTTTTTGCATTCGAGTTTTAAACACTGTTATAAAATGT
AGCCAAAGCTCACCTTTGAACAGATCCCGGTGACATTCTATTTCCAGGAAAATCCGGAACCTGATTTTAG
TTCTGTGATTTTACACTTTTTACATGTGAGATTGGACAGTTTCAGAGGCCTTATTTTGTCATACTAAGTG
TCTCCTGTAATTTTCAGGAAGATGATTTGTTCTTTCCAGAAGAGGAGACAAAAGCAAGATAGCCAAATGT
GACATCAAGCTCCATTGTTTCGGAAATCCAGGATTTTGAATTCGAGATGAAACAACCAGCAATCACAGTT
AAATCTTAACTTTGCCTGCACTCTTTGTAGGAATGATCAGAAATTTATCTTTATCATTCTGAGTGCTTCA
GGAGTACAATAGGAAGAAAGATACTGGAGAAAGCACTAATGTAATCACCATGAAGTCTGACAACAGGAGC
CCATTATTTGCGTACTGTCCCACCCTGTATCATGGTTCTCTGGGAACAAGCTTTATGATTCTCATTAGAG
TTTATTTGTTGATTGTCAGTAGTTGCGACTTTTAAATTATATTTCCCCCACTCAAAGAATGGTATCTTTA
TATATCAATGACATTCAATAAATGTGTATTATTTCTAATGAGAA

Throughout this document, various references are mentioned. All such references are incorporated herein by reference, including the references set forth in the following list:

### REFERENCES

1. Swanton J K, Rovira A, Tintore M, Altmann D R, Barkhof F, Filippi M et al. MRI criteria for multiple sclerosis in patients presenting with clinically isolated syndromes: a multicentre retrospective study. Lancet Neurol 2007; 6(8): 664-665.
2. Polman C H, Reingold S C, Edan G, Filippi M, Hartung H P, Kappos L et al. Diagnostic criteria for multiple sclerosis: 2005 revisions to the “McDonald Criteria”. Ann Neurol. 2005; 58(6): 840-846.
3. McDonald W I, Compston A, Edan G, Goodkin D, Hartung H P, Lublin F D et al. Recommended diagnostic criteria for multiple sclerosis: guidelines from the International Panel on the diagnosis of multiple sclerosis. Ann Neurol. 2001; 50(1): 121-127.
4. Awad A, Hemmer B, Hartung H P, Kieseier B, Bennett J L, Stuve O. Analyses of cerebrospinal fluid in the diagnosis and monitoring of multiple sclerosis. J Neuroimmunol. 2009; 219(1-2): 1-7.
5. LInk H, Huang Y-M. Oligoclonal bands in multile sclerosis cerebrospinal fluid: An update on methodology and clinical usefulness. Journal of Neuroimmunology 2006; 180(1-2): 17-28.
6. Consortium TWTCC. Genome-wide association study of 14,000 cases of seven common diseases and 3,000 shared controls. Nature 2007; 447(7145): 661-78.
7. Axtell R C, de Jong B A, Boniface K, van der Voort L F, Bhat R, De Sarno P et al. T helper type 1 and 17 cells determine efficacy of interferon-beta in multiple sclerosis and experimental encephalomyelitis. Nat Med. 2010; 16(4): 406-412.
8. Keller A, Leidinger P, Lange J, Borries A, Schroers H, Scheffler M et al. Multiple sclerosis: microRNA expression profiles accurately differentiate patients with relapsing-remitting disease from healthy controls. PLoS One. 2009; 13(10): e7440.
9. Harris V K, Sadiq S A. Disease biomarkers in multiple sclerosis: potential for use in therapeutic decision making. Mol. Diagn. Ther. 2009; 13(4): 225-244.
10. Quintana F J, Farez M F, Viglietta V, Iglesias A H, Merbl Y, Izquierdo G et al. Antigen microarrays identify unique serum autoantibody signatures in clinical and pathologic subtypes of multiple sclerosis. Proc Natl Acad Sci USA. 2008; 105(48): 18889-18894.
11. Kostka D, Spang R. Microarray Based Diagnosis Profits from Better Documentation of Gene Expression Signatures. PLoS Comput Biol 2008; 4(2): e22.
12. Ray S, Britschgi M, Herbert C, Takeda-Uchimura Y, Boxer A, Blennow K et al. Classification and prediction of clinical Alzheimer's diagnosis based on plasma signaling proteins. Nature Med. 2007; 13(11): 1359-1362.
13. Quackenbush J. Microarray Analysis and Tumor Classification. N. Engl. J. Med. 2006; 354(23): 2463-2472.
14. Hofman P. DNA Microarrays. Nephron Physiol 2005; 99(3): 85-89.
15. Gregersen P K, Brehrens T W. Fine mapping the phenotype in autoimmune disease: the promise and pitfalls of DNA microarray technologies. Genes and Immunity 2003; 4(3): 175-176.
16. Bomprezzi R, Ringnér M, Kim S, Bittner M L, Khan J, Chen Y et al. Gene expression profile in multiple sclerosis patients and healthy controls: identifying pathways relevant to disease. Hum Mol Genet. 2003; 12(17): 2191-2199.
17. Brynedal B, Khademi M, Wallstrom E, Hillert J, Olsson T, Duvefelt K. Gene expression profiling in multiple sclerosis: A disease of the central nervous system, but with relapses triggered in the periphery?. Neurobiology of Disease 2010 37(3): 613-621
18. Harris V K, Sadiq S A. Disease biomarkers: Potential for use in therapeutic decision making. Mol. Diagn. Ther. 2009; 13(4): 225-244.
19. Maas K, Chan S, Parker J, Slater A, Moore J, Olsen N et al. Cutting edge: molecular portrait of human autoimmune disease. J Immunol. 2002; 169(1): 5-9.
20. Liu Z, Maas K, Aune T M. Identification of gene expression signatures in autoimmune disease without the influence of familial resemblance. Hum Mol Genet. 2006; 15(3): 501-509.
21. Maas K, Chen H, Shyr Y, Olsen N J, Aune T. Shared gene expression profiles in individuals with autoimmune disease and unaffected first-degree relatives of individuals with autoimmune disease. Hum Mol Genet. 2005; 14(10): 1305-1314.
22. Fossey S C, Vnencak-Jones C L, Olsen N J, Sriram S, Garrison G, Deng X et al. Identification of molecular biomarkers for multiple sclerosis. J Mol Diagn. 2007; 9(2): 197-204.
23. Weyand C M, Fujii H, Shao L, J. G J. Rejuvenating the immune system in rheumatoid arthritis. Nat Rev Rheumatol. 2009; 5(10): 583-588.
24. Shao L, Fujii H, Colmegna I, Oishi H, Goronzy J J, Weyand C M. Deficiency of the DNA repair enzyme ATM in rheumatoid arthritis. J Exp Med. 2009; 206(6): 1435-1449.
25. Deng X, Ljunggren-Rose A, Maas K, Sriram S. Defective ATM-p53-mediated apoptotic pathway in multiple sclerosis. Ann Neurol. 2005; 58(4): 577-584.
26. Maas K, Westfall M, Pietenpol J, Olsen N J, Aune T. Reduced p53 in peripheral blood mononuclear cells from patients with rheumatoid arthritis is associated with loss of radiation-induced apoptosis. Arthritis Rheum. 2005; 52(4): 1047-1057.
27. Butowski N. Immunostimulants for malignant gliomas. Neurosurg Clin N Am. 2010; 21(1): 53-65.
28. Readinger J A, Mueller K L, Venegas A M, Horai R, Schwartzberg P L. Tec kinases regulate T-lymphocyte development and function: new insights into the roles of Itk and Rlk/Txk. Immunol Rev. 2009; 228(1): 93-114.
29. Buchner D A, Meisler M H. TSRC1, a widely expressed gene containing seven thrombospondin type I repeats. Gene 2003; 307: 23-30.
30. Abdi H. The Bonferonni and Sidak corrections for multiple comparisons, Sage: Thousand Oaks, Calif., 2007.
31. Chiappa K H, Ropper A H. Evoked potentials in clinical medicine (second of two parts). N Engl J Med 1982; 306(20): 1205-11.
32. Chiappa K H, Ropper A H. Evoked potentials in clinical medicine (first of two parts). N Engl J Med 1982; 306(19): 1140-50.
33. O'Riordan J I, Thompson A J, Kingsley D P, MacManus D G, Kendall B E, Rudge P et al. The prognostic value of brain MRI in clinically isolated syndromes of the CNS. A 10-year follow-up. Brain 1998; 121 (Pt 3): 495-503.
34. Comi G, Martinelli V, Rodegher M, Moiola L, Bajenaru O, Carra A et al. Effect of glatiramer acetate on conversion to clinically definite multiple sclerosis in patients with clinically isolated syndrome (PreCISe study): a randomised, double-blind, placebo-controlled trial. Lancet 2009; 374(9700): 1503-11.
35. Jacobson D L, Gange S J, Rose N R, Graham N M. Epidemiology and estimated population burden of selected autoimmune diseases in the United States. Clin Immunol Immunopathol 1997; 84(3): 223-43.
36. Comi G, Filippi M, Barkhof F, Durelli L, Edan G, Fernandez O et al. Effect of early interferon treatment on conversion to definite multiple sclerosis: a randomised study. Lancet 2001; 357(9268): 1576-82.
37. Koch M W, Mostert J P, de Vries J J, De Keyser J. Treatment with interferon beta-lb delays conversion to clinically definite and McDonald MS in patients with clinically isolated syndromes. Neurology 2007; 68(14): 1163; author reply 1163-4.
38. Kappos L, Polman C H, Freedman M S, Edan G, Hartung H P, Miller D H et al. Treatment with interferon beta-lb delays conversion to clinically definite and McDonald MS in patients with clinically isolated syndromes. Neurology 2006; 67(7): 1242-9.
39. Dalton C M, Brex P A, Miszkiel K A, Hickman S J, MacManus D G, Plant G T et al. Application of the new McDonald criteria to patients with clinically isolated syndromes suggestive of multiple sclerosis. Ann Neurol 2002; 52(1): 47-53.
40. Tossberg J T, Crooke P S, Henderson M A, Sriram S, Mrelashvili D, Chitnis S et al. Gene-expression signatures: biomarkers toward diagnosing multiple sclerosis. Genes and Immunity 2012; 13(2): 146-54.
41. Tijssen P (1993) Laboratory Techniques in Biochemistry and Molecular Biology-Hybridization with Nucleic Acid Probes. Elsevier, N.Y.

1. A method for characterizing multiple sclerosis in a subject, comprising:
(a) providing a biological sample from the subject;
(b) determining expression levels of at least two genes in the biological sample;
(c) calculating one or more ratios of the expression levels of the at least two genes; and
(d) comparing each ratios to a reference, wherein the multiple sclerosis is characterized based on a difference in the ratios of the expression values of the at least two genes in the biological sample from the subject as compared to the references.

2. The method of claim 1, wherein the determining is of the expression levels of at least two genes represented by SEQ ID NOs: 1-47.

3. (canceled)

4. The method of claim 1, wherein the determining is of the expression levels of at least two genes corresponding to those set forth in Table A.

5. (canceled)

6. The method of claim 1, wherein the determining is of the expression levels of the genes corresponding to CD55, FOS, JUN, PMAIP1, SPIB, TAF11, and TBP.

7. The method of claim 1, wherein the determining is of the expression levels of the genes corresponding to ACTB, CDKN1B, CTSS, GAPDH-1, KRAS, PGK1, and TBP.

8. The method of claim 1, wherein the determining is of the expression levels of at least 2 genes corresponding to those set forth in Table B.

9. The method claim 1, wherein the one or more ratios are ratios of expression levels of genes corresponding to those set forth in Table A, wherein each ratio is calculated by dividing the expression level of a first gene in Table A by the expression level of a second gene in Table A.

10. The method of claim 1, wherein the one or more ratios are ratios are selected from those set forth in Table B.

11-15. (canceled)

16. The method of claim 1, wherein the reference is a reference ratio of a comparator group, a standard reference ratio, or a healthy control.

17. (canceled)

18. The method of claim 1, and further comprising comparing each ratio to a second reference.

19. (canceled)

20. The method of claim 18, wherein the second reference is not a healthy control or wherein the second reference comprises other neurologic disorders (OND).

21-22. (canceled)

23. The method of claim 1, wherein the characterization comprises diagnosing or prognosticating MS.

24. The method of claim 1, wherein MS is predicted.

25. (canceled)

26. The method of claim 1, wherein the characterization comprises an exclusion of a diagnosis of MS.

27. The method of claim 1, and further comprising providing a series of biological sample obtained from the subject; and determining a presence of any change in the ratios in each of the biological samples from the series.

28-32. (canceled)

33. The method of claim 1, wherein the determining comprises a technique selected from the group consisting of a reverse transcription-polymerase chain reaction (RT-PCR), hybridization to nucleotide probes, and a Northern blot.

34-39. (canceled)

40. A kit, comprising primer pairs for determining expression levels of at least two genes in a biological sample, wherein the at least two genes are represented by SEQ ID NOs: 1-47 or are set forth in Table A.

41-44. (canceled)

45. The kit of claim 40, comprising primer pairs for determining expression levels of the genes corresponding to CD55, FOS, JUN, PMAIP1, SPIB, TAF11, and TBP; or comprising primer pairs for determining expression levels of the genes corresponding to ACTB, CDKN1B, CTSS, GAPDH-1, KRAS, PGK1, and TBP.

46-47. (canceled)

48. A device, comprising probes for detecting at least two genes in a biological sample, wherein the at least two genes are represented by SEQ ID NOs: 1-47 or are set forth in Table A.

49-52. (canceled)

53. The device of claim 48, comprising probes for detecting each of the genes corresponding to CD55, FOS, JUN, PMAIP1, SPIB, TAF11, and TBP; or comprising probes for detecting each of the genes corresponding to ACTB, CDKN1B, CTSS, GAPDH-1, KRAS, PGK1, and TBP.

54-55. (canceled)


SEQUENCE LISTING (47 sequences)

SEQ ID NO: 1 - DNA, 5603 bp, Homo sapiens
ggactgcagagggaacttgccttgaagaggcctggtccttaaagagacacagcacacacggcccgaccggcagccccagagcagaggctccactgatggcaggcgcccctggctaggctctgaggttcctttgccctcgccttgctgaatggtgagccgctgcctctcggagcccgtctccttgacagcctgccctcggctcctgcagccactcctgggcctgatggggacagggccagcctggtgggtggtgtcagaggtcctggcagagcagcgtaggcctgggatgcgtctgcagaattctggctgaacgagcgaggagcacggccagcttcggggccgtcgtgaccacaggagggcagagggccagcccgtgagctctgaccccagctggacgtgctcttgtttcccttggggctaaggagattggagccactgaactgaatctctgggttttggagacttagagaatccattggactcttctgctggcgtctttctgaatgctgatggggacttggtgacttcagctacgggacggacgagtacgacggagaggggaatgaggagcagaaggggcccccggagggctcagagaccatgccgtacatcgatgagtcgcccaccatgtccccgcagctcagcgcccgcagccagggcgggggggatggcgtctccccgactccacctgagggactggctcctggggtggaagcagggaaaggcctggagatgaggaagctggttctctcggggttcttggccagcgaagagatctacattaaccagctggaagccctgttgctgcccatgaaacccctgaaggccaccgccaccacctcccagcccgtgctcaccatccagcagatcgagaccatcttctacaagatccaggacatctatgagatccacaaggagttctatgacaacctgtgccccaaggtgcaacagtgggacagccaggtcaccatgggccacctcttccagaagctggccagccagctcggtgtgtacaaagcgtttgtcgataactataaagtcgctctggagacagctgagaagtgcagccagtccaacaaccagttccagaagatctcagaggaactcaaagtgaaaggtcccaaggactccaaggacagccacacgtctgtcaccatggaagctctgctctacaagcccattgaccgggtcactcggagcaccctagtcctacacgacctgctgaagcacacacctgtggaccaccccgactacccgctgctgcaggatgccctccgcatctcccagaacttcctgtccagcatcaacgaggacatcgacccccgccggactgcagtgacaacgcccaagggggagacgcgacagctggtgaaggacggcttcctggtggaagtgtcagagagctcccggaagctgcggcacgtcttcctctttacagatgtcctactgtgtgccaagctgaagaagacctctgcagggaagcaccagcagtatgactgtaagtggtacatccccctggccgacctggtgtttccatcccccgaggagtctgaggccagcccccaggtgcaccccttcccagaccatgagctggaggacatgaagatgaagatctctgccctcaagagtgaaatccagaaggagaaagccaacaaaggccagagccgggccatcgagcgcctgaagaagaagatgtttgagaatgagttcctgctgctgctcaactcccccacaatcccgttcaggatccacaatcggaatggaaagagttacctgttcctactgtcctcggactacgagaggtcagagtggagagaagcaattcagaaactacagaagaaggatctccaggcctttgtcctgagctcagtggagctccaggtgctcacaggatcctgtttcaagcttaggactgtacacaacattcctgtcaccagcaataaagacgacgatgagtctccaggactctatggcttccttcatgtcatcgtccactctgccaagggatttaagcaatcagccaacctgtactgtaccctggaggtggattccttcggctattttgtcagcaaagccaaaaccagggtgttccgggacacagcggagcccaagtgggatgaggagtttgagatcgagctggagggctcccagtccctgaggatcctgtgctatgagaagtgctatgacaagaccaaggtcaacaaggacaacaatgagatcgtggacaagatcatgggcaaaggacagatccagctggacccacaaaccgtggagaccaagaactggcacacggacgtgattgagatgaacgggatcaaagtggaattttccatgaaattcaccagccgagatatgagcctgaagaggaccccgtccaaaaagcagaccggcgtcttcggtgtgaagatcagcgtggtgacgaagcgggagcgctccaaggtgccctacatcgtccggcagtgtgtggaggaggtggagaagaggggtatcgaggaggttggcatctacaggatatcgggcgtggccacggacatccaggcgctcaaggccgtcttcgatgccaataacaaggacatcctgctgatgctgagtgacatggacatcaacgccatcgccgggacgctcaagctgtacttccgggaactgcccgagccgctcctcacggaccgactctacccagccttcatggagggcatcgccctgtcagaccctgctgccaaggaaaactgcatgatgcacctgctccgctccctgcccgaccccaacctcatcaccttcctcttcctgctggaacacttgaaaagggttgccgagaaggagcccatcaacaaaatgtcacttcacaacctggctaccgtgtttggacccacgttactgagaccctcagaagtggagagcaaagcacacctcacctcggctgcggacatctggtcccatgacgtcatggcgcaggtccaggtcctcctctactacctgcagcacccccccatttccttcgcagaactcaagcggaacacactgtacttctccaccgacgtgtagcccgaggcagggtggctgcgggcgggtggtggaaccagcccctccagcctggggtccaactcagacttgaaagactgcaatagaaaactcccaaacccagcactccagactcgagggaagccagcttccaagaactggaatgcgtacgtcttttgtgccaccttgtacaaagccggctgcccagccccagcctcaccaccgcatcccacctcctgccctccatacctctagttgtgtctgatgctccgtgctgttcgggaattgttttatgtacacttgtcaggcagaaaaggtagtgaccggcccggcgtgggcacacagacagcccgctttgttctttcatttcctccagcactttctttccgcctgagtccagcccaaggccttttattttgcgctgtgtaactgctgccagcttctctcttggccctgctcccagatggcggtctcctggcagcctcccctcagtcttcctccacccgctcttccttcccagcctgcctgcatgcatgtgcacccttggtcttcgctccatcgccttgaaagctctgaagaggccctgggttgccgcggcagcagtggtctgtttgatgctgccgtttgccgctgccggcccctcctcagactccgcctttgggagcacacctgctttgccttgctgcctgtgcaaatgttggacaagcagacacactcacactcgtccccagcttagcacagagctggagcgcccatttctggaattttccgtttgggaatctccacttctggggtttacctgttcggcctcctgtctatcagtgaggcatctctgactgtttcttctactgcttttcagttcccttccctgctgttctatttcctttgagtgtaaagactcacaggtgacctgctatcgagatagccagagggtcaggagagaatgggggaggaggcggtcaggctgctgaggaaacaccacaggctgaacgggggaggaatgcacatgccacgctgggtgtcccgggtcgcggggaggcagctcagctcttaggagcaagttgtgggggcttttcaagaggggccaggcttcctggagggtgactgatgtggccgaagcaggtgtccaggcaggtaggctgcagccaggagctccctggcaccgcaggacctcgtggtactcttgccttagattttacacacactccacagccaagcactgccacggtcctccaggacctgggaagcaaaggcacaggcccacggtggccagccattgtggtgccgccccagcttctggatacagccttttgggtaaacactgggaactccagaagttgtggggagagtggggaatcagacagccgcctctaggggctgggttctgctggggcctccttgttggtgctgtaggcacccgccagggagcagggacccgacttgcagacgcattgcccggtactaggaaggagtgaggtgtgttcccaccgtacacttcccacacgagctgcggctgccagcctcgggccatcagcctaggagagcagatgcagctccaggggctcgacttatagccagttacagctccccggctcttctgtgtggcagagcgtcgtttccgggccctcagggctggggagctcagttcccattgcttgtgctcagggctgagtcttaaagaagggtttgccggccctaacgctgcagcgcgtgcgcggtgagaggccctttttgagcctgtttactcctgtggccttgggcagaacagtaaatactctgtgcacggaggaaagacatgcccaagaggaaggaagtactgaccatcggctgcctgtgagcagcttagcaaggagcccttgctccctgggaaaggcggtgaacttgagtctaaagatgcagtgcctggcccttcctaaggtccctgcctggcatccgagtgtcggtgtgtggcacagaaggctcctgcttgcttccaaagtgatggacaggaaggggcagagtgagtcacggcccagactgggcaccttcgcgtctcagcctcagggagccccacagccccaagctcgctgaggcaacgtgagaacaggctatgggaaggctgcaaaggctgagaaatgcaaaggctcatatttataaatcccacccccagagtggggagggtcaggtgccagacctggactaaactgcaccaaggaaacacccagcagggtctcctgtgagccggggaccatgcagcccgaaacctccagtcactgcgcccggcaggagtcaggagccagggactgtgcagcctggaacctccagtcactgtgcccagcagggtgggctgtgcccagcaggagtcaggctaagaaacgccaggtctgcctgttcttgctgggcaatggctgatggctgccagtttctgctgatacacaggtaggatgggacccttcatgaatatctgactttaataagttggtaaggatatatttttttgtctatgttctgtttcaacttatgtagattattataaattgatgtaaaccacgtgagaggaaaatgttaataaaaaatgcaaagccccatcatttgcacaaaactca

SEQ ID NO: 2 - DNA, 1852 bp, homo sapiens
accgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaaaaaaaaaaaaaaaaaaaaaaaaaaaaaaaaaaaaaaaaa

SEQ ID NO: 3 - DNA, 2891 bp, Homo sapiens
gctccctcgccgccctgaaccggcggctagactgcgcatgcgtgtcagtggcgctagcggcggacccggctgggcagttccttccccagaaggagagattcctctgccatggagtcctacgatgtgatcgccaaccagcctgtcgtgatcgacaacggatccggtgtgattaaagctggttttgctggtgatcagatccccaaatactgctttccaaactatgtgggccgacccaagcacgttcgtgtcatggcaggagcccttgaaggcgacatcttcattggccccaaagctgaggagcaccgagggctgctttcaatccgctatcccatggagcatggcatcgtcaaggattggaacgacatggaacgcatttggcaatatgtctattctaaggaccagctgcagactttctcagaggagcatcctgtgctcctgactgaggcgcctttaaacccacgaaaaaaccgggaacgagctgccgaagttttcttcgagaccttcaatgtgcccgctcttttcatctccatgcaagctgtactcagcctttacgctacaggcaggaccacaggggtggtgctggattctggggatggagtcacccatgctgtgcccatctatgagggctttgccatgccccactccatcatgcgcatcgacatcgcgggccgggacgtctctcgcttcctgcgcctctacctgcgtaaggagggctacgacttccactcatcctctgagtttgagattgtcaaggccataaaagaaagagcctgttacctatccataaacccccaaaaggatgagacgctagagacagagaaagctcagtactacctgcctgatggcagcaccattgagattggtccttcccgattccgggcccctgagttgctcttcaggccagatttgattggagaggagagtgaaggcatccacgaggtcctggtgttcgccattcagaagtcagacatggacctgcggcgcacgcttttctctaacattgtcctctcaggaggctctaccctgttcaaaggttttggtgacaggctcctgagtgaagtgaagaaactagctccaaaagatgtgaagatcaggatatctgcacctcaggagagactgtattccacgtggattgggggctccatccttgcctccctggacacctttaagaagatgtgggtctccaaaaaggaatatgaggaagacggtgcccgatccatccacagaaaaaccttctaatgtcgggacatcatcttcacctctctctgaagttaactccactttaaaactcgctttcttgagtcggagtgtttgcgaggaactgcctgtgtgtgagtgcgtgtgtggatatgagtgtgtgtgcacatgcgagtgccgtgtggccctgggaccctgggcccagaaaggacgatgaactacctgcagtggtgatggcctgaggcctggggttgaccactaactggctcctgacagggaagagcgctggcagaggctgtgctccctcctcaggtggcctctggctggctgtgggggactccgtttactaccacagggagacagagggaggtaagccatcccccgggagaccttgctgctgaccatcctaggctgggctggccccaccctcacccccacccccagggtgccctgaggccccaggcagctgctgcctccactatcgatgcctcctgactgcacactgaggactgggactggggttgagttctgtctggttttgttgccattttggtttgggaggctggaaaagcaccccaagagctattacagagactggagtcaggagagagcaggaggccctcatgttcaccagggaacaggaccacaccggccactggaggagggcaggagcagtcctcactctgaatggctgcagagttaatgttcccagcccagtcccctttcgggggccttgggagagtttaaggcacctgctggttccaggacctcgctttccatctgttcttgttgcaatgccatcttcaaaccgttttatttattgaagtgtttgttcagttaggggctggagagagggagcttgctgcctcctgccttgctacactaatgtttacagcacctaagcttagcctccagggccccacctctcccagctgatggtgagctgacagtgtccacaggttccaggaccatttgagattggaagctacactcaaagacactcccaccaggctctttctcccttttcctcttgctcactgccctggaatcaacaggctggttgctggttagattttctgaaacaggaggtaaaatttttctttggcagaggcccctaagcaagggaggggtgttggagagccagtgcccttaagactggagaaagctgcaatttaccaagttgccttttgccactgtagctgaccaggggactaggttgtagaggtgggaaggccccctctgggctgatcttgtgccattcttgaccttggacctgcttggttaaggagggagtgggccagaccagagtgccaggagctaatggagccaggcctgactcctaggagtggtccaaaggccttcagcctagatggtgcaaagctggggccagcctgtcttcaccggcaccctcacctgtgacaccaagacccaccccaatcccagacttcacacagtattctcccccacgccgtcctatgaccaaaggcccctgccaggtgtgggccacagcagcaggtatgtgtgaaagcaacgtagcgccccgcggactgcagtgcgcttaaccaactcacctcccttctcttagcccaagcctgtccctcgcacagcctcgcacaaaccacattgcctggtggggcccagtgtactgaaataaagtcgttccgatagacacgtcaaaaaaaaaaaaaaaaaaa

SEQ ID NO: 4 - DNA, 4209 bp, Homo sapiens
ccgccgcggagcgaggttgcctggagagagcgcctgggcgcagaagggttaacgggccaccgggggctcgcagagcaggagggtgctctcggacggtgtgtcccccactgcactcctgaacttggaggacagggtcgccgcgagggacgcagagagcaccctccacgcccagatgcctgcgtagtttttgtgaccagtccgctcctgcctccccctggggcagtagagggggagcgatggagaactggactggcaggccctggctgtatctgctgctgcttctgtccctccctcagctctgcttggatcaggaggtgttgtccggacactctcttcagacacctacagaggagggccagggccccgaaggtgtctggggaccttgggtccagtgggcctcttgctcccagccctgcggggtgggggtgcagcgcaggagccggacatgtcagctccctacagtgcagctccacccgagtctgcccctccctccccggcccccaagacatccagaagccctcctcccccggggccagggtcccagaccccagacttctccagaaaccctccccttgtacaggacacagtctcggggaaggggtggcccacttcgaggtcccgcttcccacctagggagagaggagacccaggagattcgagcggccaggaggtcccggcttcgagaccccatcaagccaggaatgttcggttatgggagagtgccctttgcattgccactgcaccggaaccgcaggcaccctcggagcccacccagatctgagctgtccctgatctcttctagaggggaagaggctattccgtcccctactccaagagcagagccattctccgcaaacggcagcccccaaactgagctccctcccacagaactgtctgtccacaccccatccccccaagcagaacctctaagccctgaaactgctcagacagaggtggcccccagaaccaggcctgcccccctacggcatcaccccagagcccaggcctctggcacagagcccccctcacccacgcactccttaggagaaggtggcttcttccgtgcatcccctcagccacgaaggccaagttcccagggttgggccagtccccaggtagcagggagacgccctgatccttttccttcggtccctcggggccgaggccagcagggccaagggccttggggaacgggggggactcctcacgggccccgcctggagcctgaccctcagcacccgggcgcctggctgcccctgctgagcaacggcccccatgccagctccctctggagcctctttgctcccagtagccctattccaagatgttctggggagagtgaacagctaagagcctgcagccaagcgccctgcccccctgagcagccagacccccgggccctgcagtgcgcagcctttaactcccaggaattcatgggccagctgtatcagtgggagcccttcactgaagtccagggctcccagcgctgtgaactgaactgccggccccgtggcttccgcttctatgtccgtcacactgaaaaggtccaggatgggaccctgtgtcagcctggagcccctgacatctgtgtggctggacgctgtctgagccccggctgtgatgggatccttggctctggcaggcgtcctgatggctgtggagtctgtgggggtgatgattctacctgtcgccttgtttcggggaacctcactgaccgagggggccccctgggctatcagaagatcttgtggattccagcgggagccttgcggctccagattgcccagctccggcctagctccaactacctggcacttcgtggccctgggggccggtccatcatcaatgggaactgggctgtggatccccctgggtcctacagggccggcgggaccgtctttcgatataaccgtcctcccagggaggagggcaaaggggagagtctgtcggctgaaggccccaccacccagcctgtggatgtctatatgatctttcaggaggaaaacccaggcgttttttatcagtatgtcatctcttcacctcctccaatccttgagaaccccaccccagagccccctgtcccccagcttcagccggagattctgagggtggagcccccacttgctccggcaccccgcccagcccggaccccaggcaccctccagcgtcaggtgcggatcccccagatgcccgccccgccccatcccaggacacccctggggtctccagctgcgtactggaaacgagtgggacactctgcatgctcagcgtcctgcgggaaaggtgtctggcgccccattttcctctgcatctcccgtgagtcgggagaggaactggatgaacgcagctgtgccgcgggtgccaggcccccagcctcccctgaaccctgccacggcaccccatgccccccatactgggaggctggcgagtggacatcctgcagccgctcctgtggccccggcacccagcaccgccagctgcagtgccggcaggaatttggggggggtggctcctcggtgcccccggagcgctgtggacatctcccccggcccaacatcacccagtcttgccagctgcgcctctgtggccattgggaagttggctctccttggagccagtgctccgtgcggtgcggccggggccagagaagccggcaggttcgctgtgttgggaacaatggtgatgaagtgagcgagcaggagtgtgcgtcaggccccccgcagccccccagcagagaggcctgtgacatggggccctgtactactgcctggttccacagcgactggagctccaagtgctcagccgagtgtgggacgggaatccagcggcgctctgtggtctgccttgggagtggggcagccctcgggccaggccagggggaagcaggagcaggaactgggcagagctgtccaacaggaagccggccccctgacatgcgcgcctgcagcctggggccctgtgagagaacttggcgctggtacacagggccctggggtgagtgctcctccgaatgtggctctggcacacagcgtagagacatcatctgtgtatccaaactggggacggagttcaacgtgacttctccgagcaactgttctcacctccccaggccccctgccctgcagccctgtcaagggcaggcctgccaggaccgatggttttccacgccctggagcccatgttctcgctcctgccaagggggaacgcagacacgggaggtccagtgcctgagcaccaaccagaccctcagcacccgatgccctcctcaactgcggccctccaggaagcgcccctgtaacagccaaccctgcagccagcgccctgatgatcaatgcaaggacagctctccacattgccccctggtggtacaggcccggctctgcgtctacccctactacacagccacctgttgccgctcttgcgcacatgtcctggagcggtctccccaggatccctcctgaaaggggtccggggcaccttcacggttttctgtgccaccatcggtcacccattgatcggcccactctgaaccccctggctctccagcctgtcccagtctcagcagggatgtcctccaggtgacagagggtggcaaggtgactgacacaaagtgactttcagggctgtggtcaggcccatgtggtggtgtgatgggtgtgtgcacatatgcctcaggtgtgcttttgggactgcatggatatgtgtgtgctcaaacgtgtatcacttttcaaaaagaggttacacagactgagaaggacaagacctgtttccttgagactttcctaggtggaaaggaaagcaagtctgcagttccttgctaatctgagctacttagagtgtggtctccccaccaactccagttttgtgccctaagcctcatttctcatgttcagacctcacatcttctaagccgccctgtgtctctgaccccttctcatttgcctagtatctctgcccctgcctccctaattagctagggctggggtcagccactgccaatcctgccttactcaggaaggcaggaggaaagagactgcctctccagagcaaggcccagctgggcagagggtgaaaaagagaaatgtgagcatccgctcccccaccaccccgcccagcccctagccccactccctgcctcctgaaatggttcccacccagaactaatttattttttattaaagatggtcatgacaaatgagaaaaaaaaaa

SEQ ID NO: 5 - DNA, 6329 bp, Homo sapiens
cgcgtccatttgaacgtctcgcacgccttcctgccattagcactcgagcccgctgctgttgcccgttcttcctccagaataggggagggagagggaatgagaagctgctgcggcccaagagtcactgtgaaggaccccgccgctgccctcgggcctcctcggcccctgcgcctccggggagcagccggggctcgccgcgcctgacgcgtcccgagttatacagaaataatgttgatatttggaacccatgtcgaacttctatgaagaaaggacaacgatgattgcagcaagggatttgcaggaatttgttccttttggtcgagaccactgcaagcaccaccctaatgctttgaaccttcaacttcgccagctgcagccagcttctgaattatggtcttctgatggtgctgctggcttggtgggatcccttcaggaggttacaatccacgagaaacagaaggaaagctggcagttaaggaaaggagtaagtgaaattggagaagatgtggactatgatgaggaactctatgttgctggaaatatggtgatatggagcaaaggaagtaaaagccaggcattggcagtttataaagcatttacagttgacagtcctgttcagcaggcattgtggtgtgacttcattatatcacaggataagtctgaaaaggcctacagtagcaatgaagtagaaaaatgcatatgtatattgcaaagctcatgtattaacatgcatagcatagaaggaaaggattacatagcttcattaccatttcaggttgcaaatgtttggcccactaaatatggattgctgtttgaacgaagcgcttcttcacatgaagtacctccaggttcacccagagaacctttacctactatgttcagcatgctgcacccactagatgaaataactccacttgtttgtaaatctggaagtctttttggttcatcacgggtgcaatatgttgtagatcatgcaatgaaaattgttttcctcaatactgacccctctattgtaatgacttatgatgctgttcaaaatgtgcattctgtgtggactctccggagagtcaaatcagaggaagagaatgttgttttaaagttctctgaacaggggggaaccccacagaatgtggccactagcagctccctcacagcacatctcagaagcctctccaaaggagattcccctgtgacttcacctttccagaattactcctccattcacagccagagtcgctcaacctcatcacccagtctacattctcgctcaccttctatttccaacatggcagctctaagtcgtgctcattctcctgcgttaggagtgcactctttttcaggggtgcaaaggttcaacatttcaagccataatcagtctccaaagagacatagtatttctcattctccaaatagtaattctaatggctcctttcttgcaccagaaacggagccaattgttcctgaactgtgtattgaccatttgtggacagaaacgattactaatataagagagaaaaattcacaagcctcaaaagtgtttattacatctgacctatgtgggcaaaagttcctgtgctttttagtagagtcccagctccagttacgctgtgtaaagtttcaagagagtaatgataaaacccagctcatctttggttcagtgaccaacataccagcaaaggatgcagcaccagtggagaaaatagacaccatgctggtcttggaaggcagtggaaacctggtgctatacacaggagtggttcgggtgggaaaggtttttattcctggactgccagctccctctctgacgatgtccaacacaatgcctcggcccagtactccactagatggcgttagtactccaaagcctcttagtaaactccttggatcattggacgaggttgttctgttgtccccagttccagaactgagggattcttcaaaacttcatgattctctctataatgaggattgtactttccaacagcttggaacttacattcattctatcagagatcctgtccataacagagtcaccctggaactgagtaatggctccatggttaggatcactattcctgaaattgccacctctgagttagtacaaacgtgtttgcaagcaattaagtttatcctgccaaaagaaatagcagttcagatgcttgtcaagtggtacaatgtccacagtgctccaggaggacccagttatcactcagagtggaatttatttgtgacttgtctcatgaacatgatgggttataacacagaccgcttagcatggactagaaattttgactttgaaggatcactttctcctgtcattgcgcccaaaaaagcaaggccttccgagactggatctgatgatgactgggaatatttactaaattcagactaccaccagaatgttgagtctcatcttttgaacagatctttatgtctgagtccttcagaagcttcacagatgaaggatgaggatttttcacagaatctcagtctggattcttctacacttctctttactcacatacctgcaatttttttcgttcttcaccttgtgtatgaggagcttaagttgaatactctaatgggagaaggaatttgttcacttgttgaacttctcgttcagttggcaagggacttaaaattggggccttatgtagatcattactatagagactacccaacgcttgtcagaactactggacaagtgtgcacaattgatccaggtcaaacaggatttatgcatcatccatcattttttacgtctgagccaccaagtatttatcagtgggtgagttcttgtctgaagggtgaaggaatgccaccttatccttacctccctggaatctgtgaaagaagcagacttgtagtcttgagtattgcactgtacatacttggtgatgagagcttggtttctgatgaatcctcacagtatttaaccagaataactatagccccccagaagttgcaagtagaacaagaggaaaacaggtttagtttcaggcattctacatctgtttctagtctagctgaaagattggttgtctggatgactaatgtaggattcactttaagagatttggaaactcttccctttggaattgctcttcccatcagagatgcaatttatcactgtcgtgagcagcctgcctcagactggccagaagctgtctgtctcttgattggacgtcaggatctttccaagcaggcctgcgaaggaaacttacccaaagggaagtctgtgctctcatcagatgttccttcaggaacagaaactgaggaggaagatgacggcatgaatgacatgaatcacgaggtcatgtcattaatatggagtgaagatttaagggtgcaggatgtgcgaaggcttcttcagagtgcgcatcctgtccgtgtcaacgtagtgcagtacccagagctcagtgaccacgagttcatcgaggaaaaggaaaacagattgctccaattgtgtcagcgaactatggctcttcctgtaggacgaggaatgtttaccttgttttcgtaccatcctgttccaacagagccattgcctattcctaaattgaatctgactgggcgtgcccctcctcggaacacaacagtagaccttaatagtggaaacatcgatgtgcctcccaacatgacaagctgggccagctttcataatggtgtggctgctggcctgaagatagctcctgcctcccagatcgactcagcttggattgtttacaataagcccaagcatgctgagttggccaatgagtatgctggctttctcatggctctgggtttgaatgggcaccttaccaagctggcgactctcaatatccatgactacttgaccaagggccatgaaatgacaagcattggactgctacttggtgtttctgctgcaaaactaggcaccatggatatgtctattactcggcttcttagcattcacattcctgctctcttacccccaacgtccacagagctggatgttcctcacaatgtccaagtggctgcagtggttggcattggccttgtatatcaagggacagctcacagacatactgcagaagtcctgttggctgagataggacggcctcctggtcctgaaatggaatactgcactgacagagagtcatactccttagctgctggcttggccctgggcatggtctgcttggggcatggcagcaatttgataggtatgtctgatctcaatgtgcctgagcagctctatcagtacatggttggaggacataggcgctttcaaacaggaatgcatagggagaaacataaatcaccaagttatcaaatcaaagaaggagataccataaatgtggatgtgacttgtccaggtgctactctagctttggctatgatctacttaaaaaccaataacagatctattgcagattggctccgagcccctgacaccatgtatttgttggactttgtgaagccagaatttctcttgcttaggacacttgctcgatgcctgattttgtgggatgatattttaccaaattccaagtgggttgacagcaatgttcctcaaattataagagaaaatagtatctctctcagtgaaatcgaattgccgtgctcagaggatttgaatttggaaactttgtcccaagcacatgtctacataattgcaggagcctgcttgtctctgggttttcgatttgctggctcagaaaacttatcagcatttaactgtttgcataaatttgccaaagattttatgacttatttgtccgcacctaatgcttctgttacaggtcctcataacctagaaacttgtctgagcgtggtgctgctgtctctcgccatggtcatggctggctcaggaaacctaaaggttttgcagctttgtcgcttcttacacatgaaaacgggtggtgaaatgaactatggttttcacttagcccaccacatggcccttggacttctatttttgggaggaggaaggtactctttgagcacatcaaattcttccattgccgctcttctctgtgccctttatccgcacttcccagctcacagcactgacaaccggtatcatctccaggctctccggcacctctatgtgctggccgcggagcccaggcttctagtgcctgtggatgtggacacaaacacgccctgctatgccctcttagaagttacctacaagggcactcagtggtatgaacaaaccaaagaagaattgatggctcctacccttcttccagaactccatcttttaaagcagattaaagtaaaaggcccaagatactgggaactgctcatagatttaagcaaaggaacacaacacttgaagtccatcctttccaaggatggggttttatatgttaaactccgggcgggtcagctctcctacaaagaagatccaatgggatggcaaagtttgttggctcagactgttgctaacaggaactctgaagcccgggctttcaagccagaaacaatctcagcattcacttctgatccagcacttctgtcatttgctgaatatttctgcaagccaactgtgaacatgggtcagaaacaggaaattctggatctcttttcttcagtactctatgaatgtgttacccaggagaccccagagatgttgcctgcatacatagcaatggatcaggctataagaagacttgggagaagagaaatgtctgagacttctgaactttggcagataaagttggtgttagagtttttcagctcccgaagccatcaggagcggctgcagaaccaccctaagcgggggctctttatgaactcggaattcctccctgttgtgaagtgcaccattgataataccctggaccagtggctacaagtcgggggtgatatgtgtgtgcacgcctacctcagcgggcagcccttggaggaatcacagctgagcatgctggcctgcttcctcgtctaccactctgtgccagctccacagcacctgccacctataggactagaagggagcacaagctttgctgaactgctcttcaaatttaagcagctaaaaatgccagtgcgagctttgctgagattggctcctttgcttcttggaaatccacagccaatggtgatgtgaccgtgtctggcggtgaacctaccctgaaacgtgacttctgcacaacaaacgtgaccaaacatcaaagctaaagcaatgtttataaagttttatggtataactagggggaaatgagctgcacaaacctcaatgtattttaaatctgttgctgtcatcattaacggtatatgacatataaaagcaagttaaaatttacttttgtaaataaagtttttggtttgtttccaaaaaaaaaaaaaaaaaaaaa

SEQ ID NO: 6 - DNA, 4706 bp, homo sapiens
ttccctttgcaattgccttgggtcctgccgcacagagcggcctgtctttatcagaggtccctctgccagggggagggccccagagaaaaccagaaagagggtgagagactgaggaagataaagcgtcccagggcctcctacaccagcgcctgagcaggaagggggaggggccatgactacgaggccctgggaggtcactttagggagggctgtcctgaaacctggagcctggagcagaaagtgaaaccctggtgctccagacaaagatcttagtcgggactagccggccaaggatgaagcctcacttcagaaacacagtggagcgaatgtatcgagacacattctcctacaacttttataatagacccatcctttctcgtcggaataccgtctggctgtgctacgaagtgaaaacaaagggtccctcaaggccccgtttggacgcaaagatctttcgaggccaggtgtattcccagcctgagcaccacgcagaaatgtgcttcctctcttggttctgtggcaaccagctgcctgcttacaagtgtttccagatcacctggtttgtatcctggaccccctgcccggactgtgtggcgaagctggccgaattcctggctgagcaccccaatgtcaccctgaccatctccgccgcccgcctctactactactgggaaagagattaccgaagggcgctctgcaggctgagtcaggcaggggcccgcgtgaagattatggacgatgaagaatttgcatactgctgggaaaactttgtgtacagtgaaggtcagccattcatgccttggtacaaattcgatgacaattatgcattcctgcaccgcacgctaaaggagattctcagaaacccgatggaggcaatgtatccacacatattctacttccactttaaaaacctacgcaaagcctatggtcggaacgaaagctggctgtgcttcaccatggaagttgtaaagcaccactcacctgtctcctggaagaggggcgtcttccgaaaccaggtggatcctgagacccattgtcatgcagaaaggtgcttcctctcttggttctgtgacgacatactgtctcctaacacaaactacgaggtcacctggtacacatcttggagcccttgcccagagtgtgcaggggaggtggccgagttcctggccaggcacagcaacgtgaatctcaccatcttcaccgcccgcctctactacttctgggatacagattaccaggaggggctccgcagcctgagtcaggaaggggcctccgtggagatcatgggctacaaagattttaaatattgttgggaaaactttgtgtacaatgatgatgagccattcaagccttggaaaggactaaaatacaactttctattcctggacagcaagctgcaggagattctcgagtgaggggtctccccgggcctcatggtctgtctcctctagcctcctgctcatgttgtgcaggcctcccctccatcctggaccagctgtgcttttgcctggtcatcctgagcccctcctggcctcagggccattccatagtgctcccctgcctcaccacctcctctccgctctcccaggctcttcctgcagaggcctctttctgcctccatggctatccatccacccaccaagaccctgttccctgagcctgcatgcccctaacctgccttttcccatctccccagcataacctaatattttttttttttttttgagacggaatttcgctctgtcacccagactggagtgcaatggcttgatcttggctcactgcaaactctgcctaccaggttcaagcgattctcctgcctccgcctcccgagtagctggaattacagacgcctgccaccacgcacagctaactttttttttttttgtatttttagtagtgactgggtttcaccatgttggccaggctggtcttgaactcctgacctcaggtgatccgcctatctcagcctcccaaagtgctgggattacaggcgtgagccactggcccggcggcacaaccaaatcttattaaactcaccctaggctggccgcggtgactcatgcctataatcccccagcaatttgggaggcagaggtgagagaatcgcttgagcccaggaattcgagaccagcctgggccacatgacaaagccccatctctacaaaaaaattacaaaaaaaaaaaaaacaggtgtggtggcatgcacctgtagttgaagctacttggaaggatgaagtgggaggattgcttgagccggggaggtggaggctgcagtgaactgagatcacgtcactgaactccagtctgagcaacagatcgagaccctgcctgaaaataaatcaataaataaactcaaccgaaatgggtatgaaagttgaaatgggtatgtaagttgaaaaccagaagttttgagaaacatcctttgttaactttcatcctacaaattgggtcattcatgtcctacgcagctaaaacagagcccaggagccagggaggaaaagcagtcaggccacacaccattgctcccaaaatggacttctctgcaagcctgactcctgaaactgtgcattgtaccctgaaaccagctttatccatagcttctgcaataaatggctgtaagtcttggactccttgctataatcgcagctattcagcaatggaacctcccagttcccaacccttcctagtgcccatgggctttcccataggacaagagaacatttctccttttctttttttttttctttgaaatggagtctcgccctgtcacccaggctggagtgcaatggtgcggtctcggctcactgcaacctctgcctcccttgttcaagtgattctcctgtctcagcctcccgagtagctgggattacaggcgtccaccaccaaaccaggctaatttttgtatttttcataaaaacgggtttcatcatgtttcccaggctggtcttatttttattttattttttgagatggagtcttgctctgttgcccaggctggggtgcagtggtgcaatctgggttcactgcagcctctgccgcctgagttcaagctattttcctacctaagcctcccaagtagctgggattacatgcgcgtgccaccacgcctagctaatttttgtgtttttagtagagacggggtttcaacatcttgaccaggctggtcttgaactcctgacctcgtgatccacccgtctcggcctcccaaagtgctgggattacaggcgtgagccacctggccaggcttaggctggtcttaaactcctgacctcaagtgatccaacctccttggcctcccaaattgctgggattgctggtgtgagccacagcgcctagcccatttctccttttaataggacctgttgctgtctctgttctcccaacatggtgaacaccacccggactgcgtgtatgtcccaaattacaattctttctttgcaaatgaaatgtgaaatttagaggcccttctccacactttaaatttgacttgacattttctaggcagatataagttattagagaatgagattctctataaaaatgatcccttcatgctgtggcctccacagaagatgccctgggccaggtgcccacatgaataatgcgggccacaggcaggcatttattttctcacagatatggaggctacaagtccaaggtggaggggtcggcggggttgtttgctctgaggccgctcctcctggatggcagggatcccttctggctgtgtcctctgtggcctttcctctatgaacctgtactgtacctctggggtctctctgcttccaaatatcttttttttttttttcagacagttttgctcttgttttctaggctggagtgcaatggcacaatctcagctcactgcaacctctgccttccgagttcaagcgattctcgtgcctcagcctcctgagtagctgggactacaggcgtgtgccaccacgcctggctaattttgtagttttagtagagacggggtttctccatgttgctcaggctggtcttgaactcatgagctcaggcgatccactctcctcagcctcccaaagtgctgggattacagatataagccaccatacacaacttttttttttttttgagatggagtttcactctgttgcccaggctggagtgctaaatagcagaatcactgctcactgcaacctctgcctgctgggttcaagcaattctcccacctcagcctcctgagtagctgggattacagatgcccagaaccaatctctgctaatttttctattttttagtagagatggggtttcactgaggaaggagaccacctctctcattgtctcctatttcagaaggaagcaaaaagttagaaagatgcagaagtaagatcaatggccagactgtttggcgctgctacctgggcctggtagttaaagatcaactcctgacctgaccgcttgttttatctaaagattccagacattgtatgaggaagcattgtgaaactttctggtctgttctgctagcccccaccactgatgcatgtagccccccagtcacgtagcccacgcttgcacaatctatcacgaccctttcacgtggaccccttagaattgtaagcccttaaaagggccagggacttcttcagggagctccaatcttcagatgcaagtctgtcaacgctcccagctgattaaagcctcttccttcctaaaaaaaaaaaaaaaaaa

SEQ ID NO: 7 - DNA, 2061 bp, homo sapiens
ccaggcggaggtgagtgcgcggcggccggatgggcgggacgggcgtggaggacgccgagcaccgtggcgcgcgctcacgtccgcgtccccaagggctgcgctccctcaagcgcagtgcccagaactcggagccagcccggcccgggggaccctgctggccaaggaggtcgtcagtccggtcttgtcttccagacccggaggaccgaagcttccggacgacgaggaaccgcccaacatggcctcggagagtgggaagctttggggtggccggtttgtgggtgcagtggaccccatcatggagaagttcaacgcgtccattgcctacgaccggcacctttgggaggtggatgttcaaggcagcaaagcctacagcaggggcctggagaaggcagggctcctcaccaaggccgagatggaccagatactccatggcctagacaaggtggctgaggagtgggcccagggcaccttcaaactgaactccaatgatgaggacatccacacagccaatgagcgccgcctgaaggagctcattggtgcaacggcagggaagctgcacacgggacggagccggaatgaccaggtggtcacagacctcaggctgtggatgcggcagacctgctccacgctctcgggcctcctctgggagctcattaggaccatggtggatcgggcagaggcggaacgtgatgttctcttcccggggtacacccatttgcagagggcccagcccatccgctggagccactggattctgagccacgccgtggcactgacccgagactctgagcggctgctggaggtgcggaagcggatcaatgtcctgcccctggggagtggggccattgcaggcaatcccctgggtgtggaccgagagctgctccgagcagaactcaactttggggccatcactctcaacagcatggatgccactagtgagcgggactttgtggccgagttcctgttctgggcttcgctgtgcatgacccatctcagcaggatggccgaggacctcatcctctactgcaccaaggaattcagcttcgtgcagctctcagatgcctacagcacgggaagcagcctgatgccccagaagaaaaaccccgacagtttggagctgatccggagcaaggctgggcgtgtgtttgggcggtgtgccgggctcctgatgaccctcaagggacttcccagcacctacaacaaagacttacaggaggacaaggaagctgtgtttgaagtgtcagacactatgagtgccgtgctccaggtggccactggcgtcatctctacgctgcagattcaccaagagaacatgggacaggctctcagccccgacatgctggccactgaccttgcctattacctggtccgcaaagggatgccattccgccaggcccacgaggcctccgggaaagctgtgttcatggccgagaccaagggggtcgccctcaaccagctgtcactgcaggagctgcagaccatcagccccctgttctcgggcgacgtgatctgcgtgtgggactacgggcacagtgtggagcagtatggtgccctgggcggcactgcgcgctccagcgtcgactggcagatccgccaggtgcgggcgctactgcaggcacagcaggcctaggtcctcccacacctgccccctaataaagtgggcgcgagaggaggctgctgtgtgtttcctgccccagcctggctccctcgttgctgggctttcggggctggccagtggggacagtcagggactggagaggcagggcagggtggcctgtaatcccagcactttggaagggcaaggtgcgaggatgcttgaggccaggagtttgacacagcctgggcaacacagggagacccccatctctactcaataataaaacaaatagcctggcgtggtggcccatgcatatagtcccagctacttgtaaggctgaggtgagaggacacttgtgcccaggagtggaggctgcagtgagctatgatcacgccactgcattccagcctggataacagagtgagaacctatctctaaaaataaataaataaacgaaaaataaa

SEQ ID NO: 8 - DNA, 987 bp, homo sapiens
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 9 - DNA, 7128 bp, Homo sapiens
agataactgggcccctgcgctcaggaggccttcaccctctgctctgggtaaaggtagtagagtcccgggaaagggacagggggcccaagtgatgctctggggtactggcgtgggagagtggatttccgaagctgacagatggttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 10 - DNA, 2812 bp, Homo sapiens
agcgagctcctcctccttcccctccccactctccccgagtctagggcccccggggcgtatgacgccggagccctctgaccgcacctctgaccacaacaaacccctactccacccgtcttgtttgtcccacccttggtgacgcagagccccagcccagaccccgcccaaagcactcatttaactggtattgcggagccacgaggcttctgcttactgcaactcgctccggccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcatttaggatgctttcattgtctttaagatgtgttaggaatgtcaacagagcaaggagaaaaaaggcagtcctggaatcacattcttagcacacctacacctcttgaaaatagaacaacttgcagaattgagagtgattcctttcctaaaagtgtaagaaagcatagagatttgttcgtatttagaatgggatcacgaggaaaagagaaggaaagtgatttttttccacaagatctgtaatgttatttccacttataaaggaaataaaaaatgaaaaacattatttggatatcaaaagcaaataaaaacccaattcagtctcttctaagcaaaattgctaaagagagatgaaccacattataaagtaatctttggctgtaaggcattttcatctttccttcgggttggcaaaatattttaaaggtaaaacatgctggtgaaccaggggtgttgatggtgataagggaggaatatagaatgaaagactgaatcttcctttgttgcacaaatagagtttggaaaaagcctgtgaaaggtgtcttctttgacttaatgtctttaaaagtatccagagatactacaatattaacataagaaaagattatatattatttctgaatcgagatgtccatagtcaaatttgtaaatcttattcttttgtaatatttatttatatttatttatgacagtgaacattctgattttacatgtaaaacaagaaaagttgaagaagatatgtgaagaaaaatgtatttttcctaaatagaaataaatgatcccattttttggtatcatgtagtatgtgaaatttattcttaaacgtgactactttatttctaaataagaaattccctacctgcttcctacaagcagttcagaatgccatgccttggttgtcctagtgtgaataattttcagctactttaaaattatattgtactttctcaagcatgtcatatcctttcctattagagtatctatattacttgttactgatttacctgaaggcaatctgattaatttctaggtttttaccatattcttgtcatcttgccaattacattttaagtgttagactagactaagatgtactagttgtatagaatataactagatttattatggcaatgtttattttgtcattttgcttcatctgttttgttgttgaagtactttaaatttcatacgttcatggcatttcactgtaaagactttaatgtgtatttcttaaaataaaactttttttcctccttaaaaaaaaaaaaaaaaaa

SEQ ID NO: 11 - DNA, 4815 bp, homo sapiens
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgctggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgccttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaatttttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaaccat

SEQ ID NO: 12 - DNA, 2413 bp, Homo sapiens
cttcttcgtcagcctcccttccaccgccatattgggccactaaaaaaagggggctcgtcttttcggggtgtttttctccccctcccctgtccccgcttgctcacggctctgcgactccgacgccggcaaggtttggagagcggctgggttcgcgggacccgcgggcttgcacccgcccagactcggacgggctttgccaccctctccgcttgcctggtcccctctcctctccgccctcccgctcgccagtccatttgatcagcggagactcggcggccgggccggggcttccccgcagcccctgcgcgctcctagagctcgggccgtggctcgtcggggtctgtgtcttttggctccgagggcagtcgctgggcttccgagaggggttcgggctgcgtaggggcgctttgttttgttcggttttgtttttttgagagtgcgagagaggcggtcgtgcagacccgggagaaagatgtcaaacgtgcgagtgtctaacgggagccctagcctggagcggatggacgccaggcaggcggagcaccccaagccctcggcctgcaggaacctcttcggcccggtggaccacgaagagttaacccgggacttggagaagcactgcagagacatggaagaggcgagccagcgcaagtggaatttcgattttcagaatcacaaacccctagagggcaagtacgagtggcaagaggtggagaagggcagcttgcccgagttctactacagacccccgcggccccccaaaggtgcctgcaaggtgccggcgcaggagagccaggatgtcagcgggagccgcccggcggcgcctttaattggggctccggctaactctgaggacacgcatttggtggacccaaagactgatccgtcggacagccagacggggttagcggagcaatgcgcaggaataaggaagcgacctgcaaccgacgattcttctactcaaaacaaaagagccaacagaacagaagaaaatgtttcagacggttccccaaatgccggttctgtggagcagacgcccaagaagcctggcctcagaagacgtcaaacgtaaacagctcgaattaagaatatgtttccttgtttatcagatacatcactgcttgatgaagcaaggaagatatacatgaaaattttaaaaatacatatcgctgacttcatggaatggacatcctgtataagcactgaaaaacaacaacacaataacactaaaattttaggcactcttaaatgatctgcctctaaaagcgttggatgtagcattatgcaattaggtttttccttatttgcttcattgtactacctgtgtatatagtttttaccttttatgtagcacataaactttggggaagggagggcagggtggggctgaggaactgacgtggagcggggtatgaagagcttgctttgatttacagcaagtagataaatatttgacttgcatgaagagaagcaattttggggaagggtttgaattgttttctttaaagatgtaatgtccctttcagagacagctgatacttcatttaaaaaaatcacaaaaatttgaacactggctaaagataattgctatttatttttacaagaagtttattctcatttgggagatctggtgatctcccaagctatctaaagtttgttagatagctgcatgtggcttttttaaaaaagcaacagaaacctatcctcactgccctccccagtctctcttaaagttggaatttaccagttaattactcagcagaatggtgatcactccaggtagtttggggcaaaaatccgaggtgcttgggagttttgaatgttaagaattgaccatctgcttttattaaatttgttgacaaaattttctcattttcttttcacttcgggctgtgtaaacacagtcaaaataattctaaatccctcgatatttttaaagatctgtaagtaacttcacattaaaaaatgaaatattttttaatttaaagcttactctgtccatttatccacaggaaagtgttatttttcaaggaaggttcatgtagagaaaagcacacttgtaggataagtgaaatggatactacatctttaaacagtatttcattgcctgtgtatggaaaaaccatttgaagtgtacctgtgtacataactctgtaaaaacactgaaaaattatactaacttatttatgttaaaagattttttttaatctagacaatatacaagccaaagtggcatgttttgtgcatttgtaaatgctgtgttgggtagaataggttttcccctcttttgttaaataatatggctatgcttaaaaggttgcatactgagccaagtataattttttgtaatgtgtgaaaaagatgccaattattgttacacattaagtaatcaataaagaaaacttccatagctatt

SEQ ID NO: 13 - DNA, 1991 bp, Homo sapiens
gcaggtttagcgccactctgctggctgaggctgcggagagtgtgcggctccaggtgggctcacgcggtcgtgatgtctcgggagtcggatgttgaggctcagcagtctcatggcagcagtgcctgttcacagccccatggcagcgttacccagtcccaaggctcctcctcacagtcccagggcatatccagctcctctaccagcacgatgccaaactccagccagtcctctcactccagctctgggacactgagctccttagagacagtgtccactcaggaactctattctattcctgaggaccaagaacctgaggaccaagaacctgaggagcctacccctgccccctgggctcgattatgggcccttcaggatggatttgccaatcttgagacagagtctggccatgttacccaatctgatcttgaactcctgctgtcatctgatcctcctgcctcagcctcccaaagtgctgggataagaggtgtgaggcaccatccccggccagtttgcagtctaaaatgtgtgaatgacaactactggtttgggagggacaaaagctgtgaatattgctttgatgaaccactgctgaaaagaacagataaataccgaacatacagcaagaaacactttcggattttcagggaagtgggtcctaaaaactcttacattgcatacatagaagatcacagtggcaatggaacctttgtaaatacagagcttgtagggaaaggaaaacgccgtcctttgaataacaattctgaaattgcactgtcactaagcagaaataaagtttttgtcttttttgatctgactgtagatgatcagtcagtttatcctaaggcattaagagatgaatacatcatgtcaaaaactcttggaagtggtgcctgtggagaggtaaagctggctttcgagaggaaaacatgtaagaaagtagccataaagatcatcagcaaaaggaagtttgctattggttcagcaagagaggcagacccagctctcaatgttgaaacagaaatagaaattttgaaaaagctaaatcatccttgcatcatcaagattaaaaacttttttgatgcagaagattattatattgttttggaattgatggaagggggagagctgtttgacaaagtggtggggaataaacgcctgaaagaagctacctgcaagctctatttttaccagatgctcttggctgtgcagtaccttcatgaaaacggtattatacaccgtgacttaaagccagagaatgttttactgtcatctcaagaagaggactgtcttataaagattactgattttgggcactccaagattttgggagagacctctctcatgagaaccttatgtggaacccccacctacttggcgcctgaagttcttgtttctgttgggactgctgggtataaccgtgctgtggactgctggagtttaggagttattctttttatctgccttagtgggtatccacctttctctgagcataggactcaagtgtcactgaaggatcagatcaccagtggaaaatacaacttcattcctgaagtctgggcagaagtctcagagaaagctctggaccttgtcaagaagttgttggtagtggatccaaaggcacgttttacgacagaagaagccttaagacacccgtggcttcaggatgaagacatgaagagaaagtttcaagatcttctgtctgaggaaaatgaatccacagctctaccccaggttctagcccagccttctactagtcgaaagcggccccgtgaaggggaagccgagggtgccgagaccacaaagcgcccagctgtgtgtgctgctgtgttgtgaactccgtggtttgaacacgaaagaaatgtaccttctttcactctgtcatctttcttttctttgagtctgtttttttatagtttgtattttaattatgggaataattgctttttcacagtcactgatgtacaattaaaaacctgatggaacctggaaaa

SEQ ID NO: 14 - DNA, 3500 bp, Homo sapiens
gagtactgtgaagatgtggtccccaaggctagagctgaaaagaggcttagggccgggtgagccttccagccagggcctgcctccaagtgatgctcccccagggcagggggcataaggatggcacccagccaggtgggagcctgggccctgcccagcctcaaagctttgagctcaggaaatccggaggcaggggagggggacatcgttgccacattccccagccctttaagacccccaaggcaggaaggctgcccgggcctcaccagcttccctcacaggctccttcctgggaggaaggggctgcctgtgccctcgaaggcgcaagggagggcaggagggaggctcggaaggtgttgcaatccccagcccccgggcctgtcagaggctgagccattaacgacagagctcggggagagaagctggactgcagctggtttcaggaacttctcttgacgagaagagagaccaaggaggccaagcaggggctgggccagaggtgccaacatggggaaactgaggctcggctcggaaaggtgaagtaacttgtccaagatcacaaagctggtgaacatcaagttggtgctatggcaaggctgggaaactgcagcctgacttgggctgccctgatcatcctgctgctccccggaagtctggaggagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacggatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgtcccactccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcctcctcccgtggctttgtcctccatggcctggagcccgccagtctgtatcacatccacctcatggctgccagccaggctggggccaccaacagtacagtcctcaccctgatgaccttgaccccagaggggtcggagctacacatcatcctgggcctgttcggcctcctgctgttgctcacctgcctctgtggaactgcctggctctgttgcagccccaacaggaagaatcccctctggccaagtgtcccagacccagctcacagcagcctgggctcctgggtgcccacaatcatggaggagctgcccggacccagacagggacagtggctggggcagacatctgaaatgagccgtgctctcaccccacatccttgtgtgcaggatgccttccagctgcccggccttggcacgccacccatcaccaagctcacagtgctggaggaggatgaaaagaagccggtgccctgggagtcccataacagctcagagacctgtggcctccccactctggtccagacctatgtgctccagggggacccaagagcagtttccacccagccccaatcccagtctggcaccagcgatcaggtcctttatgggcagctgctgggcagccccacaagcccagggccagggcactatctccgctgtgactccactcagcccctcttggcgggcctcacccccagccccaagtcctatgagaacctctggttccaggccagccccttggggaccctggtaaccccagccccaagccaggaggacgactgtgtctttgggccactgctcaacttccccctcctgcaggggatccgggtccatgggatggaggcgctggggagcttctagggcttcctggggttcccttcttgggcctgcctcttaaaggcctgagctagctggagaagaggggagggtccataagcccatgactaaaaactaccccagcccaggctctcaccatctccagtcaccagcatctccctctcctcccaatctccataggctgggcctcccaggcgatctgcatactttaaggaccagatcatgctccatccagccccacccaatggccttttgtgcttgtttcctataacttcagtattgtaaactagtttttggtttgcagtttttgttgttgtttatagacactcttgggtgtaaaaaaaaaaa

SEQ ID NO: 15 - DNA, 4107 bp, Homo sapiens
gacaagggctcttcttgatggcttactgtatccactttgtccccaagaccatagggaaatgactagaggtgactgtactagctagattttaaatgaaactgaaatgaaagttcacttcctcattttgagtacctcatgtgacaagttccaatttcttttcaagtcaattgaactgaaatctccttgttgctttgaaatcttagaagagagcccactaattcaaggactcttactgtgggagcaactgctggttctatcacaatgaaacggctggtttgtgtgctcttggtgtgctcctctgcagtggcacagttgcataaagatcctaccctggatcaccactggcatctctggaagaaaacctatggcaaacaatacaaggaaaagaatgaagaagcagtacgacgtctcatctgggaaaagaatctaaagtttgtgatgcttcacaacctggagcattcaatgggaatgcactcatacgatctgggcatgaaccacctgggagacatgaccagtgaagaagtgatgtctttgatgagttccctgagagttcccagccagtggcagagaaatatcacatataagtcaaaccctaatcggatattgcctgattctgtggactggagagagaaagggtgtgttactgaagtgaaatatcaaggttcttgtggtgcttgctgggctttcagtgctgtgggggccctggaagcacagctgaagctgaaaacaggaaagctggtgtctctcagtgcccagaacctggtggattgctcaactgaaaaatatggaaacaaaggctgcaatggtggcttcatgacaacggctttccagtacatcattgataacaagggcatcgactcagacgcttcctatccctacaaagccatggatcagaaatgtcaatatgactcaaaatatcgtgctgccacatgttcaaagtacactgaacttccttatggcagagaagatgtcctgaaagaagctgtggccaataaaggcccagtgtctgttggtgtagatgcgcgtcatccttctttcttcctctacagaagtggtgtctactatgaaccatcctgtactcagaatgtgaatcatggtgtacttgtggttggctatggtgatcttaatgggaaagaatactggcttgtgaaaaacagctggggccacaactttggtgaagaaggatatattcggatggcaagaaataaaggaaatcattgtgggattgctagctttccctcttacccagaaatctagaggatctctcctttttataacaaatcaagaaatatgaagcactttctcttaacttaatttttcctgctgtatccagaagaaataattgtgtcatgattaatgtgtatttactgtactaattagaaaatatagtttgaggccgggcacggtggctcacgcctgtaatcccagtacttgggaggccaaggcaggcatatcaacttgaggccaggagttaaagagcagcctggctaacatggtgaaaccccatctctactaaaaatacaaaaaattagccgagcacggtggtgcatgcctgtaatcccagctacttgggaggctgaggcacgagattccttgaacccaagaggttgaggctatgttgagctgagatcacaccactgtactccagcctggatgacagagtggagactctgtttcaaaaaaacagaaaagaaaatatagtttgattcttcatttttttaaatttgcaaatctcaggataaagtttgctaagtaaattagtaatgtactatagatataactgtacaaaaattgttcaacctaaaacaatctgtaattgcttattgttttattgtatactctttgtctttttaagacccctaatagccttttgtaacttgatggcttaaaaatacttaataaatctgccatttcaaatttctatcattgccacataccattcttattcctaggcaactattaataatctatcctgagaatattaattgtggtattctggtgatggggtttagcaactttgatggaagaaaatattaggctataaatgtcctaaggactcagattgtatctttgtacagaagaggattcaaaacgccacgtgtagtggctcatgcctgtaatcccaacactttgggaggctgaagtaggaggatcgtcttgagcccaggagttcaagaccagcctggacaacatagtgagaccttgtctccacaaaaataaaaaagaaactatccaggagtggtggtgtgtgcctgtggtccctgctatgcagatgtctaagacaggaggatcacaagagcccaggaggttgagaatgcagtgagcttgtaattgcaccactgcactccagcctgggtgacagagcaagaccctgtcttaaaaaaagaggattcaacacatatttttatattatgttaaagtaaagaaatgcataaaagacaagcactttggaagaattattttaatgatcaacaatttaatgtattagtccaaattatttttacgtagtcatcaacaatttgaccagggcctttatttggcaaataactgagccaaccagaataaaataaccaatactccactgctcatatttttatctaattcagatggatcttccttacaactgctctagattagtagatgcatctaagcaggcagcaggaactttaaattttttaagttcatgtctatgacatgaacaatgtgtgggataatgtcattaatatatcctaaattaacctaaacgtatttcactaactctggctccttctccataaagcacattttaaggaacaagaattgctaaatataaaaacataaataataccataatacatggctatcatcaaaagtgtatagaatattatagtttaaaagtatttagttgattacttttcagttttgttttgttttttgagacggagtctcactctgttgcccaggctggagtgcagtggcaccatctcagttcactgcaacttctgcctcccgagttcaagcgattctcctgcctcagcctcccgagtagctggaattataggcgtgcaccaccacgcccagctaatttttgtatttttagtaaagacagggttttgccacattagccaggctggtctcaaactcctgacctcaggtgatccacccaccccagcctcccaaagtgctaagattacaggcgtgagccactgagcccagcctacttttcagtttttaacataatttttgttttatccacaacttttcaagtattgaaagtagaataaaaacatgggttcttagtctttagctatctgttaaagcctatgaatgccttcttaaaatcatgtttttaaatgcataaaatatataggattacaaaggaatctaattatatcgaaatacagttattaaaatgttaaaagataagtttgttatatattaatatgcatgcttctttataaatgcattaaataagagttaatagctatcctaaatttgaaatagtgataagcataatgaaaatagatgcaaaaaactaatgtgatatgaaaatatctgggtttttcttttgatgatgaagtattgctaatattaccgtggtttatgaactatgttcagaattgaagaaaatcctaactttcagttagaggttagtgacggggttcaggacaccctacacaaaatacagcactttgacatattgaatattttaagctgaaggcatttgaggaaattgcagaagcaggaaggtgactctgaccttctgcctgctgttctccccagaagcagccataaaacctgggaaggattttctgaccttcccctgaagtagatcataagactgtcatgtaagaggtgctctcctggcacccagagaaaaggagcatccttacctccaaaagcacagggacacaaagaggaatctaaacaaacaggcctctcagtttcccccagtttattacatttagcttgttcacactttgccctatgacatttctacatcactggctgctcttcatcaaacctactataaaaaacattcaagttcaactgtttctttgggcctttatttccttatggagcccctcgtgtcgtgtaaaacttatattaaataaatgtgcatgcttt

SEQ ID NO: 16 - DNA, 2196 bp, Homo sapiens
ctgggcgggtcatgcgccctggccttcgcgcatctcccaggttagctgcgtgtccgggtgctaggctgcagacccgccgccatgacgctgcgcgcggccgtcttcgaccttgacggggtgctggcgctgccagcggtgttcggcgtcctcggccgcacggaggaggccctggcgctgcccagaggacttctgaatgatgctttccagaaagggggaccagagggtgccactacccggcttatgaaaggagagatcacactttcccagtggataccactcatggaagaaaactgcaggaagtgctccgagaccgctaaagtctgcctccccaagaatttctccataaaagaaatctttgacaaggcgatttcagccagaaagatcaaccgccccatgctccaggcagctctcatgctcaggaagaaaggattcactactgccatcctcaccaacacctggctggacgaccgtgctgagagagatggcctggcccagctgatgtgtgagctgaagatgcactttgacttcctgatagagtcgtgtcaggtgggaatggtcaaacctgaacctcagatctacaagtttctgctggacaccctgaaggccagccccagtgaggtcgtttttttggatgacatcggggctaatctgaagccagcccgtgacttgggaatggtcaccatcctggtccaggacactgacacggccctgaaagaactggagaaagtgaccggaatccagcttctcaataccccggcccctctgccgacctcttgcaatccaagtgacatgagccatgggtacgtgacagtaaagcccagggtccgtctgcattttgtggagctgggctccggccctgctgtgtgcctctgccatggatttcccgagagttggtattcttggaggtaccagatccctgctctggcccaggcaggttaccgggtcctagctatggacatgaaaggctatggagagtcatctgctcctcccgaaatagaagaatattgcatggaagtgttatgtaaggagatggtaaccttcctggataaactgggcctctctcaagcagtgttcattggccatgactggggtggcatgctggtgtggtacatggctctcttctaccccgagagagtgagggcggtggccagtttgaatactcccttcataccagcaaatcccaacatgtcccctttggagagtatcaaagccaacccagtatttgattaccagctctacttccaagaaccaggagtggctgaggctgaactggaacagaacctgagtcggactttcaaaagcctcttcagagcaagcgatgagagtgttttatccatgcataaagtctgtgaagcgggaggactttttgtaaatagcccagaagagcccagcctcagcaggatggtcactgaggaggaaatccagttctatgtgcagcagttcaagaagtctggtttcagaggtcctctaaactggtaccgaaacatggaaaggaactggaagtgggcttgcaaaagcttgggacggaagatcctgattccggccctgatggtcacggcggagaaggacttcgtgctcgttcctcagatgtcccagcacatggaggactggattccccacctgaaaaggggacacattgaggactgtgggcactggacacagatggacaagccaaccgaggtgaatcagatcctcattaagtggctggattctgatgcccggaacccaccggtggtctcaaagatgtagaacgcagcgtgtgcccacgctcagcaggtgtgccatccttccacctgctggggcaccattcttagtatacagaggtggccttacacacatcttgcatggatggcagcattgttctgaaggggtttgcagaaaaaaaagattttctttacataaagtgaatcaaatttgacattattttagatcccagagaaatcaggtgtgattagttctccaggcatgaatgcatcgtccctttatctgtaagaacccttagtgtcctgtagggggacagaatggggtggccaggtggtgatttctctttgaccaatgcatagtttggcagaaaaatcagccgttcatttagaagaatcttagcagagattgggatgccttactcaataaagctaagatgactatgctgctggctgtctttgttcttggagaggtggagtgactgttcacggagaa

SEQ ID NO: 17 - DNA, 3651 bp, Homo sapiens
ctgtctgagcatttcactgcggagcctgagcgcgcctgcctgggaaaacactgcagcggtgctcggactcctcctgtccagcaggaggcgcggcccggcagctcccgcatgcgcagtgcgctcggtgtcagacggcccggatcccggttaccggcccctcgctcgctgctcgccagcccagactcggccctggcagtggcggctggcgattcggaccgatccgacctgggcggaggtggcccgcgccccgcggcatgagccggtgaccaagctcggggccgagcgggaggcagccgtggccgaggagtgtgaggaagaggctgtctgtgtcattatgtgtgcgtcggtcaagtataatatccggggtcctgccctcatcccaagaatgaagaccaagcaccgaatctactatatcaccctcttctccattgtcctcctgggcctcattgccactggcatgtttcagttttggccccattctatcgagtcctcaaatgactggaatgtagagaagcgcagcatccgtgatgtgccggttgttaggctgccagccgacagtcccatcccagagcggggggatctcagttgcagaatgcacacgtgttttgatgtctatcgctgtggcttcaacccaaagaacaaaatcaaggtgtatatctatgctctgaaaaagtacgtggatgactttggcgtctctgtcagcaacaccatctcccgggagtataatgaactgctcatggccatctcagacagtgactactacactgatgacatcaaccgggcctgtctgtttgttccctccatcgatgtgcttaaccagaacacactgcgcatcaaggagacagcacaagcgatggcccagctctctaggtgggatcgaggtacgaatcacctgttgttcaacatgttgcctggaggtcccccagattataacacagccctggatgtccccagagacagggccctgttggctggtggcggcttttctacgtggacttaccggcaaggctacgatgtcagcattcctgtctatagtccactgtcagctgaggtggatcttccagagaaaggaccaggtccacggcaatacttcctcctgtcatctcaggtgggtctccatcctgagtacagagaggacctagaagccctccaggtcaaacatggagagtcagtgttagtactcgataaatgcaccaacctctcagagggtgtcctttctgtccgtaagcgctgccacaagcaccaggtcttcgattacccacaggtgctacaggaggctactttctgtgtggttcttcgtggagctcggctgggccaggcagtattgagcgatgtgttacaagctggctgtgtcccggttgtcattgcagactcctatattttgcctttctctgaagttcttgactggaagagagcatctgtggttgtaccagaagaaaagatgtcagatgtgtacagtattttgcagagcatcccccaaagacagattgaagaaatgcagagacaggcccggtggttctgggaagcgtacttccagtcaattaaagccattgccctggccaccctgcagattatcaatgaccggatctatccatatgctgccatctcctatgaagaatggaatgaccctcctgctgtgaagtggggcagcgtgagcaatccactcttcctcccgctgatcccaccacagtctcaagggttcaccgccatagtcctcacctacgaccgagtagagagcctcttccgggtcatcactgaagtgtccaaggtgcccagtctatccaaactacttgtcgtctggaataatcagaataaaaaccctccagaagattctctctggcccaaaatccgggttccattaaaagttgtgaggactgctgaaaacaagttaagtaaccgtttcttcccttatgatgaaatcgagacagaagctgttctggccattgatgatgatatcattatgctgacctctgacgagctgcaatttggttatgaggtctggcgggaatttcctgaccggttggtgggttacccgggtcgtctgcatctctgggaccatgagatgaataagtggaagtatgagtctgagtggacgaatgaagtgtccatggtgctcactggggcagctttttatcacaagtattttaattacctgtatacctacaaaatgcctggggatatcaagaactgggtagatgctcatatgaactgtgaagatattgccatgaacttcctggtggccaacgtcacgggaaaagcagttatcaaggtaaccccacgaaagaaattcaagtgtcctgagtgcacagccatagatgggctttcactagaccaaacacacatggtggagaggtcagagtgcatcaacaagtttgcttcagtcttcgggaccatgcctctcaaggtggtggaacaccgagctgaccctgtcctgtacaaagatgactttcctgagaagctgaagagcttccccaacattggcagcttatgaaacgtgtcattggtggaggtctgaatgtgaggctgggacagagggagagaacaaggcctcccagcactctgatgtcagagtagtaggttaagggtggaaggttgacctacttggatcttggcatgcacccacctaacccactttctcaagaacaagaacctagaatgaatatccaagcacctcgagctatgcaacctctgttcttgtatttcttatgatctctgatgggttcttctcgaaaatgccaagtggaagactttgtggcatgctccagatttaaatccagctgaggctccctttgttttcagttccatgtaacaatctggaaggaaacttcacggacaggaagactgctggagaagagaagcgtgttagcccatttgaggtctggggaatcatgtaaagggtacccagacctcacttttagttatttacatcaatgagttctttcagggaaccaaacccagaattcggtgcaaaagccaaacatcttggtgggatttgataaatgccttgggacctggagtgctgggcttgtgcacaggaagagcaccagccgctgagtcaggatcctgtcagttccatgagctattcctctttggtttggctttttgatatgattaaaattattttttattcctttttctactgtgtcttaaacaccaattcctgatagtccaaggaaccacctttctcccttgatatatttaactccgtctttggcctgacaacagtcttctgcccatgtctgggaacacacgccaggaggaatgtctgataccctctgcatcaagcgtaagaaggtcccaaatcataaccattttaagaacagatgactcagaaacctccagaggaatctgtttgcttcctgattagatccagtcaatgttttaaaggtattgtcagagaaaaacagagggtctgtactagccatgcaaggagtcgctctagctggtacccgtaaaagttgtgggaattgtgacccccatcccaaggggatgccaaaatttctctcattcttttggtataaacttaacattagccagggaggttctggctaacgttaaatgctgctatacaactgctttgcaacagttgctggtatatttaaatcattaaatttcagcatttactaatactgcaaaaaaaaaaaaaaaaaa

SEQ ID NO: 18 - DNA, 2158 bp, Homo sapiens
attcataaaacgcttgttataaaagcagtggctgcggcgcctcgtactccaaccgcatctgcagcgagcatctgagaagccaagactgagccggcggccgcggcgcagcgaacgagcagtgaccgtgctcctacccagctctgctccacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggacttctgcacggacctggccgtctccagtgccaacttcattcccacggtcactgccatctcgaccagtccggacctgcagtggctggtgcagcccgccctcgtctcctccgtggccccatcgcagaccagagcccctcaccctttcggagtccccgccccctccgctggggcttactccagggctggcgttgtgaagaccatgacaggaggccgagcgcagagcattggcaggaggggcaaggtggaacagttatctccagaagaagaagagaaaaggagaatccgaagggaaaggaataagatggctgcagccaaatgccgcaaccggaggagggagctgactgatacactccaagcggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgtagtttttcttcaacatcaatgttcattgtaatgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttccaagacattgtcaataaaagcatttaagttgaatgcgaccaa

SEQ ID NO: 19 - DNA, 1668 bp, Homo sapiens
acgggccaaggcggcgcgtctcgggggtggagcctggaggtgaccgcgccgctgcaacgcccccaccccccgcggtcgcagtggttcagcccgagaacttttcattcataaaaagaaaagactccgcacggcgcgggtgagtcagaacccagcagccgtgtaccccgcagagccgccagccccgggcatgttccgagacttcggggaacccggcccgagctccgggaacggcggcgggtacggcggccccgcgcagcccccggccgcagcgcaggcagcccagcagaagttccacctggtgccaagcatcaacaccatgagtggcagtcaggagctgcagtggatggtacagcctcatttcctggggcccagcagttaccccaggcctctgacctaccctcagtacagccccccacaaccccggccaggagtcatccgggccctggggccgcctccaggggtacgtcgaaggccttgtgaacagatcagcccggaggaagaggagcgccgccgagtaaggcgcgagcggaacaagctggctgcggccaagtgcaggaaccggaggaaggaactgaccgacttcctgcaggcggagactgacaaactggaagatgagaaatctgggctgcagcgagagattgaggagctgcagaagcagaaggagcgcctagagctggtgctggaagcccaccgacccatctgcaaaatcccggaaggagccaaggagggggacacaggcagtaccagtggcaccagcagcccaccagccccctgccgccctgtaccttgtatctccctttccccagggcctgtgcttgaacctgaggcactgcacacccccacactcatgaccacaccctccctaactcctttcacccccagcctggtcttcacctaccccagcactcctgagccttgtgcctcagctcatcgcaagagtagcagcagcagcggagacccatcctctgacccccttggctctccaaccctcctcgctttgtgaggcgcctgagccctactccctgcagatgccaccctagccaatgtctcctccccttcccccaccggtccagctggcctggacagtatcccacatccaactccagcaacttcttctccatccctctaatgagactgaccatattgtgcttcacagtagagccagcttggggccaccaaagctgcccactgtttctcttgagctggcctctctagcacaatttgcactaaatcagagacaaaatatttcccatttgtgccagaggaatcctggcagcccagagactttgtagatccttagaggtcctctggagccctaaccccttccagatcactgccacactctccatcaccctcttcctgtgatccacccaaccctatctcctgacagaaggtgccactttacccacctagaacactaactcaccagccccactgccagcagcagcaggtgattggaccaggccattctgccgccccctcctgaaccgcacagctcaggaggcgcccttggcttctgtgatgagctgatctgcggatctcagctttgagaagccttcagctccagggaatccaagcctccacagcgagggcagctgctatttattttcctaaagagagtatttttatacaaacctaccaaaatggaataaaaggcttgaagctgtg

SEQ ID NO: 20 - DNA, 7856 bp, Homo sapiens
cgcgatctgctgcagctcggccgggagacggcgcgacccggcggcggggccacccgcgagtccagcgtcgccgcagccccccaatgcggccgcgagaagcagcgggggggcaggcgatcgaaggagccttcacgtaaatgggtccagtcatgcctcccagtaagaagccagaaagctcaggaattagtgtctccagtggactgagtcagtgttacgggggcagcggtttctccaaggcccttcaggaagacgatgacctcgacttttctctgcctgacatccgattagaagagggggccatggaagatgaagagctgaccaacctgaactggctgcacgagagcaagaacttgctgaagagctttggggagtcggtcctcaggagtgtcagccccgtccaggacctggacgatgacacccccccatcccctgcccactctgacatgccctacgatgccaggcagaaccccaactgcaaacccccctactccttcagctgcctcatatttatggccatcgaggactctccaaccaagcgcctgccagtgaaggatatctacaactggatcttggaacattttccgtattttgcaaatgcacctactgggtggaaaaactcagtgagacacaatttatcattgaataagtgttttaagaaagtggacaaagagaggagtcagagtattgggaaagggtcgttgtggtgcatagacccagagtatagacaaaatctaattcaggctttgaaaaagacaccttatcacccacacccacacgtgttcaatacacctcccacctgtcctcaggcatatcaaagcacatcaggtccacccatctggccgggcagtaccttcttcaagagaaatggagcccttctccaagatcctgacattgatgctgccagtgccatgatgcttttgaatactccccctgagatacaagcaggttttcctccaggagtgatccaaaatggagcgcgggtcctgagccgagggctgtttcctggcgtgcggccgctgccaatcactcccattggggtgacagcggccatgaggaatggcatcaccagctgccggatgcggactgagagtgagccatcttgtggctccccagtggtcagcggagaccccaaggaggatcacaactacagcagtgccaagtcctccaacgcccggagcacctcgcccaccagcgactccatctcctcctcctcctcctcagccgacgaccactatgagtttgccaccaaggggagccaggagggcagcgagggcagcgaggggagcttccggagccacgagagccccagcgacacggaagaggacgacaggaagcacagccagaaggagcccaaggattctctgggggacagcgggtacgcatcccagcacaagaagcgccagcacttcgccaaggccaggaaggtccccagcgacacactgcccctcaaaaagagacgcaccgaaaagccccccgagagcgatgatgaggagatgaaagaagcggcagggtccctcctgcacttagcagggatccggtcctgtttgaataacatcaccaatcggacggcaaaggggcagaaagagcaaaaggaaaccacaaaaaattaaaaacaagtcactgatttgttttgaacttacgaccatttggtttcagcatgtcaggagatttctaatgatttgtggcaatatcagcaattttttttcttttttcttgtttttggtttggttttctttcttttcttttccttttattttgttttaatttgccccctcttctttgttttggacccttaagaattttatttttaaaggagattgaagccatagaactcatattgacactcagctgttttacaaaagcttttcattatctgaagacaaaaccgaaaaagccaaaattaccattgcttcctccagcttgtcagaaacctgtggctgaatccgcagggatgtcaacgtcaatatcacaggaacacacattcggcacctagaaggcacgtgggcaaagtaatcatcgttcaggcccaacccttaggtttaaaaagtcaggttgtccatcccattgggttcactgagtgaaggcacataaagcaattgaggaggaggaggaacccctcgtccccctaggagcagacccaagcttgtggcaccaggcatctgatggtgccaggaaagccactggaattgtcacacggcgagcacagagggccggccaccagtcctcgatgcttctgaaccctgaagcccgatgacatcttacgaggtggacgttggactgttcatgcgcatcgggtgtcagtgactcatggagaagaaatggggtaaatttttagtgatgttgctaatcattgaattctgttctctattaaattaagaaaatgttccaaaagccataagcctgaagattggccctgtgcacgcacgcacacacacacacacacacacacacacacacacacacacacgaaggagagagagagaaaactgatggggaaaacaagctgtgtcttcttaactgcccaagtgaaaagcaaccaagtccaggaaattacaatagctgttaaggaaaggaaataatggtacagatctttttctgtctatcaaaactatttgatccaagtgaaaaaaaaaaaaaaactagaaagctacggaacctgccattagtattgtggtgtatttttaagattaaaggtacactgatggacaaaaaaaaaaagtaaaacatggcaaaaaataaaataactcctatactgccctcaaaatggagtttgcaattaatatcaggatttatctttgcaaaaatcagtgatttccacattcagccagtatagccagcagaaatttctgatccacaatgcatggattcctttgaagaaaaaaaagaaaaagagaaaaaaatcacaaaaacaaactttttttattcaaaagtaacaaagttcttgtaaggtaaataatgtatttagcatgaagcatgaattattttcatataaatatagaaaatagagaaaaggctatgcctgtaatttttaagcccttaggcttagagtttcttttggttttcttcttttttctttccttttctttgctttctttttttcctttttgtttttgtttttgttttttgtttttgtttttttttcgggttattttgttttggttttttgaagcaggtgtttaaggtttaaccttcttcagggacaaattctgactgttggggaacttactctgcaatataaaaatatcttcatgctctggtagggcttggatggttgaactctgtactgccttgtgtgcacttcagccccgaccccctctgattctctgttgaaaagtgtgtcctttctctctgtctgtacatgtttaacatgacgcaataatttgagggcaaacttagtagtgagtgtgtatgatagaatcaagagaattatgggacgcttacttgagaaaatcattaccatgatttggttctaggaaaaaggcagtgaataattatgcaaattagccagaagaaggggaaccgtgctaatgggccttattgggtgaggggacgagatggggttcatgtgaaggaggaagcgatgccgaggtaggaaaggccagccccagacatcctatcgccacaatgccatgtcgcaataggaagcaggggccggccatcgctaccttcagcacactgaccaacctggaattaagaccacctagattgcgagagctgaatttagaaaccagacaacgtcatgcagcccagaaactcctgttgttacctttgcctaagaaattttctttaatggcgggggcggggggcgggggtacaaagagaaatctctaaaagaatatgatcttccatccaagtggagggaaactttaaaacaaaaacacccagtactgtggctcaggatatgatgcgtgaggagagggagggaacagagatgaccttaacttttaaaaaagggactgctgtgggccaaagccaagcccatctgccaggacgaggtaatgtcagagctccatcagcccggacagtgggaactaactggtgcattccccacacttaccttccggtgggttgctgatgagagaacctgaaaaaacctacacctctacagcaggtcgaattcatgacctgaagctgaatacttccagcatatttattcagggtgtaggtgggaataaagtatcttcgcagtgctctgttccctccgtctccccagacatctgacaccctaaaagccatccacagctatggaacctgagcgacaccttgatttgtgttgtcacctgaccaagcctaaagacctccagctcagtcccccaccttcatcccaccccacagatgataaaattcagacctctctcctgaaaggcagaggttcaacattcaggactgtttctggccgaggacttcttccaattaaaacccccaccgtgggctgtctcccctcatttcatttttctaaaggggcagaggcctcttttagaaaataataaaatgcaatgtgtgtgatttacttttctgatctctttgagaaatagagaaatataaaagtgtgttcttaactccagaaccactctttttgcataaatacctcatcgggcagctttctaagtgtgattttcctgagtctcccttcgttggatctgccggaagacttgtcggggaacctttagtgagggtacttcttcctatttttcttctgtttttggaggcatacacattatgcataaccaaaacaatggctcaattgtgtttaactttgtattttgattgttgagaacaaaaacaaaaagtatcaatgtgtatgtggctgtttgtagtgaatttattggagaatgaggttgtccgtgtccttaacaagccaaggggcaggaggcaccctctcttatcccctcctccaagagcagtagagaatttaagcacaagcctatttgtgaaagaatattttgcttaagtgtcattcactttagtcttggaattccttcccaaacgtcaggtgttcttttagcttccaaactagcatatgtatccattagtctgacagatcgcctgaacaccattaagaggtgtggcgtttttgctttcatttctcctgctgggagaagtggcggttcatgtgtcattccagtatctcacatactcacacggggcaggggggagggggaaacggggaactatagcaatatttaaagatgctttggaaaccaaccgtgaacacatcaacaccacgacgtctacgattacttgctattggccctcggatacatttaagagaaagagacagtcactcttttttttcttaaatgatatacatataaacagttatttttatcctattataattgtcttttgtctttatctagtactatgtggaaagggtttgcatcatagatttttcccagccttataatataccataagctcctacttccctgcccctccctaatcagtattctttcaagagttctttggtgaagccatctatctgaaactaaaatgaaccaaacccatatttcactggtggttggagaaaaccatggccaaaacgattgtggcaggtctcaatcttgggagtttttaagaaggaatgtgccagaggccgattcccaagaacagagttttcttttgttttgcagaggcattcaatgtgtctagtgcttgctggccacagcagttactaccacagagccttctgggaggggccgttgtgttgaaggaggctcctgcctgagggacagcatcaggcagtgggctctgtagagtgagaaccaggtggaggccttctgtgcccagctcagagttctgcaccacgccaggactgcccaggccaagggctactgacgcaagttccactcattccactctgtggggggcgccttgggcctctcctggaagggctcttggagaaggaattggagttacgtacaagtgacctaaatgggaagcttttctagatgagattggattaaattccatgtgatttctctttccctttaatccaggttgggactcgtttctttctggtggatcacagctgcccagatgttgcaattgatttttatgtttctgtagagaagtatttttctttcatcttcaggattttttttgccaccaaaagaaaacattggaactctgtgtttcctcttgattgtgacttcccagtgttgacagttaagtccttagtgtcgtaggtcccagcccaccaatactatatcaaacactgttatgcacataatgcagcactgtgatctaatttaaataatacttttttattatttatactactatatataatatacatcaacacttttgctatataacctaagtgataaccctcttttagttacctgccaaactctggacttggtttatattgcagttaacacagttacaaagctgtaatggtgtctttttttcctttgtaacggaatgtgtaaatcaaagtatatacattgtgtggtgttcctgtttctggagtttcatgaggatttacacatggcattcagtgttctgtatagatctgcctacctttgtgaattcatctgttaacccctcttcctttgagagagcaccggcgatggtggttaactccttgtgttttctctctctcctactggttattcttgaattaagcacagactcgtcagctcggttgctttatcatgaataatgtgtgtgaccttgcagttcttccacagttcagcaaacaagtgctagcttcactgaccaaaaattaaggaaggaaaacacagtttttaaaacgatccatcttttaacagccgaaaccgatgtgtctatggtgctgcaccttgctgttgtacttctgaaatcagacgtgtgtgaacgatcatttctgacttaaccgtgagatgctcacgagtacccttcctgttgttttgttagcattgaaatcgagactatttatttggaatatatacaacagtgtttttccactgtatttcatttgcaaaagttgagaactgctttctctaccttttgcaaaataattgatattccatattggattctcaaagacttcgatatggtgaacctattaaacctagaaattgtattcatcctttcatgactgtggcctgagttccccagcccctctcctccttttttttagatgagatttagcacactctcagttatttaaacatgcaacatttcttgagtatgtatgttgaggccatctgagctcatagctgattcagtaaccagtttcatgctgtgtcattcacactcactacttaatactgccatggtgaaaatgtggaggaaaaatgtatccatgtgtgtctgggaagcatatacacttgtacattttttaatactctgattctgtaacatttctgagttttgttttgttttacagaaaaaaaaaaaaagtgataaagcaatcagaagaccaagaggtttactattgatgcttagggtcgtctgaccttggctggccaatagacctacacggccaaattaatttacgagagtaataatttttcaaaagccaattttttttctgtattttctgtatgaaactgccaatatcatgaatagaaagggagaaccataaaggagaaagaacgtgatgttctgttatgttcatgtaaacctaaagaaacagtgtggaggcaggcgcgatcagccgaactctagggacttggtgttgcttggaaggcatccatacctgcattttgcattcttcgtatgtaatcatattgccaaagacaaactatttcatcatttattgtaaataacacttttccccagacctaccataaagtttctgtgatgtattgtcttccagttgcaataaaaattactgagttgcatcaattgaagaaaaacaccaaaaa

SEQ ID NO: 21 - DNA, 1310 bp, Homo sapiens
aaattgagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc

SEQ ID NO: 22 - DNA, 1310 bp, Homo sapiens; identical to SEQ ID NO: 21

SEQ ID NO: 23 - DNA, 3070 bp, Homo sapiens
ggcgccgtcttgatactttcagaaagaatgcattccctgtaaaaaaaaaaaaaaaatactgagagagggagagagagagagaagaagagagagagacggagggagagcgagacagagcgagcaacgcaatctgaccgagcaggtcgtacgccgccgcctcctcctcctctctgctcttcgctacccaggtgacccgaggagggactccgcctccgagcggctgaggaccccggtgcagaggagcctggctcgcagaattgcagagtcgtcgcccctttttacaacctggtcccgttttattctgccgtacccagtttttggatttttgtcttccccttcttctctttgctaaacgacccctccaagataatttttaaaaaaccttctcctttgctcacctttgcttcccagccttcccatccccccaccgaaagcaaatcattcaacgacccccgaccctccgacggcaggagccccccgacctcccaggcggaccgccctccctccccgcgcgcgggttccgggcccggcgagagggcgcgagcacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacagaaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaatgctgaacattgcatataacttatattgtaagaaatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaataaaaaaaagaaaaaagagaaaagaaaaaaaaagaaaaaagttgtaggcgaatcatttgttcaaagctgttggcctctgcaaaggaaataccagttctgggcaatcagtgttaccgttcaccagttgccgttgagggtttcagagagcctttttctaggcctacatgctttgtgaacaagtccctgtaattgttgtttgtatgtataattcaaagcaccaaaataagaaaagatgtagatttatttcatcatattatacagaccgaactgttgtataaatttatttactgctagtcttaagaactgctttctttcgtttgtttgtttcaatattttccttctctctcaatttttggttgaataaactagattacattcagttggcctaaggtggttgtgctcggagggtttcttgtttcttttccattttgtttttggatgatatttattaaatagcttctaagagtccggcggcatctgtcttgtccctattcctgcagcctgtgctgagggtagcagtgtatgagctaccagcgtgcatgtcagcgaccctggcccgacaggccacgtcctgcaatcggcccggctgcctcttcgccctgtcgtgttctgtgttagtgatcactgcctttaatacagtctgttggaataatattataagcataataataaagtgaaaatattttaaaactacaa

SEQ ID NO: 24 - DNA, 2979 bp, Homo sapiens
ccggggacggctgctggagcggcgcccgccgcggctcagcgcattcccgctctccgcttccctctccgctgcgtccccgcgcgaagatggcaaccgaggggctgcacgagaacgagacgctggcgtcgctgaagagcgaggccgagagcctcaagggcaagctggaggaggagcgagccaagctgcacgatgtggagctgcaccaggtggcggagcgggtggaggccctggggcagtttgtcatgaagaccagaaggaccctcaaaggccacgggaacaaagtcctgtgcatggactggtgcaaagataagaggaggatcgtgagctcgtcacaggatgggaaggtgatcgtgtgggattccttcaccacaaacaaggagcacgcggtcaccatgccctgcacgtgggtgatggcatgtgcttatgccccatcgggatgtgccattgcttgtggtggtttggataataagtgttctgtgtaccccttgacgtttgacaaaaatgaaaacatggctgccaaaaagaagtctgttgctatgcacaccaactacctgtcggcctgcagcttcaccaactctgacatgcagatcctgacagcgagcggcgatggcacatgtgccctgtgggacgtggagagcgggcagctgctgcagagcttccacggacatggggctgacgtcctctgcttggacctggccccctcagaaactggaaacaccttcgtgtctgggggatgtgacaagaaagccatggtgtgggacatgcgctccggccagtgcgtgcaggcctttgaaacacatgaatctgacatcaacagtgtccggtactaccccagtggagatgcctttgcttcagggtcagatgacgctacgtgtcgcctctatgacctgcgggcagatagggaggttgccatctattccaaagaaagcatcatatttggagcatccagcgtggacttctccctcagtggtcgcctgctgtttgctggatacaatgattacactatcaacgtctgggatgttctcaaagggtcccgggtctccatcctgtttggacatgaaaaccgcgttagcactctacgagtttcccccgatgggactgctttctgctctggatcatgggatcataccctcagagtctgggcctaatcatcttctgacagtgcactcatgtatacctgagaatttgaaatcttcacatgtaaatagatattacttctagaggagcttagagtttattgcagtgtagcttaggggagcaacccatggctcacaggtcactaagcgtctccaatatgactattaaaactgtcacctctggaaatacactagtgtgagccttcagcactgcgagaataccttcaagtacagtatttttcttttggaacactttttaaaatgtatctgtttttaaggttattctaaattatagtagcctcaactcattctgtcaccagtagaattcagcagttaatatattccatattatttctttgaatcaattcattttcagagcactttaaagtctgatatttctcgatgtgcactgtgatgcctggaaccttcctctggaagtgctgattttatggactgaggactggtgactggtctgtgatagaagcaaattccaattccaaatgtaattagacaaaaatcatttttttagaatgtgtttttattgtaaaagtatctttttcagcttcctgttctattgtcttttttcagatacaacatttttgtctatggtgaactgctgtaaatgacgcagagaaatgcctaaaaaggacaggtggtttgactcatggatgatgatgatgtcactgtgccacttggacagggcgttttctctgaattgaagggaaagccaatggtgtttgtaaacaaatgcttctgagagcaaagaaaagtcttctgtgtgggaacacaagatagtaaacttatttaaaaacctattagtagaattagtggaaacacttaggttaaagtgaatcttgtccatataaattatattcatggccgggcgcggtggctcacgcttgtaatcccagcactttgggaggccgaggcgggcggatcacgaggtcaggagttcgagaccacggtgaaaccctgtctctactaaaaaatacaaaaaattagccgggcgtggtggcgggcgcctgtagtcccagctactcggagaggctgaggcaggagaatggcgtgaacccgggaggtggagcttgcagtgagccgaggtcgagccactgcagcctgggtgacaaagcgagactccgtctcaaaaaaaaaaaaaaattatattcatatgtattgcattgcaattataattacatatgcagattgattgatagtcatgaataataacgtctgctcctcttacatagaaaaacgatattaaaagaagatcttctctttatttgagactcagaattccttctagaagaaggaagtgctttttgttataggatcccttcttttcctttttttgtttttttgtaagatgtagatgcttattctttgctttagaaaacttctcacttaaaaagatggcatgcacctaggggaataaaaggtcacctcagacaccaggtgtcattcctggtgaggcctgcctcgtcggtggcctggggtctgccggcaggttctggctgcacctgaaggctgcgtgcaccttgtcccctggacaggtctcctttcctggccctgctccagcccagcccttcttctagtggtagctctggctttgcaggcccagctccaggccctgctcctcagagagactcttccagagctggagctgggcacagccataagacaggactggaccagatgctcctgtaaacatccaggggtgtgccaggcccaccctcacaactgcttgttcaggtatcgtgatgggccactcggtccaaaatcagccaggccatcttttccatcatctcacttcaaataaacataataattatatttgatcatttgc

SEQ ID NO: 25 - DNA, 1372 bp, Homo sapiens
aagctggcgaggccgagcccctcctagtgcttccggaccttgctccctgaacactcggaggtggcggtggatcttactccttccagccagtgaggatccagcaacctgctccgtgcctcccgcgcctgttggttggaagtgacgaccttgaagatcggccggttggaagtgacgaccttgaagatcggcgggcgcagcggggccgagggggcgggtctggcgctaggtccagcccctgcgtgccgggaaccccagaggaggtcgcagttcagcccagctgaggcctgtctgcagaatcgacaccaaccagcatcatgtccatgacactggggtactgggacatccgcgggctggcccacgccatccgcctgctcctggaatacacagactcaagctacgaggaaaagaagtatacgatgggggacgctcctgactatgacagaagccagtggctgaatgaaaaattcaagctgggcctggactttcccaatctgccctacttgattgatggggctcacaagatcacccagagcaacgccatcctgtgctacattgcccgcaagcacaacctgtgtggggagacagaagaggagaagattcgtgtggacattttggagaaccaggctatggacgtctccaatcagctggccagagtctgctacagccctgactttgagaaactgaagccagaatacttggaggaacttcctacaatgatgcagcacttctcacagttcctggggaagaggccatggtttgttggagacaagatcacctttgtagatttcctcgcctatgatgtccttgacctccaccgtatatttgagcccaactgcttggacgcctttccaaatctgaaggacttcatctcccgctttgaggtttcctgtggcataatgtgatggtcaattttctgcatcaacttgactgggctaagggatgctcagatggcaggtaaaatcattgtgcttgtgagggtgtttccagaagagatttgcctttgaatcagaagacagcaaagatttccttcagcaatgaaggaggcatccaccaaactgtcagggcccagagagaagaaaaagacaggaagggtgaatttgacctctctgactgggacatccatctctgcctatcctgggacctccacactcctggttctctggccttcagacttgatcagggactaacaccatcgcctcccacccccacctttgttctgaggcctttagcctctgaatgataccactggctttcctgcttctctatcctgcagtcggcagatcatgggacttcttcactccaaaattgtgtgagccaattcccataacagatagataaatttataaataaacacacaaatttcctacagcct

SEQ ID NO: 26 - DNA, 1312 bp, Homo sapiens
ttttaatggtcagactctattacaccccacattctcttttcttttattcttgtctgttctgcctcactcccgagctctactgactcccaacagagcgcccaagaagaaaatggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaaggcacatggaggtgatggtgtttcttagagagaagatcactgaagaaacttctgctttaatggctttacaaagctggcaatattacaatccttgacctcagtgaaagcagtcatcttcagcattttccagccctatagccaccccaagtgtggatatgcctcttcgattgctccgtactctaacatctagctggcttccctgtctattgccttttcctgtatctattttcctctatttcctatcattttattatcaccatgcaatgcctctggaataaaacatacaggagtctgtctctgctatggaatgccccatggggcatctcttgtgtacttattgtttaaggtttcctcaaactgtgatttttctgaacacaataaactattttgatgatcttgggtggaaaaaaaaaaaaaaaaaaaaaaaaaaaaaaaaaa

SEQ ID NO: 27 - DNA, 1169 bp, Homo sapiens
tgccctgcgcccgcaacccgagccgcacccgccgcggacggagcccatgcgcggggcgaaccgcgcgcccccgcccccgccccgccccggcctcggccccggccctggccccgggggcagtcgcgcctgtgaacggtggggcaggagaccctgtaggaggaccccgggccgcaggcccctgaggagcgatgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctgacgcaggtgagggggactcccagggcggccgccacgcccaccggatgaccccggctccccgcccctgccggtctcctggcctgcggtcagcagcctcccttgtgccccgcccagcacaagctcaggacatggaggtgccggatgcaggaaggaggtgcagacggaaggaggaggaaggaaggacggaagcaaggaaggaaggaagggctgctggagcccagtcaccccgggaccgtgggccgaggtgactgcagaccctcccagggaggctgtgcacagactgtcttgaacatcccaaatgccaccggaaccccagcccttagctcccctcccaggcctctgtgggcccttgtcgggcacagatgggatcacagtaaattattggatggtcttgaaaaaaaaaaaaaaaaaa

SEQ ID NO: 28 - DNA, 665 bp, Homo sapiens
gggaacacatccaagcttaagacggtgaggtcagcttcacattctcaggaactctccttctttgggtctggctgaagttgaggatctcttactctctaggccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggccatggcggctgtgcccatggtgctcagtgccatgggcttcactgcggcgggaatcgcctcgtcctccatagcagccaagatgatgtccgcggcggccattgccaatgggggtggagttgcctcgggcagccttgtggctactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctggggtgaaatataccaaattctgcatctccagaggaaaataagaaataaagatgaattgttgcaactcttcaaaa

SEQ ID NO: 29 - DNA, 1760 bp, Homo sapiens
agagggcgagggcgagggcagagggcgctggcggcagcggccgcggaagatgagcagcagctgctcagggctgagcagggtcctggtggccgtggctacagccctggtgtctgcctcctccccctgcccccaggcctggggccccccaggggtccagtatgggcagccaggcaggtccgtgaagctgtgttgtcctggagtgactgccggggacccagtgtcctggtttcgggatggggagccaaagctgctccagggacctgactctgggctagggcatgaactggtcctggcccaggcagacagcactgatgagggcacctacatctgccagaccctggatggtgcacttgggggcacagtgaccctgcagctgggctaccctccagcccgccctgttgtctcctgccaagcagccgactatgagaacttctcttgcacttggagtcccagccagatcagcggtttacccacccgctacctcacctcctacaggaagaagacagtcctaggagctgatagccagaggaggagtccatccacagggccctggccatgcccacaggatcccctaggggctgcccgctgtgttgtccacggggctgagttctggagccagtaccggattaatgtgactgaggtgaacccactgggtgccagcacacgcctgctggatgtgagcttgcagagcatcttgcgccctgacccaccccagggcctgcgggtagagtcagtaccaggttacccccgacgcctgcgagccagctggacataccctgcctcctggccgtgccagccccacttcctgctcaagttccgtttgcagtaccgtccggcgcagcatccagcctggtccacggtggagccagctggactggaggaggtgatcacagatgctgtggctgggctgccccatgctgtacgagtcagtgcccgggactttctagatgctggcacctggagcacctggagcccggaggcctggggaactccgagcactgggaccataccaaaggagataccagcatggggccagctacacacgcagccagaggtggagcctcaggtggacagccctgctcctccaaggccctccctccaaccacaccctcggctacttgatcacagggactctgtggagcaggtagctgtgctggcgtctttgggaatcctttctttcctgggactggtggctggggccctggcactggggctctggctgaggctgagacggggtgggaaggatggatccccaaagcctgggttcttggcctcagtgattccagtggacaggcgtccaggagctccaaacctgtagaggacccaggagggcttcggcagattccacctataattctgtcttgctggtgtggatagaaaccaggcaggacagtagatccctatggttggatctcagctggaagttctgtttggagcccatttctgtgagaccctgtatttcaaatttgcagctgaaaggtgcttgtacctctgatttcaccccagagttggagttctgctcaaggaacgtgtgtaatgtgtacatctgtgtccatgtgtgaccatgtgtctgtgaggcagggaacatgtattctctgcatgcatgtatgtaggtgcctggggagtgtgtgtgggtccttggctcttggcctttccccttgcaggggttgtgcaggtgtgaataaagagaataaggaagttcttggaaaaaaaaaaaaaaaaaaaaaaaaaaaaaaaaaaaaaaaaaaaaaaaaaaaaaa

SEQ ID NO: 30 - DNA, 3338 bp, Homo sapiens
gacatcatgggctatttttaggggttgactggtagcagataagtgttgagctcgggctggataagggctcagagttgcactgagtgtggctgaagcagcgaggcgggagtggaggtgcgcggagtcaggcagacagacagacacagccagccagccaggtcggcagtatagtccgaactgcaaatcttattttcttttcaccttctctctaactgcccagagctagcgcctgtggctcccgggctggtgtttcgggagtgtccagagagcctggtctccagccgcccccgggaggagagccctgctgcccaggcgctgttgacagcggcggaaagcagcggtacccacgcgcccgccgggggaagtcggcgagcggctgcagcagcaaagaactttcccggctgggaggaccggagacaagtggcagagtcccggagcgaacttttgcaagcctttcctgcgtcttaggcttctccacggcggtaaagaccagaaggcggcggagagccacgcaagagaagaaggacgtgcgctcagcttcgctcgcaccggttgttgaacttgggcgagcgcgagccgcggctgccgggcgccccctccccctagcagcggaggaggggacaagtcgtcggagtccgggcggccaagacccgccgccggccggccactgcagggtccgcactgatccgctccgcggggagagccgctgctctgggaagtgagttcgcctgcggactccgaggaaccgctgcgcccgaagagcgctcagtgagtgaccgcgacttttcaaagccgggtagcgcgcgcgagtcgacaagtaagagtgcgggaggcatcttaattaaccctgcgctccctggagcgagctggtgaggagggcgcagcggggacgacagccagcgggtgcgtgcgctcttagagaaactttccctgtcaaaggctccggggggcgcgggtgtcccccgcttgccagagccctgttgcggccccgaaacttgtgcgcgcagcccaaactaacctcacgtgaagtgacggactgttctatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttgaagagagaccgtcgggggctgaggggcaacgaagaaaaaaaataacacagagagacagacttgagaacttgacaagttgcgacggagagaaaaaagaagtgtccgagaactaaagccaagggtatccaagttggactgggttgcgtcctgacggcgcccccagtgtgcacgagtgggaaggacttggcgcgccctcccttggcgtggagccagggagcggccgcctgcgggctgccccgctttgcggacgggctgtccccgcgcgaacggaacgttggacttttcgttaacattgaccaagaactgcatggacctaacattcgatctcattcagtattaaaggggggagggggagggggttacaaactgcaatagagactgtagattgcttctgtagtactccttaagaacacaaagcggggggagggttggggaggggcggcaggagggaggtttgtgagagcgaggctgagcctacagatgaactctttctggcctgccttcgttaactgtgtatgtacatatatatattttttaatttgatgaaagctgattactgtcaataaacagcttcatgcctttgtaagttatttcttgtttgtttgtttgggtatcctgcccagtgttgtttgtaaataagagatttggagcactctgagtttaccatttgtaataaagtatataatttttttatgttttgtttctgaaaattccagaaaggatatttaagaaaatacaataaactattggaaagtactcccctaacctcttttctgcatcatctgtagatactagctatctaggtggagttgaaagagttaagaatgtcgattaaaatcactctcagtgcttcttactattaagcagtaaaaactgttctctattagactttagaaataaatgtacctgatgtacctgatgctatggtcaggttatactcctcctcccccagctatctatatggaattgcttaccaaaggatagtgcgatgtttcaggaggctggaggaaggggggttgcagtggagagggacagcccactgagaagtcaaacatttcaaagtttggattgtatcaagtggcatgtgctgtgaccatttataatgttagtagaaattttacaataggtgcttattctcaaagcaggaattggtggcagattttacaaaagatgtatccttccaatttggaatcttctctttgacaattcctagataaaaagatggcctttgcttatgaatatttataacagcattcttgtcacaataaatgtattcaaataccaaaaaaaaaaaaaaaaa

SEQ ID NO: 31 - DNA, 5312 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 32 - DNA, 2619 bp, Homo sapiens
gcttcctgggcttcccatctctggcgggaagcgctccccgacgcattctctacctaggggacacccccaaggcaggagcccgggccgacggagaggacttaacgacactatcggaccctctgggaaaagaggggagacgtcgtgacccaggccccgccccaccttgccgcctcgtgcccggcgctaagacccagcgggcgcgccgcccgcccggggcccggccctgtccccttccgtccgcggggcagccagctcagctccggagagccggcggcgcggcgggcatggctcgggtggcgtgggggctgctgtggttgctgctgggcagcgccggggcgcagtacgagaagtacagcttccggggcttcccgcccgaggacctgatgccgctggccgcggcgtacgggcacgctctggagcagtacgagggagagagctggcgcgagagcgcgcgctacctggaggcggcgctgcggctgcaccggctcctgcgcgacagcgaggccttctgccacgccaactgcagcggccccgcgcccgcggccaagcccgatcccgacggcggccgcgcagacgagtgggcctgcgagctgcggctcttcggccgcgtcctggagcgagccgcctgcctgcggcgctgcaagcggacgctgcccgccttccaggtgccctacccgccgcggcagctgctgcgtgacttccagagccgcctgccctaccagtacctgcactacgcgctgttcaaggctaaccggctggagaaggcggtggcggcggcctacaccttcctccagaggaacccgaagcacgagctgaccgccaagtatctcaactactatcaggggatgctggacgtcgccgacgagtccctcacggacctagaggcccagccctacgaggccgtgttcctccgggctgtgaagctctacaacagcggggatttccgcagcagcacggaggacatggagcgggccttgtcagagtacctggcagtctttgcccggtgcctggccggctgtgaaggggcccatgagcaggtggacttcaaggacttctacccggccatagcagatctctttgcagagtccctgcagtgcaaggtggactgtgaggccaatttgacccccaatgtgggtggctacttcgtggacaagttcgtggccaccatgtaccactacctgcagtttgcctactataagttgaatgatgtgcgccaggctgcccgcagcgccgccagctacatgctcttcgaccccaaggacagcgtcatgcagcagaacctggtgtattaccggttccaccgggctcgctggggcctggaagaggaggacttccagccccgggaggaggccatgctctaccacaaccagaccgccgagctgcgggagctgctggagttcacccacatgtacctgcagtcagatgatgagatggagctggaggagacagaaccgcccctggagcctgaggatgccctatctgacgccgagtttgagggggagggtgactacgaggagggcatgtatgctgactggtggcaggagccggatgccaagggtgacgaggccgaggctgagccagagcctgaactcgcatgagaaggggacaccccacaccgctcaagcttgggaagcctggtgccgatggccccaccctcaccagcctgggcagcagcaagaactatttattaaaaacttaagatgggccaggtgcggtggctcacacctgtaatcccagcattttgggaggccaaggtgggtggatcacttgaggccaggagttcaagaccagcctggccaacatgatgagacctccgtctctactaaaatacataaattagccgggtgtggtggcaggcgcctgaaatcccagctactcaagaggctgaggcaggagaatcgcttgaacctgggaggcaaaggttgcagtgaactgagattgcgccaccgcactccagcctgggcgacagagcgagactccatctttaaaaaaaaacaagacgggccggcacggtggctcacgcctgtaatcccagcactgagaggccgatcacttgaggtcaggagttcaagaccagcctggccaacatggtgaaaccccatctctactaaaaaatacaaaaattagccaggcatggtggcacacacctgtaatcgtagctgaggcaggagaatcgcctgaacccaggaggcggagcttgcagtgagccgagatcgtgccactgcactccagcctgggcgacagagtgagactccatctcaaaaaaaaaaaaaaaaacttaagatggacacagctgactggacccccatcctgcctcacccatgggtgctgcaccccagacccatcctgccacttctatgtctctggaccacaggatggtggtggcattgcaggttggcaagtgggctgatggggtccgccctcctcactgctgagctcctcacctggacagtctcctggacaaggagtttccagctgctggctggagtctcaggccaaattgcagagggtcctccagggtcctgaagagcactggactaagagtctagtggttccagggccctgaccagtaggtgctcaataaatgtttgttgttgaatgaaaaaaaaaaaaaaaaaa

SEQ ID NO: 33 - DNA, 1440 bp, Homo sapiens
ggaggtgagcaggaaggagacggccgcccagcagcccgtgggcaggcgcggcggagcgagcggggccggcggcgggcgccgagggacgccgaggcctcgggcgggggctggcccggggttccaggtctccagtgggggctgcagactaagcaaaatgaggcggttcctgaggccagggcatgaccctgtgcgggagaggctcaagcgggacctgttccagtttaacaagacggtggagcatggcttcccgcaccagcccagcgccctcggctacagcccgtccctgcgcatcctggccatcggcacccgttctggagccatcaagctctacggagccccaggcgtggagttcatggggctgcaccaggagaacaacgctgtgacgcagatccacctcctgcccggccagtgccagctggtcaccctgctggatgacaacagcctgcacctttggagcctgaaggtcaagggcggggcatcggagctgcaggaggatgagagcttcacactgcgtggacccccaggggctgcccccagtgccacacagatcaccgtggtcctgccacattcctcctgcgagctgctctacctgggcaccgagagtggcaacgtgtttgtggtgcagctgccagcttttcgtgcgctggaggaccggaccatcagctcggacgcggtgctgcagcggttgccagaggaggcccgccaccggcgtgtgttcgagatggtggaggcactgcaggagcaccctcgagaccccaaccagatcctgatcggctacagccgaggcctcgttgtcatctgggacctacagggcagccgcgtgctctaccacttcctcagcagccagcaactggagaacatctggtggcagcgggacggccgcctgctcgtcagctgtcactctgacggcagctactgccagtggcccgtgtccagcgaagcccagcaaccagagcccctccgcagcctcgtgccttacggtccctttccttgcaaagcgattaccagaatcctctggctgaccactaggcaggggttgcccttcaccatcttccagggtggcatgccacgggccagctacggggaccgccactgcatctcagtgatccacgatggccagcagacggccttcgacttcacctcccgtgtcatcggcttcactgtcctcacagaggcagaccctgcagccagtaggagagcttcgggagtgggtgcccagggttaggtgtgggaggcatggggcaggaccatcagtaaagacagggccaggtgcagtggctcctgcctgtaaccccagtgctgtgggaggccaaggtggtaggatcgcttgaacccaggagttcaagtccagcctggacaacgtagggagacccttgtctctacaaaaaataaaaaaattagccaggaaaaaaaaaaaaaaaaaaaaaaaaaaaaaaaaaa

SEQ ID NO: 34 - DNA, 4454 bp, Homo sapiens
gaaacgtcccgtgtgggaggggcgggtctgggtgcggcctgccgcatgactcgtggttcggaggcccacgtggccggggcggggactcaggcgcctggggcgccgactgattacgtagcgggcggggccggaagtgccgctccttggtgggggctgttcatggcggttccggggtctccaacatttttcccggctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttccctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaactttcacagtgaagtgcctttttcctagaagtggtttgtagacttcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcacatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaattacattttcactgtatatcatggtatcttaatgatgtatataattgccttcaatccccttctcaccccaccctctacagcttcccccacagcaataggggcttgattatttcagttgagtaaagcatggtgctaatggaccagggtcacagtttcaaaacttgaacaatccagttagcatcacagagaaagaaattcttctgcatttgctcattgcaccagtaactccagctagtaattttgctaggtagctgcagttagccctgcaaggaaagaagaggtcagttagcacaaaccctttaccatgactggaaaactcagtatcacgtatttaaacatttttttttcttttagccatgtagaaactctaaattaagccaatattctcatttgagaatgaggatgtctcagctgagaaacgttttaaattctctttattcataatgttctttgaagggtttaaaacaagatgttgataaatctaagctgatgagtttgctcaaaacaggaagttgaaattgttgagacaggaatggaaaatataattaattgatacctatgaggatttggaggcttggcattttaatttgcagataataccctggtaattctcatgaaaaatagacttggataacttttgataaaagactaattccaaaatggccactttgttcctgtctttaatatctaaatacttactgaggtcctccatcttctatattatgaattttcatttattaagcaaatgtcatattaccttgaaattcagaagagaagaaacatatactgtgtccagagtataatgaacctgcagagttgtgcttcttactgctaattctgggagctttcacagtactgtcatcatttgtaaatggaaattctgcttttctgtttctgctccttctggagcagtgctactctgtaattttcctgaggcttatcacctcagtcatttcttttttaaatgtctgtgactggcagtgattctttttcttaaaaatctattaaatttgatgtcaaattagggagaaagatagttactcatcttgggctcttgtgccaatagcccttgtatgtatgtacttagagttttccaagtatgttctaagcacagaagtttctaaatggggccaaaattcagacttgagtatgttctttgaataccttaagaagttacaattagccgggcatggtggcccgtgcctgtagtcccagctacttgagaggctgaggcaggagaatcacttcaacccaggaggtggaggttacagtgagcagagatcgtgccactgcactccagcctgggtgacaagagagacttgtctccaaaaaaaaagttacacctaggtgtgaattttggcacaaaggagtgacaaacttatagttaaaagctgaataacttcagtgtggtataaaacgtggtttttaggctatgtttgtgattgctgaaaagaattctagtttacctcaaaatccttctctttccccaaattaagtgcctggccagctgtcataaattacatattccttttggtttttttaaaggttacatgttcaagagtgaaaataagatgttctgtctgaaggctaccatgccggatctgtaaatgaacctgttaaatgctgtatttgctccaacggcttactatagaatgttacttaatacaatatcatacttattacaatttttactataggagtgtaataggtaaaattaatctctattttagtgggcccatgtttagtctttcaccatcctttaaactgctgtgaatttttttgtcatgacttgaaagcaaggatagagaaacactttagagatatgtggggtttttttaccattccagagcttgtgagcataatcatatttgctttatatttatagtcatgaactcctaagttggcagctacaaccaagaaccaaaaaatggtgcgttctgcttcttgtaattcatctctgctaataaattataagaagcaaggaaaattagggaaaatattttatttggatggtttctataaacaagggactataattcttgtacattatttttcatctttgctgtttctttgagcagtctaatgtgccacacaattatctaaggtatttgttttctataagaattgttttaaaagtattcttgttaccagagtagttgtattatatttcaaaacgtaagatgatttttaaaagcctgagtactgacctaagatggaattgtatgaactctgctctggagggaggggaggatgtccgtggaagttgtaagacttttatttttttgtgccatcaaatataggtaaaaataattgtgcaattctgctgtttaaacaggaactattggcctccttggccctaaatggaagggccgatattttaagttgattattttattgtaaattaatccaacctagttctttttaatttggttgaatgttttttcttgttaaatgatgtttaaaaaataaaaactggaagttcttggcttagtcataattctt

SEQ ID NO: 35 - DNA, 1575 bp, Homo sapiens
tcccttctgaggaaacgaaaccaacagcagtccaagctcagtcagcagaagagataaaagcaaacaggtctgggaggcagttctgttgccactctctctcctgtcaatgatggatctcagaaataccccagccaaatctctggacaagttcattgaagactatctcttgccagacacgtgtttccgcatgcaaatcaaccatgccattgacatcatctgtgggttcctgaaggaaaggtgcttccgaggtagctcctaccctgtgtgtgtgtccaaggtggtaaagggtggctcctcaggcaagggcaccaccctcagaggccgatctgacgctgacctggttgtcttcctcagtcctctcaccacttttcaggatcagttaaatcgccggggagagttcatccaggaaattaggagacagctggaagcctgtcaaagagagagagcattttccgtgaagtttgaggtccaggctccacgctggggcaacccccgtgcgctcagcttcgtactgagttcgctccagctcggggagggggtggagttcgatgtgctgcctgcctttgatgccctgggtcagttgactggcggctataaacctaacccccaaatctatgtcaagctcatcgaggagtgcaccgacctgcagaaagagggcgagttctccacctgcttcacagaactacagagagacttcctgaagcagcgccccaccaagctcaagagcctcatccgcctagtcaagcactggtaccaaaattgtaagaagaagcttgggaagctgccacctcagtatgccctggagctcctgacggtctatgcttgggagcgagggagcatgaaaacacatttcaacacagcccagggatttcggacggtcttggaattagtcataaactaccagcaactctgcatctactggacaaagtattatgactttaaaaaccccattattgaaaagtacctgagaaggcagctcacgaaacccaggcctgtgatcctggacccggcggaccctacaggaaacttgggtggtggagacccaaagggttggaggcagctggcacaagaggctgaggcctggctgaattacccatgctttaagaattgggatgggtccccagtgagctcctggattctgctgacccagcacactccaggcagcatccaccccacaggcagaagaggactggacctgcaccatcctctgaatgccagtgcatcttgggggaaagggctccagtgttatctggaccagttccttcattttcaggtgggactcttgatccagagaggacaaagctcctcagtgagctggtgtataatccaggacagaacccaggtctcctgactcctggccttctatgccctctatcctatcatagataacattctccacagcctcacttcattccacctattctctgaaaatattccctgagagagaacagagagatttagataagagaatgaaattccagccttgactttcttctgtgcacctgatgggagggtaatgtctaatgtattatcaataacaataaaaataaagcaaataccatttaaaaaaaaaaa

SEQ ID NO: 36 - DNA, 3177 bp, Homo sapiens
acggtctgggggcggggccacgccgattggcgcgaagttttcttttctccttccaccttcttttcatttctagtgagacacacgctttggtcctggctttcggcccgtagttgtagaaggagccctgctggtgcaggttagaggtgccgcatcccccggagctctcgaagtggaggcggtaggaaacggagggcttgcggctagccggaggaagctttggagccggaagccatggcacactaccccacaaggctgaagaccagaaaaacttattcatgggttggcaggcccttgttggatcgaaaactgcactaccaaacctatagagaaatgtgtgtgaaaacagaaggttgttccaccgagattcacatccagattggacagtttgtgttgattgaaggggatgatgatgaaaacccgtatgttgctaaattgcttgagttgttcgaagatgactctgatcctcctcctaagaaacgtgctcgagtacagtggtttgtccgattctgtgaagtccctgcctgtaaacggcatttgttgggccggaagcctggtgcacaggaaatattctggtatgattacccggcctgtgacagcaacattaatgcggagaccatcattggccttgttcgggtgatacctttagccccaaaggatgtggtaccgacgaatctgaaaaatgagaagacactctttgtgaaactatcctggaatgagaagaaattcaggccactttcctcagaactatttgcggagttgaataaaccacaagagagtgcagccaagtgccagaaacccgtgagagccaagagtaagagtgcagagagcccttcttggaccccagcagaacatgtggccaaaaggattgaatcaaggcactccgcctccaaatctcgccaaactcctacccatcctcttaccccaagagccagaaagaggctggagcttggcaacttaggtaaccctcagatgtcccagcagacttcatgtgcctccttggattctccaggaagaataaaacggaaagtggccttctcggagatcacctcaccttctaagagatctcagcctgataaacttcaaaccttgtctccagctctgaaagccccagagaaaaccagagagactggactctcttatactgaggatgacaagaaggcttcacctgaacatcgcataatcctgagaacccgaattgcagcttcgaaaaccatagacattagagaggagagaacacttacccctatcagtgggggacagagatcttcagtggtgccatccgtgattctgaaaccagaaaacatcaaaaagagggatgcaaaagaagcaaaagcccagaatgaagcgacctctactccccatcgtatccgcagaaagagttctgtcttgactatgaatcggattaggcagcagcttcggtttctaggtaatagtaaaagtgaccaagaagagaaagagattctgccagcagcagagatttcagactctagcagtgacgaagaagaggcttccacaccgccccttccaaggagagcacccagaactgtgtccaggaacctgcgatcttccttgaagtcatccttacataccctcacgaagctcaagcctagaacgccacgttgtgccgctcctcagatccgtagtcgaagcctggctgcccaggagccagccagtgtgctggaggaagcccgactgaggctgcatgtttctgctgtacctgagtctcttccctgtcgggaacaggaattccaagacatctacaattttgtggaaagcaaactccttgaccataccggagggtgcatgtacatctccggtgtccctgggacagggaagactgccactgttcatgaagtgatacgctgcctgcagcaggcagcccaagccaatgatgttcctccctttcaatacattgaggtcaatggcatgaagctgacggagccccaccaagtctatgtgcaaatcttgcagaagctaacaggccaaaaagcaacagccaaccatgcggcagaactgctggcaaagcaattctgcacccgagggtcacctcaggaaaccaccgtcctgcttgtggatgagctcgaccttctgtggactcacaaacaagacataatgtacaatctctttgactggcccactcataaggaggcccggcttgtggtcctggcaattgccaacacaatggacctgccagagcgaatcatgatgaaccgggtgtccagccgactgggtcttaccaggatgtgcttccagccctatacatatagccagctgcagcagatcctaaggtcccggctcaagcatctaaaggcctttgaagatgatgccatccagctggtagccaggaaggtagcagcactgtctggagatgcacgacggtgcctggacatctgcaggcgtgccacagagatctgtgagttctcccagcagaagcctgactcccctggcctggtcaccatagcccactcaatggaagctgtggatgagatgttttcatcatcatacatcacggccatcaaaaattcctctgttctggaacagagcttcctgagagccatcctcgcagagttccgtcgatcaggactggaggaagccacgtttcaacagatatatagtcaacatgtggcactgtgcagaatggagggactgccgtaccccaccatgtcagagaccatggccgtgtgttctcacctgggctcctgtcgcctcctgcttgtggagcccagcaggaacgatctgctccttcgggtgcggctcaacgtcagccaggatgatgtgctgtatgcgctgaaagacgagtaaaggggcttcacaagttaaaagactggggtcttgctgggttttgttttttgagacagggtcttgctctgtcgcccaggctggagtgcagtggcacgatcatggctcactgcagccttgacttctcaggcttaggtgaccccccaacctcatcctcccaggtggctgaaactacaggcacatgccaccatgcccagctgattttttgtagagacagggcttcaccatgttgccaagctagtctacaaagcatctgattttggaagtacatggaattgttgtaacaaagtatattgaatggaaatggctctcatgtattttggaattttccattaaataatttgctttttcctgaaaaaaaaaaaaaaaaaaaaaaaa

SEQ ID NO: 37 - DNA, 2439 bp, Homo sapiens
gagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagctgtatttccaaaatgtcgctttctaacaagctgacgctggacaagctggacgttaaagggaagcgggtcgttatgagagtcgacttcaatgttcctatgaagaacaaccagataacaaacaaccagaggattaaggctgctgtcccaagcatcaaattctgcttggacaatggagccaagtcggtagtccttatgagccacctaggccggcctgatggtgtgcccatgcctgacaagtactccttagagccagttgctgtagaactcaaatctctgctgggcaaggatgttctgttcttgaaggactgtgtaggcccagaagtggagaaagcctgtgccaacccagctgctgggtctgtcatcctgctggagaacctccgctttcatgtggaggaagaagggaagggaaaagatgcttctgggaacaaggttaaagccgagccagccaaaatagaagctttccgagcttcactttccaagctaggggatgtctatgtcaatgatgcttttggcactgctcacagagcccacagctccatggtaggagtcaatctgccacagaaggctggtgggtttttgatgaagaaggagctgaactactttgcaaaggccttggagagcccagagcgacccttcctggccatcctgggcggagctaaagttgcagacaagatccagctcatcaataatatgctggacaaagtcaatgagatgattattggtggtggaatggcttttaccttccttaaggtgctcaacaacatggagattggcacttctctgtttgatgaagagggagccaagattgtcaaagacctaatgtccaaagctgagaagaatggtgtgaagattaccttgcctgttgactttgtcactgctgacaagtttgatgagaatgccaagactggccaagccactgtggcttctggcatacctgctggctggatgggcttggactgtggtcctgaaagcagcaagaagtatgctgaggctgtcactcgggctaagcagattgtgtggaatggtcctgtgggggtatttgaatgggaagcttttgcccggggaaccaaagctctcatggatgaggtggtgaaagccacttctaggggctgcatcaccatcataggtggtggagacactgccacttgctgtgccaaatggaacacggaggataaagtcagccatgtgagcactgggggtggtgccagtttggagctcctggaaggtaaagtccttcctggggtggatgctctcagcaatatttagtactttcctgccttttagttcctgtgcacagcccctaagtcaacttagcattttctgcatctccacttggcattagctaaaaccttccatgtcaagattcagctagtggccaagagatgcagtgccaggaacccttaaacagttgcacagcatctcagctcatcttcactgcaccctggatttgcatacattcttcaagatcccatttgaattttttagtgactaaaccattgtgcattctagagtgcatatatttatattttgcctgttaaaaagaaagtgagcagtgttagcttagttctcttttgatgtaggttattatgattagctttgtcactgtttcactactcagcatggaaacaagatgaaattccatttgtaggtagtgagacaaaattgatgatccattaagtaaacaataaaagtgtccattgaaaccgtgatttttttttttttcctgtcatactttgttaggaagggtgagaatagaatcttgaggaacggatcagatgtctatattgctgaatgcaagaagtggggcagcagcagtggagagatgggacaattagataaatgtccattctttatcaagggcctactttatggcagacattgtgctagtgcttttattctaacttttatttttatcagttacacatgatcataatttaaaaagtcaaggcttataacaaaaaagccccagcccattcctcccattcaagattcccactccccagaggtgaccactttcaactcttgagtttttcaggtatatacctccatgtttctaagtaatatgcttatattgttcacttcttttttttttattttttaaagaaatctatttcataccatggaggaaggctctgttccacatatatttccacttcttcattctctcggtatagttttgtcacaattatagattagatcaaaagtctacataactaatacagctgagctatgtagtatgctatgattaaatttacttatgtaaaaaaaaaaaaaaaaaa

SEQ ID NO: 38 - DNA, 1954 bp, Homo sapiens
actggacaaaagcgtggtctctggcgcggggatctcagagtttcccgggcactcaccgtgtgtagttggcatctccgcgcgtccggacacccgatcccagcatccctgcctgcaggactgttcgtgttcagctcgcgtcctgcagctgtccgaggtgctccagttggaggctgaggttcccgggctctgtagctgagtgggcggcggcaccggcggagatgcctgggaagaaggcgcgcaagaacgctcaaccgagccccgcgcgggctccagcagagctggaagtcgagtgtgctactcaactcaggagatttggagacaaactgaacttccggcagaaacttctgaatctgatatccaaactcttctgctcaggaacctgactgcatcaaaaacttgcatgaggggactccttcaaaagagttttctcaggaggtgcacgtttcatcaatttgaagaaagactgcattgtaattgagaggaatgtgaaggtgcattcatgggtgcccttggaaacggaagatggaatacatcaaagtgaatttctgttcaagttttcccagattatcattctttgggatgagagaacattataaaaccactttgtttattttaaagcaagaatggaagacccttgaaaataaagaagtaattattgacacatttcttttttacttagagaatcgttctagtgtttttgccgaagattaccgctggcctactgtgaagggagatgacctgtgattagactgggcggctggggagaaacagttcagtgcattgttgttgttgctgtttttggtgttttgcttttcagtgccaactcagcacattgtatatgattcggtttatacatattaccttgttataatgaaaaaactcattctgagaacactgaaatgttatactcagtgttgatttcttcggtcactacacaacgtaaaatcatttgtttcttttgactcaaattgtattgcttctgttcagatgatctttcattcaatgtgttcctgttgggcgttactagaaactatggaaaactggaaaataactttgaaaaaattggataaagtataggagggttacttggggccagtaaatcagtagactgaacattcaatataataaaagaacatggggattttgtataaccagggataataaaaagaaaaaagaagttaatttttaattgatgtttttgaaacttagtagaacaaatattcagaagtaacttgataagatatgaatgtttctaaagaagtttctaaaggttcggaaaatgctccttgtcacattagtgtgcatcctacaaaaagtgatctcttaatgtaaattaagaatattttcataattggaatatacttttcttaaaaaaaaggaacagttagttctcatctagaatgaaagttccatatatgcattggtgaatatatatgtatacacatacttacatacttatatgggtatctgtatagataatttgtattagagtattatatagcttcttagtagggtctcaagtaagtttcattttttttatctgggctatatacagtcctcaaataaataatgtcttgattttatttcagcaggaataattttatttattttgcctatttataattaaagtatttttctttagtttgaaaatgtgtattaaagttacatttttgagttacaagagtcttataactacttgaatttttagttaaaatgtcttaatgtaggttgtagtcactttagatggaaaattacctcacatctgttttcttcagtattacttaagattgtttatttagtggtagagagttttttttttcagcctagaggcagctattttaccatctggtatttatggtctaatttgtatttaaacatatgcacacatataaaagttgatactgtggcagtaaactattaaaagttttcactgttcaaaaaaaaaaaaaaaaaa

SEQ ID NO: 39 - DNA, 5173 bp, Homo sapiens
aatcggtggccgccagacacccgcggcgaaggcggctcgggctcgggctccggatgtgctaggtgtgggccggcccccacccgaccctgacaagtgaccatggatcctggagccgggtcagagacatctctgactgtcaatgagcaggtcatcgtgatgtcaggtcatgagaccatccgagtgctggaagtcggagtggatgcccaactccctgctgaggaagagagcaaaggactggagggtgtggccgccgagggctcccagagcggagaccctgctgaagccagtcaagctgctggtgaagctgggccagacaacctgggctcctctgcagaggcaactgtgaagtcacccccggggatccctccgagccctgcccctgccattgccaccttcagccaagccccaagccagcctcaggcatcgcagaccctgacgccactggctgtacaagctgccccccagtattgcaggtcaagtggctggtcagcaggggctggccgtgtggacaattcctacagcaactgtggctgccctcccaggactgaccgctgcttctcctacggggggagtgttcaagccacctttagccggtctccaagcagctgctgtgctgaacaccgctcttccggcaccggtacaagctgccgcaccagtacaggcctcctcgacggcccaaccccggccaccagcccagccccagacgctgttccagacccagccgctgctgcagaccacacctgccatcctcccgcagcccactgctgccaccgctgctgcccctacccccaagccagtggacacccccccacagatcaccgtccagcctgcaggcttcgcatttagcccaggaatcatcagtgctgcttccctcgggggacagacccagatcctggggtccctcactacagctccagtcattaccagcgccattcccagcatgccagggatcagcagtcagatcctcaccaatgctcagggacaggttattggaacccttccatgggtagtgaactcagctagtgtggcggccccagcaccagcccaaagcctgcaggtccaggccgtgaccccccagctgttgttgaacgcccagggccaggtgattgcgaccctggctagcagccccctgcctccacctgtggctgtccggaagccaagcacacctgagtcccctgctaagagtgaggtgcagcccatccagcccacaccaaccgtgccccagcctgctgtggtcattgccagcccagctccagccgccaagccatctgcctctgctcctatcccaattacctgctcagagacccccaccgtcagccagttggtgtccaagccacatactccaagtctggatgaggatgggatcaacttagaagagatccgggagtttgccaagaactttaagatccggcggctctcgctgggccttacacagacccaggtgggtcaggctctgactgcaacggaaggtccagcctacagccagtcagccatctgccggttcgagaagctagacatcacacccaagagtgcccagaagctaaagccggtgctggaaaagtggctaaacgaagctgaactgcggaaccaggaaggccagcagaacctgatggagtttgtgggaggcgagccctccaagaaacgcaaacgccgcacctccttcaccccccaggccatagaggctctcaatgcctattttgagaagaacccactgcccacaggccaggagatcactgaaattgctaaggagctcaactacgaccgtgaggtagtgcgggtctggttctgcaatcggcgccagacgctcaagaacaccagcaagctgaacgtctttcagatcccttagggctcagcccctggccctgtgttctagcactttgtccatttcccgtggcatccggctgcagccactgccatgacagcacctgtcattttgccacgtgcagctgtgctcaccccaggtcatcagactccaccgtgtgcatgtgcatcaatgtccctcttttctcccacacatctcacatcatggggaggccagagggggccacacgagagctccaggctctgggctggtcactccgaagaagaggatttgtgacgtcacttagagaagcaccttgctagcatggtttctgaagggtgaattctggtggggaaccagaaactccctgtctttggggcagggctaaagcagctcctaaggaccactggccattagctcttgcttttgatggcattctctttccaccttgtcttctcctttgctcctctgtgttagtgtggcaggtatgacaactcatccagtggaaacacagcctcacactgcccttccgccccccacactttgcctgcaggtgcaccgaaaggacctgggagataaaattcaaaaaagtgtgatgtgctgctcagaaggtcagactccatgtctgccttgacctcaaggtcagaaggttcccaaacccctggggctggaacatgggatctcctcttccacctcttcctggttcctttgcggggaaaattgcactaaaacagaaccttttcttaatccatgttggaaggaagcaacagtgaactctacctgttctggagttctcctgggtctgcagaaggttgggaatttagaaaataaggctgttctttcatattttaatttaatctctgtcaatggccatccctcccacaaaaaaacgtgggttaagagaacttgcagactggatatgcaagcaaacgggcaactctggagaaaaataaggaaaggaatgctgactttctctttctttctcttgtccccacacccattcccaacccaatactggggccttctcaaaaggagcaaattaaacaataaaccagacagcaaggccctgggggaaaggacaacatcctgaaataaatgatggagcccaggaaggtctcttgtggaagttgacttaactctaattttctttgtaactttaagccttggatacgggaggagaaatctcattttgtcgagtctcagaccatgtctgtgtgtaagcaatccccacagtgtcctctgagccaaggacacccccagatcagattgagttttgcttctagacggggtagctatggtaccttgggggttagctctcatccaagctgttaagtgagtttccagcctcactgtggctggaaagcccctaaaattcagtatgtaactccaggaagtcaggagagaactgagatttgcctagatgaccacaggcttgcggtgtagattatccctaaagggccccaagtcacgggggtcaaccacccctgtcttcagtactcttatccttacagaggctggtctctaacagctgcctccagtggacctcccatgatccaccctgagggaaggaccgtcagctggggacacatcaccacctctgtcagtcactggtgcagagccacctcctagcctagcttcctctggtgtcctgtttcctttcccacttactgttggtgcctcccaggccctgcagtgccagcgtggccaccctcttggtagcctggccagtaagaggaggacagttgtgtgctgaattagcacacgcacgtgcagcgcgcacagacgcgcgcacacacacacacatacacgctctgctgcatttggacaaaccatgcctgccagagtgtagcagaggtgaggaagcaggtgggcagcttgcctgacccagcttttcaggagagcgtgtctccaacagagagtctccacactctagttcagggttatcgacctgcctcaatgagatgacagactcatttgggaggggtgttgcaaacaagttttcagtgagaatagttaagttccagagcttgtaaaggattcagtgactgacacttcagtaaattaggccaggcacattggcttatgcctgtaattccaacactttggaaggccgaggtgggcggatcatttgaggtctggagttcgagaccagcctgaccaacatggtgaaaccccgtctctactaaaaatacaaaaattagccaggtgtggtagtgcacatctgtaatcccagctacttgggaggtggaggcaggagaattgcttgaaccctggaggttgcaatgagctgagatcacactacttcactccagcctgggtgacagagcaagactcggtctcaaacaaacaaaaacttatggcgatgcaggttttcatgctcagacgcttgcattcaggtatgctttcttttttgagagagacaaatgggtcacagctggcaccctgggaatagcacataatccagggtgtgtctgtggtggtggacgtgcaggggaacaccatctgtcctgtgtcatgatgggaaaacaatcatgaaccactggtctaaattaggcctggccatgctttctcagcccctccctcatttaaatttgtcttcccaaagctgagctaaaactaaaccatttctcctctgctggaatgatggattggtcattcagaggaacaataccaggggtgggaggtttgcaggctgagttccccaggcatgggggtgcagggtgtccctgaggtttacccaaagcacagctcgctggcctgtgacctctgcccttcctcccacagtgtaagaccccccaggaagcagctggggcctgaacctctcacctaggaggtaggtttattttattttttgttagcatcaggctctgaaggagttggtatacattttgttttgaaaacatcttctggacttacaccagagcttagtgtcgtctttactatggaaagagaggagaatggacagaaatggtttaactgtgtggagttttgtttgttttgttttaaatggaagaaagaccaaaactttcctggtggatcagctagggcctttgaccctgcattaccacggcattttatccaggtgaagtccagggaaagaactcagccaaatggactaaggaacacacgagtttggaatgcgagactctgacatttttgtgttcttggaaatccaattaccttcccatgcccagatttccttcctgcctcttggaccaggctctggcactgaggttctcactgttcccaacacagacaaagcttcctgagggctggaggggcagcaaggggagaggagaatggggaagaagcgcttgatgtagttgtgtggaataaacagtattttttcttttgtaaaaaaaaaaaaaaaaa

SEQ ID NO: 40 - DNA, 3040 bp, Homo sapiens
aaatcctcctcctccgccatcatccgccgcggtgcggagagcaggtggtgctggaagcgcgtgaggccgggagctcgagagagctaacagactagccggctggacatctggaccgctggatccggaggtggcgaccccggcctgacccggaccctaaatccgtccccgccccagagggcggaggcgcgcgctcgattccccccacgcggcggcgccgcctgtttacgtctgcagatctccaggggagcccaccagcctagtcaacatggcctcggaagacattgccaagctggcagagacacttgccaagactcaggtggccgggggacagctgagtttcaaaggcaagagcctcaaactcaacactgcagaagatgctaaagatgtgattaaagagattgaagactttgacagcttggaggctctgcgtctggaaggcaacacagtgggcgtggaagcagccagggtcatcgccaaggccttagagaagaagtcggagttgaagcgctgccactggagtgacatgttcacgggaaggctgcggaccgagatcccaccagccctgatctcactaggggaaggactcatcacagctggggctcagctggtggagctggacttaagcgacaacgcattcgggcccgacggtgtgcaaggcttcgaggccctgctcaagagctcagcctgcttcaccctgcaggaactcaagctcaacaactgtggcatgggcattggcggcggcaagatcctggctgcagctctgaccgaatgtcaccggaaatccagtgcccaaggcaagcctctggccctgaaggtctttgtggctggcagaaaccgtctggagaatgatggcgccactgccttggcagaagcttttagggtcatcgggaccctggaggaggtccacatgccacagaatgggatcaaccaccctggcatcactgccctggcccaggctttcgctgtcaaccccctgctgcgggtcatcaacctgaatgacaacaccttcactgagaagggcgccgtggccatggccgagaccttgaagaccttgcggcaggtggaggtgattaattttggggactgcctggtgcgctccaagggtgcagttgccattgcagatgccatccgcggcggcctgcccaagctaaaggagctgaacttgtcattctgtgaaatcaagagggatgctgccctggctgttgctgaggccatggcagacaaagctgagctggagaagctggacctgaatggcaacaccctgggagaagaaggctgtgaacagcttcaggaggtgctggagggcttcaacatggccaaggtgctggcgtccctcagtgatgacgaggacgaggaggaggaggaggaaggagaagaggaagaagaggaagcagaagaagaggaggaggaagatgaggaagaggaggaagaagaggaggaggaggaggaagaagagcctcagcagcgagggcagggagagaagtcagccacgccctcacggaagattctggaccctaacactggggagccagctcccgtgctgtcctccccacctcctgcagacgtctccaccttcctggcttttccctctccagagaagctgctgcgcctagggcccaagagctccgtgctgatagcccagcagactgacacgtctgaccccgagaaggtggtctctgccttcctaaaggtgtcatctgtgttcaaggacgaagctactgtgaggatggcagtgcaggatgcagtagatgccctgatgcagaaggctttcaactcctcgtccttcaactccaacaccttcctcaccaggctgctcgtgcacatgggtctgctcaagagtgaagacaaggtcaaggccattgccaacctgtacggccccctgatggcgctgaaccacatggtgcagcaggactatttccccaaggcccttgcacccctgctgctggcgttcgtgaccaagcccaacagcgccctggaatcctgctccttcgcccgccacagtctgctgcagacgctgtacaaggtctagactcaaagcctctcccatcccttggcctggaccagtgagctggggagggactcggatgaactgaggcgcagcctacgccattgccttggacaggactctggccacaggcagggcgggtctgtgtcccatgtgtcctgtcagtcccctgagtatgtgtgtgggtgtggcgcatgtgcaggtctgtgcctcctgtcgggatttgggttttaacgtcttctgctggcccagccctgctctgttgtggggagttggcccccaggggaaagggctgtgagctgctccgccattaaactcacctccacctgagggcgctctgctgatctccgcctgggccctgatggccgtccccacccacctgccttccggcccggctccctggcggagccagaacccagggagttgcccgcgtgctgtccttcccctctgtgttgtgattgggttgtttcctgccctgcctggggctgcttctcgtcaccaagccctggtcctgcggcagctgtcacccctaccatccataccactgtgctgaccgctcagcctgaagagcagagaatgccatgggtgggactgtgggggtcggatcgtggggttgttggcagagggcaaccctgggccccacaccgtgtggacaggcagacaccagattgtccaggagcaggagctgctgggactgcgctggccccggacctagtgggccttctcctggctgctgagatgtcgtctgtgactggcctggctggagggggagtgttgacaacccaaagctgttctccagtctggggagggagaggcagggtccccaatgtccgagctgcatctggacgctgctcttaaaggacctcctggggcaggggagcggtagggtctggactgggcagatgctgtatgacctccctgagcacccgtgactgccccatgctttcccctttgtgctctgtgtgtgtctgggctgtgcccgggggcttcacaaataaagtcgtgtggcagcttcaaaaaaaaaaaaaaaaaaaa

SEQ ID NO: 41 - DNA, 1493 bp, Homo sapiens
ggcaaacagcccgcccggcaccaccatgctcgccctggaggctgcacagctcgacgggccacacttcagctgtctgtacccagatggcgtcttctatgacctggacagctgcaagcattccagctaccctgattcagagggggctcctgactccctgtgggactggactgtggccccacctgtcccagccaccccctatgaagccttcgacccggcagcagccgcttttagccacccccaggctgcccagctctgctacgaaccccccacctacagccctgcagggaacctcgaactggcccccagcctggaggccccggggcctggcctccccgcataccccacggagaacttcgctagccagaccctggttcccccggcatatgccccgtaccccagccctgtgctatcagaggaggaagacttaccgttggacagccctgccctggaggtctcggacagcgagtcggatgaggccctcgtggctggccccgaggggaagggatccgaggcagggactcgcaagaagctgcgcctgtaccagttcctgctggggctactgacgcgcggggacatgcgtgagtgcgtgtggtgggtggagccaggcgccggcgtcttccagttctcctccaagcacaaggaactcctggcgcgccgctggggccagcagaaggggaaccgcaagcgcatgacctaccagaagctggcgcgcgccctccgaaactacgccaagaccggcgagatccgcaaggtcaagcgcaagctcacctaccagttcgacagcgcgctgctgcctgcagtccgccgggcctgagcacacccgaggctcccacctgcggagccgctgggggacctcacgtcccagccaggatccccctggaagaaaaagggcgtccccacactctaggtgataggacttacgcatccccaccttttggggtaaggggagtgctgccctgccataatccccaagcccagcccgggcctgtctgggattccccacttgtgcctggggtccctctgggatttctttgtcatgtacagactccctgggatcctcatgttttgggtgacaggacctatggaccactatactcggggaggcagggtagcagttcttccagaatcccaagagcttctctgggattttcttgtgatatctgattccccagtgaggcctgggacgtttttaagatcgctgtgtgtctgtaaaccctgaatctcatctggggtgggggccctgctggcaaccctgagccctgtccaaggttccctcttgtcagatctgagatttcctagttatgtctggggccctctgggagctgttatcatctcagatctcttcgcccatctatggctgtgttgtcacatctgtcccctcatttttgagatcccccaattctctggaactattctgctgcccctttttatgtgtctggagttccccaatcacatctagggctcctccaagaaaaaaaaaaaaaaaaaaaaaa

SEQ ID NO: 42 - DNA, 1599 bp, Homo sapiens
aagatcctggcctgtgcagctcgggtttccgagcttctgcctcaggcatctccgcgatctcctctcccctccaatcctatccgtgatggacgatgcccacgagtcgccctccgacaaaggtggagagacaggggagtcggatgagacggccgctgtgcccggggacccgggggctaccgacaccgatggaatcccagaggaaactgacggagacgcagatgtggacttgaaagaagctgcagcggaggaaggcgagctcgagagtcaggatgtctcagatttaacaacagttgaaagggaagactcatcattacttaatcctgcagccaaaaaactgaaaatagataccaaagaaaagaaagagaaaaagcagaaagtagatgaagatgagattcagaagatgcaaatcctggtttcttctttttctgaggagcagctgaaccgttatgaaatgtatcgccgctcagctttccctaaggcagccatcaaaaggctgatccagtccatcactggcacctctgtgtctcagaatgttgttattgctatgtctggtatttccaaggttttcgtcggggaggtggtagaagaagcactggatgtgtgtgagaagtggggagaaatgccaccactacaacccaaacatatgagggaagccgttagaaggttaaagtcaaaaggacagatccctaactcgaagcacaaaaaaatcatcttcttctagaccaaagtctagaaaggcctatgttactgacggaagaagtattggttccagacttcctataagactgtctgcattggtgctttagtatctcaggcctccaaggattccatgatgattttaatgtctttctcaaaactctgatatttgtcacacctagaaagtatgtagcctgattgatacttgccttgactaaattttgggacctcttggggcattttgaagtatttaactgtcttgaccagttggaagaagatacgtgggccataagcatcttctggacaggggaactgctttcagagagaaaacctttccaagagagttttgttttgttttggtttcgttttgtttgagatagggtcttgctctatcacctaggctggagtgcagcggcatgactgcagccttgaactcctgggcttaagtgaccctcccacctcagtctcctgagtagctaggactacaggcacacactactgtgcccagctaacttatttttattttttatggagatggggtcttgctttgttgcccaggctggtcgtgaactcctggcttcaagcagtcctcctgcctcagcctcctaaagtgccgagggctttaatggtttcacattgaagcctgaagttgctaagacttaggttgtttcttatatctggttttaagtagatgaaacaaccagaaacttttacttgtgatactctaccatgaaggatgcggtaatggcaggaatagcagaataattggtgcttgtaaacatttaagattctcctgtggattttggtgagtgatcattaaactgttttccaacttgcaaaaaaaaaaaaaaaaaaaaaaaaaaaaaaaaaaaaaaaaaaaaaaaaaaaaaaaaaaaaaaaaaa

SEQ ID NO: 43 - DNA, 1719 bp, Homo sapiens
ggcggaagtgacattatcaacgcgcgccaggggttcagtgaggtcgggcaggttcgctgtggcgggcgcctgggccgccggctgtttaacttcgcttccgctggcccatagtgatctttgcagtgacccagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttagattgttgttaaaaaaaaaaaaaaaaaa

SEQ ID NO: 44 - DNA, 4704 bp, Homo sapiens
ggagagggagaaggctctcgggcggagagaggtcctgcccagctgttggcgaggagtttcctgtttcccccgcagcgctgagttgaagttgagtgagtcactcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctggggcaggctgggccatgtccaaagaggctgcccctctcaccaaagaacagaggcagcaggaagctgcccctgaactgatgcttcctggaaaaccaagggggtcactcccctccctgtaagctgtggggataagcagaaacaacagcagcagggagtgggtgacatagagcattctatgcctttgacattgtcataggataagctgtgttagcacttcctcaggaaatgagattgatttttacaatagccaataacatttgcactttattaatgcctgtatataaatatgaatagctatgttttatatatatatatatatatctatatatgtctatagctctatatatatagccataccttgaaaagagacaaggaaaaacatcaaatattcccaggaaattggttttattggagaactccagaaccaagcagagaaggaagggacccatgacagcattagcatttgacaatcacacatgcagtggttctctgactgtaaaacagtgaactttgcatgaggaaagaggctccatgtctcacagccagctatgaccacattgcacttgcttttgcaaaataatcattccctgcctagcacttctcttctggccatggaactaagtacagtggcactgtttgaggaccagtgttcccggggttcctgtgtgcccttatttctcctggacttttcatttaagctccaagccccaaatctggggggctagtttagaaactctccctcaacctagtttagaaactctaccccatctttaataccttgaatgttttgaaccccactttttaccttcatgggttgcagaaaaatcagaacagatgtccccatccatgcgattgccccaccatctactaatgaaaaattgttctttttttcatctttcccctgcacttatgttactattctctgctcccagccttcatccttttctaaaaaggagcaaattctcactctaggctttatcgtgtttactttttcattacacttgacttgattttctagttttctatacaaacaccaatgggttccatctttctgggctcctgattgctcaagcacagtttggcctgatgaagaggatttcaactacacaatactatcattgtcaggactatgacctcaggcactctaaacatatgttttgtttggtcagcacagcgtttcaaaaagtgaagccactttataaatatttggagattttgcaggaaaatctggatccccaggtaaggatagcagatggttttcagttatctccagtccacgttcacaaaatgtgaaggtgtggagacacttacaaagctgcctcacttctcactgtaaacattagctctttccactgcctacctggaccccagtctaggaattaaatctgcacctaaccaaggtcccttgtaagaaatgtccattcaagcagtcattctctgggtatataatatgattttgactaccttatctggtgttaagatttgaagttggccttttattggactaaaggggaactcctttaagggtctcagttagcccaagtttcttttgcttatatgttaatagttttaccctctgcattggagagaggagtgctttactccaagaagctttcctcatggttaccgttctctccatcatgccagccttctcaacctttgcagaaattactagagaggatttgaatgtgggacacaaaggtcccatttgcagttagaaaatttgtgtccacaaggacaagaacaaagtatgagctttaaaactccataggaaacttgttaatcaacaaagaagtgttaatgctgcaagtaatctcttttttaaaactttttgaagctacttattttcagccaaataggaatattagagagggactggtagtgagaatatcagctctgtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggcttgttttgtttatgtttttttttcttattcaagaaaaaagaccaaggaataacattctgtagttcctaaaaatactgacttttttcactactatacataaagggaaagttttattcttttatggaacacttcagctgtactcatgtattaaaataggaatgtgaatgctatatactctttttatatcaaaagtctcaagcacttatttttattctatgcattgtttgtcttttacataaataaaatgtttattagattgaataaagcaaaatactcaggtgagcatcctgcctcctgttcccattcctagtagctaaa

SEQ ID NO: 45 - DNA, 2646 bp, Homo sapiens
gattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatgctacttgacttacgatggtgttacttcctgataaactcgtcgtaagttgaaaatattatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 46 - DNA, 2583 bp, Homo sapiens
gattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 47 - DNA, 2914 bp, Homo sapiens
gatttcagttgaaagatgtgtttttgtgagtagagcaccgcagaagaactgaagactgttgtgtgctccccgcagaaggggctaccatgatcctttcctcctataacaccatccagtcggttttctgttgctgctgttgctgttcagtgcagaagcgacaaatgagaacacagataagcctgagcacagatgaagagcttccagaaaaatacacccagcgtcgcaggccgtggctcagccaattgtcaaataagaagcaatccaacacgggccgtgtgcagccgtcaaaacgaaagccactgcctcccctcccaccctctgaggttgctgaagagaagatccaagtcaaggcactttatgattttctgcccagagaaccctgtaatttagccttaaggagagcagaagaatacctgatactggagaaatacaatcctcactggtggaaggcaagagaccgtttggggaatgaaggcttaatcccaagcaactatgtgactgaaaacaaaataactaatttagaaatatatgagtggtaccatagaaacattaccagaaatcaggcagaacatctattgagacaagagtctaaagaaggtgcatttattgtcagagattcaagacatttaggatcctacacaatttccgtatttatgggagctagaagaagtacggaggctgccataaaacattatcagataaaaaagaatgactcaggacagtggtatgtggctgaaagacacgcctttcaatcaatccctgagttaatctggtatcaccagcacaatgcagccggtctcatgactcgtctccgatatccagttgggctgatgggcagttgtttaccagccacagctgggtttagctacgaaaagtgggagatagatccatctgagttggcttttataaaggagattggaagcggtcagtttggagtggtccatttaggtgaatggcggtcacatatccaggtagctatcaaggccatcaatgaaggctccatgtctgaagaggatttcattgaagaggccaaagtgatgatgaaattatctcattcaaagctagtgcaactttatggagtctgtatacagcggaagcccctttacattgtgacagagttcatggaaaatggctgcctgcttaactatctcagggagaataaaggaaagcttaggaaggaaatgctactgagtgtatgccaggatatatgtgaaggaatggaatatctggagaggaatggctatattcatagggatttggcggcaaggaattgtttggtcagttcaacatgcatagtaaaaatttcagactttggaatgacaaggtacgttttggatgatgagtatgtcagttcttttggagccaagttcccaatcaagtggtcccctcctgaagtttttcttttcaataagtacagcagtaaatctgatgtctggtcatttggagttttaatgtgggaagtttttacagaaggaaaaatgccttttgaaaataagtcaaatttgcaagtcgtggaagctatttctgaaggcttcaggctatatcgccctcacctggcaccaatgtccatatatgaagtcatgtacagctgctggcatgagaaacctgaaggccgccctacatttgccgagctgctgcgggctgtcacagagattgcggaaacctggtgaccggaaacagaatgccaacccaaagagtcatcttgcaaaactgtcatttattgtgaatatcttcaccatatggggtcacttatggtgaatatctttcttcagagttgctgactcttgaaaacagtgcaaagatcacagtttttaaaagttttaaaaatttaagaatattcacacaatcgtttttctatgtgtgagagggatttgcacactcttatttttctgtaaaatatttcacatcccaaatgtgaagaagtgaaaaagacttcgcagcagtcttcattgtggtgctcttcatgatcatagccccaggaacccttgaggttcttcttcacaaggctgagagtgcttccttcttgaagacgagtgacattcatcacttcagtgatccatgcatagaatatgaaaataaattcttccaactcatgggataaaggggactcccttgaagaatttcatgtttttgggctgtatagctctttacagaaaatgcacctttataaatcacatgaatgttagtattctggaaatgtcttttgttaatataatcttcccatgttatttaacaaattgtttttgcacatatctgattatattgaaagcagttttttgcattcgagttttaaacactgttataaaatgtagccaaagctcacctttgaacagatcccggtgacattctatttccaggaaaatccggaacctgattttagttctgtgattttacactttttacatgtgagattggacagtttcagaggccttattttgtcatactaagtgtctcctgtaattttcaggaagatgatttgttctttccagaagaggagacaaaagcaagatagccaaatgtgacatcaagctccattgtttcggaaatccaggattttgaattcgagatgaaacaaccagcaatcacagttaaatcttaactttgcctgcactctttgtaggaatgatcagaaatttatctttatcattctgagtgcttcaggagtacaataggaagaaagatactggagaaagcactaatgtaatcaccatgaagtctgacaacaggagcccattatttgcgtactgtcccaccctgtatcatggttctctgggaacaagctttatgattctcattagagtttatttgttgattgtcagtagttgcgacttttaaattatatttcccccactcaaagaatggtatctttatatatcaatgacattcaataaatgtgtattatttctaatgagaa